SEQUENCE LISTING (861 sequences)

SEQ ID NO: 1 - DNA, 23 bp, artificial sequence
  hTCR alpha Block primer
gtgcatagacctcatgtctagca

SEQ ID NO: 2 - DNA, 23 bp, artificial sequence
  hTCR alpha RT primer
tgttgaaggcgtttgcacatgca

SEQ ID NO: 3 - DNA, 23 bp, artificial sequence
  hTCR beta Block primer
aggcagtatctggagtcattgag

SEQ ID NO: 4 - DNA, 23 bp, artificial sequence
  hTCR beta RT primer
gaactggacttgacagcggaagt

SEQ ID NO: 5 - DNA, 30 bp, artificial sequence
  TS-Oligo
aagcagtggtatcaacgcagagtacatggg

SEQ ID NO: 6 - DNA, 30 bp, artificial sequence; identical to SEQ ID NO: 5
  TS-Primer

SEQ ID NO: 7 - DNA, 64 bp, artificial sequence
  Forward TS-Tag primer v1
gtctcgtgggctcggagatgtgtataagagacagaagcagtggtatcaacgcagagtacatggg

SEQ ID NO: 8 - DNA, 51 bp, artificial sequence
  hTCR alpha Reverse Tag primer
tcgtcggcagcgtcagatgtgtataagagacaggagggtcagggttctgga

SEQ ID NO: 9 - DNA, 53 bp, artificial sequence
  hTCR alpha Reverse Tag primer/hTCA-R-TP5-1
tcgtcggcagcgtcagatgtgtataagagacagacttgtcactggatttagag

SEQ ID NO: 10 - DNA, 52 bp, artificial sequence
  hTCR beta Reverse Tag primer
tcgtcggcagcgtcagatgtgtataagagacaggctcaaacacagcgacctc

SEQ ID NO: 11 - DNA, 21 bp, artificial sequence
  P5-seq
ggcagcgtcagatgtgtataa

SEQ ID NO: 12 - DNA, 23 bp, artificial sequence
  CA3
actttgtgacacatttgtttgag

SEQ ID NO: 13 - DNA, 23 bp, artificial sequence
  CB3
actgtgcacctccttcccattca

SEQ ID NO: 14 - DNA, 444 bp, Homo sapiens
gggctgcaaaacgtttttctgctgtgggtacgtgagcaggaaacatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgcccggaacaccggtaaccagttctattttgggacagggacaagtttgacggtcattccaaata

SEQ ID NO: 15 - protein, 147 aa, Homo sapiens
  misc_feature (11)..(11): Xaa can be any naturally occurring amino acid
  UNSURE (11)..(11)
AAKRFSAVGTXAGNMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPN

SEQ ID NO: 16 - DNA, 512 bp, Homo sapiens
ggtctcagaatgacttccttgagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtcaacagacagggacgataggtggctacaccttcggttcggggaccaggttaaccgttgtagaggacctgaacaaggtgttcccaccc

SEQ ID NO: 17 - protein, 170 aa, Homo sapiens
SQNDFLESPAPLSSMHRYRRPLRHAASAMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSQQTGTIGGYTFGSGTRLTVVEDLNKVFPP

SEQ ID NO: 18 - protein, 13 aa, Homo sapiens
CAHITSGTYKYIF

SEQ ID NO: 19 - protein, 11 aa, Homo sapiens
CADPNFNKFYF

SEQ ID NO: 20 - protein, 8 aa, Homo sapiens
SSGNQFYF

SEQ ID NO: 21 - protein, 13 aa, Homo sapiens
CAVLSYNTDKLIF

SEQ ID NO: 22 - protein, 17 aa, Homo sapiens
CAMREVYGSSNTGKLIF

SEQ ID NO: 23 - protein, 13 aa, Homo sapiens
CAAKEEYGNKLVF

SEQ ID NO: 24 - protein, 18 aa, Homo sapiens
CVVTTSMYSGGGADGLTF

SEQ ID NO: 25 - protein, 11 aa, Homo sapiens
CACDDNNDMRF

SEQ ID NO: 26 - protein, 16 aa, Homo sapiens
CAVRVVVKAAGNKLTF

SEQ ID NO: 27 - protein, 12 aa, Homo sapiens
CAVMDSNYQLIW

SEQ ID NO: 28 - protein, 15 aa, Homo sapiens
CAGYILTGGGNKLTF

SEQ ID NO: 29 - protein, 14 aa, Homo sapiens
CAESMQTGANNLFF

SEQ ID NO: 30 - protein, 12 aa, Homo sapiens
CAAMDSNYQLIW

SEQ ID NO: 31 - protein, 9 aa, Homo sapiens
CVVRTALIF

SEQ ID NO: 32 - protein, 14 aa, Homo sapiens
  misc_feature (3)..(3): Xaa can be any naturally occurring amino acid
  UNSURE (3)..(3)
WGXRSGGGADGLTF

SEQ ID NO: 33 - protein, 11 aa, Homo sapiens
CARNTGNQFYF

SEQ ID NO: 34 - protein, 14 aa, Homo sapiens
CAVQAGGGADGLTF

SEQ ID NO: 35 - protein, 14 aa, Homo sapiens
CAVTPSGGYNKLIF

SEQ ID NO: 36 - protein, 13 aa, Homo sapiens
CAFPGGSARQLTF

SEQ ID NO: 37 - protein, 11 aa, Homo sapiens
CADYYGQNFVF

SEQ ID NO: 38 - protein, 14 aa, Homo sapiens
CAGNDQTGANNLFF

SEQ ID NO: 39 - protein, 13 aa, Homo sapiens
CALTPNYGQNFVF

SEQ ID NO: 40 - protein, 13 aa, Homo sapiens
CALTPNRDDKIIF

SEQ ID NO: 41 - protein, 11 aa, Homo sapiens
CAARGSYIPTF

SEQ ID NO: 42 - protein, 13 aa, Homo sapiens
CATVPGANSKLTF

SEQ ID NO: 43 - protein, 15 aa, Homo sapiens
CAMRELINYGQNFVF

SEQ ID NO: 44 - protein, 13 aa, Homo sapiens
CAASKGGSYIPTF

SEQ ID NO: 45 - protein, 14 aa, Homo sapiens
CAMSLPRGYQKVTF

SEQ ID NO: 46 - protein, 14 aa, Homo sapiens
CAHGGGTSYGKLTF

SEQ ID NO: 47 - protein, 13 aa, Homo sapiens
CAAVNSGNTPLVF

SEQ ID NO: 48 - protein, 16 aa, Homo sapiens
CAYRRYSGGGADGLTF

SEQ ID NO: 49 - protein, 12 aa, Homo sapiens
CAVRRDGQNFVF

SEQ ID NO: 50 - protein, 17 aa, Homo sapiens
  misc_feature (16)..(16): Xaa can be any naturally occurring amino acid
  UNSURE (16)..(16)
CAAPKGDGHGQESTYXF

SEQ ID NO: 51 - protein, 14 aa, Homo sapiens
CAASRKAAGNKLTF

SEQ ID NO: 52 - protein, 14 aa, Homo sapiens
CAVPMNTGRRALTF

SEQ ID NO: 53 - protein, 14 aa, Homo sapiens
CALSETNTGNQFYF

SEQ ID NO: 54 - protein, 13 aa, Homo sapiens
CAVSLSDGQKLLF

SEQ ID NO: 55 - protein, 14 aa, Homo sapiens
CAASALQGAQKLVF

SEQ ID NO: 56 - protein, 14 aa, Homo sapiens
CIVRPGGYGQNFVF

SEQ ID NO: 57 - protein, 16 aa, Homo sapiens
  misc_feature (14)..(15): Xaa can be any naturally occurring amino acid
  UNSURE (14)..(15)
CAASGQEEVLTDSXXF

SEQ ID NO: 58 - protein, 16 aa, Homo sapiens
CAEKGESGAGSYQLTF

SEQ ID NO: 59 - protein, 13 aa, Homo sapiens
CAVLTGGGNKLTF

SEQ ID NO: 60 - protein, 15 aa, Homo sapiens
CALRTSIGFGNVLHC

SEQ ID NO: 61 - protein, 15 aa, Homo sapiens
CAAGKGGTSYGKLTF

SEQ ID NO: 62 - protein, 12 aa, Homo sapiens
CAVRARDDKIIF

SEQ ID NO: 63 - protein, 12 aa, Homo sapiens
CAASRGGQKLLF

SEQ ID NO: 64 - protein, 13 aa, Homo sapiens
CAAGKSDGQKLLF

SEQ ID NO: 65 - protein, 15 aa, Homo sapiens
CAALRAETSGSRLTF

SEQ ID NO: 66 - protein, 10 aa, Homo sapiens
CAAGSYIPTF

SEQ ID NO: 67 - protein, 15 aa, Homo sapiens
CAASRSNFGNEKLTF

SEQ ID NO: 68 - protein, 14 aa, Homo sapiens
CALIRENPYNEQFF

SEQ ID NO: 69 - protein, 16 aa, Homo sapiens
CAISEFGGAYDNEQFF

SEQ ID NO: 70 - protein, 15 aa, Homo sapiens
CASSLAGTSGQTQYF

SEQ ID NO: 71 - protein, 13 aa, Homo sapiens
  misc_feature (11)..(12): Xaa can be any naturally occurring amino acid
  UNSURE (11)..(12)
CSVGRLPTSSXXF

SEQ ID NO: 72 - protein, 11 aa, Homo sapiens
CASSRRDTQYF

SEQ ID NO: 73 - protein, 15 aa, Homo sapiens
CATSRQGATNEKLFF

SEQ ID NO: 74 - protein, 15 aa, Homo sapiens
CSAKDLQENTYEQYF

SEQ ID NO: 75 - protein, 15 aa, Homo sapiens
CASSQGQHDGEPQHF

SEQ ID NO: 76 - protein, 15 aa, Homo sapiens
CASSQQTGTIGGYTF

SEQ ID NO: 77 - protein, 15 aa, Homo sapiens
CASSHSGQGRSEAFF

SEQ ID NO: 78 - protein, 13 aa, Homo sapiens
CASSESGQETQYF

SEQ ID NO: 79 - protein, 12 aa, Homo sapiens
CASSANQETQYF

SEQ ID NO: 80 - protein, 16 aa, Homo sapiens
CASSYSPDRVAGELFF

SEQ ID NO: 81 - protein, 15 aa, Homo sapiens
CASSENQGTYTEAFF

SEQ ID NO: 82 - protein, 15 aa, Homo sapiens
CASRTRDKNTGELFF

SEQ ID NO: 83 - protein, 16 aa, Homo sapiens
CASTLGGGALSYEQYF

SEQ ID NO: 84 - protein, 14 aa, Homo sapiens
CASSQGQLQETQYF

SEQ ID NO: 85 - protein, 13 aa, Homo sapiens
CASSFQGDTEAFF

SEQ ID NO: 86 - protein, 15 aa, Homo sapiens
CASSHRDRNPYGYTF

SEQ ID NO: 87 - protein, 14 aa, Homo sapiens
CASSDYGSTDTQYF

SEQ ID NO: 88 - protein, 14 aa, Homo sapiens
CASSQYRGWNEQFF

SEQ ID NO: 89 - protein, 14 aa, Homo sapiens
CASSDEQGGEKLFF

SEQ ID NO: 90 - protein, 14 aa, Homo sapiens
CASSFPGRRAEAFF

SEQ ID NO: 91 - protein, 13 aa, Homo sapiens
CASSFQGFTEAFF

SEQ ID NO: 92 - protein, 14 aa, Homo sapiens
CASKGQGDTGELFF

SEQ ID NO: 93 - protein, 16 aa, Homo sapiens
CASRQSGQEISYGYTF

SEQ ID NO: 94 - protein, 15 aa, Homo sapiens
CASRSGAGYQETQYF

SEQ ID NO: 95 - protein, 14 aa, Homo sapiens
CSAADGSSYNEQFF

SEQ ID NO: 96 - protein, 12 aa, Homo sapiens
CASETGGDEQYF

SEQ ID NO: 97 - protein, 11 aa, Homo sapiens
CSVSLGREQFF

SEQ ID NO: 98 - protein, 17 aa, Homo sapiens
  misc_feature (16)..(16): Xaa can be any naturally occurring amino acid
  UNSURE (16)..(16)
CASSLCLSHKDGRAVXF

SEQ ID NO: 99 - protein, 15 aa, Homo sapiens
CASRERTGMLHEQYF

SEQ ID NO: 100 - protein, 17 aa, Homo sapiens
CASSPVFLEGEVAEAFF

SEQ ID NO: 101 - protein, 14 aa, Homo sapiens
CASSVTGGAYGYTF

SEQ ID NO: 102 - protein, 17 aa, Homo sapiens
CASSSGTYYSGANVLTF

SEQ ID NO: 103 - protein, 13 aa, Homo sapiens
CASSEGDNYGYTF

SEQ ID NO: 104 - protein, 14 aa, Homo sapiens
CASSAPPLEETQYF

SEQ ID NO: 105 - protein, 13 aa, Homo sapiens
CASSLDFYSPLHF

SEQ ID NO: 106 - protein, 15 aa, Homo sapiens
CASSGWTETNEKLFF

SEQ ID NO: 107 - protein, 16 aa, Homo sapiens
  misc_feature (10)..(11): Xaa can be any naturally occurring amino acid
  UNSURE (10)..(11)
CASSQLDRVXXETQYF

SEQ ID NO: 108 - protein, 20 aa, Homo sapiens
CASSFGPGQGTSRARYEQYF

SEQ ID NO: 109 - protein, 15 aa, Homo sapiens
CASSLGQGSTGELFF

SEQ ID NO: 110 - protein, 13 aa, Homo sapiens
CASQDRYQETQYF

SEQ ID NO: 111 - protein, 14 aa, Homo sapiens
CASSETGGRTEAFF

SEQ ID NO: 112 - protein, 16 aa, Homo sapiens
CASSVAGGSWGNEQFF

SEQ ID NO: 113 - protein, 14 aa, Homo sapiens
CATSRGGSTDTQYF

SEQ ID NO: 114 - protein, 14 aa, Homo sapiens
CASSVQGKSSPLHF

SEQ ID NO: 115 - protein, 19 aa, Homo sapiens
CASRGTADIPAPYNSPLHF

SEQ ID NO: 116 - protein, 13 aa, Homo sapiens
CASRNRENTEAFF

SEQ ID NO: 117 - protein, 12 aa, Homo sapiens
CASNAPQGPQHF

SEQ ID NO: 118 - protein, 8 aa, Homo sapiens
SSGNQFYF

SEQ ID NO: 119 - protein, 11 aa, Homo sapiens
CARNTGNQFYF

SEQ ID NO: 120 - protein, 11 aa, Homo sapiens
CADYYGQNFVF

SEQ ID NO: 121 - protein, 13 aa, Homo sapiens
CAETPTNDYKLSF

SEQ ID NO: 122 - protein, 16 aa, Homo sapiens
  misc_feature (14)..(15): Xaa can be any naturally occurring amino acid
  UNSURE (14)..(15)
CLVGDYSQEPTNTXXF

SEQ ID NO: 123 - protein, 11 aa, Homo sapiens
CAGAVGNDMRF

SEQ ID NO: 124 - protein, 13 aa, Homo sapiens
CAAKEEYGNKLVF

SEQ ID NO: 125 - protein, 18 aa, Homo sapiens
CVVTTSMYSGGGADGLTF

SEQ ID NO: 126 - protein, 11 aa, Homo sapiens
CACDDNNDMRF

SEQ ID NO: 127 - protein, 13 aa, Homo sapiens
CAHITSGTYKYIF

SEQ ID NO: 128 - protein, 13 aa, Homo sapiens
CATDGNYGQNFVF

SEQ ID NO: 129 - protein, 12 aa, Homo sapiens
CVVNWSFNKFYF

SEQ ID NO: 130 - protein, 8 aa, Homo sapiens
SAGNQFYF

SEQ ID NO: 131 - protein, 8 aa, Homo sapiens
SSGNQFYF

SEQ ID NO: 132 - protein, 15 aa, Homo sapiens
CAASRARTNAGKSTF

SEQ ID NO: 133 - protein, 16 aa, Homo sapiens
  misc_feature (14)..(15): Xaa can be any naturally occurring amino acid
  UNSURE (14)..(15)
CAVRDQGPMQANQXXF

SEQ ID NO: 134 - protein, 13 aa, Homo sapiens
CALSYSSASKIIF

SEQ ID NO: 135 - protein, 13 aa, Homo sapiens
CAVLSYNTDKLIF

SEQ ID NO: 136 - protein, 11 aa, Homo sapiens
CARNTGNQFYF

SEQ ID NO: 137 - protein, 12 aa, Homo sapiens
CALMDSNYQLIW

SEQ ID NO: 138 - protein, 13 aa, Homo sapiens
CAVRNSGNTPLVF

SEQ ID NO: 139 - protein, 13 aa, Homo sapiens
CAASNTGRRALTF

SEQ ID NO: 140 - protein, 14 aa, Homo sapiens
CAVRDSNQGGKLIF

SEQ ID NO: 141 - protein, 15 aa, Homo sapiens
CVVSDRGSTLGRLYF

SEQ ID NO: 142 - protein, 12 aa, Homo sapiens
CASMDSNYQLIW

SEQ ID NO: 143 - protein, 17 aa, Homo sapiens
  misc_feature (14)..(15): Xaa can be any naturally occurring amino acid
  UNSURE (14)..(15)
CALSEGYRVLQGNXXTF

SEQ ID NO: 144 - protein, 11 aa, Homo sapiens
CVRNTGNQFYF

SEQ ID NO: 145 - protein, 12 aa, Homo sapiens
CAVRDSNYQLIW

SEQ ID NO: 146 - protein, 12 aa, Homo sapiens
CAVMDSNYQLIW

SEQ ID NO: 147 - protein, 10 aa, Homo sapiens
CALNDYKLSF

SEQ ID NO: 148 - protein, 12 aa, Homo sapiens
CAAMDSNYQLIW

SEQ ID NO: 149 - protein, 13 aa, Homo sapiens
CAAKSGGSYIPTF

SEQ ID NO: 150 - protein, 11 aa, Homo sapiens
CAGATGNQFYF

SEQ ID NO: 151 - protein, 14 aa, Homo sapiens
CAAPVGQGAQKLVF

SEQ ID NO: 152 - protein, 8 aa, Homo sapiens
  misc_feature (7)..(7): Xaa can be any naturally occurring amino acid
  UNSURE (7)..(7)
CRQHAHXF

SEQ ID NO: 153 - protein, 16 aa, Homo sapiens
CALSGAAETSGSRLTF

SEQ ID NO: 154 - protein, 11 aa, Homo sapiens
CAGALGNKLVF

SEQ ID NO: 155 - protein, 13 aa, Homo sapiens
CVVRDNYGQNFVF

SEQ ID NO: 156 - protein, 15 aa, Homo sapiens
CALSGSTDSWGKFQF

SEQ ID NO: 157 - protein, 14 aa, Homo sapiens
  misc_feature (13)..(13): Xaa can be any naturally occurring amino acid
  UNSURE (13)..(13)
CALSEAGWPEAAXF

SEQ ID NO: 158 - protein, 13 aa, Homo sapiens
CATVPGANSKLTF

SEQ ID NO: 159 - protein, 12 aa, Homo sapiens
CAAQWGANNLFF

SEQ ID NO: 160 - protein, 13 aa, Homo sapiens
CALTQAPDDKIIF

SEQ ID NO: 161 - protein, 14 aa, Homo sapiens
  misc_feature (12)..(13): Xaa can be any naturally occurring amino acid
  UNSURE (12)..(13)
CAVPRMQATCSXXF

SEQ ID NO: 162 - protein, 12 aa, Homo sapiens
CAVGLNTDKLIF

SEQ ID NO: 163 - protein, 14 aa, Homo sapiens
CAATNGGSQGNLIF

SEQ ID NO: 164 - protein, 13 aa, Homo sapiens
CAASRSDDYKLSF

SEQ ID NO: 165 - protein, 11 aa, Homo sapiens
CALEIDDKIIF

SEQ ID NO: 166 - protein, 16 aa, Homo sapiens
  misc_feature (13)..(14): Xaa can be any naturally occurring amino acid
  UNSURE (13)..(14)
CAVSERTAVLPRXXIF

SEQ ID NO: 167 - protein, 18 aa, Homo sapiens
CALSEAGYSGAGSYQLTF

SEQ ID NO: 168 - protein, 15 aa, Homo sapiens
CASSQQTGTIGGYTF

SEQ ID NO: 169 - protein, 13 aa, Homo sapiens
CASSFQGFTEAFF

SEQ ID NO: 170 - protein, 16 aa, Homo sapiens
CASSSETELLYYGYTF

SEQ ID NO: 171 - protein, 13 aa, Homo sapiens
  misc_feature (11)..(12): Xaa can be any naturally occurring amino acid
  UNSURE (11)..(12)
CSVGRLPTSSXXF

SEQ ID NO: 172 - protein, 15 aa, Homo sapiens
CASSQGTSHSYEQYF

SEQ ID NO: 173 - protein, 18 aa, Homo sapiens
CASSLRPDGDPSGNTIYF

SEQ ID NO: 174 - protein, 14 aa, Homo sapiens
CASSPTGEDYGYTF

SEQ ID NO: 175 - protein, 11 aa, Homo sapiens
CAWSENTEAFF

SEQ ID NO: 176 - protein, 16 aa, Homo sapiens
CASSQDPGQGSDTQYF

SEQ ID NO: 177 - protein, 16 aa, Homo sapiens
CASSQITGTGYYGYTF

SEQ ID NO: 178 - protein, 14 aa, Homo sapiens
CALIRENPYNEQFF

SEQ ID NO: 179 - protein, 15 aa, Homo sapiens
CATSRQGATNEKLFF

SEQ ID NO: 180 - protein, 19 aa, Homo sapiens
CASSQGAGQGYGSNQPQHF

SEQ ID NO: 181 - protein, 15 aa, Homo sapiens
CASSLEGRVTYEQYF

SEQ ID NO: 182 - protein, 14 aa, Homo sapiens
CAWSGDYNQETQYF

SEQ ID NO: 183 - protein, 16 aa, Homo sapiens
CASSELAEALNNEQFF

SEQ ID NO: 184 - protein, 16 aa, Homo sapiens
CASSVGASGSIDEQFF

SEQ ID NO: 185 - protein, 15 aa, Homo sapiens
CASSRTSGDTGELFF

SEQ ID NO: 186 - protein, 16 aa, Homo sapiens
CASSSSGTVAKNIQYF

SEQ ID NO: 187 - protein, 15 aa, Homo sapiens
CASSQQTGTIGGYTF

SEQ ID NO: 188 - protein, 15 aa, Homo sapiens
CASSLAGTSGQTQYF

SEQ ID NO: 189 - protein, 17 aa, Homo sapiens
CASRFGASGDRLHTQYF

SEQ ID NO: 190 - protein, 15 aa, Homo sapiens
CANSQQTGTIGGYTF

SEQ ID NO: 191 - protein, 14 aa, Homo sapiens
CASSLAVNTGELFF

SEQ ID NO: 192 - protein, 13 aa, Homo sapiens
CASRTGDGTEAFF

SEQ ID NO: 193 - protein, 15 aa, Homo sapiens
CASSELAAVYNEQFF

SEQ ID NO: 194 - protein, 14 aa, Homo sapiens
CASTGAGGNQPQHF

SEQ ID NO: 195 - protein, 14 aa, Homo sapiens
CASSTQGGHQPQHF

SEQ ID NO: 196 - protein, 15 aa, Homo sapiens
CVSSQQTGTIGGYTF

SEQ ID NO: 197 - protein, 20 aa, Homo sapiens
CASSFGPGQGTSRARYEQYF

SEQ ID NO: 198 - protein, 18 aa, Homo sapiens
CSARDEEPRDSNYNEQFF

SEQ ID NO: 199 - protein, 18 aa, Homo sapiens
  misc_feature (8)..(8): Xaa can be any naturally occurring amino acid
  UNSURE (8)..(8)
CASSNPYXSGGLSYEQYF

SEQ ID NO: 200 - protein, 15 aa, Homo sapiens
CASSTGTDNTGELFF

SEQ ID NO: 201 - protein, 14 aa, Homo sapiens
CASSFSSGANVLTF

SEQ ID NO: 202 - protein, 14 aa, Homo sapiens
CASSPGQGIREQFF

SEQ ID NO: 203 - protein, 13 aa, Homo sapiens
CSAPVPPYNEQFF

SEQ ID NO: 204 - protein, 19 aa, Homo sapiens
CASSDTGLAGGGYTDTQYF

SEQ ID NO: 205 - protein, 11 aa, Homo sapiens
CASSLWTQQFF

SEQ ID NO: 206 - protein, 15 aa, Homo sapiens
CASSDAGKENTEAFF

SEQ ID NO: 207 - protein, 13 aa, Homo sapiens
CASSLYRGYEQYF

SEQ ID NO: 208 - protein, 14 aa, Homo sapiens
CASSPTGKQETQYF

SEQ ID NO: 209 - protein, 11 aa, Homo sapiens
CASSETSEQYF

SEQ ID NO: 210 - protein, 14 aa, Homo sapiens
CSVDLDTSSYEQYF

SEQ ID NO: 211 - protein, 16 aa, Homo sapiens
CASRPREGRATDTQYF

SEQ ID NO: 212 - protein, 16 aa, Homo sapiens
CASSDGGGSGADTQYF

SEQ ID NO: 213 - protein, 15 aa, Homo sapiens
CASSHSGQGRSEAFF

SEQ ID NO: 214 - protein, 14 aa, Homo sapiens
CASNRDRGYNEQFF

SEQ ID NO: 215 - protein, 13 aa, Homo sapiens
CASSPGGSYEQYF

SEQ ID NO: 216 - protein, 13 aa, Homo sapiens
CASSFLMNTEAFF

SEQ ID NO: 217 - protein, 15 aa, Homo sapiens
CASSLDRGANTEAFF

SEQ ID NO: 218 - protein, 8 aa, Homo sapiens
SSGNQFYF

SEQ ID NO: 219 - protein, 11 aa, Homo sapiens
CARNTGNQFYF

SEQ ID NO: 220 - protein, 11 aa, Homo sapiens
CADYYGQNFVF

SEQ ID NO: 221 - protein, 13 aa, Homo sapiens
CAETPTNDYKLSF

SEQ ID NO: 222 - protein, 16 aa, Homo sapiens
  misc_feature (14)..(15): Xaa can be any naturally occurring amino acid
  UNSURE (14)..(15)
CLVGDYSQEPTNTXXF

SEQ ID NO: 223 - protein, 8 aa, Homo sapiens
SSGNQFYF

SEQ ID NO: 224 - protein, 11 aa, Homo sapiens
CARNTGNQFYF

SEQ ID NO: 225 - protein, 8 aa, Homo sapiens
SAGNQFYF

SEQ ID NO: 226 - protein, 11 aa, Homo sapiens
CAFITGNQFYF

SEQ ID NO: 227 - protein, 11 aa, Homo sapiens
CVRNTGNQFYF

SEQ ID NO: 228 - protein, 12 aa, Homo sapiens
  misc_feature (11)..(11): Xaa can be any naturally occurring amino acid
  UNSURE (11)..(11)
CARNTGNQFYXW

SEQ ID NO: 229 - protein, 15 aa, Homo sapiens
  misc_feature (13)..(14): Xaa can be any naturally occurring amino acid
  UNSURE (13)..(14)
CAVRDMGTPTSSXXF

SEQ ID NO: 230 - protein, 11 aa, Homo sapiens
CARNTGNQFYL

SEQ ID NO: 231 - protein, 11 aa, Homo sapiens
CAQNTGNQFYF

SEQ ID NO: 232 - protein, 11 aa, Homo sapiens
CARNTGNKFYF

SEQ ID NO: 233 - protein, 10 aa, Homo sapiens
CAVRYQKVTF

SEQ ID NO: 234 - protein, 11 aa, Homo sapiens
CDRNTGNQFYF

SEQ ID NO: 235 - protein, 11 aa, Homo sapiens
CARNTDNQFYF

SEQ ID NO: 236 - protein, 11 aa, Homo sapiens
CARNTSNQFYF

SEQ ID NO: 237 - protein, 11 aa, Homo sapiens
CAWNTGNQFYF

SEQ ID NO: 238 - protein, 11 aa, Homo sapiens
CTRNTGNQFYF

SEQ ID NO: 239 - protein, 15 aa, Homo sapiens
CLVGDYSQEPTNTSW

SEQ ID NO: 240 - protein, 12 aa, Homo sapiens
CAVRDTSARLMF

SEQ ID NO: 241 - protein, 11 aa, Homo sapiens
CARKTGNQFYF

SEQ ID NO: 242 - protein, 10 aa, Homo sapiens
CPSSGNQFYF

SEQ ID NO: 243 - protein, 10 aa, Homo sapiens
CARNTGNQFF

SEQ ID NO: 244 - protein, 10 aa, Homo sapiens
ARNTGNQFYF

SEQ ID NO: 245 - protein, 11 aa, Homo sapiens
  misc_feature (6)..(6): Xaa can be any naturally occurring amino acid
  UNSURE (6)..(6)
CARNRXNQFYF

SEQ ID NO: 246 - protein, 11 aa, Homo sapiens
YARNTGNQFYF

SEQ ID NO: 247 - protein, 11 aa, Homo sapiens
CARNTGNQFCF

SEQ ID NO: 248 - protein, 11 aa, Homo sapiens
RARNTGNQFYF

SEQ ID NO: 249 - protein, 4 aa, Homo sapiens
  misc_feature (2)..(3): Xaa can be any naturally occurring amino acid
  UNSURE (2)..(3)
CXXF

SEQ ID NO: 250 - protein, 11 aa, Homo sapiens
CARNTGNQLYF

SEQ ID NO: 251 - protein, 11 aa, Homo sapiens
  misc_feature (10)..(10): Xaa can be any naturally occurring amino acid
  UNSURE (10)..(10)
CARNTGNQFXF

SEQ ID NO: 252 - protein, 11 aa, Homo sapiens
CARNIGNQFYF

SEQ ID NO: 253 - protein, 6 aa, Homo sapiens
CNQFYF

SEQ ID NO: 254 - protein, 11 aa, Homo sapiens
CARNNGNQFYF

SEQ ID NO: 255 - protein, 11 aa, Homo sapiens
CVDYYGQNFVF

SEQ ID NO: 256 - protein, 11 aa, Homo sapiens
CARNTCNQFYF

SEQ ID NO: 257 - protein, 11 aa, Homo sapiens
CARDTGNQFYF

SEQ ID NO: 258 - protein, 11 aa, Homo sapiens
SARNTGNQFYF

SEQ ID NO: 259 - protein, 11 aa, Homo sapiens
CARSTGNQFYF

SEQ ID NO: 260 - protein, 11 aa, Homo sapiens
CARNAGNQFYF

SEQ ID NO: 261 - protein, 11 aa, Homo sapiens
CARNTGNQYYF

SEQ ID NO: 262 - protein, 13 aa, Homo sapiens
CAEAPTNDYKLSF

SEQ ID NO: 263 - protein, 11 aa, Homo sapiens
WARNTGNQFYF

SEQ ID NO: 264 - protein, 11 aa, Homo sapiens
CSRNTGNQFYF

SEQ ID NO: 265 - protein, 11 aa, Homo sapiens
CARNTGSQFYF

SEQ ID NO: 266 - protein, 11 aa, Homo sapiens
CAGYYGQNFVF

SEQ ID NO: 267 - protein, 15 aa, Homo sapiens
CASSQQTGTIGGYTF

SEQ ID NO: 268 - protein, 13 aa, Homo sapiens
CASSFQGFTEAFF

SEQ ID NO: 269 - protein, 16 aa, Homo sapiens
CASSSETELLYYGYTF

SEQ ID NO: 270 - protein, 15 aa, Homo sapiens
CASSQQTGTIGGYTF

SEQ ID NO: 271 - protein, 15 aa, Homo sapiens
CASSQQTGTIGGYTF

SEQ ID NO: 272 - protein, 13 aa, Homo sapiens
CASSFQGFTEAFF

SEQ ID NO: 273 - protein, 15 aa, Homo sapiens
CANSQQTGTIGGYTF

SEQ ID NO: 274 - protein, 15 aa, Homo sapiens
CVSSQQTGTIGGYTF

SEQ ID NO: 275 - protein, 16 aa, Homo sapiens
CASSSETELLYYGYTF

SEQ ID NO: 276 - protein, 13 aa, Homo sapiens
CANSFQGFTEAFF

SEQ ID NO: 277 - protein, 13 aa, Homo sapiens
RASSFQGFTEAFF

SEQ ID NO: 278 - protein, 14 aa, Homo sapiens
CASSQTGTIGGYTF

SEQ ID NO: 279 - protein, 13 aa, Homo sapiens
CVSSFQGFTEAFF

SEQ ID NO: 280 - protein, 13 aa, Homo sapiens
CTSSFQGFTEAFF

SEQ ID NO: 281 - protein, 15 aa, Homo sapiens
CASSQQTGTIGGHTF

SEQ ID NO: 282 - protein, 15 aa, Homo sapiens
CTSSQQTGTIGGYTF

SEQ ID NO: 283 - protein, 15 aa, Homo sapiens
CASSQQTGAIGGYTF

SEQ ID NO: 284 - protein, 15 aa, Homo sapiens
CASRQQTGTIGGYTF

SEQ ID NO: 285 - protein, 15 aa, Homo sapiens
CASSQQTGMIGGYTF

SEQ ID NO: 286 - protein, 13 aa, Homo sapiens
CASSFQGFTEDFF

SEQ ID NO: 287 - protein, 15 aa, Homo sapiens
CASSQQTGTIGGYTL

SEQ ID NO: 288 - protein, 15 aa, Homo sapiens
CASSQQTGTIGGYAF

SEQ ID NO: 289 - protein, 13 aa, Homo sapiens
CASSFQGFTEAFL

SEQ ID NO: 290 - protein, 13 aa, Homo sapiens
CASSFQGFIEAFF

SEQ ID NO: 291 - protein, 13 aa, Homo sapiens
CASSLQGFTEAFF

SEQ ID NO: 292 - protein, 13 aa, Homo sapiens
CASSFRGFTEAFF

SEQ ID NO: 293 - protein, 16 aa, Homo sapiens
CASSQITGTGYYGYTF

SEQ ID NO: 294 - protein, 15 aa, Homo sapiens
CASSQQTGTMGGYTF

SEQ ID NO: 295 - protein, 13 aa, Homo sapiens
CASSFQGFNEAFF

SEQ ID NO: 296 - protein, 15 aa, Homo sapiens
WASSQQTGTIGGYTF

SEQ ID NO: 297 - protein, 15 aa, Homo sapiens
RASSQQTGTIGGYTF

SEQ ID NO: 298 - protein, 15 aa, Homo sapiens
CASSQQTGTIGGYPF

SEQ ID NO: 299 - protein, 15 aa, Homo sapiens
CASSQQTGTIGGYTI

SEQ ID NO: 300 - protein, 15 aa, Homo sapiens
CASNQQTGTIGGYTF

SEQ ID NO: 301 - protein, 65 aa, Homo sapiens
  misc_feature (28)..(28): Xaa can be any naturally occurring amino acid
  UNSURE (28)..(28)
CASSFQGFTEAFFGQGTRLTVVGLKFYXKEQERTWQVDLGRQEWKAAGRGFPSSLCCATNEKLFF

SEQ ID NO: 302 - protein, 13 aa, Homo sapiens
CASRFQGFTEAFF

SEQ ID NO: 303 - protein, 15 aa, Homo sapiens
CASSQQAGTIGGYTF

SEQ ID NO: 304 - protein, 14 aa, Homo sapiens
ASSQQTGTIGGYTF

SEQ ID NO: 305 - protein, 15 aa, Homo sapiens
CSSSQQTGTIGGYTF

SEQ ID NO: 306 - protein, 15 aa, Homo sapiens
CASSQQTVTIGGYTF

SEQ ID NO: 307 - protein, 15 aa, Homo sapiens
CASSQQTETIGGYTF

SEQ ID NO: 308 - protein, 13 aa, Homo sapiens
CASSWDGLYGYTF

SEQ ID NO: 309 - protein, 13 aa, Homo sapiens
CASSFQGLTEAFF

SEQ ID NO: 310 - protein, 13 aa, Homo sapiens
CASSFQEFTEAFF

SEQ ID NO: 311 - protein, 15 aa, Homo sapiens
CASSQQTGTIGGYSF

SEQ ID NO: 312 - protein, 15 aa, Homo sapiens
CASSQQTGTIGDYTF

SEQ ID NO: 313 - protein, 16 aa, Homo sapiens
CASTESTGVSTGELFF

SEQ ID NO: 314 - protein, 15 aa, Homo sapiens
CASSKQTGTIGGYTF

SEQ ID NO: 315 - protein, 15 aa, Homo sapiens
CASSQQTRTIGGYTF

SEQ ID NO: 316 - protein, 15 aa, Homo sapiens
CASSQQTGTIGGYIF

SEQ ID NO: 317 - protein, 13 aa, Homo sapiens
CAAKEEYGNKLVF

SEQ ID NO: 318 - protein, 11 aa, Homo sapiens
CAGAVGNDMRF

SEQ ID NO: 319 - protein, 11 aa, Homo sapiens
CACDDNNDMRF

SEQ ID NO: 320 - protein, 18 aa, Homo sapiens
CVVTTSMYSGGGADGLTF

SEQ ID NO: 321 - protein, 12 aa, Homo sapiens
CAVRDSNYQLIW

SEQ ID NO: 322 - protein, 15 aa, Homo sapiens
CALSNYGGSQGNLIF

SEQ ID NO: 323 - protein, 11 aa, Homo sapiens
CAGATGNQFYF

SEQ ID NO: 324 - protein, 12 aa, Homo sapiens
CAVMDSNYQLIW

SEQ ID NO: 325 - protein, 11 aa, Homo sapiens
CAGGTGNQFYF

SEQ ID NO: 326 - protein, 13 aa, Homo sapiens
CATDGNYGQNFVF

SEQ ID NO: 327 - protein, 12 aa, Homo sapiens
CVVNWSFNKFYF

SEQ ID NO: 328 - protein, 15 aa, Homo sapiens
CALSEAGTSYGKLTF

SEQ ID NO: 329 - protein, 16 aa, Homo sapiens
CAEKGESGAGSYQLTF

SEQ ID NO: 330 - protein, 17 aa, Homo sapiens
CALSELTGGTSYGKLTF

SEQ ID NO: 331 - protein, 16 aa, Homo sapiens
  misc_feature (13)..(14): Xaa can be any naturally occurring amino acid
  UNSURE (13)..(14)
CAVSERTAVLPRXXIF

SEQ ID NO: 332 - protein, 12 aa, Homo sapiens
CASMDSNYQLIW

SEQ ID NO: 333 - protein, 15 aa, Homo sapiens
CAASRARTNAGKSTF

SEQ ID NO: 334 - protein, 16 aa, Homo sapiens
CAASIRGNSGNTPLVF

SEQ ID NO: 335 - protein, 13 aa, Homo sapiens
CAAGNSGNTPLVF

SEQ ID NO: 336 - protein, 11 aa, Homo sapiens
CAASNNNDMRF

SEQ ID NO: 337 - protein, 17 aa, Homo sapiens
CAAPKSISGGSNYKLTF

SEQ ID NO: 338 - protein, 16 aa, Homo sapiens
  misc_feature (11)..(11): Xaa can be any naturally occurring amino acid
  UNSURE (11)..(11)
CAVTLRNGHRXNQFYF

SEQ ID NO: 339 - protein, 13 aa, Homo sapiens
CVTFSGGYNKLIF

SEQ ID NO: 340 - protein, 14 aa, Homo sapiens
CAVRDEGSNYQLIW

SEQ ID NO: 341 - protein, 12 aa, Homo sapiens
CAVTDSNYQLIW

SEQ ID NO: 342 - protein, 15 aa, Homo sapiens
CADPWGHNAGNMLTF

SEQ ID NO: 343 - protein, 9 aa, Homo sapiens
SEGNNRLAF

SEQ ID NO: 344 - protein, 11 aa, Homo sapiens
CAGAVGNKLVF

SEQ ID NO: 345 - protein, 12 aa, Homo sapiens
CAVIDSNYQLIW

SEQ ID NO: 346 - protein, 16 aa, Homo sapiens
  misc_feature (11)..(11): Xaa can be any naturally occurring amino acid
  UNSURE (11)..(11)
CILRDVPLWKXEKLTF

SEQ ID NO: 347 - protein, 13 aa, Homo sapiens
CAVSTQGAQKLVF

SEQ ID NO: 348 - protein, 13 aa, Homo sapiens
CAWVGDSSYKLIF

SEQ ID NO: 349 - protein, 14 aa, Homo sapiens
CAMSASGGSYIPTF

SEQ ID NO: 350 - protein, 12 aa, Homo sapiens
CAVGLNTDKLIF

SEQ ID NO: 351 - protein, 8 aa, Homo sapiens
SSGNQFYF

SEQ ID NO: 352 - protein, 13 aa, Homo sapiens
CAHITSGTYKYIF

SEQ ID NO: 353 - protein, 14 aa, Homo sapiens
  misc_feature (13)..(13): Xaa can be any naturally occurring amino acid
  UNSURE (13)..(13)
CARVWRKPRKSHXF

SEQ ID NO: 354 - protein, 11 aa, Homo sapiens
CAVNVFNKFYF

SEQ ID NO: 355 - protein, 11 aa, Homo sapiens
CAVRNDYKLSF

SEQ ID NO: 356 - protein, 19 aa, Homo sapiens
  misc_feature (17)..(18): Xaa can be any naturally occurring amino acid
  UNSURE (17)..(18)
CALSDHGDSREEETNSXXF

SEQ ID NO: 357 - protein, 16 aa, Homo sapiens
CAVRDILGGATNKLIF

SEQ ID NO: 358 - protein, 18 aa, Homo sapiens
  misc_feature (15)..(16): Xaa can be any naturally occurring amino acid
  UNSURE (15)..(16)
CAMGLMLVVLAMESXXTF

SEQ ID NO: 359 - protein, 13 aa, Homo sapiens
CAVRVDSSYKLIF

SEQ ID NO: 360 - protein, 12 aa, Homo sapiens
CALSEAADGLTF

SEQ ID NO: 361 - protein, 15 aa, Homo sapiens
CAGYILTGGGNKLTF

SEQ ID NO: 362 - protein, 14 aa, Homo sapiens
CAVNRGSTLGRLYF

SEQ ID NO: 363 - protein, 13 aa, Homo sapiens
CATVPGANSKLTF

SEQ ID NO: 364 - protein, 16 aa, Homo sapiens
  misc_feature (14)..(15): Xaa can be any naturally occurring amino acid
  UNSURE (14)..(15)
CAVRDRAQDTAPSXXF

SEQ ID NO: 365 - protein, 11 aa, Homo sapiens
CARPLGQNFVF

SEQ ID NO: 366 - protein, 13 aa, Homo sapiens
CAVRSDSNYQLIW

SEQ ID NO: 367 - protein, 16 aa, Homo sapiens
CASSQDPGQGSDTQYF

SEQ ID NO: 368 - protein, 18 aa, Homo sapiens
CASSLRPDGDPSGNTIYF

SEQ ID NO: 369 - protein, 14 aa, Homo sapiens
CASSPTGEDYGYTF

SEQ ID NO: 370 - protein, 15 aa, Homo sapiens
CASSQGTSHSYEQYF

SEQ ID NO: 371 - protein, 11 aa, Homo sapiens
CAWSENTEAFF

SEQ ID NO: 372 - protein, 15 aa, Homo sapiens
CATSRQGATNEKLFF

SEQ ID NO: 373 - protein, 14 aa, Homo sapiens
CALIRENPYNEQFF

SEQ ID NO: 374 - protein, 14 aa, Homo sapiens
CASRGGRVRETQYF

SEQ ID NO: 375 - protein, 16 aa, Homo sapiens
CASSLMGSSYNSPLHF

SEQ ID NO: 376 - protein, 15 aa, Homo sapiens
CASSEGLAGADTQYF

SEQ ID NO: 377 - protein, 16 aa, Homo sapiens
CAISEVEGSGANVLTF

SEQ ID NO: 378 - protein, 15 aa, Homo sapiens
CASSPPSGGPNEQFF

SEQ ID NO: 379 - protein, 15 aa, Homo sapiens
CASSHSGQGRSEAFF

SEQ ID NO: 380 - protein, 16 aa, Homo sapiens
CASSEIGGLHNEEQFF

SEQ ID NO: 381 - protein, 14 aa, Homo sapiens
CSAKTQGDTGELFF

SEQ ID NO: 382 - protein, 13 aa, Homo sapiens
CASSLSTVGELFF

SEQ ID NO: 383 - protein, 14 aa, Homo sapiens
CASSPTGKQETQYF

SEQ ID NO: 384 - protein, 14 aa, Homo sapiens
CASSAPLSQETQYF

SEQ ID NO: 385 - protein, 13 aa, Homo sapiens
  misc_feature (11)..(12): Xaa can be any naturally occurring amino acid
  UNSURE (11)..(12)
CSVGRLPTSSXXF

SEQ ID NO: 386 - protein, 13 aa, Homo sapiens
CASSLFRGYEQYF

SEQ ID NO: 387 - protein, 14 aa, Homo sapiens
  misc_feature (10)..(10): Xaa can be any naturally occurring amino acid
  UNSURE (10)..(10)
CASSLDRGHXEAFF

SEQ ID NO: 388 - protein, 16 aa, Homo sapiens
CASSLSGRSSYNEQFF

SEQ ID NO: 389 - protein, 15 aa, Homo sapiens
CASSEKASGADEQFF

SEQ ID NO: 390 - protein, 15 aa, Homo sapiens
CASSLTSGSGAEQFF

SEQ ID NO: 391 - protein, 16 aa, Homo sapiens
CASSQEFSDRGPEAFF

SEQ ID NO: 392 - protein, 16 aa, Homo sapiens
CASSSSGTVAKNIQYF

SEQ ID NO: 393 - protein, 14 aa, Homo sapiens
CASSVTGGAYGYTF

SEQ ID NO: 394 - protein, 12 aa, Homo sapiens
CSVETDGYEQYF

SEQ ID NO: 395 - protein, 15 aa, Homo sapiens
CASSPIAGGADTQYF

SEQ ID NO: 396 - protein, 16 aa, Homo sapiens
CASFGLAGHSYNEQFF

SEQ ID NO: 397 - protein, 15 aa, Homo sapiens
CASSPFYQGDDEQYF

SEQ ID NO: 398 - protein, 13 aa, Homo sapiens
CASSPNRGGGYTF

SEQ ID NO: 399 - protein, 18 aa, Homo sapiens
CASSQESVSSFSNQPQHF

SEQ ID NO: 400 - protein, 13 aa, Homo sapiens
CASRTGDTGELFF

SEQ ID NO: 401 - protein, 14 aa, Homo sapiens
CAWSGDYNQETQYF

SEQ ID NO: 402 - protein, 11 aa, Homo sapiens
CAWSGNTEAFF

SEQ ID NO: 403 - protein, 14 aa, Homo sapiens
CASSAWDRGAEAFF

SEQ ID NO: 404 - protein, 15 aa, Homo sapiens
CASSPGPGTSYEQYF

SEQ ID NO: 405 - protein, 17 aa, Homo sapiens
CASSFRLAGSTYNEQFF

SEQ ID NO: 406 - protein, 16 aa, Homo sapiens
CASSQAYGTGASEAFF

SEQ ID NO: 407 - protein, 15 aa, Homo sapiens
CASSFGTGNTGELFF

SEQ ID NO: 408 - protein, 17 aa, Homo sapiens
CASSPRPGQGEDNEQFF

SEQ ID NO: 409 - protein, 12 aa, Homo sapiens
CASSTGNTEAFF

SEQ ID NO: 410 - protein, 11 aa, Homo sapiens
CASSQGTEAFF

SEQ ID NO: 411 - protein, 15 aa, Homo sapiens
CASSLRADGYNEQFF

SEQ ID NO: 412 - protein, 13 aa, Homo sapiens
CASSPNTGGEQYF

SEQ ID NO: 413 - protein, 14 aa, Homo sapiens
CASSLRGRNYEQYF

SEQ ID NO: 414 - protein, 14 aa, Homo sapiens
CASSPTGGVQPQHF

SEQ ID NO: 415 - protein, 11 aa, Homo sapiens
CSVEVGRELFF

SEQ ID NO: 416 - protein, 17 aa, Homo sapiens
CASSTRGAGRTYNEQFF

SEQ ID NO: 417 - protein, 11 aa, Homo sapiens
CARNTGNQFYF

SEQ ID NO: 418 - protein, 12 aa, Homo sapiens
CAISGNTDKLIF

SEQ ID NO: 419 - protein, 12 aa, Homo sapiens
CAISGNTDKLIF

SEQ ID NO: 420 - protein, 12 aa, Homo sapiens
CAISGNTDKLIF

SEQ ID NO: 421 - protein, 12 aa, Homo sapiens
CAISGNTDKLIF

SEQ ID NO: 422 - protein, 12 aa, Homo sapiens
CAISGNTDKLIF

SEQ ID NO: 423 - protein, 11 aa, Homo sapiens
CARNTGNQFYF

SEQ ID NO: 424 - protein, 12 aa, Homo sapiens
CAISGNTDKLIF

SEQ ID NO: 425 - protein, 11 aa, Homo sapiens
CARNTGNQFYF

SEQ ID NO: 426 - protein, 11 aa, Homo sapiens
CARNTGNQFYF

SEQ ID NO: 427 - protein, 12 aa, Homo sapiens
CAISGNTDKLIF

SEQ ID NO: 428 - protein, 11 aa, Homo sapiens
CARNTGNQFYF

SEQ ID NO: 429 - protein, 11 aa, Homo sapiens
CARNTGNQFYF

SEQ ID NO: 430 - protein, 12 aa, Homo sapiens
CAISGNTDKLIF

SEQ ID NO: 431 - protein, 12 aa, Homo sapiens
CAISGNTDKLIF

SEQ ID NO: 432 - protein, 12 aa, Homo sapiens
CAISGNTDKLIF

SEQ ID NO: 433 - protein, 12 aa, Homo sapiens
CAISGNTDKLIF

SEQ ID NO: 434 - protein, 11 aa, Homo sapiens
CARNTGNQFYF

SEQ ID NO: 435 - protein, 12 aa, Homo sapiens
CAISGNTDKLIF

SEQ ID NO: 436 - protein, 11 aa, Homo sapiens
CARNTGNQFYF

SEQ ID NO: 437 - protein, 12 aa, Homo sapiens
CAISGNTDKLIF

SEQ ID NO: 438 - protein, 12 aa, Homo sapiens
CAISGNTDKLIF

SEQ ID NO: 439 - protein, 12 aa, Homo sapiens
CAISGNTDKLIF

SEQ ID NO: 440 - protein, 12 aa, Homo sapiens
CAISGNTDKLIF

SEQ ID NO: 441 - protein, 11 aa, Homo sapiens
CARNTGNQFYF

SEQ ID NO: 442 - protein, 12 aa, Homo sapiens
CAISGNTDKLIF

SEQ ID NO: 443 - protein, 15 aa, Homo sapiens
CASSYQTGASYGYTF

SEQ ID NO: 444 - protein, 15 aa, Homo sapiens
CASSYQTGASYGYTF

SEQ ID NO: 445 - protein, 20 aa, Homo sapiens
CSARREYGTVAGEGYNEQFF

SEQ ID NO: 446 - protein, 20 aa, Homo sapiens
CSARREYGTVAGEGYNEQFF

SEQ ID NO: 447 - protein, 20 aa, Homo sapiens
CSARREYGTVAGEGYNEQFF

SEQ ID NO: 448 - protein, 20 aa, Homo sapiens
CSARREYGTVAGEGYNEQFF

SEQ ID NO: 449 - protein, 20 aa, Homo sapiens
CSARREYGTVAGEGYNEQFF

SEQ ID NO: 450 - protein, 20 aa, Homo sapiens
CSARREYGTVAGEGYNEQFF

SEQ ID NO: 451 - protein, 20 aa, Homo sapiens
CSARREYGTVAGEGYNEQFF

SEQ ID NO: 452 - protein, 15 aa, Homo sapiens
CASSYQTGASYGYTF

SEQ ID NO: 453 - protein, 20 aa, Homo sapiens
CSARREYGTVAGEGYNEQFF

SEQ ID NO: 454 - protein, 20 aa, Homo sapiens
CSARREYGTVAGEGYNEQFF

SEQ ID NO: 455 - protein, 20 aa, Homo sapiens
CSARREYGTVAGEGYNEQFF

SEQ ID NO: 456 - protein, 20 aa, Homo sapiens
CSARREYGTVAGEGYNEQFF

SEQ ID NO: 457 - protein, 20 aa, Homo sapiens
CSARREYGTVAGEGYNEQFF

SEQ ID NO: 458 - protein, 15 aa, Homo sapiens
CASSYQTGASYGYTF

SEQ ID NO: 459 - protein, 20 aa, Homo sapiens
CSARREYGTVAGEGYNEQFF

SEQ ID NO: 460 - protein, 15 aa, Homo sapiens
CASSYQTGASYGYTF

SEQ ID NO: 461 - protein, 15 aa, Homo sapiens
CASSYQTGASYGYTF

SEQ ID NO: 462 - protein, 15 aa, Homo sapiens
CASSYQTGASYGYTF

SEQ ID NO: 463 - protein, 20 aa, Homo sapiens
CSARREYGTVAGEGYNEQFF

SEQ ID NO: 464 - protein, 20 aa, Homo sapiens
CSARREYGTVAGEGYNEQFF

SEQ ID NO: 465 - protein, 20 aa, Homo sapiens
CSARREYGTVAGEGYNEQFF

SEQ ID NO: 466 - protein, 20 aa, Homo sapiens
CSARREYGTVAGEGYNEQFF

SEQ ID NO: 467 - protein, 15 aa, Homo sapiens
CASSYQTGASYGYTF

SEQ ID NO: 468 - protein, 20 aa, Homo sapiens
CSARREYGTVAGEGYNEQFF

SEQ ID NO: 469 - protein, 20 aa, Homo sapiens
CSARREYGTVAGEGYNEQFF

SEQ ID NO: 470 - protein, 20 aa, Homo sapiens
CSARREYGTVAGEGYNEQFF

SEQ ID NO: 471 - protein, 20 aa, Homo sapiens
CSARREYGTVAGEGYNEQFF

SEQ ID NO: 472 - protein, 20 aa, Homo sapiens
CSARREYGTVAGEGYNEQFF

SEQ ID NO: 473 - protein, 20 aa, Homo sapiens
CSARREYGTVAGEGYNEQFF

SEQ ID NO: 474 - protein, 20 aa, Homo sapiens
CSARREYGTVAGEGYNEQFF

SEQ ID NO: 475 - protein, 20 aa, Homo sapiens
CSARREYGTVAGEGYNEQFF

SEQ ID NO: 476 - protein, 20 aa, Homo sapiens
CSARREYGTVAGEGYNEQFF

SEQ ID NO: 477 - protein, 20 aa, Homo sapiens
CSARREYGTVAGEGYNEQFF

SEQ ID NO: 478 - protein, 15 aa, Homo sapiens
CASSYQTGASYGYTF

SEQ ID NO: 479 - protein, 15 aa, Homo sapiens
CASSYQTGASYGYTF

SEQ ID NO: 480 - protein, 20 aa, Homo sapiens
CSARREYGTVAGEGYNEQFF

SEQ ID NO: 481 - protein, 20 aa, Homo sapiens
CSARREYGTVAGEGYNEQFF

SEQ ID NO: 482 - protein, 15 aa, Homo sapiens
CASSYQTGASYGYTF

SEQ ID NO: 483 - protein, 15 aa, Homo sapiens
CASSYQTGASYGYTF

SEQ ID NO: 484 - protein, 15 aa, Homo sapiens
CASSEVAGVPGELFF

SEQ ID NO: 485 - protein, 20 aa, Homo sapiens
CSARREYGTVAGEGYNEQFF

SEQ ID NO: 486 - protein, 11 aa, Homo sapiens
CARNTGNQFYF

SEQ ID NO: 487 - protein, 12 aa, Homo sapiens
CAISGNTDKLIF

SEQ ID NO: 488 - protein, 12 aa, Homo sapiens
CAISGNTDKLIF

SEQ ID NO: 489 - protein, 11 aa, Homo sapiens
CARNTGNQFYF

SEQ ID NO: 490 - protein, 11 aa, Homo sapiens
CARNTGNQFYF

SEQ ID NO: 491 - protein, 12 aa, Homo sapiens
CAISGNTDKLIF

SEQ ID NO: 492 - protein, 12 aa, Homo sapiens
CAISGNTDKLIF

SEQ ID NO: 493 - protein, 12 aa, Homo sapiens
CAISGNTDKLIF

SEQ ID NO: 494 - protein, 11 aa, Homo sapiens
CARNTGNQFYF

SEQ ID NO: 495 - protein, 11 aa, Homo sapiens
CARNTGNQFYF

SEQ ID NO: 496 - protein, 12 aa, Homo sapiens
CAISGNTDKLIF

SEQ ID NO: 497 - protein, 12 aa, Homo sapiens
CAISGNTDKLIF

SEQ ID NO: 498 - protein, 11 aa, Homo sapiens
CARNTGNQFYF

SEQ ID NO: 499 - protein, 12 aa, Homo sapiens
CAISGNTDKLIF

SEQ ID NO: 500 - protein, 12 aa, Homo sapiens
CAISGNTDKLIF

SEQ ID NO: 501 - protein, 12 aa, Homo sapiens
CAISGNTDKLIF

SEQ ID NO: 502 - protein, 11 aa, Homo sapiens
CARNTGNQFYF

SEQ ID NO: 503 - protein, 11 aa, Homo sapiens
CARNTGNQFYF

SEQ ID NO: 504 - protein, 12 aa, Homo sapiens
CAISGNTDKLIF

SEQ ID NO: 505 - protein, 12 aa, Homo sapiens
CAISGNTDKLIF

SEQ ID NO: 506 - protein, 16 aa, Homo sapiens
  misc_feature (13)..(14): Xaa can be any naturally occurring amino acid
  UNSURE (13)..(14)
CAILRPTRQELLXXIF

SEQ ID NO: 507 - protein, 12 aa, Homo sapiens
CAISGNTDKLIF

SEQ ID NO: 508 - protein, 12 aa, Homo sapiens
CAISGNTDKLIF

SEQ ID NO: 509 - protein, 12 aa, Homo sapiens
CAISGNTDKLIF

SEQ ID NO: 510 - protein, 25 aa, Homo sapiens
  misc_feature (18)..(18): Xaa can be any naturally occurring amino acid
  UNSURE (18)..(18)
CNSQKQWYQRRVHGASGXSNQPQHF

SEQ ID NO: 511 - protein, 15 aa, Homo sapiens
CASSYQTGASYGYTF

SEQ ID NO: 512 - protein, 20 aa, Homo sapiens
CSARREYGTVAGEGYNEQFF

SEQ ID NO: 513 - protein, 20 aa, Homo sapiens
CSARREYGTVAGEGYNEQFF

SEQ ID NO: 514 - protein, 15 aa, Homo sapiens
CVSSYQTGASYGYTL

SEQ ID NO: 515 - protein, 20 aa, Homo sapiens
CSARREYGTVAGEGYNEQFF

SEQ ID NO: 516 - protein, 15 aa, Homo sapiens
  misc_feature (13)..(14): Xaa can be any naturally occurring amino acid
  UNSURE (13)..(14)
CASSYQTGASRYXXF

SEQ ID NO: 517 - protein, 15 aa, Homo sapiens
CASSYQTGASYGYTF

SEQ ID NO: 518 - protein, 20 aa, Homo sapiens
CSARREYGTVAGEGYNEQFF

SEQ ID NO: 519 - protein, 22 aa, Homo sapiens
  misc_feature (20)..(21): Xaa can be any naturally occurring amino acid
  UNSURE (20)..(21)
CGQLDVNTGPYRERDTMSSXXF

SEQ ID NO: 520 - protein, 20 aa, Homo sapiens
CSARREYGTVAGEGYNEQFF

SEQ ID NO: 521 - protein, 20 aa, Homo sapiens
CSARREWGTVGGQGTNEQYF

SEQ ID NO: 522 - protein, 20 aa, Homo sapiens
RAARREYGGLPGEGTNEQFF

SEQ ID NO: 523 - protein, 20 aa, Homo sapiens
CSARREYGTVAGEGYNEQFF

SEQ ID NO: 524 - protein, 21 aa, Homo sapiens
  misc_feature (19)..(20): Xaa can be any naturally occurring amino acid
  UNSURE (19)..(20)
CSARREYGTVAGEGYNEQXXF

SEQ ID NO: 525 - protein, 20 aa, Homo sapiens
RAAPVFVLGLQAVSTDTQYF

SEQ ID NO: 526 - protein, 20 aa, Homo sapiens
RAAPVFVLGLQAVSTDTQYF

SEQ ID NO: 527 - protein, 20 aa, Homo sapiens
CSARREYGTVAGEGYNEQFF

SEQ ID NO: 528 - protein, 20 aa, Homo sapiens
CSARREYGTVAGEGYNEQFF

SEQ ID NO: 529 - protein, 20 aa, Homo sapiens
CSARREYGTVAGEGYNEQFF

SEQ ID NO: 530 - protein, 16 aa, Homo sapiens
  misc_feature (11)..(12): Xaa can be any naturally occurring amino acid
  UNSURE (11)..(12)
CASSYQTGARXXKYTF

SEQ ID NO: 531 - protein, 15 aa, Homo sapiens
CASSYQTGASYGYTF

SEQ ID NO: 532 - protein, 15 aa, Homo sapiens
CASSYQTGASYGYTF

SEQ ID NO: 533 - protein, 20 aa, Homo sapiens
CSARREYGTVAGEGYNEQFF

SEQ ID NO: 534 - protein, 20 aa, Homo sapiens
CSARREYGTVAGEGYNEQFF

SEQ ID NO: 535 - protein, 15 aa, Homo sapiens
CASSYQTGASYGYTF

SEQ ID NO: 536 - protein, 20 aa, Homo sapiens
CSARREYGTVAGEGYNEQFF

SEQ ID NO: 537 - protein, 15 aa, Homo sapiens
WASSYQTGASYGYTF

SEQ ID NO: 538 - protein, 20 aa, Homo sapiens
CSARREYGTVAGEGYNEQFF

SEQ ID NO: 539 - protein, 15 aa, Homo sapiens
CASSYQTGASYGYTF

SEQ ID NO: 540 - protein, 20 aa, Homo sapiens
RAARGEVGGLQGESTNKQYF

SEQ ID NO: 541 - protein, 14 aa, Homo sapiens
CRPFPGGSYNEQFF

SEQ ID NO: 542 - protein, 28 aa, Homo sapiens
  misc_feature (17)..(18): Xaa can be any naturally occurring amino acid
  UNSURE (17)..(18)
CIRDRSSGINAEYMGVXXIYNQGGKLIF

SEQ ID NO: 543 - protein, 14 aa, Homo sapiens
CALGGTSGTYKYIF

SEQ ID NO: 544 - protein, 14 aa, Homo sapiens
CAGTDGGSQGNLIF

SEQ ID NO: 545 - protein, 12 aa, Homo sapiens
CAGGGSQGNLIF

SEQ ID NO: 546 - protein, 12 aa, Homo sapiens
CAVDGSQGNLIF

SEQ ID NO: 547 - protein, 13 aa, Homo sapiens
CAGASGNTGKLIF

SEQ ID NO: 548 - protein, 14 aa, Homo sapiens
CAGAGGGSQGNLIF

SEQ ID NO: 549 - protein, 15 aa, Homo sapiens
CAETRGGGSQGNLIF

SEQ ID NO: 550 - protein, 12 aa, Homo sapiens
CAVDGSQGNLIF

SEQ ID NO: 551 - protein, 15 aa, Homo sapiens
CAETRGGGSQGNLIF

SEQ ID NO: 552 - protein, 12 aa, Homo sapiens
CAGGGSQGNLIF

SEQ ID NO: 553 - protein, 15 aa, Homo sapiens
CAETRGGGSQGNLIF

SEQ ID NO: 554 - protein, 14 aa, Homo sapiens
CAGNYGGSQGNLIF

SEQ ID NO: 555 - protein, 15 aa, Homo sapiens
CAETRGGGSQGNLIF

SEQ ID NO: 556 - protein, 13 aa, Homo sapiens
CMVSIGNAGKSTF

SEQ ID NO: 557 - protein, 14 aa, Homo sapiens
CAGNYGGSQGNLIF

SEQ ID NO: 558 - protein, 15 aa, Homo sapiens
CAETRGGGSQGNLIF

SEQ ID NO: 559 - protein, 14 aa, Homo sapiens
SAGAGGGSQGNLIF

SEQ ID NO: 560 - protein, 14 aa, Homo sapiens
CAGAGGGSQGNLIF

SEQ ID NO: 561 - protein, 15 aa, Homo sapiens
CSAPGTVLAKNIQYF

SEQ ID NO: 562 - protein, 15 aa, Homo sapiens
CSAPGTVLAKNIQYF

SEQ ID NO: 563 - protein, 13 aa, Homo sapiens
CASSIFGGAEAFF

SEQ ID NO: 564 - protein, 13 aa, Homo sapiens
CASSIRSTDTQYF

SEQ ID NO: 565 - protein, 13 aa, Homo sapiens
CASSLRSSYEQYF

SEQ ID NO: 566 - protein, 15 aa, Homo sapiens
CSAPGTVLAKNIQYF

SEQ ID NO: 567 - protein, 19 aa, Homo sapiens
  misc_feature (5)..(5): Xaa can be any naturally occurring amino acid
  UNSURE (5)..(5)
CTKLXNIAGTPWGDEKLFF

SEQ ID NO: 568 - protein, 13 aa, Homo sapiens
CASSPRAGVEQFF

SEQ ID NO: 569 - protein, 13 aa, Homo sapiens
CASSIRSTDTQYF

SEQ ID NO: 570 - protein, 13 aa, Homo sapiens
CASSPRAGVEQFF

SEQ ID NO: 571 - protein, 15 aa, Homo sapiens
CSAPGTVLAKNIQYF

SEQ ID NO: 572 - protein, 14 aa, Homo sapiens
CASSIFGGNQPQHF

SEQ ID NO: 573 - protein, 13 aa, Homo sapiens
CASSIRSTDTQYF

SEQ ID NO: 574 - protein, 13 aa, Homo sapiens
CASSLRSTGELFF

SEQ ID NO: 575 - protein, 13 aa, Homo sapiens
CASSPRAGVEQFF

SEQ ID NO: 576 - protein, 13 aa, Homo sapiens
CASSPRAGVEQFF

SEQ ID NO: 577 - protein, 13 aa, Homo sapiens
CASSGRSTDTQYF

SEQ ID NO: 578 - protein, 13 aa, Homo sapiens
CASSPRSTDTQYF

SEQ ID NO: 579 - protein, 13 aa, Homo sapiens
CASSIRSSDEQFF

SEQ ID NO: 580 - protein, 12 aa, Homo sapiens
CAISGNTDKLIF

SEQ ID NO: 581 - protein, 11 aa, Homo sapiens
CARNTGNQFYF

SEQ ID NO: 582 - protein, 12 aa, Homo sapiens
CAISGNTDKLIF

SEQ ID NO: 583 - protein, 12 aa, Homo sapiens
CAISGNTDKLIF

SEQ ID NO: 584 - protein, 14 aa, Homo sapiens
CAGTDGGSQGNLIF

SEQ ID NO: 585 - protein, 15 aa, Homo sapiens
CAETRGGGSQGNLIF

SEQ ID NO: 586 - protein, 20 aa, Homo sapiens
CSARREYGTVAGEGYNEQFF

SEQ ID NO: 587 - protein, 15 aa, Homo sapiens
CASSYQTGASYGYTF

SEQ ID NO: 588 - protein, 20 aa, Homo sapiens
CSARREYGTVAGEGYNEQFF

SEQ ID NO: 589 - protein, 20 aa, Homo sapiens
CSARREYGTVAGEGYNEQFF

SEQ ID NO: 590 - protein, 13 aa, Homo sapiens
CASSIFGGAEAFF

SEQ ID NO: 591 - protein, 13 aa, Homo sapiens
CASSLRSSYEQYF

SEQ ID NO: 592 - protein, 13 aa, Homo sapiens
CASSPRAGVEQFF

SEQ ID NO: 593 - protein, 12 aa, Homo sapiens
CAISGNTDKLIF

SEQ ID NO: 594 - protein, 11 aa, Homo sapiens
CARNTGNQFYF

SEQ ID NO: 595 - protein, 12 aa, Homo sapiens
CAISGNTDKLIF

SEQ ID NO: 596 - protein, 14 aa, Homo sapiens
CAGTDGGSQGNLIF

SEQ ID NO: 597 - protein, 15 aa, Homo sapiens
CAETRGGGSQGNLIF

SEQ ID NO: 598 - protein, 20 aa, Homo sapiens
CSARREYGTVAGEGYNEQFF

SEQ ID NO: 599 - protein, 15 aa, Homo sapiens
  misc_feature (13)..(14): Xaa can be any naturally occurring amino acid
  UNSURE (13)..(14)
CASSYQTGASRYXXF

SEQ ID NO: 600 - protein, 22 aa, Homo sapiens
  misc_feature (20)..(21): Xaa can be any naturally occurring amino acid
  UNSURE (20)..(21)
CGQLDVNTGPYRERDTMSSXXF

SEQ ID NO: 601 - protein, 13 aa, Homo sapiens
CASSIFGGAEAFF

SEQ ID NO: 602 - protein, 13 aa, Homo sapiens
CASSLRSSYEQYF

SEQ ID NO: 603 - protein, 13 aa, Homo sapiens
CASSPRAGVEQFF

SEQ ID NO: 604 - DNA, 19 bp, artificial sequence
  hBCR micro Block primer
tcctgtgcgaggcagccaa

SEQ ID NO: 605 - DNA, 20 bp, artificial sequence
  hBCR micro RT primer
tgatgtcagagttgttcttg

SEQ ID NO: 606 - DNA, 18 bp, artificial sequence
  hBCR Kappa Block primer
ctgtactttggcctctct

SEQ ID NO: 607 - DNA, 19 bp, artificial sequence
  hBCR Kappa RT primer
ttgtgtttctcgtagtctg

SEQ ID NO: 608 - DNA, 16 bp, artificial sequence
  hBCR Lambda Block primer
ccgggtagaagtcact

SEQ ID NO: 609 - DNA, 19 bp, artificial sequence
  hBCR Lambda RT primer
tgtcttctccacggtgctc

SEQ ID NO: 610 - DNA, 53 bp, artificial sequence
  hBCR micro Reverse Tag primer
tcgtcggcagcgtcagatgtgtataagagacaggtatccgacggggaattctc

SEQ ID NO: 611 - DNA, 51 bp, artificial sequence
  hBCR Kappa Reverse Tag primer
tcgtcggcagcgtcagatgtgtataagagacagttattcagcaggcacaca

SEQ ID NO: 612 - DNA, 51 bp, artificial sequence
  hBCR Lambda Reverse Tag primer
tcgtcggcagcgtcagatgtgtataagagacagtgtggccttgttggcttg

SEQ ID NO: 613 - protein, 18 aa, Homo sapiens
CASGRTGYYDSSGYHDYW

SEQ ID NO: 614 - protein, 17 aa, Homo sapiens
CTADHDYGALGLLGWYW

SEQ ID NO: 615 - protein, 10 aa, Homo sapiens
CANQAWLHYW

SEQ ID NO: 616 - protein, 20 aa, Homo sapiens
CAGLDVTGDCNDDWSYFHYW

SEQ ID NO: 617 - protein, 13 aa, Homo sapiens
CARGPVEMAKLYW

SEQ ID NO: 618 - protein, 23 aa, Homo sapiens
CAKVESYSDRRRGYYPNRFFDYW

SEQ ID NO: 619 - protein, 12 aa, Homo sapiens
CAIEFPIHYNSW

SEQ ID NO: 620 - protein, 10 aa, Homo sapiens
CANQAWLHYW

SEQ ID NO: 621 - protein, 20 aa, Homo sapiens
CAREARYCSGGSCYPYPDYW

SEQ ID NO: 622 - protein, 17 aa, Homo sapiens
CTADHDYGALGLLGWYW

SEQ ID NO: 623 - protein, 10 aa, Homo sapiens
CANQAWLHYW

SEQ ID NO: 624 - protein, 20 aa, Homo sapiens
WARMDVTGDWKDDWYYFEDW

SEQ ID NO: 625 - protein, 16 aa, Homo sapiens
CTRVGVDGYNYFGNYW

SEQ ID NO: 626 - protein, 18 aa, Homo sapiens
CAKSRPLKVVAATFLDYW

SEQ ID NO: 627 - protein, 19 aa, Homo sapiens
CARDEGQQLPLRPYGMDVW

SEQ ID NO: 628 - protein, 20 aa, Homo sapiens
CARIGLGGYSYYYYYGMDVW

SEQ ID NO: 629 - protein, 18 aa, Homo sapiens
CASGRTGYYDSSGYHDYW

SEQ ID NO: 630 - protein, 20 aa, Homo sapiens
CARLDVTGDWNDDWYYFDYW

SEQ ID NO: 631 - protein, 17 aa, Homo sapiens
CASGGDRWELLPYFDYW

SEQ ID NO: 632 - protein, 20 aa, Homo sapiens
GARREGGGDWNDQLYYFDYW

SEQ ID NO: 633 - protein, 18 aa, Homo sapiens
CASGRTGYYDSSGYHDYW

SEQ ID NO: 634 - protein, 18 aa, Homo sapiens
CARLSSSWYYYYYGMDVW

SEQ ID NO: 635 - protein, 20 aa, Homo sapiens
CARIGLGGYSYYYYYGMDVW

SEQ ID NO: 636 - protein, 15 aa, Homo sapiens
CARQTMIRHRWFDPW

SEQ ID NO: 637 - protein, 17 aa, Homo sapiens
CTADHDYGALGLLGWYW

SEQ ID NO: 638 - protein, 10 aa, Homo sapiens
CANQAWLHYW

SEQ ID NO: 639 - protein, 20 aa, Homo sapiens
CARLDVTGDWNDDWYYFDYW

SEQ ID NO: 640 - protein, 23 aa, Homo sapiens
CARHLVAPQSPRRNYYYYGMDVW

SEQ ID NO: 641 - protein, 23 aa, Homo sapiens
CAKVESYSDRRRGYYPNRFFDYW

SEQ ID NO: 642 - protein, 18 aa, Homo sapiens
CARLSSSWYYYYYGMDVW

SEQ ID NO: 643 - protein, 20 aa, Homo sapiens
CARIGLGGYSYYYYYGMDVW

SEQ ID NO: 644 - protein, 18 aa, Homo sapiens
CASGRTGYYDSSGYHDYW

SEQ ID NO: 645 - protein, 17 aa, Homo sapiens
CTADHDYGALGLLGWYW

SEQ ID NO: 646 - protein, 10 aa, Homo sapiens
CANQAWLHYW

SEQ ID NO: 647 - protein, 20 aa, Homo sapiens
CARLDVTGDWNDDWYYFDYW

SEQ ID NO: 648 - protein, 20 aa, Homo sapiens
CARIGLGGYSYYYYYGMDVW

SEQ ID NO: 649 - protein, 18 aa, Homo sapiens
CASGRTGYYDSSGYHDYW

SEQ ID NO: 650 - protein, 11 aa, Homo sapiens [sequence omitted]

SEQ ID NO: 651 - protein, 13 aa, Homo sapiens [sequence omitted]

SEQ ID NO: 652 - protein, 12 aa, Homo sapiens [sequence omitted]

SEQ ID NO: 653 - protein, 11 aa, Homo sapiens
  misc_feature (10)..(10): Xaa can be any naturally occurring amino acid
  UNSURE (10)..(10)
CQQRSNWGRXF

SEQ ID NO: 654 - protein, 11 aa, Homo sapiens [sequence omitted]

SEQ ID NO: 655 - protein, 11 aa, Homo sapiens [sequence omitted]

SEQ ID NO: 656 - protein, 11 aa, Homo sapiens [sequence omitted]

SEQ ID NO: 657 - protein, 11 aa, Homo sapiens
  misc_feature (10)..(10): Xaa can be any naturally occurring amino acid
  UNSURE (10)..(10)
CQQRSNWGRXF

SEQ ID NO: 658 - protein, 11 aa, Homo sapiens [sequence omitted]

SEQ ID NO: 659 - protein, 13 aa, Homo sapiens [sequence omitted]

SEQ ID NO: 660 - protein, 11 aa, Homo sapiens [sequence omitted]

SEQ ID NO: 661 - protein, 11 aa, Homo sapiens [sequence omitted]

SEQ ID NO: 662 - protein, 11 aa, Homo sapiens [sequence omitted]

SEQ ID NO: 663 - protein, 11 aa, Homo sapiens [sequence omitted]

SEQ ID NO: 664 - protein, 11 aa, Homo sapiens [sequence omitted]

SEQ ID NO: 665 - protein, 11 aa, Homo sapiens
  misc_feature (10)..(10): Xaa can be any naturally occurring amino acid
  UNSURE (10)..(10)
CQQRSNWGRXF

SEQ ID NO: 666 - protein, 10 aa, Homo sapiens [sequence omitted]

SEQ ID NO: 667 - protein, 12 aa, Homo sapiens [sequence omitted]

SEQ ID NO: 668 - protein, 11 aa, Homo sapiens [sequence omitted]

SEQ ID NO: 669 - protein, 11 aa, Homo sapiens [sequence omitted]

SEQ ID NO: 670 - protein, 11 aa, Homo sapiens [sequence omitted]

SEQ ID NO: 671 - protein, 11 aa, Homo sapiens [sequence omitted]

SEQ ID NO: 672 - protein, 11 aa, Homo sapiens
  misc_feature (10)..(10): Xaa can be any naturally occurring amino acid
  UNSURE (10)..(10)
CQQRSNWGRXF

SEQ ID NO: 673 - protein, 11 aa, Homo sapiens [sequence omitted]

SEQ ID NO: 674 - protein, 11 aa, Homo sapiens [sequence omitted]

SEQ ID NO: 675 - protein, 11 aa, Homo sapiens [sequence omitted]

SEQ ID NO: 676 - protein, 11 aa, Homo sapiens
  misc_feature (10)..(10): Xaa can be any naturally occurring amino acid
  UNSURE (10)..(10)
CQQRSNWGRXF

SEQ ID NO: 677 - protein, 13 aa, Homo sapiens [sequence omitted]

SEQ ID NO: 678 - protein, 11 aa, Homo sapiens [sequence omitted]

SEQ ID NO: 679 - protein, 12 aa, Homo sapiens [sequence omitted]

SEQ ID NO: 680 - protein, 11 aa, Homo sapiens
  misc_feature (10)..(10): Xaa can be any naturally occurring amino acid
  UNSURE (10)..(10)
CQQRSNWGRXF

SEQ ID NO: 681 - protein, 11 aa, Homo sapiens [sequence omitted]

SEQ ID NO: 682 - protein, 13 aa, Homo sapiens [sequence omitted]

SEQ ID NO: 683 - protein, 12 aa, Homo sapiens [sequence omitted]

SEQ ID NO: 684 - protein, 13 aa, Homo sapiens [sequence omitted]

SEQ ID NO: 685 - protein, 11 aa, Homo sapiens [sequence omitted]

SEQ ID NO: 686 - protein, 13 aa, Homo sapiens [sequence omitted]

SEQ ID NO: 687 - protein, 13 aa, Homo sapiens [sequence omitted]

SEQ ID NO: 688 - protein, 14 aa, Homo sapiens [sequence omitted]

SEQ ID NO: 689 - protein, 12 aa, Homo sapiens [sequence omitted]

SEQ ID NO: 690 - protein, 12 aa, Homo sapiens [sequence omitted]

SEQ ID NO: 691 - protein, 13 aa, Homo sapiens [sequence omitted]

SEQ ID NO: 692 - protein, 14 aa, Homo sapiens
  misc_feature (12)..(13): Xaa can be any naturally occurring amino acid
  UNSURE (12)..(13)
CCSYPRGTSFGXXF

SEQ ID NO: 693 - protein, 12 aa, Homo sapiens [sequence omitted]

SEQ ID NO: 694 - protein, 12 aa, Homo sapiens [sequence omitted]

SEQ ID NO: 695 - protein, 13 aa, Homo sapiens [sequence omitted]

SEQ ID NO: 696 - protein, 14 aa, Homo sapiens [sequence omitted]

SEQ ID NO: 697 - protein, 11 aa, Homo sapiens
CQTSDSRITVF

SEQ ID NO: 698 - protein, 12 aa, Homo sapiens
CVSYMGSGTWVF

SEQ ID NO: 699 - protein, 13 aa, Homo sapiens [sequence omitted]

SEQ ID NO: 700 - protein, 13 aa, Homo sapiens [sequence omitted]

SEQ ID NO: 701 - protein, 12 aa, Homo sapiens [sequence omitted]

SEQ ID NO: 702 - protein, 13 aa, Homo sapiens
CSSYTGSTTLDVF

SEQ ID NO: 703 - protein, 13 aa, Homo sapiens [sequence omitted]

SEQ ID NO: 704 - protein, 14 aa, Homo sapiens [sequence omitted]

SEQ ID NO: 705 - protein, 12 aa, Homo sapiens [sequence omitted]

SEQ ID NO: 706 - protein, 12 aa, Homo sapiens [sequence omitted]

SEQ ID NO: 707 - protein, 14 aa, Homo sapiens [sequence omitted]

SEQ ID NO: 708 - protein, 12 aa, Homo sapiens [sequence omitted]

SEQ ID NO: 709 - protein, 12 aa, Homo sapiens [sequence omitted]

SEQ ID NO: 710 - protein, 12 aa, Homo sapiens [sequence omitted]

SEQ ID NO: 711 - protein, 14 aa, Homo sapiens [sequence omitted]

SEQ ID NO: 712 - protein, 15 aa, Homo sapiens [sequence omitted]

SEQ ID NO: 713 - protein, 12 aa, Homo sapiens [sequence omitted]

SEQ ID NO: 714 - protein, 12 aa, Homo sapiens [sequence omitted]

SEQ ID NO: 715 - protein, 12 aa, Homo sapiens [sequence omitted]

SEQ ID NO: 716 - protein, 12 aa, Homo sapiens [sequence omitted]

SEQ ID NO: 717 - protein, 11 aa, Homo sapiens [sequence omitted]

SEQ ID NO: 718 - protein, 13 aa, Homo sapiens [sequence omitted]

SEQ ID NO: 719 - protein, 12 aa, Homo sapiens [sequence omitted]

SEQ ID NO: 720 - protein, 15 aa, Homo sapiens [sequence omitted]

SEQ ID NO: 721 - protein, 13 aa, Homo sapiens [sequence omitted]

SEQ ID NO: 722 - protein, 12 aa, Homo sapiens [sequence omitted]

SEQ ID NO: 723 - protein, 11 aa, Homo sapiens [sequence omitted]

SEQ ID NO: 724 - protein, 12 aa, Homo sapiens [sequence omitted]

SEQ ID NO: 725 - protein, 13 aa, Homo sapiens [sequence omitted]

SEQ ID NO: 726 - protein, 20 aa, Homo sapiens
CAREARYCSGGSCYPYPDYW

SEQ ID NO: 727 - protein, 16 aa, Homo sapiens
CTRVGVDGYNYFGNYW

SEQ ID NO: 728 - protein, 18 aa, Homo sapiens
CAKSRPLKVVAATFLDYW

SEQ ID NO: 729 - protein, 20 aa, Homo sapiens
CARLDVTGDWNDDWYYFDYW

SEQ ID NO: 730 - protein, 17 aa, Homo sapiens
CASGGDRWELLPYFDYW

SEQ ID NO: 731 - protein, 18 aa, Homo sapiens
CARLSSSWYYYYYGMDVW

SEQ ID NO: 732 - protein, 15 aa, Homo sapiens
CARQTMIRHRWFDPW

SEQ ID NO: 733 - protein, 20 aa, Homo sapiens
CARIGLGGYSYYYYYGMDVW

SEQ ID NO: 734 - protein, 18 aa, Homo sapiens
CASGRTGYYDSSGYHDYW

SEQ ID NO: 735 - protein, 11 aa, Homo sapiens [sequence omitted]

SEQ ID NO: 736 - protein, 10 aa, Homo sapiens [sequence omitted]

SEQ ID NO: 737 - protein, 12 aa, Homo sapiens [sequence omitted]

SEQ ID NO: 738 - protein, 11 aa, Homo sapiens
  misc_feature (10)..(10): Xaa can be any naturally occurring amino acid
  UNSURE (10)..(10)
CQQRSNWGRXF

SEQ ID NO: 739 - protein, 13 aa, Homo sapiens [sequence omitted]

SEQ ID NO: 740 - protein, 12 aa, Homo sapiens [sequence omitted]

SEQ ID NO: 741 - protein, 13 aa, Homo sapiens [sequence omitted]

SEQ ID NO: 742 - protein, 14 aa, Homo sapiens [sequence omitted]

SEQ ID NO: 743 - protein, 12 aa, Homo sapiens [sequence omitted]

SEQ ID NO: 744 - protein, 12 aa, Homo sapiens [sequence omitted]

SEQ ID NO: 745 - DNA, 37 bp, artificial sequence
  TS-AdpD3EV
tggtggaattctgcagataagcagtggtatcaacgca

SEQ ID NO: 746 - DNA, 18 bp, artificial sequence
  hTCRa-VX-F-AdpD3EV
tggtggaattctgcagat

SEQ ID NO: 747 - DNA, 39 bp, artificial sequence
  hTCRa-V24-F-AdpD3EV
tggtggaattctgcagattttctgctgtgggtacgtgag

SEQ ID NO: 748 - DNA, 18 bp, artificial sequence
  hTCRb-VX-F-AdpD3EV
tggtggaattctgcagat

SEQ ID NO: 749 - DNA, 38 bp, artificial sequence
  hTCRb-V6.5-F-AdpD3EV
tggtggaattctgcagatgagagtcctgctcccctttc

SEQ ID NO: 750 - DNA, 37 bp, artificial sequence
  TS-AdpMXEI
ccagtgtggtggtacgggaagcagtggtatcaacgca

SEQ ID NO: 751 - DNA, 18 bp, artificial sequence
  hTCRa-VX-F-AdpMXEI
ccagtgtggtggtacggg

SEQ ID NO: 752 - DNA, 18 bp, artificial sequence
  hTCRb-VX-F-AdpMXEI
ccagtgtggtggtacggg

SEQ ID NO: 753 - DNA, 39 bp, artificial sequence
  hTCRa-V24-F-AdpMXEI
ccagtgtggtggtacgggtttctgctgtgggtacgtgag

SEQ ID NO: 754 - DNA, 38 bp, artificial sequence
  hTCRb-V6.5-F-AdpMXEI
ccagtgtggtggtacggggagagtcctgctcccctttc

SEQ ID NO: 755 - DNA, 20 bp, artificial sequence
  hTCRa-C-R
agggtcagggttctggatat

SEQ ID NO: 756 - DNA, 20 bp, artificial sequence
  hTCRb-C1-R
gaacaccttgttcaggtcct

SEQ ID NO: 757 - DNA, 20 bp, artificial sequence
  hTCRb-C2-R
gaacacgtttttcaggtcct

SEQ ID NO: 758 - DNA, 21 bp, artificial sequence
  hTCRaC-F0
tctgcctattcaccgattttg

SEQ ID NO: 759 - DNA, 21 bp, artificial sequence
  hTCRaC-F1
ggacttcaagagcaacagtgc

SEQ ID NO: 760 - DNA, 21 bp, artificial sequence
  hTCRaC-F2
tgtcaagctggtcgagaaaag

SEQ ID NO: 761 - DNA, 21 bp, artificial sequence
  hTCRaC-F3
acgccttcaacaacagcatta

SEQ ID NO: 762 - DNA, 21 bp, artificial sequence
  hTCRaC-R1v1
cttttctcgaccagcttgaca

SEQ ID NO: 763 - DNA, 21 bp, artificial sequence
  hTCRaC-R2
ccactttcaggaggaggattc

SEQ ID NO: 764 - DNA, 21 bp, artificial sequence
  hTCRaC-R4
caaaatcggtgaataggcaga

SEQ ID NO: 765 - DNA, 22 bp, artificial sequence
  hTCRaC-R5
atagacctcatgtctagcacag

SEQ ID NO: 766 - DNA, 21 bp, artificial sequence
  hTCR-CB-F2
gctgtgtttgagccatcagaa

SEQ ID NO: 767 - DNA, 23 bp, artificial sequence
  hTCRbC-F5
tgaatgggaaggaggtgcacagt

SEQ ID NO: 768 - DNA, 21 bp, artificial sequence
  hTCR-CB-R2
ttctgatggctcaaacacagc

SEQ ID NO: 769 - DNA, 23 bp, artificial sequence
  hTCR-CB2
aggcagtatctggagtcattgag

SEQ ID NO: 770 - DNA, 23 bp, artificial sequence
  hTCR-CB3
actgtgcacctccttcccattca

SEQ ID NO: 771 - DNA, 22 bp, artificial sequence
  hTCRbC2-CloR1
gtttagcctatttcgtacttgg

SEQ ID NO: 772 - DNA, 23 bp, artificial sequence
  hTCRbC1-R1
agtcacttaggcatgctaaggtc

SEQ ID NO: 773 - DNA, 443 bp, artificial sequence
  hTCRaCFull-AdpD3EV
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgaatccagcacagtggcggc

SEQ ID NO: 774 - DNA, 443 bp, artificial sequence
  hTCRaCFull_noSTOP-AdpD3EV
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcttaatccagcacagtggcggc

SEQ ID NO: 775 - DNA, 551 bp, artificial sequence
  hTCRbC1Full-AdpD3EV
aggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaatccagcacagtggcggc

SEQ ID NO: 776 - DNA, 551 bp, artificial sequence
  hTCRbC1Full_noSTOP-AdpD3EV
aggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcttaatccagcacagtggcggc

SEQ ID NO: 777 - DNA, 557 bp, artificial sequence
  hTCRbC2Full-AdpD3EV
aggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagatccagcacagtggcggc

SEQ ID NO: 778 - DNA, 557 bp, artificial sequence
  hTCRbC2Full_noSTOP-AdpD3EV
aggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcttaatccagcacagtggcggc

SEQ ID NO: 779 - DNA, 443 bp, artificial sequence
  hTCRaCFull-AdpMXEI
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgaccagctgagcgccggtcg

SEQ ID NO: 780 - DNA, 551 bp, artificial sequence
  hTCRbC1Full-AdpMXEI
aggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctgaccagctgagcgccggtcg

SEQ ID NO: 781 - DNA, 557 bp, artificial sequence
  hTCRbC2Full-AdpMXEI
aggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagccagctgagcgccggtcg

SEQ ID NO: 782 - DNA, 37 bp, artificial sequence; identical to SEQ ID NO: 745
  TS-AdpD3EV

SEQ ID NO: 783 - DNA, 18 bp, artificial sequence
  hBCRH-VX-F-AdpD3EV
tggtggaattctgcagat

SEQ ID NO: 784 - DNA, 39 bp, artificial sequence
  hBCRH-V3.30-F-AdpD3EV
tggtggaattctgcagatactagaagtcggcggtgtttc

SEQ ID NO: 785 - DNA, 39 bp, artificial sequence
  hBCRH-V2.70-F-AdpD3EV
tggtggaattctgcagatacagtgaatcctgctccccac

SEQ ID NO: 786 - DNA, 18 bp, artificial sequence
  hBCRLam-VX-F-AdpD3EV
tggtggaattctgcagat

SEQ ID NO: 787 - DNA, 38 bp, artificial sequence
  hBCRLam-V2.14-F-AdpD3EV
tggtggaattctgcagattctcaggaggcagcgctctc

SEQ ID NO: 788 - DNA, 18 bp, artificial sequence
  hBCRKap-VX-F-AdpD3EV
tggtggaattctgcagat

SEQ ID NO: 789 - DNA, 38 bp, artificial sequence
  hBCRK-V3.15-F-AdpD3EV
tggtggaattctgcagataggaactgctcagttaggac

SEQ ID NO: 790 - DNA, 20 bp, artificial sequence
  hBCRM-C-R
gttggggcggatgcactccc

SEQ ID NO: 791 - DNA, 18 bp, artificial sequence
  hBCRLam-C2-R
ggcagccttgggctgacc

SEQ ID NO: 792 - DNA, 21 bp, artificial sequence
  hBCRKap-C1-R
cagatggtgcagccacagttc

SEQ ID NO: 793 - DNA, 341 bp, artificial sequence
  hBCRKapCFull-AdpD3EV
gaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagatccagcacagtggcggc

SEQ ID NO: 794 - DNA, 339 bp, artificial sequence
  hBCRLamC2Full-AdpD3EV
ggtcagcccaaggctgccccctcggtcactctgttcccgccctcctctgaggagcttcaagccaacaaggccacactggtgtgtctcataagtgacttctacccgggagccgtgacagtggcttggaaagcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagatccagcacagtggcggc

SEQ ID NO: 795 - DNA, 1380 bp, artificial sequence
  hBCRCFull-AdpD3EV
gggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcggccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccggtaaacccaccctgtacaacgtgtccctggtcatgtccgacacagctggcacctgctactgaatccagcacagtggcggc

SEQ ID NO: 796 - DNA, 528 bp, Homo sapiens
ggcactagaagtcggcggtgtttccattcggtgatcagcactgaacacagaggactcaccatggagtttgggctgagctgggttttcctcgttgctcttttaaggggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgccaaatcccgacccctgaaggtggtagctgctacttttcttgactactggggccagggaaccctggtcaccgtctcctcagggagtgcatccgccccaacccttttccccctcgtctcctgt

SEQ ID NO: 797 - protein, 176 aa, Homo sapiens
GTRSRRCFHSVISTEHRGLTMEFGLSWVFLVALLRGVQCQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKSRPLKVVAATFLDYWGQGTLVTVSSGSASAPTLFPLVSC

SEQ ID NO: 798 - DNA, 486 bp, Homo sapiens
gggggtctcaggaggcagcgctctcgggacgtctccaccatggcctgggctctgctattcctcaccctcctcactcagggcacagggtcctgggcccagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactctcggggtattcggcggagggaccaagctgaccgtcctaggtcagcccaaggctgccccctcggtcactctgttcccgccctcctctgaggagctt

SEQ ID NO: 799 - protein, 162 aa, Homo sapiens
GGLRRQRSRDVSTMAWALLFLTLLTQGTGSWAQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLGVFGGGTKLTVLGQPKAAPSVTLFPPSSEEL

SEQ ID NO: 800 - DNA, 506 bp, Homo sapiens
gcctcgcacagtgaatcctgctccccaccatggacatactttgttccacgctcctgctactgactgtcccgtcctgggtcttatcccaggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcacgcattgattgggatgatgataaatactacagcacatctctgaagaccaggctcaccatctccaaggacacctccaaaaaccaggtggtccttacaatgaccaacatggaccctgtggacacagccacgtattactgtgcacggataggtctaggtggctattcttactactactactacggtatggacgtctggggccaagggaccacggtcaccgtctcctcagggagtgcatccgccccaacccttttccccctcgtctcctgt

SEQ ID NO: 801 - protein, 168 aa, Homo sapiens
  misc_feature (4)..(4): Xaa can be any naturally occurring amino acid
  UNSURE (4)..(4)
LAQXILLPTMDILCSTLLLLTVPSWVLSQVTLRESGPALVKPTQTLTLTCTFSGFSLSTSGMCVSWIRQPPGKALEWLARIDWDDDKYYSTSLKTRLTISKDTSKNQVVLTMTNMDPVDTATYYCARIGLGGYSYYYYYGMDVWGQGTTVTVSSGSASAPTLFPLVSC

SEQ ID NO: 802 - DNA, 495 bp, Homo sapiens
ggaggaactgctcagttaggacccagacggaaccatggaagccccagcgcagcttctcttcctcctgctactctggctcccagataccactggagaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccggagtacacttttggccaggggaccaagctggagatcaaacgaactgtggctgcaccatctgtcttcatcttcccgccatctgatgagcagttgaaatctggaactgcctctgt

SEQ ID NO: 803 - protein, 164 aa, Homo sapiens
  misc_feature (6)..(6): Xaa can be any naturally occurring amino acid
  UNSURE (6)..(6)
EELLSXDPDGTMEAPAQLLFLLLLWLPDTTGEIVMTQSPATLSVSPGERATLSCRASQSVSSNLAWYQQKPGQAPRLLIYGASTRATGIPARFSGSGSGTEFTLTISSLQSEDFAVYYCQQYNNWPPEYTFGQGTKLEIKRTVAAPSVFIFPPSDEQLKSGTAS

SEQ ID NO: 804 - protein, 561 aa, Cytomegalovirus human betaherpesvirus 5
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYIKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYCIKGKLEYRHTWDRHDEGAAQGDDDVWTSGSDSDEELVTTERKTPRVTGGGAMAGASTSAGRKRKSASSATACTSGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 805 - protein, 252 aa, Alphainfluenzavirus influenza A
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFK

SEQ ID NO: 806 - DNA, 21 bp, artificial sequence
  hTCR alpha RT primer
atagcagggcctcgataatga

SEQ ID NO: 807 - DNA, 22 bp, artificial sequence
  hTCR alpha RT primer
aggacctagagcccaagagaac

SEQ ID NO: 808 - DNA, 21 bp, artificial sequence
  hTCR alpha PCR primer
ggagcacaggctgtcttacaa

SEQ ID NO: 809 - DNA, 21 bp, artificial sequence
  hTCR beta RT primer
ctgggttagggagatttcagc

SEQ ID NO: 810 - DNA, 21 bp, artificial sequence
  hTCR beta RT primer
gttgggagctcaatcttcagg

SEQ ID NO: 811 - DNA, 20 bp, artificial sequence
  hTCR beta PCR primer
ctaaggtccccctgggttag

SEQ ID NO: 812 - DNA, 21 bp, artificial sequence
  hTCR beta PCR primer
ctagcctctggaatcctttct

SEQ ID NO: 813 - DNA, 37 bp, artificial sequence
  hTCRaV38.2-F1-AdpD3EV
tggtggaattctgcagatagcagggacctgtgagcat

SEQ ID NO: 814 - DNA, 38 bp, artificial sequence
  hTCRbV14-F1-AdpD3EV
tggtggaattctgcagatgggtcctgccatggtttcca

SEQ ID NO: 815 - DNA, 39 bp, artificial sequence
  hTCRaV13.2-F2-AdpD3EV
tggtggaattctgcagatggctggagattgcaggtttat

SEQ ID NO: 816 - DNA, 40 bp, artificial sequence
  hTCRbV4.1-F1-AdpD3EV
tggtggaattctgcagataggctagcatgggctgcaggct

SEQ ID NO: 817 - DNA, 39 bp, artificial sequence
  hTCRaV27-F3-AdpD3EV
tggtggaattctgcagatggctctttcaggagcagctaa

SEQ ID NO: 818 - DNA, 38 bp, artificial sequence
  hTCRbV7.6-F1-AdpD3EV
tggtggaattctgcagatggtaaagccctcatcctgtc

SEQ ID NO: 819 - DNA, 39 bp, artificial sequence
  hTCRaC-AdpD3EV
gccgccactgtgctggattcagctggaccacagccgcag

SEQ ID NO: 820 - DNA, 38 bp, artificial sequence
  hTCRaC-NT-AdpD3EV
ccgccactgtgctggattaagctggaccacagccgcag

SEQ ID NO: 821 - DNA, 39 bp, artificial sequence
  hTCRbC1-AdpD3EV
gccgccactgtgctggattcagaaatcctttctcttgac

SEQ ID NO: 822 - DNA, 39 bp, artificial sequence
  hTCRbC1-NT-AdpD3EV
gccgccactgtgctggattaagaaatcctttctcttgac

SEQ ID NO: 823 - DNA, 39 bp, artificial sequence
  hTCRbC2-AdpD3EV
gccgccactgtgctggatctagcctctggaatcctttct

SEQ ID NO: 824 - DNA, 39 bp, artificial sequence
  hTCRbC2-NT-AdpD3EV
gccgccactgtgctggattaagcctctggaatcctttct

SEQ ID NO: 825 - DNA, 20 bp, artificial sequence
  hTCRa-C-F
atatccagaaccctgaccct

SEQ ID NO: 826 - DNA, 20 bp, artificial sequence
  hTCRb-C1-F
aggacctgaacaaggtgttc

SEQ ID NO: 827 - DNA, 20 bp, artificial sequence
  hTCRb-C2-F
aggacctgaaaaacgtgttc

SEQ ID NO: 828 - DNA, 21 bp, artificial sequence
  hTCR-CB2-R2
ttctgatggctcaaacacagc

SEQ ID NO: 829 - DNA, 848 bp, artificial sequence
  Base sequence for No.1 TCR alpha PCR product with stop codon
agcagggacctgtgagcatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcccggtattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 830 - protein, 276 aa, artificial sequence
  Amino acid sequence for No.1 TCR alpha PCR product with stop codon
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCARYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 831 - DNA, 848 bp, artificial sequence
  Base sequence for No.1 TCR alpha PCR product without stop codon
agcagggacctgtgagcatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcccggtattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctta

SEQ ID NO: 832 - protein, 277 aa, artificial sequence
  Amino acid sequence for No.1 TCR alpha PCR product without stop codon
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCARYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSL

SEQ ID NO: 833 - DNA, 943 bp, artificial sequence
  Base sequence for No.1 TCR beta PCR product with stop codon
gggtcctgccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaagatcgcatcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 834 - protein, 310 aa, artificial sequence
  Amino acid sequence for No.1 TCR beta PCR product with stop codon
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQDRIEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 835 - DNA, 943 bp, artificial sequence
  Base sequence for No.1 TCR beta PCR product without stop codon
gggtcctgccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaagatcgcatcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctta

SEQ ID NO: 836 - protein, 311 aa, artificial sequence
  Amino acid sequence for No.1 TCR beta PCR product without stop codon
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQDRIEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGL

SEQ ID NO: 837 - DNA, 876 bp, artificial sequence
  Base sequence for No.3 TCR alpha PCR product with stop codon
ggctggagattgcaggtttatgactgatcctatttgggaagaacaatgatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagacctcccccttttcagatggccagaagctgctctttgcaagggggaccatgttaaaggtggatcttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 838 - protein, 275 aa, artificial sequence
  Amino acid sequence for No.3 TCR alpha PCR product with stop codon
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAETSPFSDGQKLLFARGTMLKVDLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 839 - DNA, 876 bp, artificial sequence
  Base sequence for No.3 TCR alpha PCR product without stop codon
ggctggagattgcaggtttatgactgatcctatttgggaagaacaatgatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagacctcccccttttcagatggccagaagctgctctttgcaagggggaccatgttaaaggtggatcttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctta

SEQ ID NO: 840 - protein, 276 aa, artificial sequence
  Amino acid sequence for No.3 TCR alpha PCR product without stop codon
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAETSPFSDGQKLLFARGTMLKVDLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSL

SEQ ID NO: 841 - DNA, 944 bp, artificial sequence
  Base sequence for No.3 TCR beta PCR product with stop codon
aggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaggggcggagaaactacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 842 - protein, 311 aa, artificial sequence
  Amino acid sequence for No.3 TCR beta PCR product with stop codon
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGRRNYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 843 - DNA, 944 bp, artificial sequence
  Base sequence for No.3 TCR beta PCR product without stop codon
aggctagcatgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaggggcggagaaactacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctta

SEQ ID NO: 844 - protein, 312 aa, artificial sequence
  Amino acid sequence for No.3 TCR beta PCR product without stop codon
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGRRNYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGL

SEQ ID NO: 845 - DNA, 873 bp, artificial sequence
  Base sequence for No.4 TCR alpha PCR product with stop codon
ggctctttcaggagcagctaaagtcaggggccatgtccaccatgtgatagaaagacaagatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcctatccgaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 846 - protein, 295 aa, artificial sequence
  Amino acid sequence for No.4 TCR alpha PCR product with stop codon
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAIRQERTVLSTSLRPSLVIQASTSVPIRGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 847 - DNA, 873 bp, artificial sequence
  Base sequence for No.4 TCR alpha PCR product without stop codon
ggctctttcaggagcagctaaagtcaggggccatgtccaccatgtgatagaaagacaagatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcctatccgaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctta

SEQ ID NO: 848 - protein, 296 aa, artificial sequence
  Amino acid sequence for No.4 TCR alpha PCR product without stop codon
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAIRQERTVLSTSLRPSLVIQASTSVPIRGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSL

SEQ ID NO: 849 - DNA, 976 bp, artificial sequence
  Base sequence for No.4 TCR beta PCR product with stop codon
ggtaaagccctcatcctgtcctgaccctgccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttttgacttacttcaattatgaagcccaacaagacaaatcagggctgcccaatgatcggttttttgcagagaggcctgagggatccatctccactctgacgatccagcgcacagagcagcgggactcggccatgtatcgctgtgccagcagctcctctagcgggagctcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 850 - protein, 314 aa, artificial sequence
  Amino acid sequence for No.4 TCR beta PCR product with stop codon
MGTSLLCWVVLGFLGTDHTGAGVSQSPRYKVTKRGQDVALRCDPISGHVSLYWYRQALGQGPEFLTYFNYEAQQDKSGLPNDRFFAERPEGSISTLTIQRTEQRDSAMYRCASSSSSGSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 851 - DNA, 976 bp, artificial sequence
  Base sequence for No.4 TCR beta PCR product without stop codon
ggtaaagccctcatcctgtcctgaccctgccatgggcaccagtctcctatgctgggtggtcctgggtttcctagggacagatcacacaggtgctggagtctcccagtctcccaggtacaaagtcacaaagaggggacaggatgtagctctcaggtgtgatccaatttcgggtcatgtatccctttattggtaccgacaggccctggggcagggcccagagtttttgacttacttcaattatgaagcccaacaagacaaatcagggctgcccaatgatcggttttttgcagagaggcctgagggatccatctccactctgacgatccagcgcacagagcagcgggactcggccatgtatcgctgtgccagcagctcctctagcgggagctcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctta

SEQ ID NO: 852 - protein, 315 aa, artificial sequence
  Amino acid sequence for No.4 TCR beta PCR product without stop codon
MGTSLLCWVVLGFLGTDHTGAGVSQSPRYKVTKRGQDVALRCDPISGHVSLYWYRQALGQGPEFLTYFNYEAQQDKSGLPNDRFFAERPEGSISTLTIQRTEQRDSAMYRCASSSSSGSSYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGL

SEQ ID NO: 853 - DNA, 20 bp, artificial sequence
  T7
taatacgactcactataggg

SEQ ID NO: 854 - DNA, 21 bp, artificial sequence
  CMV-Fwd2
cgcaaatgggcggtaggcgtg

SEQ ID NO: 855 - DNA, 18 bp, artificial sequence
  BGH-Reverse
tagaaggcacagtcgagg

SEQ ID NO: 856 - DNA, 968 bp, artificial sequence
  pcDNA3.1V5HisB/TCR alpha plasmid with stop codon
agcagggacctgtgagcatggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcccggtattcaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgaatccagcacagtggcggccgctcgagtctagagggcccgcggttcgaaggtaagcctatccctaaccctctcctcggtctcgattctacgcgtaccggtcatcatcaccatcaccattga

SEQ ID NO: 857 - protein, 316 aa, artificial sequence
  pcDNA3.1V5HisB/TCR alpha plasmid with stop codon expressing amino acid sequence
  misc_feature (277)..(277): Xaa can be any naturally occurring amino acid
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCARYSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSXIQHSGGRSSLEGPRFEGKPIPNPLLGLDSTRTGHHHHHH

SEQ ID NO: 858 - DNA, 1063 bp, artificial sequence
  pcDNA3.1V5HisB/TCR beta plasmid with stop codon
gggtcctgccatggtttccaggcttctcagtttagtgtccctttgtctcctgggagcaaagcacatagaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaagatcgcatcgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagatccagcacagtggcggccgctcgagtctagagggcccgcggttcgaaggtaagcctatccctaaccctctcctcggtctcgattctacgcgtaccggtcatcatcaccatcaccattga

SEQ ID NO: 859 - protein, 350 aa, artificial sequence
  pcDNA3.1V5HisB/TCR beta plasmid with stop codon expressing amino acid sequence
  misc_feature (311)..(311): Xaa can be any naturally occurring amino acid
MVSRLLSLVSLCLLGAKHIEAGVTQFPSHSVIEKGQTVTLRCDPISGHDNLYWYRRVMGKEIKFLLHFVKESKQDESGMPNNRFLAERTGGTYSTLKVQPAELEDSGVYFCASSQDRIEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGXIQHSGGRSSLEGPRFEGKPIPNPLLGLDSTRTGHHHHHH

SEQ ID NO: 860 - DNA, 27 bp, Artificial Sequence
  forward primer HTCRAC1E4-R2
cttccaaatcattttaatgaaggcatc

SEQ ID NO: 861 - DNA, 21 bp, Artificial Sequence
  forward primer HTCRBC2E4-R1
gcaaccaggcccaacacacaa

(19) United States
(12) Patent Application Publication
(10) Pub. No.: US 2021/0301324 A1
(43) Pub. Date: Sep. 30, 2021

(54) METHOD FOR ANALYZING FUNCTIONAL SUBUNIT PAIR GENE OF T CELL RECEPTOR AND B CELL RECEPTOR

(71) Applicant: Repertoire Genesis Incorporation, Ibaraki-shi, Osaka (JP)

(72) Inventors: Ryuji Suzuki, Machida-shi, Tokyo (JP); Hiroyuki Sato, Minoh-shi, Osaka (JP)

(21) Appl. No.: 17/270,733

(22) PCT Filed: Aug. 23, 2019
(86) PCT No.: PCT/JP2019/033095

§ 371 Date: Feb. 23, 2021

(30) Foreign Application Priority Data
Aug. 24, 2018 (JP) 2018-157760

(51) Int. Cl. C12Q 1/686; C12N 15/86; G16B 30/10; C12Q 1/6806; C12Q 1/6881; C12P 19/34

(52) U.S. Cl. CPC C12Q 1/686; C12N 15/86; C12P 19/34; C12Q 1/6806; C12Q 1/6881; G16B 30/10

(57) ABSTRACT

The present disclosure provides a method for analyzing a functional subunit pair gene of a T cell receptor (TCR) or a B cell receptor (BCR). The present disclosure provides a method comprising: (1) a step for providing a nucleic acid sample containing a nucleic acid of a TCR or BCR from a cell expressing an activated TCR or BCR; (2) a step for determining the nucleic acid sequence of the functional pair gene of the TCR or the BCR; and (3) a step for calculating the frequency of appearance of each gene and combinations thereof on the basis of the determined nucleic sequences, and identifying a functional subunit pair gene of the TCR or the BCR.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 790132_408USPC_SEQUENCE_LISTING.txt. The text file is 248 KB, was created on May 12, 2021, and is being submitted electronically via EFS-Web.

### TECHNICAL FIELD

The present disclosure relates to a method of analyzing functional subunit pair genes of a T cell receptor (TCR) or a B cell receptor (BCR) from a small amount of cells.

### BACKGROUND ART

In recent years, a rapidly advancing next generation sequence analysis technology has enabled large-scale sequencing of the base sequences of genes. PCR amplification of a TCR gene from a human sample and use of a next generation sequence analysis technology have enabled materialization of a next generation TCR repertoire analysis method for obtaining and analyzing more detailed genetic information at a clone level in lieu of conventional TCR repertoire analysis, which is small scale and obtains limited information such as V chain usage frequency. Patent Literature 1 discloses a method of quantitatively analyzing a repertoire of a variable region of a T cell receptor (TCR) or B cell receptor (BCR) using a nucleic acid sample that has been amplified in an unbiased manner with an adapter primer from a population of cells.

Patent Literature 2 discloses a method of analyzing a repertoire of a T cell receptor (TCR) or B cell receptor (BCR) utilizing a technology for performing reverse transcription template switching PCR in one step.

### CITATION LIST

### Patent Literature

[PTL 1] International Publication No. WO 2015/075939

[PTL 2] International Publication No. WO 2017/222056

### SUMMARY OF INVENTION

### Solution to Problem

The present disclosure provides a method of analyzing functional pair genes of a T cell receptor (TCR) or a B cell receptor (BCR) quickly, readily, and with high specificity from a single cell.

The present disclosure provides, for example, the following items.

### (Item 1)

A method of analyzing a sequence of functional subunit pair genes of a T cell receptor (TCR) or a B cell receptor (BCR), comprising:

(1) providing a nucleic acid sample comprising a nucleic acid of a TCR or a BCR from an activated cell expressing a TCR or a BCR;
(2) determining a nucleic acid sequence of the TCR or the BCR; and
(3) calculating a frequency of appearance of each gene or a combination thereof based on the determined nucleic acid sequence to identify TCR or BCR functional subunit pair genes.

### (Item 2)

The method of item 1, further comprising activating at least a part of the cells expressing a TCR or a BCR prior to step (1).

### (Item 3)

The method of item 1 or 2, wherein the activation comprises stimulating the cells with any antigen or an antigen MHC complex thereof, or an antigen presenting cell having an MHC bound to any antigen.

### (Item 4)

The method of item 3, wherein the antigen is selected from the group consisting of a virus constituent substance, a microbe constituent substance, a non-autologous cell constituent substance, and a cancer cell specific constituent substance.

### (Item 5)

The method of item 3, wherein the antigen is selected from the group consisting of a viral peptide, a viral peptide-glycolipid complex, a microbial peptide, a microbial peptide-glycolipid complex, a non-autologous cell peptide, a non-autologous cell peptide-glycolipid complex, a cancer cell specific peptide, and a cancer cell specific peptide-glycolipid complex.

### (Item 6)

The method of item 1 or 2, wherein the activation comprises stimulating the cells with an agent selected from the group consisting of an anti-CD3 and/or anti-CD28 antibody, a phospholipase C (PLC) activating agent, a calcium ionophore, a protein kinase C (PKC) activating agent, a phytohemagglutinin (PHA), a concanavalin A (ConA), and a toll-like receptor activating agent.

### (Item 7)

The method of any one of items 1 to 6, wherein the cells are peripheral blood mononuclear cells.

### (Item 8)

The method of any one of items 1 to 7, wherein step (1) comprises separating the activated cell from an unactivated cell.

### (Item 9)

The method of item 8, wherein the separation of the activated cell is performed with an antigen-MHC molecule complex or a polymer of 2 to 10,000 antigen-MHC molecule complexes.

### (Item 10)

The method of item 9, wherein the polymer of antigen-MHC molecule complexes is selected from the group consisting of an epitope dimer, an epitope trimer, an epitope tetramer, and an epitope pentamer.

### (Item 11)

The method of any one of items 1 to 10, wherein the nucleic acid sample is obtained from a single activated cell.

### (Item 12)

The method of any one of items 1 to 11, wherein step (1) comprises:

a) mixing an RNA of the cells, a reagent required for reverse transcription, a reagent required for template switching, and a reagent required for a polymerase chain reaction, and subjecting the mixture to a reserve transcription inducing condition to provide a cDNA comprising nucleic acid sequences of a plurality of types of TCRs or BCRs; and
b) subjecting the cDNA obtained from step a) to a polymerase chain reaction inducing condition to provide a nucleic acid sample comprising the nucleic acid sequences of the plurality of types of TCRs or BCRs; wherein

the reagent required for template switching comprises a template switching oligonucleotide, and

the reagent required for a polymerase chain reaction comprises a primer specific to a C region, a primer specific to a 3′ untranslated region, or a primer spanning a C region and a 3′ untranslated region of the TCR or the BCR, wherein the primer specific to a C region, the primer specific to a 3′ untranslated region, or the primer spanning a C region and a 3′ untranslated region is a modified oligonucleotide primer designed to partially or completely block a primer function in step a) and to clear blocking of a primer function in step b).

### (Item 13)

The method of item 12, wherein the reagent required for a polymerase chain reaction optionally further comprises a 5′ anchor oligonucleotide primer comprising at least a part of an anchor sequence contained in the template switching oligonucleotide.

### (Item 14)

The method of item 13, wherein the reagent required for a polymerase chain reaction is free of the 5′ anchor oligonucleotide primer, and the template switching oligonucleotide functions as a 5′ anchor oligonucleotide primer.

### (Item 15)

The method of any one of items 12 to 14, wherein the reagent required for reverse transcription comprises:

(i) an oligonucleotide primer that initiates reverse transcription which is complementary to a C region, a 3′ untranslated region, or a region spanning a C region and a 3′ untranslated region of TCRα and an oligonucleotide primer that initiates reverse transcription which is complementary to a C region, a 3′ untranslated region, or a region spanning a C region and a 3′ untranslated region of TCRβ;
(ii) an oligonucleotide primer that initiates reverse transcription which is complementary to a C region, a 3′ untranslated region, or a region spanning a C region and a 3′ untranslated region of TCRδ and an oligonucleotide primer that initiates reverse transcription which is complementary to a C region, a 3′ untranslated region, or a region spanning a C region and a 3′ untranslated region of TCRγ; or
(iii) an oligonucleotide primer that initiates reverse transcription which is complementary to a C region, a 3′ untranslated region, or a region spanning a C region and a 3′ untranslated region of a BCR heavy chain and an oligonucleotide primer that initiates reverse transcription which is complementary to a C region, a 3′ untranslated region, or a region spanning a C region and a 3′ untranslated region of a BCR light chain.

### (Item 16)

The method of any one of items 12 to 15, wherein the reagent required for a polymerase chain reaction comprises:

(i) a primer specific to a C region, a primer specific to a 3′ untranslated region, or a primer spanning a C region and a 3′ untranslated region of TCRα and a primer specific to a C region, a primer specific to a 3′ untranslated region, or a primer spanning a C region and a 3′ untranslated region of TCRβ;
(ii) a primer specific to a C region, a primer specific to a 3′ untranslated region, or a primer spanning a C region and a 3′ untranslated region of TCRδ and a primer specific to a C region, a primer specific to a 3′ untranslated region, or a primer spanning a C region and a 3′ untranslated region of TCRγ; or
(iii) a primer specific to a C region, a primer specific to a 3′ untranslated region, or a primer spanning a C region and a 3′ untranslated region of a BCR heavy chain and a primer specific to a C region, a primer specific to a 3′ untranslated region, or a primer spanning a C region and a 3′ untranslated region of a BCR light chain.

### (Item 17)

The method of any one of items 12 to 16, wherein both TCRα and TCRβ, both TCRδ and TCRγ, or both a BCR heavy chain and a BCR light chain are co-amplified.

### (Item 18)

The method of any one of items 12 to 17, wherein the modified oligonucleotide primer has one or more complementary regions on a sequence of the same modified oligonucleotide primer and has a folded structure due to the complementary regions prior to initial thermal denaturation processing of PCR, or comprises a thermolabile modifying group.

### (Item 19)

The method of any one of items 12 to 18, wherein a part of a modified oligonucleotide whose primer function has not been blocked functions as an oligonucleotide primer that initiates reverse transcription by hybridizing to a template RNA.

### (Item 20)

The method of any one of items 12 to 19, wherein step (3) comprises:

(3-1) providing a reference database for each of gene regions comprising at least one of a V region, a D region, a J region, and optionally a C region;
(3-2) providing an input sequence set prepared from optionally trimming and optionally extracting a sequence with a suitable length;
(3-3) searching for homology of the input sequence set with the reference database for each of the gene regions and recording an alignment with an approximate reference allele and/or a sequence of the reference allele;
(3-4) assigning the V region and the J region for the input sequence set and extracting a nucleic acid sequence of the D region based on a result of assigning;
(3-5) translating the nucleic acid sequence of the D region into an amino acid sequence and classifying the D region by utilizing the amino acid sequence; and
(3-6) calculating a frequency of appearance or a combination for each of the V region, the D region, the J region, and optionally the C region based on the classifying in step (3-5) to identify functional subunit pair genes of a TCR or a BCR.

### (Item 21)

A system for analyzing a sequence of functional subunit pair genes of a T cell receptor (TCR) or a B cell receptor (BCR) in cells expressing a TCR or a BCR, comprising:

(A) an activator for activating at least a part of the cells;
(B) a nucleic acid treating kit for providing a nucleic acid sample comprising a nucleic acid sequence of a TCR or a BCR obtained from the activated cell;
(C) a sequencer for determining a nucleic acid sequence contained in the activated cell; and
(D) an analyzer for calculating a frequency of appearance of each gene or a combination thereof based on the determined nucleic acid sequence to identify functional subunit pair genes of a TCR or a BCR.

### (Item 22)

A method of manufacturing a cell expressing a T cell receptor (TCR) or a B cell receptor (BCR) having a sequence of functional subunit pair genes analyzed in accordance with the method of any one of items 1 to 20, comprising:

(4) providing an expression construct comprising the nucleic acid sequence of the TCR or the BCR; and
(5) introducing the expression construct into the cell.

### (Item 23)

A method of manufacturing a cell producing a virus or a phage comprising a T cell receptor (TCR) or B cell receptor (BCR) nucleic acid having a sequence of functional subunit pair genes analyzed in accordance with the method of any one of items 1 to 20, comprising:

(4) providing a virus producing vector or a phage producing vector comprising the nucleic acid sequence of the TCR or the BCR; and
(5) introducing the vector into the cell.

### (Item 24)

A method of manufacturing a virus or a phage comprising a T cell receptor (TCR) or B cell receptor (BCR) nucleic acid having a sequence of functional subunit pair genes analyzed in accordance with the method of any one of items 1 to 20, comprising:

(4) providing a virus producing vector or a phage producing vector comprising the nucleic acid sequence of the TCR or the BCR;
(5) introducing the vector into a cell; and
(6) culturing the cell to obtain a virus or a phage comprising a TCR or BCR nucleic acid from culture supernatant.

### (Item 25)

A method of manufacturing an RNA of a T cell receptor (TCR) or a B cell receptor (BCR) having a sequence of functional subunit pair genes analyzed in accordance with the method of any one of items 1 to 20, comprising:

(4) providing a vector for in vitro transcriptional RNA synthesis comprising the nucleic acid sequence of the TCR or BCR or a DNA fragment prepared from attaching an in vitro transcription promoter sequence to the nucleic acid sequence of the TCR or BCR; and
(5) subjecting the vector or the DNA fragment to a condition under which an RNA polymerase reaction starts in vitro.

### (Item 26)

A method of manufacturing a T cell receptor (TCR) or B cell receptor (BCR) protein having a sequence of functional subunit pair genes analyzed in accordance with the method of any one of items 1 to 20, comprising:

(4) providing an expression construct comprising the nucleic acid sequence of the TCR or the BCR;
(5) introducing the expression construct into a cell; and
(6) subjecting the cell to a condition under which the TCR or the BCR is expressed.

### (Item 27)

A method of manufacturing a T cell receptor (TCR) or B cell receptor (BCR) protein having a sequence of functional subunit pair genes analyzed in accordance with the method of any one of items 1 to 20, comprising:

(4) infecting a cell with a virus or a phage manufactured by the method of item 24; and
(5) subjecting the cell to a condition under which the TCR or the BCR is expressed.

### (Item 28)

A method of manufacturing a T cell receptor (TCR) or B cell receptor (BCR) protein having a sequence of functional subunit pair genes analyzed in accordance with the method of any one of items 1 to 20, comprising:

(4) providing a vector for in vitro transcriptional RNA synthesis comprising the nucleic acid sequence of the TCR or the BCR or a DNA fragment prepared from attaching an in vitro transcription promoter sequence to the nucleic acid sequence of the TCR or the BCR;
(5) subjecting the vector or the DNA fragment to a condition under which an RNA polymerase reaction starts in vitro to provide an mRNA; and
(6) subjecting the mRNA to a condition under which a protein is generated from the mRNA in vitro to provide the protein.

The present disclosure is intended so that one or more of the features described above can be provided not only as the explicitly disclosed combinations, but also as other combinations. Additional embodiments and advantages of the present disclosure are recognized by those skilled in the art by reading and understanding the following detailed description as needed.

The content of International Publication No. WO 2017/222056 and International Publication No. WO 2017/222057 is incorporated herein by reference in its entirety.

### Advantageous Effects of Invention

According to the present disclosure, sequence information on functional T cell receptor (TCR) or B cell receptor (BCR) pair genes quickly, readily, and with high specificity from a single cell by co-amplifying both TCR or BCR pair genes (e.g., TCRα/β, TCRδ/γ, or BCR heavy chain/light chain) can be obtained.

### BRIEF DESCRIPTION OF DRAWINGS

FIG. 1 shows results of separating PBMCs with a cell sorter. P1 indicates the lymphocyte gate, P2 indicates the CMV pp65 peptide MHC complex tetramer-positive CD8-positive T cell (cytotoxic T cell) gate, and P3 indicates the CMV pp65 peptide MHC complex tetramer-negative CD8-positive T cell (cytotoxic T cell) gate.

FIG. 2 shows electrophoretic images of a one-step RT-TS-PCR reaction product in Example 1. Wells 16, 32, 48, 64, and 90 indicate negative control wells with no input of cells.

FIG. 3 shows electrophoretic images of a semi-nested PCR reaction product in Example 1. Wells 16, 32, 48, 64, and 90 indicate negative control wells with no input of cells.

FIG. 4 shows results of determining a V region by sequencing and electrophoretic images of a semi-nested PCR reaction product in Example 1. Wells 16, 32, 48, 64, and 90 indicate negative control wells with no input of cells.

FIG. 5 shows electrophoretic images of a semi-nested PCR reaction product from amplifying TCRα and TCRβ in separate reaction systems in Example 2. Wells 10, 12, 15, 25 to 28, 36, 50, 53, 54, 59, 61, 65, 67, 70, 72, 75, 77, and 81 are wells in which a band was observed in both TCRα/β. Wells 4, 34, 51, 52, 68, and 85 for TCRα, and wells 1, 5 to 9, 11, 13, 19, 20, 29, 32, 33, 40, 55, 56, 58, 62, 63, 66, 73, 74, 78, 83, and 86 for TCRβ indicate wells in which a band was observed on only one side.

FIG. 6 shows electrophoretic images of a semi-nested PCR reaction product from amplifying TCRα and TCRβ in the same reaction system in Example 2. Wells 1, 5 to 13, 15, 19, 20, 25 to 29, 32 to 34, 36, 40, 50 to 56, 58, 59, 61 to 63, 65 to 68, 70, 72 to 75, 77, 78, 81, 83, 85, and 86 indicate wells in which a band was observed.

FIG. 7 shows electrophoretic images of a semi-nested PCR reaction product in cells that were stimulated with a CMV pp65 peptide and sorted in Example 3. Wells 6, 7, 12, 13, 15, 18, 20, 29, 31, 32, 33, 37, 38, 44, 52, 60, 77, 86, and 87 indicate wells in which a band was observed in both TCRα/β. Wells 1, 11, 26, 49, 50, and 74 for TCRα, and wells 1, 3, 5, 8, 10, 14, 17, 25, 27, 30, 40, 51, 54 to 58, 61, 63, 67, 76, and 78 for TCRβ indicate wells in which a band was observed on only one side.

FIG. 8 shows electrophoretic images of a semi-nested PCR reaction product in cells that were stimulated with a M1 peptide and sorted in Example 3. Wells 13, 36, 39, 42, 54, 57, 67, 82, and 90 indicate wells in which a band was observed in both TCRα/β. Wells 2, 3, 26 to 28, 31, 32, 34, 35, 40, 41, 75, 77, 85, and 89 for TCRα, and wells 8, 11, 25, 33, 37, 38, 61, 74, 76, 78, 79, 83, and 87 for TCRβ indicate wells in which a band was observed on only one side.

FIG. 9 shows electrophoretic images of a semi-nested PCR product from amplifying BCRμ, BCRκ, and BCRλ in the same reaction system in Example 4.

FIG. 10 shows electrophoretic images of a semi-nested PCR product from amplifying BCRμ, BCRκ, and BCRλ in separate reaction systems in Example 4.

FIG. 11 shows a block diagram of a repertoire analysis system of the present disclosure.

FIG. 12 shows electrophoretic images of a semi-nested PCR product from amplifying TCRα and TCRβ in separate reaction systems in Example 7. Under condition 1, wells 1 to 5 and 7 to 15 indicate wells in which a band was observed in both TCRα/β, and well 6 for TCRα indicates a well in which a band was observed on only one side. Under condition 2, wells 3 to 5, 7, 8, 11, 12, and 15 to 19 indicate wells in which a band was observed in both TCRα/β, and wells 1 and 2 for TCRα and wells 6, 10, and 13 for TCRβ indicate wells in which a band was observed on only one side. Under condition 3, wells 1, 5, 8, 12 to 17, and 19 indicate wells in which a band was observed in both TCRα/β, and well 11 for TCRα and wells 2, 7, 9, 10, and 18 for TCRβ indicate wells in which a band was observed on only one side.

FIG. 13 shows a result of PCR cloning of a TCR pair gene full length protein coding region cDNA.

FIG. 14 shows electrophoretic images of constructed pcDNA3.1 V5-His B/TCRα and pcDNA3.1 V5-His B/TCRβ expression plasmids.

FIG. 15 shows a result of PCR cloning of a TCR C region cDNA.

FIG. 16 shows a result of PCR cloning of cDNA from a TCR C region to a 3′ UTR region.

FIG. 17 shows a result of PCR cloning of a TCR pair gene V(D)J protein coding region cDNA.

### DESCRIPTION OF EMBODIMENTS

The present disclosure is described hereinafter. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the general understanding of those skilled in the art to which the present disclosure pertains. In case of a contradiction, the present specification (including the definitions) takes precedence.

### Definitions

As used herein, “about” preceding a numerical value refers to a numerical range of ±10% from the numerical value that is described subsequent to “about”.

As used herein, “activate a cell” refers to contacting any active substance that induces expression of any antigen or TCR or BCR with a cell to increase the amount of expression of mRNA of the TCR or BCR in the cell. Examples of active substances that induce expression of a TCR or BCR include, but are not limited to, an anti-CD3 and/or anti-CD28 antibody, ionomycin, PMA, PHA, ConA, and toll-like receptor activating agents. Antigens and active substances used for activating a cell are also referred to as an “activator”.

As used herein, “activation” can be typically expressed as cell growth and increase in immune action of T cells/B cells associated with an increase in a cytokine or immunity related protein. A specific determination methodology includes a cytotoxicity assay within a Petri dish for T cells. Since the number of target cells killed by T cells increases when “activated” in this methodology, activity can be measured by counting the cells.

More specifically, “activation” can also be measured biologically by an increase in clones due to cell growth (total cells increase with a non-specific activating agent, while only T cells or B cells binding to a specific antigen increase for a specific antigen), quantification of the amount of expression of mRNA or protein of a cytokine such as an interferon or interleukin that elevates immunological activity, or an increase in the amount of TCR or BCR/immunoglobulin protein. Specifically, as a more simple and convenient assay system for the measurement thereof, a methodology such as determination of an increase in the amount of mRNA by gene expression analysis or blastocyting of cells (cells growing and aggregating like a bunch of grapes) by microscope observation can be utilized. A methodology of detecting cytotoxic T lymphocyte markers CD8, CD103, and the like can also be used. Activation of B cells can also be determined by quantifying the amount of expression of protein of an immunoglobulin or the amount of expression of mRNA.

As used herein, “functional subunit pair genes” of a TCR or BCR refer to pair genes constituting a TCR or a BCR that actually functions in view of proteins translated and biosynthesized from pair genes (subunits such as TCRα/β, TCRδ/γ, or BCR heavy chain/light chain) associating to form a TCR protein complex or BCR/immunoglobulin protein complex when expressed in a cell.

As use herein, “T cell receptor (TCR)” refers to an antigen recognizing receptor, which is expressed on a cell membrane of a T cell that plays a central role in the immune system. TCRs have an α chain, β chain, γ chain, and δ chain, with which an αβ or γδ dimer is constituted. TCRs consisting of the combination of the former are known as αβ TCRs, and TCRs consisting of the combination of the latter are known as γδ TCRs. T cells having such TCRs are respectively known as αβ T cells and γδ T cells. TCRs are structurally very similar to a Fab fragment of an antibody produced by B cells and recognize antigen molecules bound to an MHC molecule. Since a TCR gene of a mature T cell has undergone gene rearrangement, an individual has highly diverse TCRs that enable recognition of various antigens. TCRs also form a complex by binding to a non-variable CD3 molecule that is present on the cell membrane. CD3 has an amino acid sequence known as ITAM (immunoreceptor tyrosine-based activation motif) in the intracellular region. This motif is considered to be involved in intracellular signaling. Each TCR chain is comprised of a variable domain (V) and a constant domain (C). A constant domain has a short cytoplasm section penetrating the cell membrane. A variable domain is present outside the cell and binds to an antigen-MHC complex. A variable domain has three regions known as hypervariable domains or complementarity-determining regions (CDRs), which bind to an antigen-MHC complex. The three CDRs are known as CDR1, CDR2, and CDR3. For TCRs, it is understood that CDR1 and CDR2 bind to an MHC, and CDR3 binds to an antigen. TCR gene rearrangement is similar to the process of B cell receptors known as immunoglobulins. Gene rearrangement of αβ TCRs entails VDJ recombination of a β chain, followed by VJ recombination of an α chain. Since the gene of the δ chain is deleted from a chromosome upon rearrangement of the α chain, a T cell having an αβ TCR would never concurrently have a γδ TCR. In contrast, a signal via a γδ TCR in a T cell having the TCR suppresses the expression of a β chain, so that a T cell having a γδ TCR would never concurrently have an αβ TCR.

As used herein, “B cell receptor (BCR)” refers to a receptor comprised of an Igα/Igβ (CD79a/CD79b) heterodimer (α/β) associated with a membrane bound immunoglobulin (mIg) molecule. An mIg subunit binds to an antigen to induce aggregation of receptors, while an α/β subunit transmits a signal toward the cell. Aggregation of BCRs is understood to quickly activate Src family kinases Lyn, Blk, and Fyn in the same manner as tyrosine kinases Syk and Btk. Many different results are produced depending on the complexity of BCR signaling. Examples thereof include survival, resistance (anergy; lack of hypersensitive reaction to an antigen) or apoptosis, cell division, differentiation into an antibody producing cell or memory B cell, and the like.

As used herein, “repertoire” or “repertoire of a variable region” refers to a collection of V(D)J regions created in any manner by gene rearrangement in a TCR or BCR. The terms such as TCR repertoire and BCR repertoire are used, which are also referred to as, for example, T cell repertoire, B cell repertoire, or the like. For instance, “T cell repertoire” refers to a collection of lymphocytes characterized by expression of a T cell receptor (TCR) serving an important role in antigen recognition. A change in a T cell repertoire provides a significant indicator of an immune status in a disease condition and physiological condition. Thus, a T cell repertoire has been analyzed to identify an antigen specific T cell involved in the development of a disease and diagnosis of abnormality in T lymphocytes. Comparison of the variable region usage by fluorescence activated cell sorter analysis which uses a larger panel of a TCR variable region specific antibody (van den Beemd R et al. (2000) Cytometry 40: 336-345; MacIsaac C et al. (2003) J Immunol Methods 283: 9-15; Tembhare P et al. (2011) Am J Clin Pathol 135: 890-900; Langerak A W et al. (2001) Blood 98: 165-173), polymerase chain reaction (PCR) using multiple primers (Rebai N et al. (1994) Proc Natl Acad Sci USA 91: 1529-1533), or enzyme-linked immunosorbent assay based on PCR (Matsutani T et al. (1997) Hum Immunol 56: 57-69; Matsutani T et al. (2000) Br J Haematol 109: 759-769) have been extensively used to detect a change in a T cell repertoire. Analysis of α chain length distribution known as CDR3 spectratyping is based on the addition of a nontemplate nucleotide in a V-(D)-J region and has been used to assess clonality and diversity of T cells (Matsutani T et al. (2007) Mol Immunol 44: 2378-2387; Matsutani T et al. (2011) Mol Immunol 48: 623-629). To further identify the antigen specificity of a T cell, PCR cloning of a TCR clone type and subsequence sequencing of an antigen recognition region and CDR3 were required. Such a conventional approach is commonly used, but is a time and labor intensive method for studying a TCR repertoire.

As used herein, “polymer of antigen-MHC molecule complexes” refers to multiple complexes of an antigen (e.g., peptide fragment) and MHC molecule that are polymerized. Non-limiting examples of a method of making a polymer of antigen-MHC molecule complexes include complexing an antigen and an MHC molecule to form a monomer antigen-MHC molecule complex, then biotinylating a lysine residue at the C-terminus of the monomer complex, and then forming a tetramer using a biotin-avidin bond. A polymer can be fluorescently labeled for separation with a cell sorter. Examples of polymers of antigen-MHC molecule complexes include, but are not limited to, an epitope dimer, an epitope trimer, an epitope tetramer, an epitope pentamer, and the like.

As used herein, “co-amplification” refers to amplification of two or more nucleic acid molecules at the same occasion. Co-amplification is not intended for only amplification at the same time. Even if molecules are not strictly amplified at the same time, it is sufficient that two or more nucleic acid molecules result in amplification compared to before “co-amplification” at least at a certain point after “co-amplification”. Co-amplification also encompasses amplification of two or more nucleic acid molecules at different times in the same reaction system. Examples of two or more nucleic acid molecules include, but are not limited to, TCRα and TCRβ, TCRδ and TCRγ, a BCR heavy chain and a BCR light chain, and the like.

As used herein, “database” refers to any database related to genes, and particularly to a database comprising T cell receptor and B cell receptor repertoires in the present disclosure. Examples of such databases include, but are not limited to, the IMGT (the international ImMunoGeneTics information system, www.imgt.org) database, DNA Data Bank of Japan (DDBJ, DNA Data Bank of Japan, www.ddbj.nig.ac.jp) database, GenBank (National Center for Biotechnology Information, www.ncbi.nlm.nih.gov/genbank/) database, ENA (EMBL (European Molecular Biology Laboratory), www.ebi.ac.uk/ena) database, and the like.

As use herein, “repertoire of a variable region” refers to a collection of V(D)J regions created in any manner by gene rearrangement in a TCR or BCR.

As used herein, “index sequence” is a sequence for imparting uniqueness so that an amplicon can be identified. Therefore, an index sequence is preferably different from a sequence of interest. Such a sequence is preferable a sequence that does not affect amplification. The baseline of determination and representative examples of an index sequence are the following. Specifically with regard to the baseline of determination of an index sequence, an index sequence is a base sequence added to identify each sample when a plurality of samples are mixed and sequenced simultaneously. A single index sequence is associated with a read derived from a single sample, so that an acquired read sequence can be identified as to which sample the sequence is derived from. An index sequence is any sequence of four types of bases A, C, G, and T. Theoretically, about a million types of sequences can be created with 10 bases, and about a trillion types of sequences can be created with 20 bases. A base sequence length is preferably 2 bases or greater and 40 bases or less, and more preferably 6 bases or greater and 10 bases or less. It is desirable to use a sequence that does not comprise consecutive sequences (AA, CC, GG, or TT) simultaneously.

As used herein, “isotype” refers to IgM, IgA, IgG, IgE, IgD, and the like, which belong to the same type but have different sequences from one another. Isotypes are denoted using various gene abbreviations and symbols.

As used herein, “subtype” is a type within a type in IgA and IgG for BCRs. IgG has IgG1, IgG2, IgG3, and IgG4, and IgA has IgA1 and IgA2. Subtypes are also known to be in β and γ chains for TCRs, having TRBC1 and TRBC2, and TRGC1 and TRGC2, respectively.

As used herein, “homology” of genes refers to the degree of identity of two or more genetic sequences with one another. Generally, having “homology” refers to having a high degree of identity or similarity. Therefore, two genes having higher homology have higher sequence identity or similarity. Whether two genes have homology can be found by direct comparison of sequences, or by hybridization under stringent conditions for nucleic acids. When directly comparing two genetic sequences, the genes are homologous typically when DNA sequences between the genetic sequences are at least 50% identical, preferably at least 70% identical, and more preferably at least 80%, 90%, 95%, 96%, 97%, 98% or 99% identical. Thus, as used herein, “homolog” or “homologous gene product” refers to a protein in another species, preferably a mammal, exerting the same biological function as a protein constituent of a complex, which will be described further herein.

As used herein, “subject” refers to the origin of a sample for analysis of the present disclosure or a target of diagnosis of the present disclosure. Examples of subjects include mammals (e.g., humans, mice, rats, hamsters, rabbits, cats, dogs, cows, horses, sheep, monkeys, and the like), but primates are preferable and humans are particularly preferable.

As used herein, “sample” refers to any substance obtained from a subject or the like, including, for example, nucleic acids (e.g., RNA), cells, tissue, organs, and the like. Those skilled in the art can appropriately select a preferred sample based on the descriptions herein.

As used herein, “means” refers to anything that can be a tool for accomplishing an objective.

As used herein, “diagnosis” refers to identifying various parameters associated with a disease, disorder, condition, or the like in a subject to determine the current or future state of such a disease, disorder, or condition. The condition within the body can be examined by using the method, apparatus, or system of the present disclosure. Such information can be used to select and determine various parameters of a disease, disorder, or condition in a subject, a formulation or method for treatment or prevention to be administered, or the like. As used herein, “diagnosis” when narrowly defined, refers to diagnosis of the current state, but when broadly defined includes “early diagnosis”, “predictive diagnosis”, “prediagnosis”, and the like. Since the diagnostic method of the present disclosure in principle can utilize what comes out from a body and can be conducted away from a medical practitioner such as a physician, the method is industrially useful. In order to clarify that the method can be conducted away from a medical practitioner such as a physician, the term as used herein may be particularly referred to as “assisting” “predictive diagnosis, prediagnosis or diagnosis”.

The formulation procedure for a diagnostic agent or the like of the present disclosure as a medicament or the like is known in the art. The procedure is described, for example, in the Japanese Pharmacopoeia, the United States Pharmacopeia, pharmacopeia of other countries, or the like. Thus, those skilled in the art can determine the amount to be used without undue experimentation with the descriptions herein.

As used herein, “trimming” refers to removal of an unsuitable portion in gene analysis. Trimming is performed by removing low quality regions from both ends of a read, partial sequence of an artificial nucleic acid sequence imparted in an experimental procedure, or both. Trimming can be performed with a software known in the art or by referring to references (for example, cutadapt http://journal.embnet.org/index.php/embnetjournal/article/vie w/200/(EMBnet.journal, 2011); fastq-mcf Aronesty E., The Open Bioinformatics Journal (2013) 7, 1-8 (DOI: 10.2174/1875036201307010001); and fastx-toolkit http://hannonlab.cshl.edu/fastx_toolkit/ (2009)).

As used herein, “suitable length” refers to a length that is compatible with analysis when analyzing an alignment or the like in the gene analysis of the present disclosure. For example, such a length can be determined to be a length including 100 bases toward a D region on a V region from a sequencing initiation position on a C region or greater. In the present disclosure, a suitable length can be, but is not limited to, 200 nucleotides or longer and preferably 250 nucleotides or longer for TCRs and 300 nucleotides or longer and preferably 350 nucleotides or longer for BCRs.

As used herein, “input sequence set” refers to a set of target sequences of TCR or BCR analysis in the gene analysis of the present disclosure.

As used herein, “gene region” refers to each of V region, D region, J region, C region, and the like. Such gene regions are known in the art and can be appropriately determined by referring to a database or the like. As used herein, “homology” of genes refers to the degree of identity of 2 or more genetic sequences to one another. In general, having “homology” refers to having a high degree of identity or similarity. Thus, a higher homology of two genes results in a higher identity or similarity of the sequences thereof. Whether two types of genes are homologous can be examined by direct comparison of sequences or by hybridization under stringent conditions for nucleic acids. As used herein, “homology search” refers to a search for homology. Preferably, homology can be searched in silico by using a computer.

As used herein, “approximate” refers to having a high degree of homology when homology search is performed. A software for homology search (BLAST, FASTA, or the like), when executed, generally lists results in order of high degree of homology. Thus, approximation can be performed by appropriately selecting a result that is highly ranked.

As used herein, “closest” refers to the highest degree of homology when homology search is performed. When homology is searched with software, the result displayed as ranking number one is selected.

As used herein, “reference allele” refers to a reference allele that results in a match in a reference database when homology search is performed.

As used herein, “alignment” (or align) in bioinformatics refers to similar regions of a primary structure of a biomolecule such as DNA, RNA, or protein arranged in alignment to be identifiable or the act of such arranging. Alignment can provide a clue for understanding the functional, structural, or evolutionary relationship of sequences.

As used herein, “assign” refers to allocating specific information such as a gene name, function, or characteristic region (e.g., V region, J region, or the like) to a certain sequence (e.g., nucleic acid sequence, protein sequence, or the like). Specifically, this can be accomplished by inputting or linking specific information to a certain sequence or the like.

As used herein, “CDR3” refers to the third complementarity-determining region (CDR). In this regard, CDR is a region that directly contacts an antigen and undergoes a particularly large change among variable regions, and refers to such a hypervariable region. Each variable region of a light chain and a heavy chain has three CDRs (CDR1 to CDR3) and four FRs (FR1 to FR4) surrounding the three CDRs. Since a CDR3 region is considered to span across the V region, D region and J region, it is considered as an important key for a variable region, and is thus used as a subject of analysis.

As used herein, “front of CDR3 on a reference V region” refers to a sequence corresponding to the front of CDR3 in a V region targeted by the present disclosure.

As used herein, “end of CDR3 on a reference J” refers to a sequence corresponding to the end of CDR3 in a J region targeted by the present disclosure.

As used herein, “condition tolerating random mutations to be scattered throughout” refers to any condition which results in random mutations being scattered. For example, such a condition is often expressed by the following condition for BLAST/FASTA optimal parameters: tolerates a maximum mismatch of 33% across the full length of an alignment; and tolerates a maximum nonconsecutive mismatch of 60% for any 30 bp therein. A functional equivalent such as an isotype of a molecule, e.g. IgG, used in the present disclosure can be found by searching a database or the like. As used herein, “search” refers to utilizing a certain nucleic acid base sequence electronically, biologically, or by another method, preferably electronically, to find another nucleic acid base sequence having a specific function and/or property. Examples of electronic search include, but are not limited to, BLAST (Altschul et al., J. Mol. Biol. 215: 403-410 (1990)), FASTA (Pearson & Lipman, Proc. Natl. Acad. Sci., USA 85: 2444-2448 (1988)), Smith and Waterman method (Smith and Waterman, J. Mol. Biol. 147: 195-197 (1981)), Needleman and Wunsch method (Needleman and Wunsch, J. Mol. Biol. 48: 443-453 (1970)), and the like. BLAST is typically used. Examples of biological search include, but are not limited to, stringent hybridization, a macroarray with a genomic DNA applied to a nylon membrane or the like or a microarray with a genomic DNA applied to a glass plate (microarray assay), PCR, in situ hybridization, and the like. Herein, a gene used in the present disclosure is intended to include corresponding genes identified by such electronic search or biological search.

### Preferred Embodiments

Preferred embodiments of the present disclosure are described below. Embodiments described below are provided to facilitate the understanding of the present disclosure. It is understood that the scope of the present disclosure should not be limited to the following descriptions. Thus, it is apparent that those skilled in the art can make appropriate modifications within the scope of the present disclosure by referring to the descriptions herein. Those skilled in the art can appropriately combine any of the embodiments.

(Analysis Method)

In one aspect, the present disclosure provides a method of analyzing a sequence of functional subunit pair genes of a T cell receptor (TCR) or a B cell receptor (BCR), comprising: (1) providing a nucleic acid sample comprising a nucleic acid of a TCR or a BCR from an activated cell expressing a TCR or a BCR; (2) determining a nucleic acid sequence of the TCR or the BCR; and (3) calculating a frequency of appearance of each gene or a combination thereof based on the determined nucleic acid sequence to identify functional subunit pair genes of TCR or BCR. The method of the present disclosure can obtain sequence information on TCR or BCR functional pair genes from a single cell by using an activated cell. Since conventional analysis technologies (Patent Literatures 1 and 2) use bulk cells, it was unexpected that sequence information on TCR or BCR functional pair genes can be obtained from a single cell with a low absolute amount of TCR/BCR. Sequence information on TCR/BCR pair genes obtained from bulk cells can simply elucidate frequency of appearance of each gene in a plurality of cells, but cannot identify functional TCR/BCR forming pair genes. Meanwhile, information obtained from a single cell is even better in terms of being based on TCR/BCR that is actually functionally expressed. The method of the present disclosure also enables co-amplification of TCR/BCR pair genes. Since TCRα and TCRβ were amplified by separate PCR reactions in the methods disclosed in Patent Literatures 1 and 2, it is unexpected that TCR/BCR pair genes can be co-amplified. In this manner, the method of the present disclosure can obtain more beneficial information (e.g., sequence information on functional pair genes) more quickly and readily relative to conventional technologies.

Functional pair genes are determined with respect to a baseline of whether pair genes (e.g., TCRα and TCRβ or the like) both have a sequence that can express a full length protein. Specifically, pair genes can be determined to be functional pair genes if a sequence of the pair genes has a sequence that can express a full-length protein, with a target CDR3 region connected in-frame by gene rearrangement and no insertion of other stop codon until the stop codon in the C region.

A test for confirming that a resulting sequence of pair genes is functional can be additionally or alternatively performed. When subunit pair genes are simultaneously expressed in lymphocytes or cells that are equivalent thereto in a validation experiment, it is confirmed that the sequence can form a TCR complex/BCR complex localized on the membrane after a protein is synthesized (and optionally subjected to post-translation modification such as glycosylation of BCR), can be secreted as an immunoglobulin when the complex is a BCR complex, and/or can bind to an antigen when the antigen is known.

In one embodiment of the present disclosure, the method can further comprise activating at least a part of the cells expressing a TCR or BCR prior to step (1). In another embodiment, a cell that has been already activated by a third party can be used. An activated cell can be cryopreserved. Activation can be performed by stimulating the cells with any antigen or an antigen MHC complex thereof, or an antigen presenting cell (dendritic cell, macrophage, helper T cell, or the like) having an MHC bound to any antigen. Examples of cells expressing a TCR or BCR include, but are not limited to, T cell or B cell containing blood cells, peripheral blood mononuclear cells, lymphocytes in the bone marrow, lymphocytes within the spleen, lymphocytes within the thymus, lymphocytes within the liver, lymphocytes within the kidney, lymphocytes within other tissue, cancer tissue infiltrated lymphocytes, disease tissue infiltrated lymphocytes, lymphocytes in allergic reactive tissue, and the like.

In a specific embodiment, activation can be performed for 12 hours, 1 day, 2 days, 3 days, 4 days, 5 days, 6 days, 1 week, 10 days, 2 weeks, 3 weeks, or 4 weeks. In a preferred embodiment, activation is performed for any period within the range of 4 to 10 days and is more preferably 7 days. The period of activation can be appropriately changed in accordance with the extent of cell growth or cytokine production.

In one embodiment of the present disclosure, the antigen is selected from, but is not limited to, the group consisting of a virus constituent substance, a microbe constituent substance, a non-autologous cell constituent substance, and a cancer cell specific constituent substance. In a specific embodiment, an antigen can be selected from, but is not limited to, a group consisting of a viral peptide, a viral peptide-glycolipid complex, a microbial peptide, a microbial peptide-glycolipid complex, a non-autologous cell peptide (e.g., animal or plant cell or human cell from a person who is not the donor), a non-autologous cell peptide-glycolipid complex, a cancer cell specific peptide, and a cancer cell specific peptide-glycolipid complex. Many proteins (especially membrane proteins) are modified with a saccharide or lipid. It is conceivable that a substance formed by a saccharide or lipid binding to a protein or peptide would be a target of TCR or BCR as an epitope.

In another embodiment of the present disclosure, activation can be performed by stimulating with an agent selected from the group consisting of an anti-CD3 and/or anti-CD28 antibody, a phospholipase C (PLC) activating agent such as 2,4,6-trimethyl-N-(m-3-trifluoromethylphenyl)benzenesulfonamide (m-3M3FBS), a calcium ionophore such as ionomycin, a protein kinase C (PKC) activating agent such as phorbol myristate acetate (PMA), a phytohemagglutinin (PHA), a concanavalin A (ConA), and a toll-like receptor activating agent. These agents can activate cells in an antigen non-specific manner.

TCR pair/BCR pair sequences in T cells/B cells in a specimen can be uniformly and comprehensively obtained by activating cells in a non-specific manner. A trend of what sequences form a pair can be found by constructing a database of pair gene information obtained from the comprehensive analysis and performing analysis with a computer utilizing AI or the like. This is expected to enable modeling (mathematical expression). It is understood that a database can be constructed for antigen sequence information in addition to pair genes by cloning the uniformly obtained pair genes, inducing proteins to be expressed in cells and purifying, and systematically and comprehensively analyzing binding affinity with a protein or peptide (a method using equipment such as BIACORE equipment or FRET analyzer can be used: manual for analyzing binding affinity, textbook: “Bunshikan Sogosayo Kaiseki Handobukku [Intramolecular Interaction Analysis Handbook], Yodosha, Editors: Toshiaki Isobe, Keiichi Nakayama, Ryuji Ito”, “Tanpakushitsu Jikken Handobukku [Protein Experiment Handbook], Yodosha, Editor: Tadaomi Takenawa”), or by inducing the protein to be expressed in a suitable cell and performing a ligand binding assay or signal activation assay with a protein or ligand (e.g., FLIPR intracellular calcium detection assay or the like). It is expected that a model can be created for such data as to what sequences form a pair and what antigen the pair binds to by performing analysis with a computer utilizing AI or the like. It is understood that a pair gene sequence targeting a specific antigen can ultimately be designed on a computer.

Examples of toll-like receptor activating agents include, but are not limited to, Resiquimod (TLR7 activating agent), lipopolysaccharide (LPS) (TLR4 activating agent), and the like. There are 10 types of toll-like receptors, i.e., TLR1 to TLR10, in humans, with different tissue or cell expression specificities. Activation thereof can activate lymphocytes randomly without going through TCR or BCR of a specific sequence pair. Other toll-like receptor activating agents such as Imiquimod and Gardiquimod (TLR7 activating agent), CpG synthetic oligonucleotide ODN-2006 (sequence: tcgtcgttttgtcgttttgtcgtt, TLR9 activating agent), lipoproteins (TLR1, TLR2, or TLR6 activating agent), peptidoglycan (TLR2 activating agent), lipoteichoic acid (TLR2 activating agent), fungal polysaccharide (TLR2 activating agent), viral glycoprotein (TLR2 or TLR4 activating agent), double stranded RNA (TLR3 activating agent), synthetic nucleic acid poly I/poly C (TLR3 activating agent), flagellin (TLR5 activating agent), single stranded RNA (TLR7 or TLR8 activating agent), non-methylated CpG DNA (TLR9 activating agent), and the like can also be used in the same manner.

In some embodiments of the present disclosure, step (1) can comprise separating the activated cell from an unactivated cell. The separation of the activated cell can be performed with, for example, an antigen-MHC molecule complex or a polymer of 2 to 10,000 antigen-MHC molecule complexes. Examples of the polymer of antigen-MHC molecule complexes include, but are not limited to, an epitope dimer, an epitope trimer, an epitope tetramer, and an epitope pentamer. In a preferred embodiment, the polymer of antigen-MHC molecule complexes is an epitope tetramer. A polymer of antigen-MHC molecule complexes may be labeled. Examples of separation means include, but are not limited to, a cell sorter. An activated cell may be separated further using CD3, CD4, CD8, CD19, CD20, CD24, CD34, CD45, or CD103. A preferable indicator for separating activated T cells is CD8. A preferable indicator for separating activated B cells is CD19.

Step (1) of the present disclosure can be performed by one-step reverse transcription template switching PCR. Specifically, step (1) can comprise a) mixing an RNA of the cells, a reagent required for reverse transcription, a reagent required for template switching, and a reagent required for a polymerase chain reaction, and subjecting the mixture to a reserve transcription inducing condition to provide a cDNA comprising nucleic acid sequences of a plurality of types of TCRs or BCRs; and b) subjecting the cDNA obtained from step a) to a polymerase chain reaction inducing condition to provide a nucleic acid sample comprising the nucleic acid sequences of the plurality of types of TCRs or BCRs. The reagent required for template switching can comprise a template switching oligonucleotide, and the reagent required for a polymerase chain reaction can comprise a primer specific to a C region, a primer specific to a 3′ untranslated region, or a primer spanning a C region and a 3′ untranslated region of the TCR or the BCR, wherein the primer specific to a C region, the primer specific to a 3′ untranslated region, or the primer spanning a C region and a 3′ untranslated region can be a modified oligonucleotide primer designed to partially or completely block a primer function in step a) and to clear blocking of a primer function in step b). One-step reverse transcription template switching PCR is described in detail in International Publication No. WO 2017/222056, which is incorporated herein by reference.

Reverse transcription template switching PCR is a technique that enables RT-PCR amplification using an RNA as a template, even if the sequence of the 5′ terminus of the template RNA is unknown or lacks a common sequence. Reverse transcription template switching PCR utilizes a phenomenon, where a specific short sequence is automatically added to the 3′ terminus of a newly synthesized cDNA by the terminal transferase activity of a reverse transcriptase when the reverse transcriptase reaches the 5′ terminus of a template RNA. For example, a Moloney Murine Leukemia Virus derived reverse transcriptase (MMLV RT) adds a short cytosine-rich sequence (e.g., CC, CCC, or CCCC) to the 3′ terminus of the synthesized cDNA. If an oligonucleotide (template switching oligonucleotide) comprising a nucleotide sequence with a sequence that is complementary to the short added sequence added to the 3′ terminus of a specific anchor sequence (first anchor sequence) is added to a system upon reverse transcription, the template switching oligonucleotide would hybridize to the 3′ terminus of the synthesized cDNA, via the interaction between the sequence added to the 3′ terminus of the cDNA and the complementary sequence of the sequence added to the 3′ terminus of the template switching oligonucleotide, to extend the template for a reverse transcriptase. As a result thereof, a reverse transcriptase, after reaching the 5′ terminus of the template RNA, switches a template to a template switching oligonucleotide and continues cDNA synthesis to the 5′ terminus thereof, so that a sequence that is complementary to the anchor sequence (first anchor sequence) of the template switching oligonucleotide is added to the 3′ terminus of the cDNA. By using an oligonucleotide primer comprising a sequence that is complementary to a specific sequence in a template RNA or an oligonucleotide primer with a specific anchor sequence (second anchor sequence) added to the 5′ terminus (random primer, oligo (dT) primer, or the like) as a reverse transcription primer, the newly synthesized cDNA would also comprise a known sequence at the 5′ terminus. As a result, the PCR amplification using a newly synthesized cDNA as a template is made possible by using a primer set comprising an oligonucleotide primer comprising the known sequence and an oligonucleotide primer comprising at least a part of the first anchor sequence.

In the method of the present disclosure, reverse transcription template switching PCR is performed in “one step (one stage)”. “One-step reverse transcription template switching PCR (RT-TS-PCR)” refers to a method for amplifying a nucleic acid from a reverse transcription reaction, characterized by having all reagents required for reverse transcription, template switching, and PCR mixed as of the start of a reaction and proceeding with a reaction in the same reaction system without adding any additional reagent required for reverse transcription, reagent required for template switching, or reagent required for PCR amplification, and preferably without opening the reaction system (e.g., without opening/closing a tube or adding a reagent).

In the method of the present disclosure, reverse transcription template switching PCR (RT-TS-PCR) can be performed in “semi-one-step”. “Semi-one-step reverse transcription template switching PCR (RT-TS-PCR)” refers to a method of amplifying a nucleic acid from a reverse transcription reaction, characterized by adding, after reverse transcription and template switching reactions, the same buffer used in the reverse transcription and template switching reactions, comprising a reagent (primer or the like) required for PCR amplification, to the same reaction system without exchanging buffer to proceed with a subsequent PCR reaction in the same reaction system.

In one embodiment, the reagent required for template switching can comprise a template switching oligonucleotide. In still another embodiment, a reagent required for a polymerase chain reaction can, but does not need to comprise a 5′ anchor oligonucleotide primer comprising at least a part of an anchor sequence comprised in a template switching oligonucleotide. As demonstrated in the Examples herein, a template switching oligonucleotide (TS-Oligo) can also unexpectedly function as a forward primer in PCR amplification. Therefore, a reagent required for a polymerase chain reaction can be free of the 5′ anchor oligonucleotide primer described above or comprises a smaller amount than an amount that is commonly used.

PCR amplification with high specificity can be achieved even after adding only the modified oligonucleotide primer described above without adding a reverse transcription primer to perform reverse transcription PCR. Although not wishing to be bound by any theory, a part of a modified oligonucleotide primer does not have the function blocked at the time of a reverse transcription reaction, so that a part of the modified oligonucleotide primer whose function is not blocked can function as a reserve transcription primer, or a function of the modified oligonucleotide primer is partially blocked at the time of a reverse transcription reaction, so that the modified oligonucleotide primer whose function is partially blocked can function as a reverse transcription primer in a limited capacity. Therefore in some embodiments, a reagent required for reverse transcription does not need to comprise an oligonucleotide primer that initiates reverse transcription. Even if it is comprised, the oligonucleotide primer that initiates reverse transcription used in this method can be contained at a smaller amount than an amount that is commonly used. In some embodiments, the concentration of the oligonucleotide primer that initiates reverse transcription in the composition described above is, for example, about 40 nM or less, preferably about 20 nM or less, about 10 nM or less, about 2.5 nM or less, about 2.0 nM or less, about 0.63 nM or less, about 0.2 nM or less, about 0.16 nM or less, about 0.02 nM or less, about 2.0 pM or less, about 0.2 pM or less, or about 0.02 pM or less. In another embodiment, the oligonucleotide primer that initiates reverse transcription in the composition described above is comprised at a molar ratio of about 1:10 or less relative to a modified oligonucleotide primer, preferably about 1:20 or less, about 1:40 or less, about 1:160 or less, about 1:200 or less, about 1:635 or less, about 1:2000 or less, about 1:2500 or less, about 1:20,000 or less, about 1:200,000 or less, about 1:2,000,000 or less, or about 1:20,000,000 or less.

In reverse transcription template switching PCR in the method of the present disclosure, at least one of the oligonucleotide primers in PCR has a primer function in reverse transcription that is partially or completely blocked, and the blocking of the primer function is cleared in the nucleic acid amplification step of PCR due to a modification. This can accomplish functional separation of primers that are used in each of the reverse transcription reaction stage and the nucleic acid amplification stage of PCR while being in the same reaction system, and is characterized by significant differentiation in primer concentrations at each reaction stage.

Examples of means for blocking/clearing a primer function include the following approaches. 1) A primer function is blocked at the time of reverse transcription by a primer designed to comprise a thermolabile modifying group or to retain a turn structure in the reverse transcription reaction stage. After a reverse transcription reaction, blocking of a primer function is cleared by detachment of a thermolabile modifying group or dissolution of a turn structure by heating. 2) A primer comprising an artificial base blocks the function as a primer at the reverse transcription reaction stage.

A reverse transcription reaction results in synthesis of a cDNA incorporating a nucleic acid forming a pair with an artificial base contained in a primer from a template RNA by the reverse transcription reaction, and allowing the primer to be annealed to the artificial nucleic acid, thus clearing the blocking of the primer function.

In one-step RT-PCR, all reagents required for reverse transcription of a template RNA into cDNA and all reagents required for PCR using the resulting cDNA as a template are generally included within a reaction system as of the start of reverse transcription. Since Tm of a PCR primer is generally set to 50° C. or higher, specificity of the primer may not be sufficiently exhibited in a temperature zone where reverse transcription can progress (e.g., 42° C.). Further, since the number of copies of a template increases exponentially in PCR, the required PCR primer concentration is dramatically higher than the reverse transcription primer concentration. Therefore, there is a risk of a PCR primer mis-annealing to a template RNA to cause non-specific reverse transcription due to the mis-annealing as the starting point and ultimately producing non-specific PCR products in reverse transcription. In the present disclosure, non-specific reverse transcription can be suppressed by using, as a reverse primer in PCR, a modified oligonucleotide primer, which has a primer function in reverse transcription partially or completely blocked by a modification and acquires a primer function in PCR using the reverse transcription product as a template as a result of the reverse transcription or by thermal denaturation.

As used herein, “oligonucleotide”, “primer”, or “oligonucleotide primer” generally refers to a single stranded polynucleotide. This may be naturally-occurring or synthetic. This is generally comprised of a sequence of about 5 to about 50 nucleotides, more preferably about 10 to about nucleotides, or more preferably about 15 to about 25 nucleotides. Oligonucleotides encompass DNA, RNA, and DNA/RNA chimeras.

As used herein, the term “forward primer” refers to an oligonucleotide primer that anneals to an antisense strand when the template RNA in RT-PCR is a sense strand. “Reverse primer” refers to an oligonucleotide primer that anneals to a sense strand.

In one embodiment, the modified oligonucleotide primer used in the present disclosure comprises a sequence that is complementary to a partial sequence of a template RNA. Although the length of the partial sequence is not particularly limited, the length is generally 10 to 40 bases, preferably 15 to 30 bases, and more preferably 18 to 25 bases. The partial sequence can be a partial sequence of the 3′ terminus of a region intended to be amplified in a template RNA. The modified oligonucleotide primer preferably comprises a sequence that is complementary to a partial sequence of a template RNA at the 3′ terminus thereof. The modified oligonucleotide primer may comprise a sequence added to the 5′ terminus of a sequence that is complementary to a partial sequence of a template RNA. Although the added sequence is not particularly limited, the sequence optimally does not comprise a sequence that is complementary to a partial sequence of a template RNA from the viewpoint of avoiding non-specific hybridization. Examples of the added sequence include specific restriction enzyme recognizing sequences. Although the length of the added sequence is not particularly limited, shorter sequences are preferred in order to avoid non-specific hybridization. The length of the added sequence is generally 1 to 50 bases, preferably 1 to 30 bases, and more preferably 1 to 10 bases. In one embodiment, the modified oligonucleotide primer consists of a sequence that is complementary to a partial sequence of a template RNA without an added sequence.

Exemplary embodiments of modifications in the modified oligonucleotide primer used in the present disclosure include the following:

(1) oligonucleotide primers comprising a thermolabile modifying group;
(2) oligonucleotide primers having one or more complementary regions on a sequence of the same modified oligonucleotide primer and having a folded structure due to the complementary regions prior to initial thermal denaturation processing of PCR to form an intermolecular hairpin loop and exhibit a structure masking a sequence that is complementary to a partial sequence of a template RNA; and
(3) oligonucleotide primers comprising an artificial base.

Each of the embodiments is described in detail below.

### (1) Oligonucleotide Primers Comprising a Thermolabile Modifying Group

In this embodiment, an oligonucleotide primer comprises a thermolabile modifying group, so that a modifying nucleotide primer cannot extend the chain along a polynucleotide to which the primer has hybridized, i.e., cannot extend due to enzyme blocking or a decrease in hybridization to a target nucleic acid. In a preferred embodiment, the 3′ terminus hydroxyl group or one or more internucleotide bonds of an oligonucleotide primer is substituted with a thermolabile modifying group. Therefore, α chain does not extend to a substantial degree unless and until a modification or modified nucleotide is removed. While the modifying group is thermolabile, the group hardly dissociates until reaching the first denaturation temperature in PCR amplification (e.g., about 80 to 105° C., preferably about 85 to 100° C., and more preferably about 90 to 96° C. (e.g., 95° C.)), so that the primer function in reverse transcription is partially or completely blocked. Once the first denaturation temperature is reached, partial or complete dissociation of a modifying group from a modified oligonucleotide primer is thermally induced to convert the modified oligonucleotide primer to a corresponding unmodified oligonucleotide primer. An unmodified oligonucleotide primer has an active phosphodiester bond and can extend with a polymerase.

Examples of oligonucleotide primers comprising a thermolabile modifying group include the modified oligonucleotide primers with a hydroxyl group at the 3′ terminus substituted with a thermolabile modifying group disclosed in U.S. Pat. No. 8,133,669 (the disclosed content is incorporated herein by reference to the same extent as the entirety thereof is explicitly described herein), modified oligonucleotide primers comprising a thermolabile modifying group in one or more internucleotide bonds disclosed in U.S. Pat. No. 8,361,753 (the disclosed content is incorporated herein by reference to the same extent as the entirety thereof is explicitly described herein), and the like.

(1-1) Modified Oligonucleotide Primers with a Hydroxyl Group at the 3′ Terminus Substituted with a Thermolabile Modifying Group (U.S. Pat. No. 8,133,669)

In one embodiment, the modifying group contained at the 3′ terminus of the modified oligonucleotide primer is one of the groups selected from the group consisting of

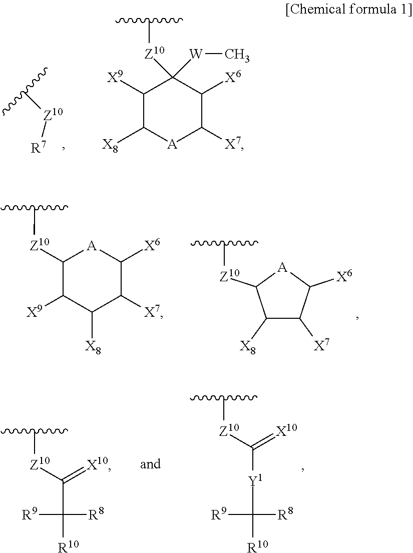

wherein

Z¹⁰ is selected from the group consisting of O, S, and Se;

each R⁷, each R⁸, each R⁹, and each R¹⁰ is independently selected from the group consisting of hydrogen, and a straight or branched optionally substituted hydrocarbyl group having from 1 to 20 carbon atoms, preferably 1 to 10 carbon atoms, and preferably 1 to 6 carbon atoms, wherein the hydrocarbyl is alkyl, alkenyl, or alkynyl which may include at least one substituent selected from the group consisting of halo, oxo, hydroxyl, alkoxy, amino, amido, cycloalkyl, heterocycloalkyl, aryl, aryloxy, and heteroaryl;

each X⁶, each X⁷, each X⁸, and each X⁹ is independently selected from any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, heterocycle, hydrocarbyl, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;

each X¹⁰ is independently selected from the group consisting of O, S, Se, NR¹¹, N—OR¹¹, and CR¹¹R¹²;

each R¹¹ and each R¹² is independently selected from any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, heterocycle, hydrocarbyl, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl; and

each Y¹ is independently selected from the group consisting of O, S, Se, NR⁶, N—OR⁶, and CR⁶R⁷.

In a preferred embodiment, the modifying group is selected from the group consisting of: O-(p-toluene)sulfonate; O-phosphate; O-nitrate; O-[4-methoxy]-tetrahydropyranyl; O-[4-methoxy]-tetrahydrothiopyranyl; O-tetrahydrothiopyranyl; O-[5-methyl]-tetrahydrofuranyl; O-[2-methyl,4-methoxy]-tetrahydropyranyl; O-[5-methyl]-tetrahydropyranyl; O-tetrahydropyranyl; O-tetrahydrofuranyl; O-phenoxyacetyl; O-methoxyacetyl; O-acetyl; O—C(O)—OCH₃; O—C(O)—CH₂CH₂CN; and O—C(S)—OCH₃. In some particularly preferred embodiments, the modifying group is selected from the group consisting of O-methoxytetrahydropyranyl; O-tetrahydropyranyl; and O-tetrahydrofuranyl.

In another embodiment, a modified oligonucleotide primer is a compound represented by Formula V

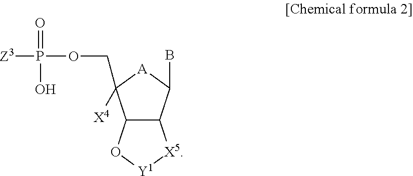

wherein

Z³ is a 3′-O-oligonucleotidyl residue or an oligonucleotide primer;

B is selected from a substituted or non-substituted purine or pyrimidine, any aza or deaza derivative thereof, or any “universal base” or “degenerate base” of any NTP analog which is preferably recognizable by a nucleic acid polymerase;

A is selected from the group consisting of O, S, Se, CR¹R², and NR¹;

each R¹ and each R² is independently selected from the group consisting of H, F, Cl, Br, I, OR³, SR³, NR³R⁴, C(Y)R⁵, substituted or non-substituted alkyl, alkenyl, alkynyl, aryl, and aralkyl, wherein any substituent may each optionally contain one or more heteroatoms;

each Y is independently selected from the group consisting of O, S, Se, CR¹R², and NR¹;

each R³ and each R⁴ is independently selected from the group consisting of H, substituted or non-substituted alkyl, substituted or non-substituted alkenyl, substituted or non-substituted alkynyl, substituted or non-substituted aryl, and substituted or non-substituted aralkyl, wherein any substituent may each optionally contain one or more heteroatoms;

each R⁵ is independently selected from the group consisting of H, F, Cl, Br, OR³, SR³, NR³R⁴, substituted or non-substituted alkyl, substituted or non-substituted alkenyl, substituted or non-substituted alkynyl, substituted or non-substituted aryl, and substituted or non-substituted aralkyl, wherein any substituent may each optionally contain one or more heteroatoms;

X⁴ is independently selected from the group consisting of R¹, F, Cl, Br, I, OR³, SR³, SeR³, NR³R⁴, NR³OR³, NR³—NR³R⁴, CN, N₃, C(Y)R⁵, NO₂, CN, and SSR³;

X⁵ is selected from the group consisting of O, S, Se, NR⁶, N—OR⁶, and CR⁶R⁷;

Y¹ is selected from the group consisting of O, S, Se, NR⁶, N—OR⁶, CR⁶R⁷, and C(Y);

each R⁶ and each R⁷ is independently selected from the group consisting of hydrogen, and a straight or branched optionally substituted hydrocarbyl group having from 1 to 20 carbon atoms, preferably 1 to 10 carbon atoms, and preferably 1 to 6 carbon atoms, wherein the hydrocarbyl is alkyl, alkenyl or alkynyl which may include at least one substituent selected from the group consisting of halo, oxo, hydroxyl, alkoxy, amino, amido, cycloalkyl, heterocycloalkyl, aryl, aryloxy, and heteroaryl; and

X⁵ and Y¹ may each be optionally covalently attached through appropriate atoms or group of atoms to X⁴, X⁵, Z³, A, W, or B portion of the NTP molecule depicted in Formula IB.

In a specific embodiment of Formula V, B is thymine, cytosine, adenine, guanine, uracil, aminoallyl-uracil, 7-deazaguanine, 7-deaza-7-methylguanine, 7-deaza-7-iodoguanine, 7-deaza-7-aminoallyl-guanine, 7-deaza-8-azaguanine, 7-deazadenine, 2,6-diaminopurine, 5-nitro-cytosine, 5-aminoallyl-cytosine, 5-(Biotin-16)-cytosine, 5-(Fluorescein-11)-cytosine, 4-methylamino-cytosine, and 2-thio-5-methyluracil, or 4-thio-5-methyluracil.

In a preferred embodiment of Formula V, B is adenine, guanine, cytosine, thymine, or uracil.

In a preferred embodiment, a modified oligonucleotide primer is one of the compounds selected from the group consisting of:

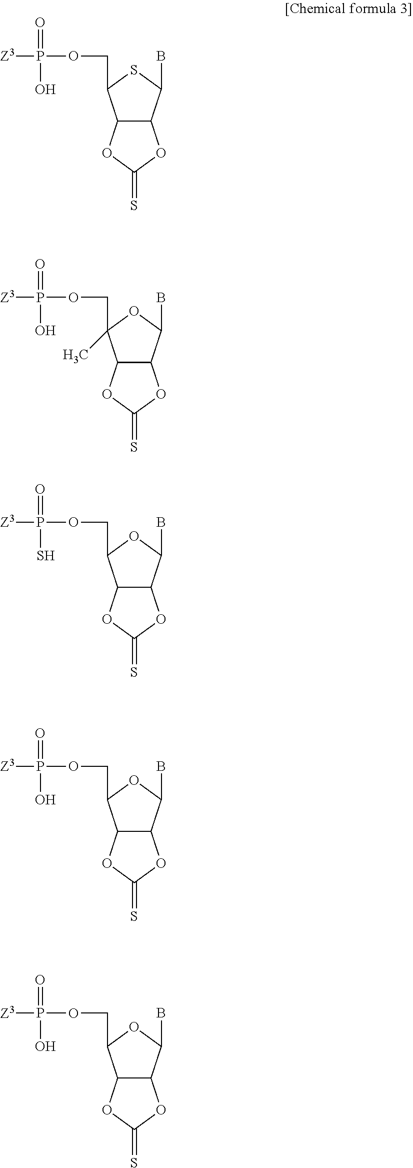

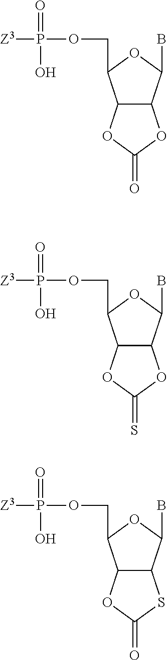

The modified oligonucleotide primer of 1-1 described above can be manufactured by the method described in U.S. Pat. No. 8,361,753.

(1-2) Modified oligonucleotide primers comprising a thermolabile modifying group in one or more internucleotide bonds (U.S. Pat. No. 8,361,753)

In one embodiment, a modifying group in the modified oligonucleotide primer comprises a compound of Formula I:

-L-X—R¹ [Chemical formula 4]

wherein

L is a straight or branched optionally substituted hydrocarbylene group having from 1 to 10 carbon atoms, preferably 2 to 5 carbon atoms, more preferably 3 to 4 carbon atoms, and still more preferably 4 carbon atoms;

X is O, S, S(O), S(O)₂, C(O), C(S), or C(O)NH; and

R¹ is hydrogen or a straight or branched optionally substituted hydrocarbyl group having from 1 to 20 carbon atoms, preferably 1 to 10 carbon atoms, and more preferably 1 to 6 carbon atoms, wherein hydrocarbyl is preferably alkyl, alkenyl or alkynyl which may optionally include at least one substituent selected from the group consisting of halo, oxo, hydroxyl, alkoxy, amino, amido, cycloalkyl, heterocycloalkyl, aryl, aryloxy, and heteroaryl.

In one embodiment, a modifying group provides a compound of Formula Ia:

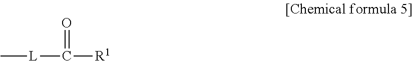

wherein

L is a straight or branched optionally substituted hydrocarbylene group having from 1 to 10 carbon atoms, preferably 2 to 5 carbon atoms, more preferably 3 to 4 carbon atoms, and still more preferably 4 carbon atoms; and

R¹ is hydrogen or a straight or branched optionally substituted hydrocarbyl group having from 1 to 20 carbon atoms, preferably 1 to 10 carbon atoms, and more preferably 1 to 6 carbon atoms, wherein hydrocarbyl is preferably alkyl, alkenyl or alkynyl which may optionally include at least one substituent selected from the group consisting of halo, oxo, hydroxyl, alkoxy, amino, amido, cycloalkyl, heterocycloalkyl, aryl, aryloxy, and heteroaryl.

Preferred embodiments of a modifying group of Formula Ia are the following:

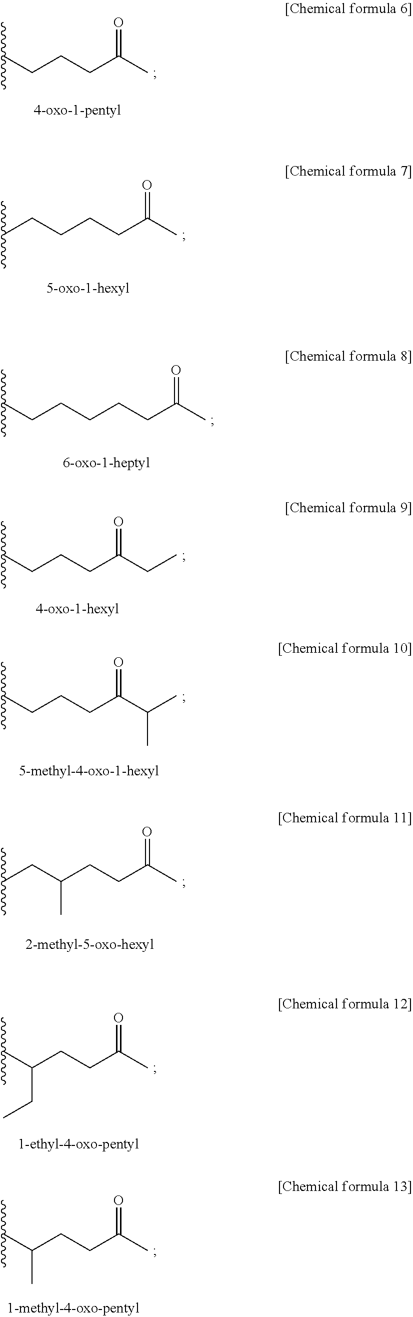

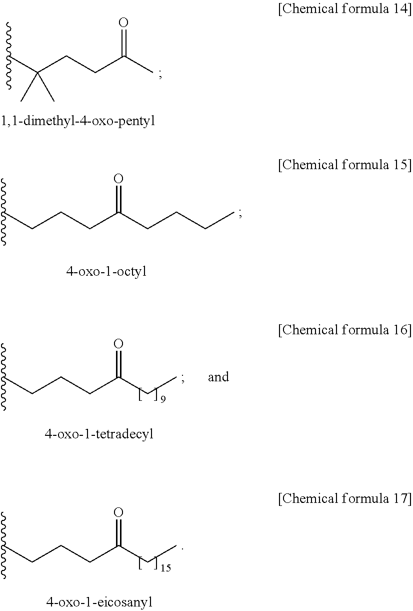

In one embodiment, a modifying group provides a compound of Formula Ib:

-L-S(O)ₖ—R¹ [Chemical formula 18]

wherein

k is an integer from 0 to 2;

L is a straight or branched optionally substituted hydrocarbylene group having from 1 to 10 carbon atoms, preferably 2 to 5 carbon atoms, more preferably 3 to 4 carbon atoms, and still more preferably 4 carbon atoms; and

R¹ is hydrogen or a straight or branched optionally substituted hydrocarbyl group having from 1 to 20 carbon atoms, preferably 1 to 10 carbon atoms, and more preferably 1 to 6 carbon atoms, wherein hydrocarbyl is preferably alkyl, alkenyl or alkynyl which may optionally include at least one substituent selected from the group consisting of halo, oxo, hydroxyl, alkoxy, amino, amido, cycloalkyl, heterocycloalkyl, aryl, aryloxy, and heteroaryl.

In a preferred embodiment, a modifying group of Formula Ib is 4-methylthio-1-butyl described below:

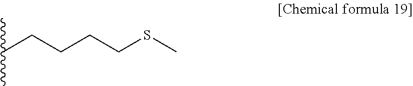 [SMILES: *CCCCSC]

In one embodiment, a modifying group provides a compound of Formula Ic:

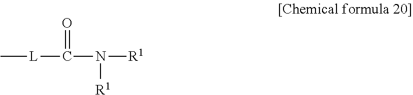

wherein

L is a straight or branched optionally substituted hydrocarbylene group having from 1 to 10 carbon atoms, preferably 2 to 5 carbon atoms, more preferably 3 to 4 carbon atoms, and still more preferably 4 carbon atoms; and

R¹ is hydrogen or a straight or branched optionally substituted hydrocarbyl group having from 1 to 20 carbon atoms, preferably 1 to 10 carbon atoms, and more preferably 1 to 6 carbon atoms, wherein hydrocarbyl is preferably alkyl, alkenyl or alkynyl which may optionally include at least one substituent selected from the group consisting of halo, oxo, hydroxyl, alkoxy, amino, amido, cycloalkyl, heterocycloalkyl, aryl, aryloxy, and heteroaryl.

In a preferred embodiment, a modifying group of Formula Ic is 3-(N-tert-butylcarboxamide)-1-propyl described below:

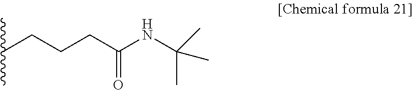 [SMILES: *CCCC(=O)NC(C)(C)C]

In one embodiment, a modifying group provides a compound of Formula Id:

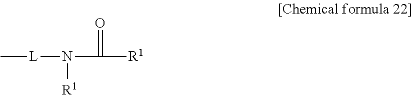

wherein

L is a straight or branched hydrocarbylene group having from 1 to 10 carbon atoms, preferably 2 to 5 carbon atoms, more preferably 3 to 4 carbon atoms, and still more preferably 4 carbon atoms; and

each R¹ is independently hydrogen or a straight or branched optionally substituted hydrocarbyl group having from 1 to 20 carbon atoms, preferably 1 to 10 carbon atoms, and more preferably 1 to 6 carbon atoms, wherein hydrocarbyl is preferably alkyl, alkenyl or alkynyl which may optionally include at least one substituent selected from the group consisting of halo, oxo, hydroxyl, alkoxy, amino, amido, cycloalkyl, heterocycloalkyl, aryl, aryloxy, and heteroaryl.

Examples of a preferred embodiment of a modifying group of Formula Id include 2-(N-formyl-N-methyl)aminoethyl and 2-(N-acetyl-N-methyl)aminoethyl (described below):

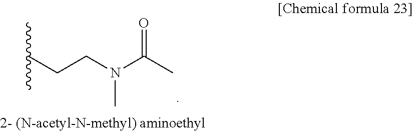 [SMILES: *CCN(C)C(C)=O]

In another embodiment, a modifying group provides a compound of Formula II:

-L-R² [Chemical formula 24]

wherein

L is a straight or branched hydrocarbylene group having from 1 to 10 carbon atoms, preferably 2 to 5 carbon atoms, more preferably 3 to 4 carbon atoms, and still more preferably 4 carbon atoms; and

R² is hydrogen, cyano, or optionally substituted carbocyclic ring, heterocycle, aryl, or heteroaryl having from 5 to 10 atoms.

In a preferred embodiment, a modifying group of Formula II is N-(2-hydroxyethyl)-phthalimide described below:

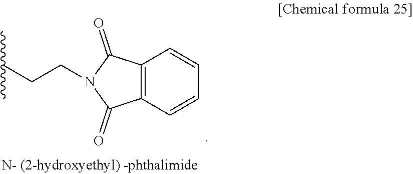 [SMILES: *CCN1C(=O)c2ccccc2C1=O]

In another embodiment, a modifying group provides a compound of Formula III:

-L^{a}-A-L^{b}-B [Chemical formula 26]

wherein

L^{a} and L^{b} are each independently selected from a single bond or a straight or branched optionally substituted hydrocarbylene group having 1 to 8 carbon atoms, preferably 2 to 5 carbon atoms, and more preferably 3 to 4 carbon atoms;

A is O, S, S(O), S(O)₂, Se, CR³R⁴, NR³, C(O), C(S), or CNR³;

B is C(O)R³, C(S)R³, C(O)NR³R⁴, OR³, or SR³; and

R³ and R⁴ are each independently hydrogen or a straight or branched optionally substituted hydrocarbyl group having from 1 to 20 carbon atoms, preferably 1 to 10 carbon atoms, and preferably 1 to 6 carbon atoms, wherein hydrocarbyl is preferably alkyl, alkenyl or alkynyl which may optionally include at least one substituent selected from the group consisting of halo, oxo, hydroxyl, alkoxy, amino, amido, cycloalkyl, heterocycloalkyl, aryl, aryloxy, and heteroaryl.

In another embodiment, a modifying group provides a compound of Formula IV:

-L^{a}-D-L^{b}-E-L^{c}-F [Chemical formula 27]

wherein

L^{a}, L^{b}, and L^{c} are each independently selected from a single bond or a straight or branched optionally substituted hydrocarbylene group having 1 to 8 carbon atoms, preferably 2 to 5 carbon atoms, and more preferably 3 to 4 carbon atoms;

D is O, S, S(O), S(O)₂, CR⁵R⁶, or NRS;

E is O, S, S(O), S(O)₂, CR⁵R⁶, or NR⁶;

F is hydrogen, C(O)R⁷, C(S)R⁷, C(O)NR⁷R⁸, OR⁷, or SR⁷;

R⁵ and R⁶ can each independently be hydrogen, aryl, alkyl, halo, oxo, hydroxyl, alkoxy, aryloxy, or amino, or R⁵ and R⁶ can cooperate to form a mono or bicyclic ring consisting 5 to 10 atoms and including D, R⁵, R⁶, E and L^{b}, provided that when R⁵ and R⁶ cooperate to form a ring, n is from 0 to 2; and

R⁷ and R⁸ are each independently selected from aryl, alkyl, halo, oxo, hydroxyl, alkoxy, aryloxy, amino, amido, optionally substituted cycloalkyl, optionally substituted heterocycloalkyl, optionally substituted aryl, optionally substituted aryloxy, or optionally substituted heteroaryl.

In one embodiment of a compound of Formula IV wherein R⁵ and R⁶ cooperate to form a ring, a modifying group is methoxymethyl-cyclohex-1,3-yl-ethyl described below:

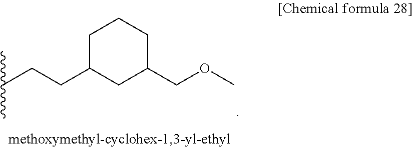 [SMILES: *CCC1CCCC(COC)C1]

In one embodiment, a modified oligonucleotide primer has a modified backbone of Structure I:

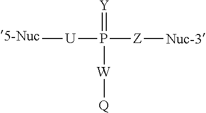

wherein

Nuc is a nucleoside in a primer sequence;

U and Z are independently O, S, Se, NR⁹, or CR⁹R¹⁰;

R⁹ and R¹⁰ are each independently hydrogen or a straight or branched optionally substituted hydrocarbyl having from 1 to 10 carbon atoms; wherein the hydrocarbyl is preferably alkyl, alkenyl or alkynyl which may each independently include at least one substituent selected from halo, oxo, hydroxyl, alkoxy, aryloxy, amino, amido, or a detectable label;

Y is O, S, or Se;

W is any chemical component that enables Q to be thermally cleaved such as O, S, S(O), S(O)₂, Se, C(O), C(S), C(O)NH, C(N)H, NH, —C(═NR¹¹)—, or NR⁹;

R¹¹ is hydrogen or an optionally substituted hydrocarbyl having 1 to 10 carbon atoms, preferably 1 to 6 carbon atoms, wherein R¹¹ is preferably H, alkyl, or lower alkyl; and

Q is a modifying group comprising one or more thermally cleavable groups.

In one embodiment, modifying group Q comprises one or more thermally cleavable groups selected from Formulas I, Ia, Ib, Ic, Id, II, III, and IV described above.

A modified oligonucleotide primer comprises one of the aforementioned modifying groups in at least one internucleotide bonds. A modified oligonucleotide primer preferably comprises one or more of the aforementioned modifying groups at the 3′ terminus thereof. A modified oligonucleotide primer preferably comprises one or more of the aforementioned modifying groups in one of the last six internucleotide bonds, preferably one of the last three internucleotide bonds, at the 3′ terminus thereof.

In another embodiment, an oligonucleotide primer can comprise a sequence with 2, 3, 4, 5, or 6 consecutive modified internucleotide bonds ending at the 3′-terminus of the oligonucleotide primer. In still another embodiment, an oligonucleotide primer may comprise a plurality of non-consecutive 3′ modified internucleotide bonds. The 5′ terminus of the modified oligonucleotide primer may also have a sequence of a nucleotide comprising a modified internucleotide bond. In yet another embodiment, all internucleotide bonds of an oligonucleotide may be modified.

In another preferred embodiment, a modified oligonucleotide primer comprises a modifying group in a 3′ n internucleotide bond of an oligonucleotide primer, wherein n is an internucleotide bond at the 3′ terminus. In yet another embodiment, a modifying group is present in the 3′ n−1, n−2, n−3, or n−4 internucleotide bond of an oligonucleotide. In yet another embodiment, an oligonucleotide has modifying groups at 2 or more of positions n, n−1, n−2, n−3, n−4, n−5, and n−6; preferably 2 or more of positions n, n−1, n−2, n−3, n−4, n−5, and n−6; preferably 3 or more of positions n, n−1, n−2, n−3, n−4, n−5, and n−6; preferably 4 or more of positions n, n−1, n−2, n−3, n−4, n−5, and n−6; preferably 5 or more of positions n, n−1, n−2, n−3, n−4, n−5, and n−6; or preferably 6 or more of positions n, n−1, n−2, n−3, n−4, n−5, and n−6.

The modified oligonucleotide primer of 1-2 can be manufactured by the method described in U.S. Pat. No. 8,361,753.

(2) Oligonucleotide Primers Having One or More Complementary Regions on a Sequence of the Same Modified Oligonucleotide Primer and Having a Folded Structure Due to the Complementary Regions Prior to Initial Thermal Denaturation Processing of PCR to Form an Intermolecular Hairpin Loop and Exhibit a Structure Masking a Sequence that is Complementary to a Partial Sequence of a Template RNA

This embodiment has one or more complementary regions on a sequence of the same modified oligonucleotide primer and has a folded structure due to the complementary regions prior to initial thermal denaturation processing of PCR to form an intermolecular hairpin loop. The complementary regions refer to a set of a first sequence comprised of one or more oligonucleotides and a second sequence comprising one or more oligonucleotides that are complementary thereto.

The first and second sequences may be positioned adjacent to each other or positioned with one or more oligonucleotides interposed therebetween. If the first sequence or the second sequence comprises a sequence that is complementary to a partial sequence of a template RNA, the sequence that is complementary to the partial sequence of the template RNA is masked by a complementary bond of the first and second sequences. Therefore in such a case, the number of oligonucleotides of the first and second sequences is not particularly limited.

If the first and second sequences do not comprise a sequence that is complementary to a partial sequence of a template RNA, a sequence that is complementary to a partial sequence of a template RNA is comprised between oligonucleotides of the first and second sequences, and the sequence that is complementary to the partial sequence of the template RNA is masked by an intermolecular hairpin loop formation.

Since a sequence that is complementary to a partial sequence of a template RNA is masked, it is unable to hybridize to a corresponding partial sequence of the template RNA upon reverse transcription, so that the primer function is partially or completely blocked. However, since a hairpin loop structure dissociates to expose the sequence that is complementary to the partial sequence of the template RNA at a denaturation temperature in PCR amplification (e.g., about to 105° C., preferably about 85 to 100° C., and more preferably about 90 to 96° C. (e.g., 95° C.)), the sequence can hybridize to a corresponding partial sequence in a cDNA at a subsequent pairing temperature (i.e., acquires a primer function). The length of the loop portion of the hairpin loop is generally about 5 to 25 bases. The nucleotide sequence of the loop portion is not particularly limited, as long as an intermolecular hairpin loop can be formed.

### (3) Oligonucleotide Primers Comprising an Artificial Base

The modified oligonucleotide primer of this embodiment comprises an artificial base (non-naturally occurring base), so that the complementary sequence of a nucleotide sequence of the modified oligonucleotide primer is substantially non-existent in a template RNA (template RNA free of an artificial base). For this reason, hybridization of the modified oligonucleotide primer to the template RNA is suppressed, so that the primer function in reverse transcription would be partially or completely blocked. In one embodiment, 1 or more bases, preferably 3 or more bases, or more bases, 10 or more bases, 12 or more bases, or preferably all 15 bases among the 15 bases at the 3′ terminus of the modified oligonucleotide primer are artificial bases. In a preferred embodiment, the base at the most 3′ end of the modified oligonucleotide primer is an artificial base.

The modified oligonucleotide primer in this embodiment is used in combination with an oligonucleotide primer for initiating reverse transcription, comprising a partial sequence comprising an artificial base of the modified oligonucleotide primer. The length of the partial sequence comprising an artificial base is 10 to 40 bases, preferably 15 to 30 bases, and more preferably 18 to 25 bases. A partial sequence comprising an artificial base, while not particularly limited, can be for example a partial sequence of the 3′ terminus of the modified oligonucleotide primer. The oligonucleotide primer for initiating reverse transcription comprises a sequence that is complementary to a partial sequence of a template RNA and the partial sequence comprising the artificial base, and the partial sequence comprising the artificial base is added to the 5′ side of the sequence that is complementary to the partial sequence of the template RNA. The length of the partial sequence of the template RNA is not particularly limited, but is generally 10 to 40 bases, preferably 15 to 30 bases, and more preferably to 25 bases. The partial sequence can be a partial sequence of the 3′ terminus of a region intended to be amplified in the template RNA. An oligonucleotide primer for initiating reverse transcription preferably comprises a sequence that is complementary to a partial sequence of a template RNA at the 3′ terminus thereof.

When such a combination is used to perform one-step reverse transcription template switching PCR, a cDNA with a partial sequence comprising an artificial base of a modified oligonucleotide primer added to the 5′ terminus is synthesized in reverse transcription. The modified oligonucleotide primer comprising an artificial base described above acquires, as a result thereof, a primer function in PCR using the cDNA as a template. In addition, a region of interest can be specifically amplified by PCR amplification using said cDNA as a template and the modified oligonucleotide primer as one of the primers.

Examples of artificial bases include, but are not limited to, Z base/F base (Proc. Natl. Acad. Sci. USA 1997, 94, 105061; Nat. Struct. Biol. 1998, 5, 950; Nat. Struct. Biol. 1998, 5, 954), Q base (J. Am. Chem. Soc. 1999, 121, 2323), iso-G base/iso-C base (J. Am. Chem. Soc. 1989, 111, 8322), 2-thio T (T^{s}) base (Nucleic Acids Res. 2005, 33, 5640), P base/Z base (Nucleic Acids Res. 2007, 35, 4238), PICS base (J. Am. Chem. Soc. 1999, 121, 11585), 5SICS base/MMO2 base/NaM base (J. Am. Chem. Soc. 2009, 131, 14620), 2-amino-6-dimethylaminopurine (x)/2-oxopyridine (y) (Proc. Natl. Acad. Sci. USA 2001, 98, 4922), 2-amino-6-(2-thienyl)purine (s) (J. Am. Chem. Soc. 2005, 127, 17286; Nucleic Acids Res. 2005, 33, e129; Biotechniques 2006, 40, 711), imidazolin-2-one (z) (J. Am. Chem. Soc. 2004, 126, 13298), Ds base/Pa base (Nat. Methods 2006, 3, 729), Pn base (J. Am. Chem. Soc. 2007, 129, 15549), Px base (Nucleic Acids Res. 2009, 37, e14), xA base, xT base (J. Am. Chem. Soc. 2004, 126, 11826), Im-N^{o} base/Na-O^{N} base, Im-O^{N} base/Na-N^{o} base (J. Am. Chem. Soc. 2009, 131, 1644; and Angew. Chem. Int. Ed. 2005, 44, 596), and the like. These artificial bases can contribute to reverse transcription and/or PCR amplification by forming the following base pairs: Z-F base pair, Q-F base pair, isoG-isoC base pair, A-T^{s} base pair, P-Z base pair, PICS-PICS base pair (self-complementary), 5SICS-MMO2 base pair, 5SICS-NaM base pair, x-y base pair, s-y base pair, s-z base pair, Ds-Pa base pair, Ds-Pn base pair, Ds-Px base pair, xA-T base pair, A-xT base pair, Im-N^{o}-Na-O^{N} base pair, and Im-O^{N}-Na-N^{o} base pair.

The method of the present disclosure is described hereinafter in further detail.

The method of the present disclosure first provides a composition comprising all reagents (excluding an oligonucleotide primer that initiates reverse transcription) that are required for template switching reverse transcription of a template RNA into a cDNA, and for PCR amplification of at least a part of the cDNA, including

i) a template switching oligonucleotide,
ii) a primer set consisting of a 5′ anchor oligonucleotide primer comprising at least a part of an anchor sequence comprised in the template switching oligonucleotide, and the modified oligonucleotide primer described above, and
iii) the template RNA.

The template switching oligonucleotide comprises an anchor sequence and a sequence that is complementary to a sequence added to the 3′ terminus of a newly synthesized cDNA (also simply referred to as an RT addition sequence) by the terminal transferase activity of a reverse transcriptase when the reverse transcriptase has reached the 5′ terminus of the template RNA, and an anchor sequence (first anchor sequence) is added to the 5′ terminus of a complementary sequence of the RT addition sequence. Preferably, the complementary sequence of the RT addition sequence is positioned at the 3′ terminus of the template switching oligonucleotide. The RT addition sequence is dependent on the type of reverse transcriptase. For example, a Moloney Murine Leukemia Virus derived reverse transcriptase (MMLV RT) adds a short cytosine-rich sequence (e.g., CC, CCC, or CCCC) to the 3′ terminus of the synthesized cDNA. Thus, a short guanine-rich sequence (e.g., GG, GGG, or GGGG), which is a complementary sequence thereof, is comprised in the template switching oligonucleotide as the complementary sequence of the RT addition sequence. An anchor sequence refers to an artificial sequence that is added to the 5′ terminus of an oligonucleotide. An anchor sequence is preferably a sequence that does not exist in nature. The length of an anchor sequence is not particularly limited, but is generally about 10 bases to 100 bases, and preferably about 15 bases to about 50 bases.

A template switching oligonucleotide may be a DNA or an RNA, or a DNA/RNA chimera. To efficiently function as a template in reverse transcription, a template switching oligonucleotide is optimally an RNA or a DNA/RNA chimera, and more preferably a DNA/RNA chimera. In one embodiment, a part of a complementary sequence of an RT addition sequence is an RNA, and a part of an anchor sequence is a DNA or a DNA/RNA chimera. A template switching oligonucleotide also functions as the 5′ anchor oligonucleotide primer described below. Therefore, in some embodiments, a 5′ anchor oligonucleotide primer can be omitted or added at a small amount.

A 5′ anchor oligonucleotide primer comprises a part or all of the anchor sequence (first anchor sequence) comprised in the template switching oligonucleotide described above. The length of a part or all of the anchor sequence is generally 10 to 40 bases, preferably 15 to 30 bases, and more preferably 18 to 25 bases. The primer is a DNA or a DNA/RNA chimera and preferably a DNA so that it can function as a primer in PCR. A 5′ anchor oligonucleotide primer can be a forward primer in PCR.

Examples of a template RNA that can be used include, but are not limited to, mRNA, rRNA, tRNA, non-coding RNA, chemically synthesized RNA, and the like. The mRNA, rRNA, and tRNA may be derived from any cell or tissue. mRNA, rRNA, and tRNA may be collected from a small amount of cell or tissue (e.g., single cell) obtained by utilizing a cell sorter or the like. mRNA, rRNA, and tRNA may be in a form contained as a part of a total RNA.

The composition described above comprises all of the reagents (excluding an oligonucleotide primer that initiates reverse transcription) that are required for template switching reverse transcription of the template RNA into a cDNA and for PCR amplification of at least a part of the cDNA. Examples of the reagents include, in addition to the aforementioned template switching oligonucleotide, primer set, and template RNA, the following:

*Reverse transcriptase (RNA dependent DNA polymerase)
*Heat resistant DNA polymerase (DNA dependent DNA polymerase)
*dNTPs mixture.

To form an RT addition sequence at the 3′ terminus of a cDNA, a reverse transcriptase that is used has terminal transferase activity. Examples of reverse transcriptases with terminal transferase activity include, but are not limited to, Moloney Murine Leukemia Virus derived reverse transcriptases (MMLV RT). Terminal transcriptase activity is preferably activity of adding a short cytosine-rich sequence (e.g., CC, CCC, or CCCC) to the 3′ terminus of a synthesized cDNA.

Representative examples of heat resistant DNA polymerases include, but are not limited to, Taq, Tth, KOD, Pfu, Bst, and the like. Various heat resistant DNA polymerases that can be used in PCR have been developed, which can all be used in the present disclosure. Heat resistant DNA polymerases that can be used in PCR are well known to, and appropriately selectable by, those skilled in the art.

In one embodiment, the composition described above further comprises an oligonucleotide primer that initiates reverse transcription. An oligonucleotide primer that initiates reverse transcription initiates reverse transcription by hybridizing to a template RNA due to comprising a sequence that is complementary to a partial sequence of a template RNA. The length of the partial sequence is not particularly limited, but is generally 10 to 40 bases, preferably 15 to 30 bases, and more preferably 18 to 25 bases. An oligonucleotide primer that initiates reverse transcription preferably comprises a sequence that is complementary to a partial sequence of a template RNA at the 3′ terminus thereof. An anchor sequence (second anchor sequence) may be added to the 5′ terminus of a sequence that is complementary to a partial sequence of a template RNA. A second anchor sequence is preferably a sequence that does not exist in nature. The length of a second anchor sequence is not particularly limited, but is generally about 10 bases to 100 bases, and preferably about 15 bases to 50 bases. A second anchor sequence is preferably non-identical to the first anchor sequence described above. In one embodiment, a second anchor sequence comprises an artificial base. In one embodiment, an oligonucleotide primer that initiates reverse transcription does not comprise a second anchor sequence. An oligonucleotide primer that initiates reverse transcription is a primer that is specific to a specific gene, an oligo dT primer that binds to a poly-A tail of mRNA, or a random primer such as a random hexamer primer, but is preferably a primer that is specific to a specific gene. Said primer comprises a sequence that is complementary to a partial sequence of an RNA (e.g., mRNA) encoding a gene of interest. An oligonucleotide primer that initiates reverse transcription is a DNA or a DNA/RNA chimera and preferably a DNA so that the primer can function as a primer in reverse transcription.

In one embodiment, a region where an oligonucleotide primer that initiates reverse transcription hybridizes and a region where the modified oligonucleotide primer described above hybridizes on a template RNA at least partially overlap. The length of an overlapping hybridization region is not particularly limited, but is generally 10 bases or greater, preferably 15 bases or greater, and more preferably 18 bases or greater. The length of an overlapping hybridization region can be, for example, 40 bases or less, 30 bases or less, or 25 bases or less.

In a preferred embodiment, the 5′ terminus of a region of a template RNA where a modified oligonucleotide primer hybridizes is positioned closer to the 5′ side (upstream) of the template RNA than the 5′ terminus of a region of the template RNA where an oligonucleotide primer that initiates reverse transcription hybridizes. Specifically, both primers are designed so that the 3′ terminus of the modified oligonucleotide primer hybridizes to the template RNA closer to the 5′ side (upstream) of the template RNA than the 3′ terminus of the oligonucleotide primer that initiates reverse transcription. Improvement in the specificity of amplification can be expected by designing the two primers described above in such a semi-nested positional relationship. In such a case, the region of the template RNA where the oligonucleotide primer that initiates reverse transcription hybridizes and the region of the template RNA where the modified oligonucleotide primer described above hybridizes may be positioned to partially overlap in a semi-nested form, or positioned in a full-nested form without overlap.

When the region of the template RNA where the oligonucleotide primer that initiates reverse transcription hybridizes is to partially overlap the region of the template RNA where the modified oligonucleotide primer described above hybridizes, both primers are preferably designed so that the 5′ terminus of the region of the template RNA where the modified oligonucleotide primer hybridizes is closer to the 5′ side (upstream) of the template RNA than the 5′ terminus of the region of the template RNA where the oligonucleotide primer that initiates reverse transcription hybridizes by, for example, 1 to 12 bases, preferably 1, 2, 3, 4, or 5 bases (i.e., so that the 3′ terminus of the modified oligonucleotide primer hybridizes closer to the 5′ side (upstream) of the template RNA than the 3′ terminus of the oligonucleotide primer that initiates reverse transcription by, for example, 1 to 10 bases, and preferably 1, 2, 3, 4 or 5 bases), but the design is not limited thereto.

In another embodiment, a region of a template RNA where an oligonucleotide primer that initiates reverse transcription hybridizes and a region of the template RNA where the modified oligonucleotide primer described above hybridizes at least partially overlap, and the 5′ terminus of the region of the template RNA where the modified oligonucleotide primer hybridizes matches the 5′ terminus of region of the template RNA where the oligonucleotide primer that initiates reverse transcription hybridizes. Specifically, the 3′ terminus of the modified oligonucleotide primer hybridizes to the template RNA at the same position as the 3′ terminus of the oligonucleotide primer that initiates reverse transcription.

In one embodiment, a region of a template RNA where an oligonucleotide primer that initiates reverse transcription hybridizes and a region of the template RNA where the modified oligonucleotide primer described above hybridizes are identical. In this embodiment, the oligonucleotide primer that initiates reverse transcription can be an unmodified oligonucleotide primer corresponding to the modified oligonucleotide primer described above.

In another embodiment, the modified oligonucleotide primer described above comprises a partial sequence of an oligonucleotide primer that initiates reverse transcription at the 3′ terminus thereof. The length of said partial sequence (hereinafter, also referred to as a common sequence) is not particularly limited, but is generally 10 bases or greater, preferably 15 bases or greater, and more preferably 18 bases or greater. The length of said 3′ terminal partial sequence can be, for example, 40 bases or less, 30 bases or less, or 25 bases or less. In one embodiment, said common sequence can be a partial sequence of the 3′ terminus of an oligonucleotide primer that initiates reverse transcription. In another embodiment, the 3′ terminus of said common sequence is positioned closer to the 5′ side than the 3′ terminus of the oligonucleotide primer that initiates reverse transcription by at least 1 base (e.g., 1 to 20 bases, 1 to 10 bases, or 1 to 8 bases). In one embodiment, said common sequence is a sequence that is complementary to a partial sequence of a template RNA, or a partial sequence thereof, comprised in an oligonucleotide primer that initiates reverse transcription. In one embodiment, said common sequence is a second anchor sequence or a partial sequence thereof. In one embodiment, said common sequence is a partial sequence of an oligonucleotide primer that initiates reverse transcription, which straddles a sequence that is complementary to a partial sequence of a template RNA and a second anchor sequence. In one embodiment, the modified oligonucleotide primer described above is an oligonucleotide primer comprising an artificial base, and an oligonucleotide primer that initiates reverse transcription comprises a second anchor sequence comprising an artificial base at the 5′ terminus, and a common sequence is a second anchor sequence or a partial sequence thereof.

If the composition described above comprises an oligonucleotide primer that initiates reverse transcription, the concentration of the oligonucleotide primer may be an amount that is sufficient for initiating reverse transcription. If one copy of a cDNA comprising a region intended to be amplified can be synthesized, this can be amplified to a detectable level by subsequent PCR. Therefore, the composition (reaction system) only needs to comprise at least one copy, preferably 10 copies or more, and more preferably 100 copies or more of oligonucleotide primer that initiates reverse transcription. If the concentration of the oligonucleotide primer that initiates reverse transcription is too high, secondary reactions due to non-specific hybridization can be induced. The concentration of the oligonucleotide primer that initiates reverse transcription in the composition described above is, for example, about 40 nM or less, preferably about 20 nM or less, about 10 nM or less, about 2.5 nM or less, about 2.0 nM or less, about 0.63 nM or less, about 0.2 nM or less, about 0.16 nM or less, about 0.02 nM or less, about 2.0 pM or less, about 0.2 pM or less, or about 0.02 pM or less.

In another embodiment, the composition described above does not comprise an oligonucleotide primer that initiates reverse transcription. In this embodiment, an oligonucleotide primer comprising a thermolabile modifying group and a sequence that is complementary to a partial sequence of a template RNA is used as the aforementioned modified oligonucleotide primer. The modified oligonucleotide primer preferably comprises a thermolabile modifying group at one or more internucleotide bonds or the 3′ terminus. A thermolabile modifying group comprised in the modified oligonucleotide primer hardly dissociates until reaching the first denaturation temperature (e.g., about 80 to 105° C., preferably about 85 to 100° C., and more preferably about 90 to 96° C. (e.g., 95° C.)) in PCR amplification. Meanwhile, the inventors have found that such a thermolabile modifying group slightly dissociates at a temperature where reverse transcription progresses (e.g., 45° C.), and a corresponding unmodified oligonucleotide generated as a result thereof can function as an oligonucleotide primer that initiates reverse transcription.

The composition described above may comprise a buffer, salt (magnesium ion or the like), or RNAase inhibitor as needed.

The concentration of a template switching oligonucleotide comprised in the composition described above is not particularly limited as long as the method of the present disclosure can be practiced, but is, for example, about 0.05 to 5.0 μM and preferably 0.1 to 1.0 μM.

The concentration of the modified oligonucleotide primer described above and 5′ anchor oligonucleotide primer comprised in the composition described above is equivalent to the primer concentration for conventional PCR, such as about 0.1 to 1.0 μM.

The concentration of other constituents (template RNA, reverse transcriptase, heat resistant DNA polymerase, dNTPs mixture, buffer, salt, and RNAase inhibitor) that can be contained in the composition described above is well known in prior art one-step RT-PCR. The concentration used in the context of the present disclosure can also be optimized from routine experimentation.

Next, the composition provided above is incubated at a temperature where reverse transcription can progress. A temperature at which reverse transcription can progress can be appropriately adjusted depending on the type of reverse transcriptase, but is generally 37° C. to 62° C. and preferably 37° C. to 55° C. Incubation time can be appropriately adjusted while considering the size of a template RNA or the like, but is generally 30 seconds to 120 minutes and preferably 5 minutes to 60 minutes. With the incubation, an oligonucleotide primer that initiates reverse transcription comprised in the composition or an unmodified oligonucleotide generated by dissociation of a thermolabile modifying group from a modified oligonucleotide primer primes reverse transcription to synthesize a cDNA (antisense strand) that is complementary to a template RNA. A reverse transcriptase, after reaching the 5′ terminus of the template RNA, switches the template to a template switching oligonucleotide and continues cDNA synthesis to the 5′ terminus thereof, thus producing a single stranded cDNA (antisense strand) to which a sequence that is complementary to an anchor sequence of the template switching oligonucleotide is added to the 3′ terminus.

Next, a reaction mixture comprising the resulting cDNA is subjected to a plurality of rounds of a thermal cycling protocol with which PCR can progress. A cycle of the thermal cycling protocol is comprised of three temperature steps, i.e., denaturation (also referred to as thermal denaturation), annealing, and extension. Denaturation is not particularly limited, as long as the temperature is sufficient for dissociating a double stranded DNA. The preferred lower limit and upper limit of the thermal denaturation temperature are 90° C. and 100° C., respectively. Annealing is a step of annealing a primer to a dissociated DNA. The temperature in this step (annealing temperature) is not particularly limited, but the lower limit of the annealing temperature is preferably 45° C. and more preferably 50° C. Meanwhile, the upper limit is preferably 75° C. and more preferably 70° C. Extension is a step of synthesizing a complementary strand with a DNA polymerase. The temperature in this step (extension temperature) is not particularly limited, but the lower limit and the upper limit of a preferred extension temperature are 50° C. and 80° C., respectively. In this cycle, the annealing temperature does not exceed the extension reaction temperature. The annealing and extension can also be performed at one temperature to configure a thermal cycling protocol as a cycle of substantially two temperature steps. In such a case, the lower limit of a temperature for annealing and extension is preferably 50° C. and more preferably 55° C. Meanwhile, the upper limit is preferably 70° C. and more preferably 65° C. Examples of incubation time in each step include 1 second to 5 minutes, but those skilled in the art can readily determine a suitable incubation time while considering the size of amplicon or the like.

A denaturation step (pre-incubation step) may be performed to inactivate a reverse transcriptase before subjecting a reaction mixture to a thermal cycling protocol. The denaturation temperature is not particularly limited as long as a reverse transcriptase can be inactivated, but the lower limit and the upper limit of a preferred thermal denaturation temperature are 90° C. and 100° C., respectively. The denaturation time is not particularly limited as long as a reverse transcriptase can be inactivated, but is generally 1 minute to 15 minutes.

If an oligonucleotide primer comprising a thermolabile modifying group is used as a modified oligonucleotide primer, a modifying group is dissociated from the modified oligonucleotide primer and the primer is converted to a corresponding unmodified oligonucleotide primer in the first denaturation step or pre-incubation step of a thermal cycling protocol. An unmodified oligonucleotide primer has an active phosphodiester bond and can prime the extension by a polymerase.

In the first annealing and extension steps of thermal cycling, a 5′ anchor oligonucleotide primer is annealed to a sequence that is complementary to an anchor sequence at the 3′ end of a single stranded cDNA (antisense strand) obtained in the reverse transcription step, leading to extension due to a polymerase and synthesis of a cDNA (sense strand) in which an anchor sequence (first anchor sequence) is added to the 5′ terminus. As a result, a double stranded cDNA in which an anchor sequence is added to the 5′ terminus of a sense strand is produced.

In addition, a reaction mixture comprising the double stranded cDNA described above is subsequently subjected to a plurality of rounds of thermal cycling protocol to amplify a region sandwiched by a 5′ anchor oligonucleotide primer and a modified oligonucleotide primer (i.e., from the 5′ terminal anchor sequence to the region where the modified oligonucleotide primer hybridizes).

The number of rounds of thermal cycling can be appropriately determined while considering the amount of template RNA or the like, but is for example 20 rounds or more, and preferably 30 rounds or more, 40 rounds or more, 45 rounds or more, 50 rounds or more, or 55 rounds or more. In a common RT-PCR, even with a low number of copies of template RNA (e.g., single copy), an amplification reaction reaches saturation after about 40 rounds of thermal cycling. Meanwhile in the method of the present disclosure (especially when using a modified oligonucleotide primer comprising a thermolabile modifying group), an amplification reaction does not reach saturation even after 45 rounds or more, 50 rounds or more, or 55 rounds or more of thermal cycling, so that further amplification can be possible. Although not wishing to be bound by any theory, the amplification efficiency per a round of thermal cycle can potentially be reduced relative to common RT-PCR in the method of the present disclosure (especially when using a modified oligonucleotide primer comprising a thermolabile modifying group). Thus, when the number of copies of a template RNA intended to be amplified is low (e.g., 100 copies or less, 10 copies or less, or a single copy), or when the method of the present disclosure is performed using an RNA (especially total RNA) isolated from a single cell as a template RNA, the number of rounds of thermal cycling is preferably 40 rounds or more, 45 rounds or more, 50 rounds or more, or 55 rounds or more.

The method of the present disclosure can be expected to perform reverse transcription template switching PCR with high specificity in one step. The specificity of reverse transcription template switching PCR can be substantially determined by only a reverse primer, but the present disclosure can amplify a gene of interest with high specificity by employing the aforementioned modified oligonucleotide primer as a reverse primer. Especially when the number of copies of a template RNA is low (e.g., when RNA from a single cell is used as a template), a specific PCR product can be expected to be amplified while minimizing secondary reactions even when the number of PCR cycles is increased. Therefore, a reverse primer that is specific to a constant region of an antigen receptor (e.g., antibody (heavy chain or light chain) or T cell receptor (α chain, β chain, γ chain, or δ chain) can be used as the aforementioned modified oligonucleotide primer to perform sequence analysis of an antigen recognition site of the antigen receptor at a single cell level.

In some embodiments, the reagent required for reverse transcription used in the present disclosure can comprises the following in addition to a reverse transcriptase: (i) an oligonucleotide primer that initiates reverse transcription which is complementary to a C region, a 3′ untranslated region, or a region spanning a C region and a 3′ untranslated region of TCRα and an oligonucleotide primer that initiates reverse transcription which is complementary to a C region, a 3′ untranslated region, or a region spanning a C region and a 3′ untranslated region of TCRβ; (ii) an oligonucleotide primer that initiates reverse transcription which is complementary to a C region, a 3′ untranslated region, or a region spanning a C region and a 3′ untranslated region of TCRδ and an oligonucleotide primer that initiates reverse transcription which is complementary to a C region, a 3′ untranslated region, or a region spanning a C region and a 3′ untranslated region of TCRγ; or (iii) an oligonucleotide primer that initiates reverse transcription which is complementary to a C region, a 3′ untranslated region, or a region spanning a C region and a 3′ untranslated region of a BCR heavy chain and an oligonucleotide primer that initiates reverse transcription which is complementary to a C region, a 3′ untranslated region, or a region spanning a C region and a 3′ untranslated region of a BCR light chain.

In one embodiment of the present disclosure, a reagent required for a polymerase chain reaction can optionally further comprise a 5′ anchor oligonucleotide primer comprising at least a part of an anchor sequence contained in the template switching oligonucleotide.

In one embodiment of the present disclosure, a reagent required for a polymerase chain reaction is free of the 5′ anchor oligonucleotide primer, and the template switching oligonucleotide can function as a 5′ anchor oligonucleotide primer.

Functional subunit pair genes can be genes of TCRα and TCRβ, TCRδ and TCRγ, or a BCR heavy chain and a BCR light chain. Therefore, in some embodiments of the present disclosure, the reagent required for reverse transcription can be: (i) an oligonucleotide primer that initiates reverse transcription which is complementary to a C region, a 3′ untranslated region, or a region spanning a C region and a 3′ untranslated region of TCRα and an oligonucleotide primer that initiates reverse transcription which is complementary to a C region, a 3′ untranslated region, or a region spanning a C region and a 3′ untranslated region of TCRβ; (ii) an oligonucleotide primer that initiates reverse transcription which is complementary to a C region, a 3′ untranslated region, or a region spanning a C region and a 3′ untranslated region of TCRδ and an oligonucleotide primer that initiates reverse transcription which is complementary to a C region, a 3′ untranslated region, or a region spanning a C region and a 3′ untranslated region of TCRγ; or (iii) an oligonucleotide primer that initiates reverse transcription which is complementary to a C region, a 3′ untranslated region, or a region spanning a C region and a 3′ untranslated region of a BCR heavy chain and an oligonucleotide primer that initiates reverse transcription which is complementary to a C region, a 3′ untranslated region, or a region spanning a C region and a 3′ untranslated region of a BCR light chain. The reagent required for a polymerase chain reaction can comprise: (i) a primer specific to a C region, a primer specific to a 3′ untranslated region, or a primer spanning a C region and a 3′ untranslated region of TCRα and a primer specific to a C region, a primer specific to a 3′ untranslated region, or a primer spanning a C region and a 3′ untranslated region of TCRβ; (ii) a primer specific to a C region, a primer specific to a 3′ untranslated region, or a primer spanning a C region and a 3′ untranslated region of TCRδ and a primer specific to a C region, a primer specific to a 3′ untranslated region, or a primer spanning a C region and a 3′ untranslated region of TCRγ; or (iii) a primer specific to a C region, a primer specific to a 3′ untranslated region, or a primer spanning a C region and a 3′ untranslated region of a BCR heavy chain and a primer specific to a C region, a primer specific to a 3′ untranslated region, or a primer spanning a C region and a 3′ untranslated region of a BCR light chain.

Surprisingly, the method of the present disclosure can co-amplify TCR or BCR pair genes, so that each gene does not need to be amplified in separate reactions. Therefore, the method of the present disclosure is characterized in that both TCRα and TCRβ, both TCRδ and TCRγ, or both a BCR heavy chain and a BCR light chain are co-amplified.

In a specific embodiment of the present disclosure, the modified oligonucleotide primer can have one or more complementary regions on a sequence of the same modified oligonucleotide primer and have a folded structure due to the complementary regions prior to initial thermal denaturation processing of PCR, or comprise a thermolabile modifying group. Further, a part of a modified oligonucleotide whose primer function has not been blocked can function as an oligonucleotide primer that initiates reverse transcription by hybridizing to a template RNA.

The step of (2) determining a nucleic acid sequence of functional pair genes of the TCR or the BCR can use any sequencing method that is known in the art. Various sequencing methods are available as shown in the Examples. The method of the present disclosure is advantageous in that a next generation sequencer can be applied. A typical sequencing method is described hereinafter.

The method of the present disclosure may further comprise a step of providing a nucleic acid sample to which a sequence that is suitable for sequence analysis is further added after performing reverse transcription template switching PCR. Those skilled in the art can select a sequence that is suitable for sequence analysis depending on the type of sequence analysis. Examples of a sequence that is suitable for sequence analysis include, but are not limited to, sequences that are suitable for sequence analysis using bridge PCR or emulsion PCR. Suitable sequences added for sequence analysis using bridge PCR include index sequences, tag sequences, sequences for immobilization to a substrate (e.g., flow cell) of sequence analysis, and the like.

In a preferred embodiment, the analysis method of the present disclosure can perform the reverse transcription template switching PCR described above (first PCR amplification reaction), tag PCR (second PCR amplification reaction), and index PCR (third PCR amplification reaction) to provide a nucleic acid sample for sequence analysis.

A 5′ anchor oligonucleotide primer used in reverse transcription template switching PCR (first PCR amplification reaction) is also referred to as a first 5′ anchor oligonucleotide primer. A first 5′ anchor oligonucleotide primer is an oligonucleotide primer comprising at least a part of an anchor sequence comprised in a template switching oligonucleotide. A first PCR amplification reaction does not need to use the first 5′ anchor oligonucleotide primer described above because a template switching oligonucleotide also functions as the 5′ anchor oligonucleotide primer.

In one embodiment, step (2) for determining a nucleic acid sequence of functional pair genes of the TCR or the BCR may further comprise c) subjecting a mixture comprising a PCR amplicon of reverse transcription template switching PCR, a second 5′ anchor oligonucleotide primer to which a first tag sequence is added, and a second primer specific to a C region, primer specific to a 3′ untranslated region, or primer spanning a C region and a 3′ untranslated region of a TCR or a BCR to which a second tag sequence is added to a polymerase chain reaction inducing condition to provide a nucleic acid sample comprising nucleic acid sequences of a plurality of types of T cell receptors (TCR) or B cell receptors (BCR) to which a tag sequence is added; and d) subjecting a mixture comprising a PCR amplicon of step c), a third 5′ anchor oligonucleotide primer, and a third primer specific to a C region, primer specific to a 3′ untranslated region, or primer spanning a C region and a 3′ untranslated region of a TCR or a BCR to a polymerase chain reaction inducing condition to provide a nucleic acid sample comprising nucleic acid sequences of plurality of types of T cell receptors (TCR) or B cell receptors (BCR) to which an index sequence is added, wherein a sequences for immobilization to a substrate of sequence analysis and an index sequence are added to the third 5′ anchor oligonucleotide primer and the third primer specific to a C region, primer specific to a 3′ untranslated region, or primer spanning a C region and a 3′ untranslated region of a TCR or a BCR.

A tag sequence is a sequence used for sequencing (e.g., sequencing by Miseq). Sequencing is initiated from a tag sequence. A tag sequence also may function as a priming site of index PCR (third PCR amplification reaction) for adding an index sequence. A first tag sequence can be a sequence added to a second 5′ anchor oligonucleotide primer, and a second tag sequence can be a sequence added to a second primer specific to a C region, primer specific to a 3′ untranslated region, or primer spanning a C region and a 3′ untranslated region of a TCR or a BCR.

As used herein, “first primer specific to a C region, primer specific to a 3′ untranslated region, or primer spanning a C region and a 3′ untranslated region of a TCR or a BCR” refers to a primer used in a PCR amplification reaction by reverse transcription template switching PCR of the present disclosure, comprising a sequence that is specific to a C region, a 3′ untranslated region, or a region spanning a C region and a 3′ untranslated region of a TCR or a BCR.

As used herein, “second primer specific to a C region, primer specific to a 3′ untranslated region, or primer spanning a C region and a 3′ untranslated region of a TCR or a BCR” refers to a primer used in a PCR amplification reaction (second PCR amplification reaction) for providing a nucleic acid sample comprising a nucleic acid sequence of TCR or BCR to which a tag is added, comprising a sequence that is specific to a C region, a 3′ untranslated region, or a region spanning a C region and a 3′ untranslated region of a TCR or a BCR. A tag sequence that is a priming site for a third PCR amplification reaction is added to a second primer specific to a C region, primer specific to a 3′ untranslated region, or primer spanning a C region and a 3′ untranslated region of a TCR or a BCR.

As used herein, “third primer specific to a C region, primer specific to a 3′ untranslated region, or primer spanning a C region and a 3′ untranslated region of a TCR or a BCR” refers to a primer used in a PCR amplification reaction (third PCR amplification reaction) for providing a nucleic acid sample comprising a nucleic acid sequence of TCR or BCR to which an index sequence is added, comprising a sequence that is specific to a C region, a 3′ untranslated region, or a region spanning a C region and a 3′ untranslated region of a TCR or a BCR. A sequence for immobilization to a substrate of sequence analysis and an index sequence are optionally added to a third primer specific to a C region, primer specific to a 3′ untranslated region, or primer spanning a C region and a 3′ untranslated region of a TCR or a BCR.

As used herein, “first 5′ anchor oligonucleotide primer” is a primer used in a PCR amplification reaction by reverse transcription template switching PCR of the present disclosure. Since a template switching oligonucleotide can also function as a 5′ anchor oligonucleotide primer, a first 5′ anchor oligonucleotide primer can be unused, or used in a small quantity.

As used herein, “second 5′ anchor oligonucleotide primer” is a primer used in a PCR amplification reaction (second PCR amplification reaction) for providing a nucleic acid sample comprising a nucleic acid sequence of TCR or BCR to which a tag sequence is added.

As used herein, “third 5′ anchor oligonucleotide primer” is a primer used in a PCR amplification reaction (third PCR amplification reaction) for providing a nucleic acid sample comprising a nucleic acid sequence of TCR or BCR to which an index sequence is added. A sequence for immobilization to a substrate of sequence analysis and an index sequence are optionally added to a third 5′ anchor oligonucleotide primer.

As used herein, “first PCR amplification reaction” is a PCR amplification reaction performed using RNA obtained from a subject by the one-step reverse transcription template switching PCR of the present disclosure as a template.

As used herein, “second PCR amplification reaction” is a PCR amplification reaction for providing a nucleic acid sample comprising a nucleic acid sequence of TCR or BCR to which a tag sequence is added, the PCR amplification reaction using a product of a first PCR amplification reaction as a template in producing a sample for the analysis of the present disclosure. In a second PCR amplification reaction, a nucleic acid sample to which a tag sequence for providing a priming site for a third PCR amplification reaction is added is provided. Such a tag sequence is also a starting point in sequence analysis.

As used herein, “third PCR amplification reaction” is a PCR amplification reaction using an amplicon of a second PCR amplification reaction as a template in producing a sample for the analysis of the present disclosure, a product thereof comprising a sequence that is suitable for use in the sequence analysis of the present disclosure. A sequence for immobilization to a substrate of sequence analysis and an index sequence are added in a third PCR amplification reaction.

In still another embodiment of the present disclosure, step (3) can comprise: (3-1) providing a reference database for each of gene regions comprising at least one of a V region, a D region, a J region, and optionally a C region; (3-2) providing an input sequence set prepared from optionally trimming and optionally extracting a sequence with a suitable length; (3-3) searching for homology of the input sequence set with the reference database for each of the gene regions and recording an alignment with an approximate reference allele and/or a sequence of the reference allele; (3-4) assigning the V region and the J region for the input sequence set and extracting a nucleic acid sequence of the D region based on a result of assigning; (3-5) translating the nucleic acid sequence of the D region into an amino acid sequence and classifying the D region by utilizing the amino acid sequence; and (3-6) calculating a frequency of appearance or a combination for each of the V region, the D region, the J region, and optionally the C region based on the classifying in step (3-5) to identify functional subunit pair genes of a TCR or a BCR.

In one embodiment where analysis is performed on functional subunit pair genes of a BCR, the primer specific to a C region, primer specific to a 3′ untranslated region, or primer spanning a C region and a 3′ untranslated region comprises a sequence that is a complete match with a C region, 3′ untranslated region, or region spanning a C region and a 3′ untranslated region of an isotype of interest selected from the group consisting of IgM, IgA, IgG, IgE, and IgD and has a sequence that is not homologous with other C regions or 3′ untranslated regions. Preferably, the primer specific to a C region, primer specific to a 3′ untranslated region, or primer spanning a C region and a 3′ untranslated region is a sequence that is a complete match with one of the subtypes IgG1, IgG2, IgG3, and IgG4 or one of IgA1 and IgA2 for IgA or IgG. In another embodiment where analysis is performed on functional subunit pair genes of a TCR or a BCR, the primer specific to a C region, primer specific to a 3′ untranslated region, or primer spanning a C region and a 3′ untranslated region is a sequence that is a complete match with a C region, a 3′ untranslated region, or a region spanning a C region and a 3′ untranslated region of α chain of interest selected from the group consisting of α chain, β chain, γ chain, and δ chain and is not homologous with other C regions.

In another embodiment, it is preferable that a part of a sequence that is a complete match with all allelic sequences of C region, 3′ untranslated region, or region spanning a C region and a 3′ untranslated region of the same isotype in the database is selected for the primer specific to a C region, primer specific to a 3′ untranslated region, or primer spanning a C region and a 3′ untranslated region. Such selection of a complete match enables highly accurate analysis.

Gene analysis can be performed using any analytic approach. For example, it is possible to use an approach of assigning V, D, J, and C sequences of each read sequence by using V, D, J, and C sequences obtained from a known IMGT (the international ImMunoGeneTics information system, http://www.imgt.org) database as a reference sequence and utilizing IMGT's HighV-Quest, or a new software (Repertoire Genesis) developed by the Applicant, which is described herein as a preferred example of an analysis system.

Different sequences can be distinguished by sequencing individual amplified molecules. Thus, sequencing has sensitivity to detect a quantitative change in clone proliferation. In summary, one provided embodiment of the present disclosure provides a method of determining a profile of a recombinant DNA sequence in a T cell and/or B cell. This method can comprise the steps of: isolating a sample from a subject; performing one or more rounds of nucleic acid amplification and spatially isolation of individual nucleic acids; and sequencing the nucleic acids.

(Analysis System)

In another aspect, the present disclosure provides a system for analyzing a sequence of functional subunit pair genes of a T cell receptor (TCR) or a B cell receptor (BCR) in cells expressing a TCR or a BCR, comprising: (A) an activator for activating at least a part of the cells; (B) a nucleic acid treating kit for providing a nucleic acid sample comprising a nucleic acid sequence of a TCR or a BCR obtained from the activated cell; (C) a sequencer for determining a nucleic acid sequence contained in the activated cell; and (D) an analyzer for calculating a frequency of appearance of each gene and/or a combination thereof based on the determined nucleic acid sequence to identify functional subunit pair genes of a TCR or a BCR. Constituent elements that materialize any specific embodiment described in (Analysis method) can be utilized as the constituent elements of the analysis system that can be used herein.

Specifically, an activator that materializes any specific embodiment described in (Analysis method) can be utilized as (A) an activator for activating at least a part of the cells. A kit that materializes any specific embodiment described in (Analysis method) can be utilized as (B) a nucleic acid treating kit for providing a nucleic acid sample comprising a nucleic acid sequence of a TCR or a BCR obtained from the activated cell, and the kit may comprise any reagent or the like for obtaining a nucleic acid from a cell. Any sequencer that determines a sequence can be utilized as (C) a sequencer for determining a nucleic acid sequence contained in the activated cell. A sequencer that materializes any specific embodiment described in (Analysis method) can be utilized such as the so-called next generation sequencer. The gene identification method of the present disclosure can be appropriately performed by referring to documents such as “Jisedai Sikuensa: Mokuteki Betsu Adobansu Mesoddo [Next Generation Sequencer: Advanced Method by Objective]” Cell Technology extra issue or Cold Spring Harb Protoc. 2015 November; 2015 (11): 951-969. An analyzer that materializes any specific embodiment described in (Analysis method) can be utilized as (D) an analyzer for calculating a frequency of appearance of each gene or a combination thereof based on the determined nucleic acid sequence to identify functional subunit pair genes of a TCR or a BCR. Any computer comprising a CPU that materializes calculating a frequency of appearance of each gene or a combination thereof based on the determined nucleic acid sequence to identify functional subunit pair genes of a TCR or a BCR or the like can be utilized.

Gene expression can be measured using any method that is known in the art. Examples of technologies for measuring the amount of gene expression include microarray, RNA-Seq, quantitative PCR, and the like. Preferably, RNA-Seq can be employed. The sequencing methodology is not limited and any method known in the art can be utilized, as long as a nucleic acid sample can be sequenced, but next generation sequencing (NGS) is preferably used. Examples of next generation sequencing include, but are not limited to, pyro sequencing, sequencing by synthesis (sequencing biosynthesis), sequencing by ligation, ion semiconductor sequencing, and the like.

In the method of the present disclosure, (1) providing a reference database for each of gene regions comprising at least one of a V region, a D region, a J region, and optionally a C region can be accomplished, for example for the V region, by appropriately selecting and providing a database comprising information on the V region.

In the method of the present disclosure, (2) providing an input sequence set prepared from optionally trimming and optionally extracting a sequence with a suitable length is accomplished by optionally trimming using a function of an appropriate software or the like and optionally selecting a length appropriately to provide an extracted input sequence set. An input sequence can be, for example, a set of amplicons amplified by a known method or a set of amplicons amplified by PCR by the reverse transcription template switching PCR of the present disclosure.

The method of the present disclosure performs (3) searching for homology of the input sequence set with the reference database for each of the gene regions and recording an alignment with an approximate reference allele and/or a sequence of the reference allele by appropriately using a software for performing homology search to perform search for homology of the input sequence set with a reference database for each of gene regions (for example, the V region and the like), and recording alignment with an approximate reference allele and/or a sequence of the reference allele obtained as a result.

In the method of the present disclosure, (4) assigning the V region and the J region for the input sequence set and extracting a nucleic acid sequence of the D region based on a result of assigning can be accomplished by determining a V region and/or J region based on known information or the like from a sequence alignment. Such extraction can be accomplished preferably by assigning the V region and the J region for the input sequence set and extracting a CDR3 sequence, with the front of CDR3 on a reference V region and end of CDR3 on reference J as guides.

In the method of the present disclosure, (5) translating the nucleic acid sequence of the D region into an amino acid sequence and classifying the D region by utilizing the amino acid sequence can be accomplished by translating into an amino acid using a known method in the art and picking out a sequence corresponding to the D region by homology search or the like on the amino acid sequence. Preferably, the nucleic acid sequence of the CDR3 can be translated into an amino acid sequence and the D region can be classified by utilizing the amino acid sequence.

In the method of the present disclosure, (6) calculating a frequency of appearance or a combination for each of the V region, the D region, and the J region, and optionally the C region based on the classifying in (5) to deduce functional subunit pair genes of a TCR or a BCR can calculate a frequency of appearance of the V region, D region, J region and/or the C region calculated in the above steps by, for example, organizing the frequencies into a list. Functional subunit pair genes of a TCR or a BCR can be deduced thereby.

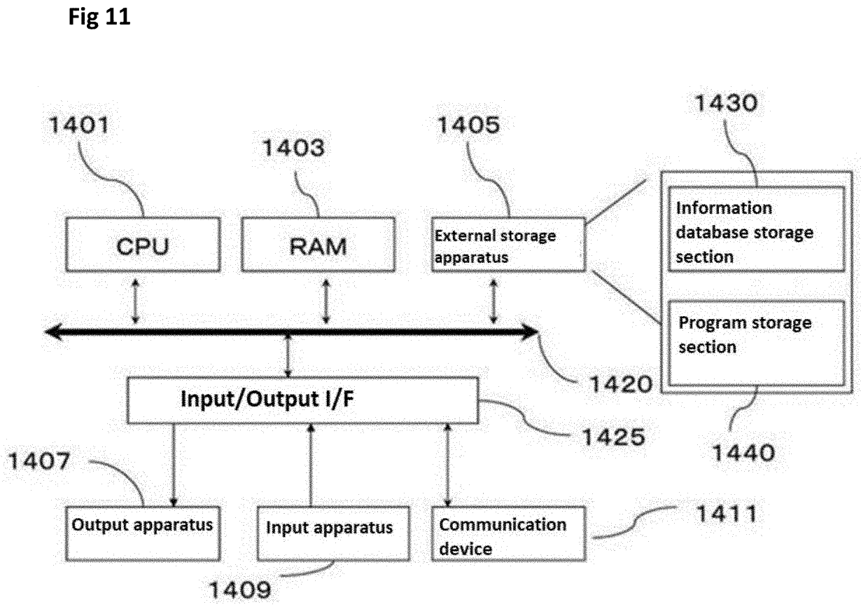

The above steps are described while referring to FIG. 11.

In S1 (step (1)), a reference database is provided. This may be stored in an external storage apparatus 1405, but can be obtained generally as a publically available database through a communication device 1411. Alternatively, an input apparatus 1409 may be used to input and optionally record a database in a RAM 1403 or the external storage apparatus 1405. In this regard, a database comprising a region of interest such as a V region is provided.

In S2 (step (2)), an input sequence set is provided. At this stage, for example, a set of sequence information obtained from a set of amplicons amplified in a PCR amplification reaction is inputted by using the input apparatus 1409 or through the communication device 1411. At this stage, an apparatus that receives an amplicon of a PCR amplification reaction and performs genetic sequence analysis thereon may be connected. Such a connection is made through a system bus 1420 or through the communication device 1411. Trimming and/or extraction of a sequence of a suitable length can be optionally performed at this stage. Such processing is performed with a CPU 1401. A program for trimming and/or extraction can be provided via each of an external storage apparatus, communication device, or input apparatus.

In S3 (step (3)), alignment is performed. This stage searches for homology of the input sequence set with the reference database for each of the gene regions. For the homology search, the reference database obtained via the communication device 1411 or the like is processed with a homology search program. The CPU 1401 performs the processing. Further, results obtained as a result thereof are analyzed for alignment with an approximate reference allele and/or a sequence of the reference allele. This is also processed by the CPU 1401. A program for the execution thereof can be provided via each of the external storage apparatus, communication device, or input apparatus.

In S4 (step (4)), nucleic acid sequence information on D is detected. This is also processed by the CPU 1401. A program for the execution thereof can be provided via each of the external storage apparatus, communication device, or input apparatus. At this stage, a V region and a J region are assigned for the input sequence set. Assignment is also processed with the CPU 1401. The CPU 1401 also extracts a nucleic acid sequence of the D region based on a result of assigning. A program for the assigning and extracting process can also be provided via each of the external storage apparatus, communication device, or input apparatus. Preferably, this can be accomplished by determining a V region and/or J region based on known information or the like from sequence alignment. Results can be stored in the RAM 1403 or external storage apparatus 1405. Preferably, such extraction can be accomplished by assigning the V region and the J region for the input sequence set and extracting a CDR3 sequence, with the front of CDR3 on a reference V region and end of CDR3 on reference J as guides. Such processing can also be performed with the CPU 1401. A program therefor can also be provided via each of the external storage apparatus, communication device, or input apparatus.

In S5 (step (5)), a D region is classified. A nucleic acid sequence of the D region is translated into an amino acid sequence and the D region is classified by utilizing the amino acid sequence. This is also processed with the CPU 1401. A program for this processing can also be provided via each of the external storage apparatus, communication device, or input apparatus. A sequence corresponding to the D region may be picked out by homology search or the like on the resulting amino acid sequence. This is also processed with the CPU 1401. A program for this processing can also be provided via each of the external storage apparatus, communication device, or input apparatus. Preferably, a nucleic acid sequence of the CDR3 can be translated into an amino acid sequence to classify the D region by utilizing the amino acid sequence. This is also processed with the CPU 1401. A program for this processing can also be provided via each of the external storage apparatus, communication device, or input apparatus.

In S6 (step (6)), a frequency of appearance for each of the V region, the D region, the J region, and optionally the C region or a frequency of appearance of a combination thereof is calculated based on the classifying described above to identify functional subunit pair genes of a TCR or a BCR. The calculating and deducing are also processed with the CPU 1401. A program for this processing can also be provided via each of the external storage apparatus, communication device, or input apparatus.

In one preferred embodiment, the gene region used in the present disclosure comprises all of the V region, the D region, the J region, and optionally the C region.

In one embodiment, the reference database is a database with a unique ID assigned to each sequence. A sequence of a gene can be analyzed based on a simple indicator, i.e., ID, by uniquely assigning an ID.

In one embodiment, the input sequence set is an unbiased sequence set. An unbiased sequence set can be implemented by PCR amplification using reverse transcription template switching PCR such as those described herein.

In another embodiment, the sequence set is trimmed. An unnecessary or unsuitable nucleic acid sequence can be removed by trimming, so that efficiency of analysis can be enhanced.

In a preferred embodiment, trimming is accomplished by the steps of: deleting low quality regions from both ends of a read; deleting a region matching 10 bp or more with a sequence of a template switching oligonucleotide from both ends of the read; and using the read as a high quality read in analysis if the remaining length is 200 bp or more (TCR) or 300 bp or more (BCR). Preferably, the low quality refers to a 7 bp moving average of QV value of less than 30.

In a preferred embodiment, the approximate sequence is the closest sequence. In a specific embodiment, the approximate sequence is determined by a ranking of 1. number of matching bases, 2. kernel length, 3. score, and 4. alignment length.

In another embodiment, the homology search is conducted under a condition tolerating random mutations to be scattered throughout. Such a condition is often expressed by, for example, the following condition for BLAST/FASTA optimal parameters: tolerates a maximum mismatch of 33% across the full length of an alignment; and tolerates a maximum nonconsecutive mismatch of 60% for any 30 bp therein. In one embodiment, the homology search comprises at least one condition from (1) shortened window size, (2) reduced mismatch penalty, (3) reduced gap penalty, and (4) a top priority ranking of an indicator is a number of matching bases, compared to a default condition.

In another embodiment, the homology search is carried out under the following conditions in BLAST or FASTA:

V mismatch penalty=−1, shortest alignment length=30, and shortest kernel length=15;

D word length=7 (for BLAST) or K-tup=3 (for FASTA), mismatch penalty=−1, gap penalty=0, shortest alignment length=11, and shortest kernel length=8;

J mismatch penalty=−1, shortest hit length=18, and shortest kernel length=10; and

C shortest hit length=30 and shortest kernel length=15.

This condition can be used, for example, as long as it is a case where a shorter (up to 200 bp) sequence is used to classify only a part of the region (a case that does not fall under the “preferred example”). This condition can also be used in a case where an Illumina sequencer is used. In such a case, the possibility of using bwa or bowtie for homology search is considered.

In a specific embodiment, the D region is classified by a frequency of appearance of the amino acid sequence.

In a further embodiment, a combination of a result of search for homology with the nucleic acid sequence of CDR3 and a result of amino acid sequence translation is used as a classification result in step (5) if there is a reference database for the D region.

In another embodiment, only the frequency of appearance of the amino acid sequence is used for classification in step (5) if there is no reference database for the D region.

In a specific embodiment, the frequency of appearance is counted in a unit of a gene name and/or a unit of an allele.

In another embodiment, step (4) comprises assigning the V region and the J region for the input sequence set and extracting a CDR3 sequence, with the front of CDR3 on a reference V region and end of CDR3 on reference J as guides.

In a further embodiment, step (5) comprises translating the nucleic acid sequence of the CDR3 into an amino acid sequence and classifying a D region by using the amino acid sequence.

In one aspect, the present disclosure provides a system for analyzing functional subunit pair genes of TCR or BCR, the system comprising: (1) means for providing a reference database for each of gene regions comprising at least one of a V region, a D region, a J region, and optionally a C region; (2) means for providing an input sequence set prepared from optionally trimming and optionally extracting a sequence with a suitable length; (3) means for searching for homology of the input sequence set with the reference database for each of the gene regions and recording an alignment with an approximate reference allele and/or a sequence of the reference allele; (4) means for assigning the V region and the J region for the input sequence set and extracting a nucleic acid sequence of the D region based on a result of assigning; (5) means for translating the nucleic acid sequence of the D region into an amino acid sequence and classifying the D region by utilizing the amino acid sequence; and (6) means for calculating a frequency of appearance for each of the V region, the D region, the J region, and optionally the C region or a frequency of appearance of a combination thereof based on the classifying in (5) to identify functional subunit pair genes of a TCR or a BCR.

In another aspect, the present disclosure provides a computer program causing a computer to execute processing of a method of analyzing functional subunit pair genes of a TCR or a BCR, the method comprising the following steps: (1) providing a reference database for each of gene regions comprising at least one of a V region, a D region, a J region, and optionally a C region; (2) providing an input sequence set prepared from optionally trimming and optionally extracting a sequence with a suitable length; (3) searching for homology of the input sequence set with the reference database for each of the gene regions and recording an alignment with an approximate reference allele and/or a sequence of the reference allele; (4) assigning the V region and the J region for the input sequence set and extracting a nucleic acid sequence of the D region based on a result of assigning; (5) translating the nucleic acid sequence of the D region into an amino acid sequence and classifying the D region by utilizing the amino acid sequence; and (6) calculating a frequency of appearance for each of the V region, the D region, the J region, and optionally the C region or a frequency of appearance of a combination thereof based on the classifying in (5) to identify functional subunit pair genes of a TCR or a BCR.

In still another aspect, the present disclosure provides a recording medium for storing a computer program causing a computer to execute processing of a method of analyzing functional subunit pair genes of a TCR or a BCR, the method comprising the following steps: (1) providing a reference database for each of gene regions comprising at least one of a V region, a D region, a J region, and optionally a C region; (2) providing an input sequence set prepared from optionally trimming and optionally extracting a sequence with a suitable length; (3) searching for homology of the input sequence set with the reference database for each of the gene regions and recording an alignment with an approximate reference allele and/or a sequence of the reference allele; (4) assigning the V region and the J region for the input sequence set and extracting a nucleic acid sequence of the D region based on a result of assigning; (5) translating the nucleic acid sequence of the D region into an amino acid sequence and classifying the D region by utilizing the amino acid sequence; and (6) calculating a frequency of appearance for each of the V region, the D region, the J region, and optionally the C region or a frequency of appearance of a combination thereof based on the classifying in (5) to identify functional subunit pair genes of a TCR or a BCR.

(System Configuration)

The configuration of a system 1 of the present disclosure is described while referring to the functional block diagram in FIG. 11. The Figure shows a case that is materialized with a single system.

The gene analysis system 1 of the present disclosure is configured by connecting a RAM 1403, external storage apparatus 1405 such as ROM, HDD, magnetic disk, or flash memory such as USB memory, and an input output interface (I/F) 1425 via a system bus 1420 to a CPU 1401 built into a computer system. An input apparatus 1409 such as a keyboard or a mouse, an output apparatus 1407 such as a display, and a communication device 1411 such as a modem are each connected to the input output I/F 1425. The external storage apparatus 1405 comprises an information database storage section 1430 and a program storage section 1440, which are both constant storage regions reserved within the external storage apparatus 1405.

Such a hardware configuration is designed to achieve a function of the invention in cooperation with an OS (operating system) by the CPU 1401 calling out, deploying, and executing a software program installed on the storage apparatus 1405 on the RAM 1403 from having various instructions (commands) being inputted via the input apparatus 1409 or from receiving a command via the communication I/F, communication device 1411, or the like.

A reference database, input sequence set, created classification data, data for a TCR or BCR repertoire or the like, or information obtained via the communication device 1411 or the like is constantly written and updated in the database storage section 1430. Information such as information ID of each gene in a reference database and each sequence in each input sequence set is managed with each master table to allow information from a sample that is subjected to accumulation to be managed using IDs defined in each master table.

As input sequence set entry information, a sample provider ID, sample information, result of nucleic acid analysis, known individual/physiological information, and result of TCR or BCR functional subunit pair gene analysis are associated with a sample ID and stored in the database storage section 1430. In this regard, the result of TCR or BCR functional subunit pair gene analysis is information obtained via processing the nucleic acid analysis result by the processing of the present disclosure.

Further, a computer program stored in the program storage section 1440 configures a computer as the processing system described above, e.g., a system for performing processing such as trimming, extraction, alignment, assignment, classification, or translation. Each of the functions is an independent computer program, module or routine thereof, or the like, which is executed by the CPU 1401 described above to configure a computer as each system or apparatus. It is assumed hereinafter that each system is constituted by cooperation of each function in each system.

(Manufacturing Method)

The present disclosure provides a method of manufacturing the following products.

A. TCR/BCR expressing cells
B. cells producing a virus or phage comprising a TCR/BCR nucleic acid
C. viruses or phages comprising a TCR/BCR nucleic acid
D. RNA having TCR/BCR or a complementary sequence thereof
E. TCR/BCR proteins (synthesized within cell)
F. TCR/BCR proteins (synthesized in vitro without cells)

Examples of manufacturing constructs used in the manufacturing method of the present disclosure include, but are not limited to, the following:

W. expression vector created by incorporating a TCR/BCR sequence (cells capable of expression vary depending on the vector structure; mammalian cell expression vector, insect cell expression vector, microbe expression vector, yeast expression vector, and the like);
X. virus or phage producing vector created by incorporating a TCR/BCR sequence
Y. homologous recombination vector created by incorporating a TCR/BCR sequence with a target genome region (mouse homologous recombination vector, human cell homologous recombination vector, and the like); and
Z. DNA fragment prepared from attaching an in vitro transcription promoter sequence such as T7 or SP6 to the 5′ terminus of a TCR/BCR sequence.

Typical examples of the manufacturing method of the present disclosure are shown below.

### A. TCR/BCR Expressing Cells

A-1 (A & W or X): manufactured by introducing “W. expression vector created by incorporating a TCR/BCR sequence” or “X. virus or phage producing vector created by incorporating a TCR/BCR sequence” into a cell of interest (lymphocyte, established culture cell, insect cell, E. coli, yeast, or the like) (in many cases, a virus producing vector can be used for gene expression in a cell in addition to a normal expression vector).

A-2 (A & C): manufactured by infecting a cell of interest (lymphocyte, established culture cell, insect cell, E. coli, yeast, or the like) with “C. viruses or phages comprising a TCR/BCR nucleic acid” manufactured in section C described below.

A-3 (A & Y): manufactured by introducing “Y. homologous recombination vector created by incorporating a TCR/BCR sequence with a target genome region into a suitable vector” to a cell of interest (lymphocyte, various primary culture cells, ES cell, various established cells, or the like of a human, mouse, or various animal models). Only cells with homologous recombination due to agent selection or the like may be selected as needed.

A-4 (A & D): created by introducing mRNA of TCR/BCR manufactured in section D described below into a cell of interest (lymphocyte, bone marrow cell, splenocyte, ES cell, various established cells, or the like of a human, mouse, or various animal models).

### B. Cells Producing a Virus or Phage Comprising a TCR/BCR Nucleic Acid

B-1 (B & X): manufactured by introducing “X. virus or phage producing vector created by incorporating a TCR/BCR sequence” to a suitable virus creation cell or microbe and optionally introducing a component protein expression vector, and applying helper phage treatment.

### C. Viruses or Phages Comprising a TCR/BCR Nucleic Acid

C-1 (C & X): manufactured by introducing “X. virus or phage producing vector created by incorporating a TCR/BCR sequence” into a suitable virus packaging cell, optionally introducing a component protein expression vector, applying helper phage treatment, then culturing the cell for a required period of time and retrieving a virus or phage comprising a TCR/BCR nucleic acid from the culture supernatant.

### D. RNA Having TCR/BCR or a Complementary Sequence Thereof

D-1 (D & W or X): RNA is manufactured through an RNA polymerase reaction in accordance with a biomolecular experiment book that is well known in the art or a commercially available kit by using a vector with an in vitro transcription promoter such as T7 or SP6 in a region that is in the immediate vicinity of the 5′ upstream of a TCR/BCR sequence or TCR/BCR expression vector incorporated into an in vitro transcriptional RNA synthesis vector among “X. virus or phage producing vector created by incorporating a TCR/BCR sequence” or “W. expression vector created by incorporating a TCR/BCR sequence”.

D-2 (D & Z): RNA is manufactured through an RNA polymerase reaction in accordance with a common biomolecular experiment book or a commercially available kit in the same manner as D-1 by using a “Z. DNA fragment prepared from attaching an in vitro transcription promoter sequence such as T7 or SP6 to the 5′ terminus of a TCR/BCR sequence”.

### E. TCR/BCR Protein (Intracellular Synthesis)

E-1 (E & W or X): a protein is produced by introducing “W. expression vector created by incorporating a TCR/BCR sequence” or “X. virus or phage producing vector created by incorporating a TCR/BCR sequence” into a cell that is suitable for protein expression (established cell, yeast, microbe, or the like), or a strain stably expressing a gene, strain highly expressing a protein, or the like among TCR/BCR expressing cells manufactured in A-1 or 2 can be grown.

In an expression system of established cells or yeasts, a membrane binding protein (TCR/BCR) is generally purified from a cell disruption solution (homogenate) by disrupting cells, and secretory immunoglobulin and TCR form to be secreted (protein with a substitution or deletion in the transmembrane region of the C region or the like) is purified from the culture supernatant through appropriate means. While protein localization varies in a microbe expression system, a protein is purified from culture as a secretory form, or an eluate or homogenate of each fraction within a cell (periplasm, cytoplasmic soluble fraction, inclusion body, or the like).

E-2 (E & Y): manufactured by introducing “Y. homologous recombination vector created by incorporating a TCR/BCR sequence with a target genome region into a suitable vector” into a cell of interest (lymphocyte, various primary culture cells, ES cell, various established cells, or the like of a human, mouse, or various animal models) to manufacture homologous recombinant cells (A-3) and purifying the manufactured cell protein.

E-3 (E & C): A cell of interest (lymphocyte, established culture cell, insect cell, yeast, microbe, or the like) is infected with “C. viruses or phages comprising a TCR/BCR nucleic acid” manufactured in the section of C-1 for gene expression and protein production. After disruption/elution or the like as needed in the same manner as section D-1, the protein is purified.

F. TCR/BCR Proteins (Synthesized In Vitro without Cells)

F-1 (E & W or X): manufactured through continuous mRNA synthesis and protein synthesis in an acellular system (synthesis system utilizing cell extract of rabbit reticulocyte, wheat germ, or E. coli is available, and a kit is also sold) by using a vector with an in vitro transcription promoter such as T7 or SP6 in a region that is in the immediate vicinity of the 5′ upstream of a TCR/BCR sequence among “X. virus or phage producing vector created by incorporating a TCR/BCR sequence” or “W. expression vector created by incorporating a TCR/BCR sequence”.

F-2 (E & Z): manufactured through continuous mRNA synthesis and protein synthesis in an acellular system (synthesis system utilizing cell extract of rabbit reticulocyte, wheat germ, or E. coli is available, and a kit is also sold) from “Z. DNA fragment prepared from attaching an in vitro transcription promoter sequence such as T7 or SP6 to the 5′ terminus of a TCR/BCR sequence”.

E-6 (E & D): manufactured through continuous protein synthesis in an in vitro translation system or an acellular system (synthesis system utilizing cell extract of rabbit reticulocyte, wheat germ, or E. coli is available, and a kit is also sold) by using the RNA manufactured in section D.

Therefore, the present disclosure provides the following in a certain aspect.

The present disclosure provides a method of manufacturing a cell expressing a T cell receptor (TCR) or a B cell receptor (BCR) having a sequence of functional subunit pair genes analyzed in accordance with the analysis method of the present disclosure, comprising: (4) providing an expression construct comprising the nucleic acid sequence of the TCR or the BCR; and (5) introducing the expression construct into the cell.

The present disclosure provides a method of manufacturing a cell producing a virus or a phage comprising a T cell receptor (TCR) or B cell receptor (BCR) nucleic acid having a sequence of functional subunit pair genes analyzed in accordance with the analysis method of the present disclosure, comprising: (4) providing a virus producing vector or a phage producing vector comprising the nucleic acid sequence of the TCR or the BCR; and (5) introducing the vector into the cell.

The present disclosure provides a method of manufacturing a virus or a phage comprising a T cell receptor (TCR) or B cell receptor (BCR) nucleic acid having a sequence of functional subunit pair genes analyzed in accordance with the analysis method of the present disclosure, comprising: (4) providing a virus producing vector or a phage producing vector comprising the nucleic acid sequence of the TCR or the BCR; (5) introducing the vector into a cell; and (6) culturing the cell to obtain a virus or a phage comprising a TCR or BCR nucleic acid from culture supernatant.

The present disclosure provides a method of manufacturing an RNA of a T cell receptor (TCR) or a B cell receptor (BCR) having a sequence of functional subunit pair genes analyzed in accordance with the analysis method of the present disclosure, comprising: (4) providing a vector for in vitro transcriptional RNA synthesis comprising the nucleic acid sequence of the TCR or BCR or a DNA fragment prepared from attaching an in vitro transcription promoter sequence to the nucleic acid sequence of the TCR or BCR; and (5) subjecting the vector or the DNA fragment to a condition under which an RNA polymerase reaction starts in vitro.

The present disclosure provides a method of manufacturing a T cell receptor (TCR) or B cell receptor (BCR) protein having a sequence of functional subunit pair genes analyzed in accordance with the analysis method of the present disclosure, comprising: (4) providing an expression construct comprising the nucleic acid sequence of the TCR or the BCR; (5) introducing the expression construct into a cell; and (6) subjecting the cell to a condition under which the TCR or the BCR is expressed.

The present disclosure provides a method of manufacturing a T cell receptor (TCR) or B cell receptor (BCR) protein having a sequence of functional subunit pair genes analyzed in accordance with the analysis method of the present disclosure, comprising: (4) infecting a cell with a virus or a phage manufactured by the method of manufacturing a virus or phage described above; and (5) subjecting the cell to a condition under which the TCR or the BCR is expressed.

The present disclosure provides a method of manufacturing a T cell receptor (TCR) or B cell receptor (BCR) protein having a sequence of functional subunit pair genes analyzed in accordance with the analysis method of the present disclosure, comprising: (4) providing a vector for in vitro transcriptional RNA synthesis comprising the nucleic acid sequence of the TCR or the BCR or a DNA fragment prepared from attaching an in vitro transcription promoter sequence to the nucleic acid sequence of the TCR or the BCR; (5) subjecting the vector or the DNA fragment to a condition under which an RNA polymerase reaction starts in vitro to provide an mRNA; and (6) subjecting the mRNA to a condition under which a protein is generated from the mRNA in vitro to provide the protein.

In some embodiments, the expression construct described above can be selected from, for example, an expression vector, a virus or phage producing vector, and a homologous recombination vector.

In still another aspect, the present disclosure provides a method of manufacturing a T cell receptor (TCR) or a B cell receptor (BCR) based on a sequence of functional subunit pair genes analyzed in accordance with the method of the present disclosure described above, comprising: (4) introducing a nucleic acid having the sequence of the functional subunit pair genes into an expression vector; (5) introducing the expression vector into a cell or a microbe; and (6) expressing the TCR or BCR under a condition where the cell or microbe can express the TCR or BCR. The step of (4) introducing a nucleic acid having the sequence of the functional subunit pair genes into an expression vector can utilize any enzyme, means, or the like that is known in the art as enzyme for any a nucleic acid treatment such as a restriction enzyme or ligase. The step of (5) introducing the expression vector into a cell can utilize any reagent for introducing a nucleic acid into a cell or microbe such as any reagent for transduction or transformation. (6) expressing the TCR or BCR under a condition where the cell or microbe can express the TCR or BCR can utilize a cell or microbe at a suitable temperature (e.g., 10 to 50° C., such as 37° C.) and utilize a cell in an environment of 5% CO₂ or the like. TCR or BCR can be manufactured by a conventional methodology described in a document such as “Mokuteki Betsu de Eraberu Kakusan Jikken no Genri to Purotokoru [Principle and Protocol for Nucleic Acid Experiment that can be Chosen Depending on Objective]”, Yodosha, Editors: Ichiro Hirao, Hitoshi Kurumizaka; “Mokuteki Betsu de Eraberu Idenshi Donyu Purotokoru [Protocol for Gene Transfer that can be Chosen Depending on Objective]”, Yodosha, Editors: Kazunori Nakajima, Yoshihiro Kitamura, Tsunenari Takeuchi; “Mokuteki Betsu de Eraberu Tanpakushitsu Hatsugen Purotokoru [Protein Expression Protocol that can be Chosen Depending on Objective]”, Yodosha, Editors: Kyosuke Nagata, Yo Okuwaki; and “Tanpakushitsu Jikken Purotokoru 1 [Protein Experiment Protocol 1]”, Gakken Medical Shujunsha, Supervised by Shigeo Ono and Yoshifumi Nishimura. Alternatively, an acellular system protein synthesis method that does not use a cell or microbe is known in the art, which synthesizes mRNA using in vitro transcription by incorporating an expression vector comprising a T7 or SP6 promoter sequence at the 5′ upstream of a genetic DNA fragment or the like and utilizes an experimental system such as an E. coli homogenate or wheat germ system from the synthesized mRNA.

A cell expressing TCR or BCR can be typically created by the following method. A nucleic acid having a sequence of functional subunit pair genes is inserted into a region with homology to a genomic sequence, and a functional protein domain or the like is optionally added, and the sequence is inserted into a homologous recombinant donor vector. Such cells can be then obtained by inducing homologous recombination at a TCR or BCR locus, and optionally separating and selecting homologous recombinant cells from cells that have not undergone homologous recombination. A homologous recombination vector, homologous recombinant cell, or homologous recombinant animal can be created by referring to “Experimental Medicine, All About Genomu Henshu [All About Genome Editing]”, Yodosha, Editors: Satoshi Mashita and Suguru Yamamoto, “Nature. 2018 July; 559 (7714): 405-409. Reprogramming human T cell function and specificity with non-viral genome targeting.”, Roth T L et al., or the like.

Descriptions in all publications including reference literatures such as scientific literatures, patents, and patent applications cited herein are incorporated herein by reference to the same extent that the entirety of each document is specifically described.

The present disclosure has been described above while providing preferred embodiments to facilitate understanding. The present disclosure is described below based on Examples. The aforementioned descriptions and the following Examples are not provided to limit the present disclosure, but for the sole purpose of exemplification. Thus, the scope of the present disclosure is not limited by the embodiments and Examples specifically described herein and is limited only by the scope of claims.

### EXAMPLES

The present disclosure is more specifically described hereinafter based on the Examples. It is understood that the specifically shown reagents as well as those available from TOYOBO, Takara Bio, Fujifilm Wako Pure Chemical, Nippon Gene, Nacalai Tesque, Thermo Fisher, New England Biolabs, Promega, or the like can be used as the various reagents used in the Examples.

### Preparation Examples

Preparation examples of reagents used in the Examples are shown below.

*List of reagents
-PrimeScript II High Fidelity One Step RT-PCR kit (R026A,
Takara Bio)
-40 U/μl RNasin Plus RNase Inhibitor (N2611, Promega)
-200 U/μl SuperScript IV Reverse Transcriptase (18090010,
Invitrogen)
-2× KAPA HiFi Hot Start Ready Mix (KK2602, Nippon Genetics)
-AMPure XP (A63881, Beckman Coulter)
-PE labeled HLA-A*02:01 CMV pp65 Tetramer (Medical &
Biological Laboratories)
-PE labeled HLA-A*02:01 Influenza M1 Tetramer (Medical &
Biological Laboratories)
-MACS buffer (130-091-221, Miltenyi Biotec)
*Oligonucleotide sequences used
-hTCRα Block primer (CA2; 23 bases):
(SEQ ID NO: 1)
GTGCATAGACCTCATGTCTAGCA
-hTCRα RT primer (CA1; 23 bases):
(SEQ ID NO: 2)
TGTTGAAGGCGTTTGCACATGCA
-hTCRβ, Block primer (CB2; 23 bases):
(SEQ ID NO: 3)
AGGCAGTATCTGGAGTCATTGAG
-hTCRβ, RT primer (CB1(3); 23 bases):
(SEQ ID NO: 4)
GAACTGGACTTGACAGCGGAA GT
-TS-Oligo (30 bases):
(SEQ ID NO: 5)
AAGCAGTGGTATCAACGCAGAGTACAT[G][G](G)*
*2^{nd} and 3^{rd} bases from the right end in H are RNA, and the
base at the right end in ( ) is LNA
-TS-Primer (30 bases):
(SEQ ID NO: 6)
AAGCAGTGGTATCAACGCAGAGTACATGGG
-Forward TS-Tag primer v1 (64 bases):
(SEQ ID NO: 7)
GTCTC{GTGGGCTCGGAGATGTGTAT}AAGAGACAGAAGCAGTGGTATCAACGCAGAGTAC
ATGGG (underlined portion is a MiSeq
sequencing tag sequence site, sequence surrounded by { } is
the Sanger sequencing primer sequence)
-hTCRα Reverse Tag primer (51 bases):
(SEQ ID NO: 8)
TCGTC{GGCAGCGTCAGATGTGTATAA}GAGACAGGAGGGTCAGGGTTCTGGA
(underlined portion is a MiSeq sequencing tag sequence site,
sequence surrounded by { } is the Sanger sequencing primer
sequence)
-hTCRα Reverse Tag primer/hTCA-R-TPS-1 (53 bases):
(SEQ ID NO: 9)
TCGTC{GGCAGCGTCAGATGTGTATAA}GAGACAGACTTGTCACTGGATTTAGAG
-hTCRβ Reverse Tag primer (52 bases):
(SEQ ID NO: 10)
TCGTC{GGCAGCGTCAGATGTGTATAA}GAGACAGGCTCAAACACAGCGACCTC
(underlined portion is a MiSeq sequencing tag sequence site,
sequence surrounded by { } is the Sanger sequencing primer
sequence)
*Sequencing primers
P5-seq (21 bases):
(SEQ ID NO: 11)
GGCAGCGTCAGATGTGTATAA
CA3 (23 bases):
(SEQ ID NO: 12)
ACTTTGTGACACATTTGTTTGAG
CB3 (23 bases):
(SEQ ID NO: 13)
ACTGTGCACCTCCTTCCCATTCA

### Example 1: Comparison of Analysis Using a Single Cell with Analysis Using Bulk Cells

### Experiment A

(Reagents)

The additional reagent used in Experiment A is the following.
APC labeled antihuman CD8 antibody (Nippon Becton Dickinson Company)

A1. Cell Preparation and Culture

A1-1. Cell Isolation

Peripheral blood was collected from a human volunteer (HLA-A*02 carrier with history of CMV infection). Peripheral blood mononuclear cells (PBMC) were isolated by a Ficoll density gradient method, and the cells were cultured for the experiment.

PBMCs (approximately a million cells) immediately after separation were dissolved in a Torizol reagent and stored at −80° C. for analysis of the entire cell population by ordinary repertoire analysis.

A1-2. Cell Culture

PBMCs were “activated” with a cytomegalovirus (CMV) pp65 protein epitope peptide, cultured for 1 week, and then used in a single cell sorting repertoire analysis experiment. In this regard, “activate” can be confirmed using, for example, cell growth activity, an increase in the amount of secretion of cytokines such as interferon γ or interleukin, an increase in cytotoxic activity on MHC-CMV pp65 peptide presenting cells, an increase in the amount of TCR proteins, or the like as an indicator.

The full length amino acid sequence of CMV (Human betaherpesvirus 5) pp65 (AKI22842.1) is the following. The 9 underlined amino acid residues are the peptide epitope sites used in this Example.

(SEQ ID NO: 804)
MESRGRRCPEMISVLGPISGHVLKAVFSRGDTPVLPHETRLLQTGIHVRV
SQPSLILVSQYTPDSTPCHRGDNQLQVQHTYFTGSEVENVSVNVHNPTGR
SICPSQEPMSIYVYALPLKMLNIPSINVHHYPSAAERKHRHLPVADAVIH
ASGKQMWQARLTVSGLAWTRQQNQWKEPDVYYTSAFVFPTKDVALRHVVC
AHELVCSMENTRATKMQVIGDQYIKVYLESFCEDVPSGKLFMHVTLGSDV
EEDLTMTRNPQPFMRPHERNGFTVLCPKNMIIKPGKISHIMLDVAFTSHE
HFGLLCPKSIPGLSISGNLLMNGQQIFLEVQAIRETVELRQYDPVAALFF
FDIDLLLQRGPQYSEHPTFTSQYCIKGKLEYRHTWDRHDEGAAQGDDDVW
TSGSDSDEELVTTERKTPRVTGGGAMAGASTSAGRKRKSASSATACTSGV
MTRGRLKAESTVAPEEDTDEDSDNEIHNPAVFTWPPWQAGILARNLVPMV
ATVQGQNLKYQEFFWDANDIYRIFAELEGVWQPAAQPKRRRHRQDALPGP
CIASTPKKHRG

To perform analysis of the entire cell population by an ordinary repertoire analysis, a part of the peptide-stimulated staining cells (1 million cells) were dissolved in a Torizol reagent and stored at −80° C.

A2. Cell Marker Staining, Epitope Staining, and Single Cell Sorting

A2-1. Staining with Cell Marker Antibody and Epitope Peptide

PBMCs treated with an epitope peptide and cultured were centrifuged and resuspended into MACS buffer for washing, and reacted for 20 minutes at room temperature with a fluorescent substance phycoerythrin (PE) labeled MHC pp65 epitope tetramer and 15 minutes at 4° C. with a fluorescent substance allophycocyanin (APC) labeled anti-CD8 antibody in MACS buffer.

A2-2. Single Cell Sorting

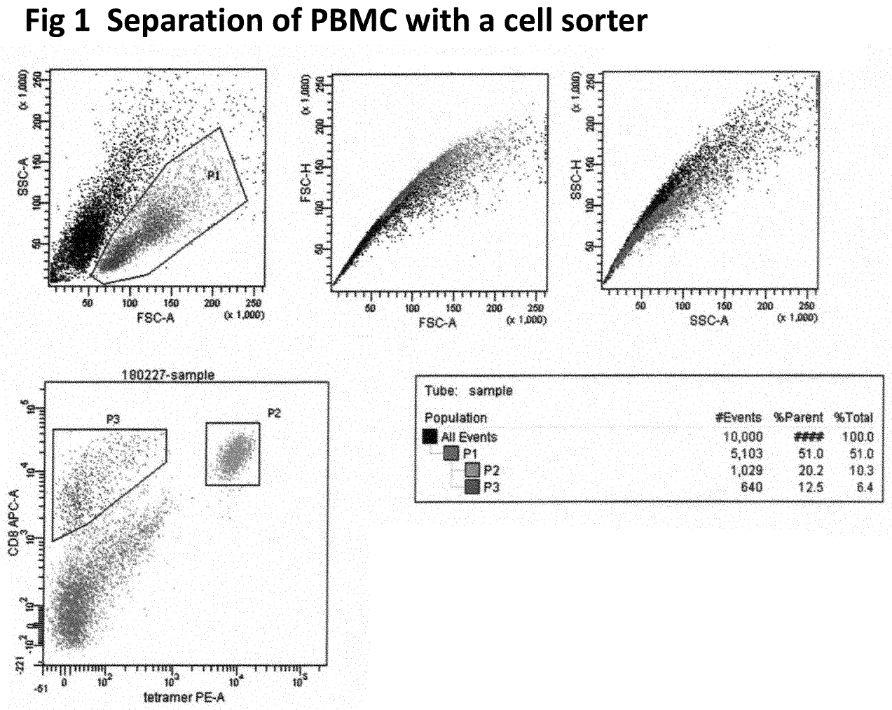

After the reaction, the cells were centrifuged and resuspended into MACS buffer for washing, and separated with a cell sorter (BD FACS Aria). PBMCs stained with a PE-labeled MHC CMV pp65 epitope tetramer and APC-labeled anti-CD8 antibody were developed in FSC-A (Forward Scatter Area; value indicating the cell size in forward scatter area) and SSC-A (Side Scatter Area; indicator of complexity of intracellular organelle or the like) in accordance with the manufacturer's manual, resulting in a lymphocyte group gate (P1) at the bottom right of the X and Y axes. P1 was further developed with PE (MHC CMV pp65 epitope tetramer positive) and APC (CD8 positive). A fraction of MHC CMV pp65 epitope tetramer reactive cytotoxic T cells was obtained at the P2 gate of PE (MHC CMV pp65 epitope tetramer positive) and APC (CD8 positive), and a fraction of MHC CMV pp65 epitope tetramer non-reactive cytotoxic T cells was obtained at the P3 gate. APC positive (CD8 positive), PE positive (MHC pp65 epitope tetramer positive) cell groups (P2 region in FIG. 1) were dispensed into a 96-well plate at one cell each and subjected to single cell repertoire analysis.

After the single cell sorting, cells at the P2 region (approximately 350 thousand cells) and APC positive (CD8 positive), PE negative (MHC pp65 epitope tetramer negative) cells at the P3 region (approximately 240 thousand cells) were sorted into a tube, dissolved into a Torizol reagent, and stored at −80° C. for normal repertoire analysis of bulk cells.

A3. Amplification of TCRα and TCRβ cDNA by One-Step RT-TS-PCR

A3-1. One-Step RT-TS-PCR Reaction

The “One-step RT-TS-PCR reaction” performed herein comprises the following steps.

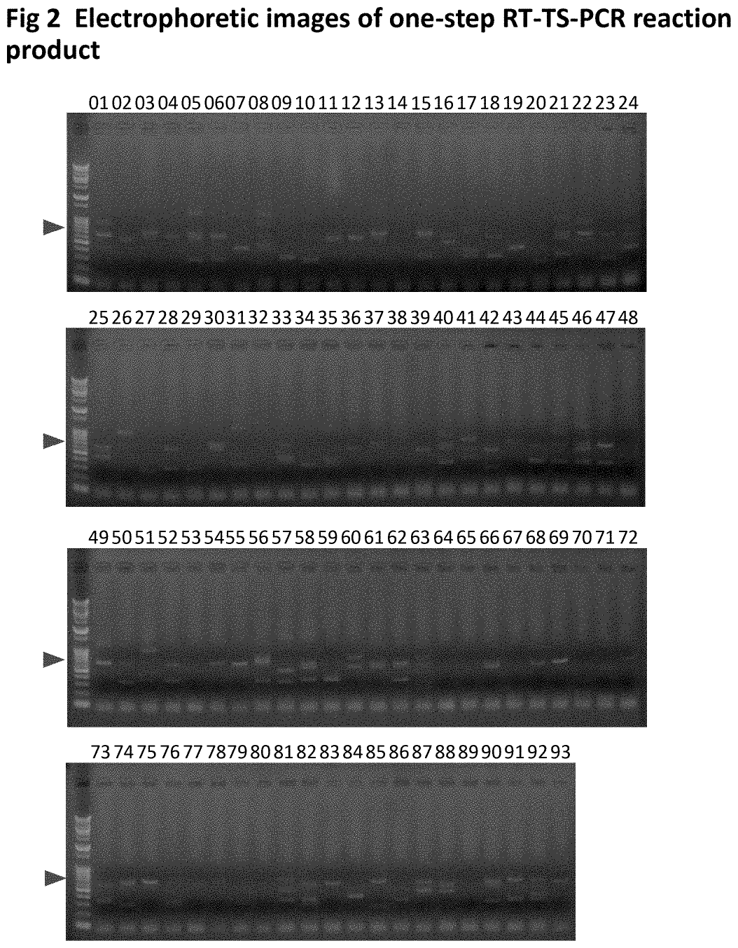

The reaction solution shown in the following Table 1 was added at 10 μl/well each to a plate to which the cells were dispensed to perform RT-PCR reaction. 2 μl of the reaction solution was fractionated to study a band by agarose gel electrophoresis (FIG. 2). As a result, a DNA band was observed in more than half the wells.

A3-2. Semi-Nested PCR

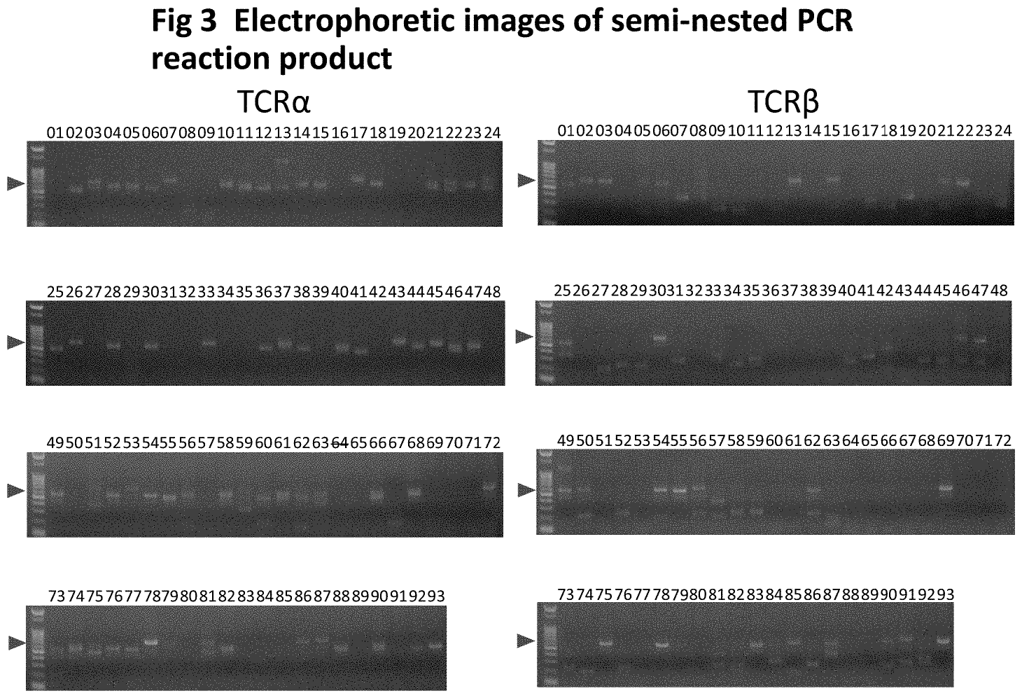

A one-step RT-TS-PCR reaction solution was fractionated, and a PCR reaction was performed in a semi-nested form using a primer added with a sequencing sequence to each of the template switching sequence on the 5′ side and to the sequence on the inside of a block primer on the 3′ side to study the amplification for each of TCRα and TCRβ separately and to add a common sequencing primer recognition sequence to a DNA fragment (Table 2). 3 μl of reaction solution was fractionated to study a band by agarose gel electrophoresis (FIG. 3). As a result, a DNA band was observed in 64 wells for TCRα and in 32 wells for TCRβ, with a well confirmed to have a product with a length of 400 base pairs or greater as positive. A cDNA band was observed for both TCRα and TCRβ in 22 of the wells.

A3-3. Sequencing-1

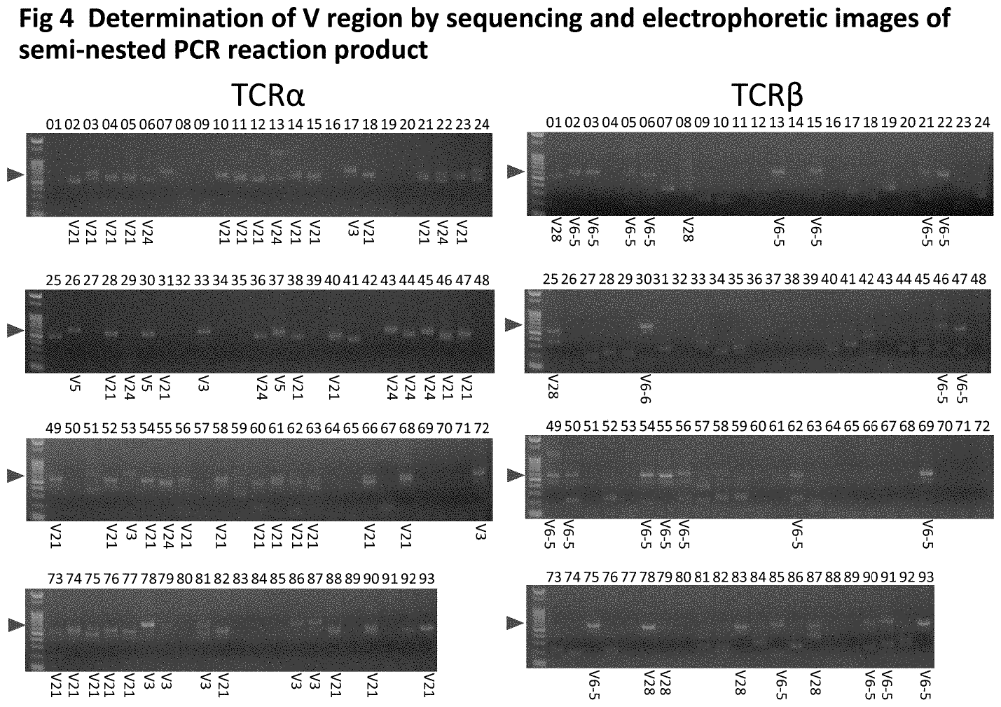

PCR-amplified TCRα and TCRβ, cDNA fragments were purified with an AMPure XP DNA kit, and Sanger sequencing was performed with a P5-seq primer. For the resulting sequences, a TCRα/β sequence database (The International Immunogenetics Information System; IMGT, http://www.imgt.org/) was downloaded, blast homology search was performed, and the sequences were analyzed with an IMGT Web repertoire sequence analysis system IMGT/V-QUEST (http://www.imgt.org/IMGT_vquest/vquest) to check for the presence/absence of TCR amplification and the amplified base sequences. FIG. 4 (under the electrophoretic image) shows the observed V region number. As a result of sequencing, amplification of TCR cDNA fragments was observed in 59 wells for TCRα and 30 wells for TCR β. Table 2A shows the determined TCRα/β sequence pair information (? means unidentified), clone ranking, and sequence example (sequencing data for well No. 3). It appeared that a specific clone (understood to be derived from CMV pp65 epitope peptide reactive memory T cells) such as the pair of TRAV21/J49 and TRBV6-5/J1-2 has significantly grown.

TABLE 2A
TCRα/β pair list

| Well No. | TCRα identification | TCRβ identification |
|---|---|---|
| 2 | TRAV21/J49 | TRBV6~5/J1~2 |
| 3 | TRAV21/J49 | TRBV6~5/J1~2 |
| 5 | TRAV21/J49 | TRBV6~5/J1~2 |
| 6 | TRAV24/J49 | TRBV6~5/J1~2 |
| 13 | TRAV24/J49 | TRBV6~5/J1~2 |
| 15 | TRAV21/J49 | TRBV6~5/? |
| 21 | TRAV21/J49 | TRBV6~5/? |
| 22 | TRAV24/J49 | TRBV6~5/J1~2 |
| 30 | TRAV5/J20 | TRBV6~5/J1~2 |
| 46 | TRAV21/J49 | TRBV6~5/? |
| 47 | TRAV21/J49 | TRBV6~5/? |
| 49 | TRAV21/J49 | TRBV6~5/J1~2 |
| 54 | TRAV21/J49 | TRBV6~5/J1~2 |
| 55 | TRAV24/J49 | TRBV6~5/J1~2 |
| 56 | TRAV21/J49 | TRBV6~5/? |
| 62 | TRAV21/J49 | TRBV6~5/β |
| 75 | TRAV21/J49 | TRBV6~5/? |
| 78 | TRAV3/J26 | TRBV28/J1~1 |
| 79 | TRAV3/J26 | TRBV28/J1~1 |
| 87 | TRAV3/J26 | TRBV28/J2~3 |
| 90 | TRAV21/J49 | TRBV6~5/J1~2 |
| 93 | TRAV21/J49 | TRBV6~5/J1~2 |

A3-4. Sequencing-2

The samples found to have a band by electrophoresis among the RT-PCR products obtained from “3-1. One-step RT-TS-PCR reaction” (this analysis targeted Well No. 1 to 30; a band was found in 20 samples, i.e., No. 1, 2, 3, 4, 5, 6, 7, 11, 12, 13, 15, 18, 19, 21, 22, 23, 24, 25, 28, and 30) were diluted 3-fold, and then DNA was purified with an AMPure XP DNA kit from 17 to 18 μl of reaction solution and recovered in 18 μl of 10 mM Tris Cl buffer. Sanger sequencing was performed on TCRα cDNA amplicon and TCR β amplicon using a TCRα C region primer (CA3 primer) in one reaction system and a TCRβ C region primer (CB3 primer, which is a common sequence of C1 and C2 regions) in the other reaction system, respectively, for the 19 samples from which the required amount of DNA was able to be recovered (samples other than No. 23). The resulting sequences were subjected to in house blast homology search using an IMGT TCRα/β sequence database, analysis with Repertoire Genesis's proprietary analysis system, and the like to study the presence/absence of TCR amplification and the amplified base sequences. Table 3 shows data for the V/J regions analyzed in this analysis and results obtained by sequencing semi-nested PCR amplicons in “A3-3 Sequencing-1” (only results for Well No. 1 to 30) for reference. The V region of TCRα was able to be determined for samples in 10 wells, V region of TCRβ was able to be determined for samples in 11 wells, and this was able to be identified for the TCRα/β pair in 6 wells. When RT-PCR-semi-nested PCR products were sequenced, the V region of TCRα was able to be determined for samples in 20 wells, V region of TCRβ was able to be determined for samples in 12 wells, and this was able to be identified for the TCRα/β pair in 9 wells in the same sample. While the analyzable ratio decreased slightly, this enabled more simple sequencing.

When performing TCR cDNA cloning in addition of sequence analysis, TCRα and TCRβ cDNA sequences that can be utilized in a protein expression experiment can be obtained by performing sequencing with this methodology, amplifying TCR cDNA fragments using a cloning primer from a one-step RT-PCR reaction solution of a well containing a sequence of interest, and performing assembly cloning (methodology that enables ligation of a plurality of DNA fragments at once at a high probability) on C region DNA fragments synthesized separately by gene synthesis or PCR reaction with cloning vectors or expression vectors.

TABLE 3

| | Using RT-PCR product | | | | Using Semi-nested PCR product | | | |
|---|---|---|---|---|---|---|---|---|
| Well No | TRAV | TRAJ | TRBV | TRBJ | TRAV | TRAJ | TRBV | TRBJ |
| 01 | | | TRBV28 | | | | TRBV28 | |
| 02 | TRAV21 | TRAJ49 | TRBV6-5 | TRBJ1-2 | TRAV21 | TRAJ49 | TRBV6-5 | TRBJ1-2 |
| 03 | TRAV21 | TRAJ49 | TRBV6-5 | TRBJ1-2 | TRAV21 | TRAJ49 | TRBV6-5 | TRBJ1-2 |
| 04 | TRAV21 | TRAJ49 | | | TRAV21 | TRAJ49 | | |
| 05 | TRAV21 | TRAJ49 | TRBV6-5 | TRBJ1-2 | TRAV21 | TRAJ49 | TRBV6-5 | TRBJ1-2 |
| 06 | | | TRBV6-5 | TRBJ1-2 | TRAV24 | TRAJ49 | TRBV6-5 | TRBJ1-2 |
| 07 | | | x | TRBJ2-3 | | | | |
| 08 | | | | | | | TRBV28 | TRBJ2-3 |
| 09 | | | | | | | | |
| 10 | | | | | TRAV21 | TRAJ49 | | |
| 11 | TRAV21 | TRAJ49 | | | TRAV21 | TRAJ49 | | |
| 12 | | | | | TRAV21 | TRAJ49 | | |
| 13 | | | TRBV6-5 | TRBJ1-2 | TRAV24 | TRAJ49 | TRBV6-5 | TRBJ1-2 |
| 14 | | | | | TRAV21 | TRAJ49 | | |
| 15 | | | TRBV6-5 | | TRAV21 | TRAJ49 | TRBV6-5 | |
| [symbol] | | | | | | | | |
| 17 | | | | | TRAV3 | TRAJ26 | | |
| 18 | TRAV21 | TRAJ49 | | | TRAV21 | TRAJ49 | | |
| 19 | | | | TRBJ2-3 | | | | |
| 20 | | | | | | | | |
| 21 | TRAV21 | TRAJ49 | TRBV6-5 | | TRAV21 | TRAJ49 | TRBV6-5 | |
| 22 | TRAV24 | TRAJ49 | TRBV6-5 | TRBJ1-2 | TRAV24 | TRAJ49 | TRBV6-5 | TRBJ1-2 |
| 23 | | | | | TRAV21 | TRAJ49 | | |
| 24 | TRAV4 | TRAJ40 | | TRBJ2-3 | | | | |
| 25 | TRAV3 | | TRBV28 | TRBJ2-3 | | | TRBV28 | TRBJ2-3 |
| 26 | | | | | TRAV5 | TRAJ20 | | |
| 27 | | | | | | | | |
| 28 | | | | | TRAV21 | TRAJ49 | | |
| 29 | | | | | TRAV24 | TRAJ49 | | |
| 30 | | | TRBV6-6 | TRBJ1-2 | TRAV5 | TRAJ20 | TRBV6-6 | TRBJ1-2 |

A3-5. Sequencing-3 (Using MiSeq Next Generation Sequencer)

Samples of 16 wells (Well No. 2, 3, 5, 6, 13, 21, 22, 30, 46, 47, 49, 54, 55, 78, 79, and 87) among the samples whose pair sequence was determined by Sanger direction sequencing were sequenced with a MiSeq next generation sequencer. To analyze the sequences of DNA fragments amplified and purified at “A3-2. Semi-nested PCR” with a next generation sequencer, index addition PCR was performed with the PCR reaction solution and reaction cycle shown in Table 4. PCR products were purified with an AMPure XP DNA kit, and sequencing was run with a MiSeq next generation sequencer in accordance with the manufacturer's protocol. The resulting sequencing data was analyzed with Repertoire Genesis software, in house blast analysis, and the like.

The results are shown in Table 5. Clear data was obtained for TCRβ sequences of Well No. 21, 46, and 47, for which some sequences were unclear, e.g., J region was undetermined, with the Sanger method. While data was somewhat unclear for the sample of Well No. 87 with the Sanger method and determined as TRBV28/J2-3, clear data was obtained with a MiSeq sequencer to reveal that TRBV28/J1-1 was correct.

It was also found that in Well No. 2, 3, 5, 21, 22, 46, and 55, both TRAV21/J49 and TRAV24/J49 are expressed, and there is only one type of corresponding pair TCRβ, i.e., TRBV6-5/J1-2. This result is difficult to analyze with the Sanger capillary sequencing method that detects results of multiple molecules collectively as a single fluorescence data. Analysis was made possible only after using a next generation sequencer that can detect results of multiple molecules as data for each individual molecule.

TABLE 5
Results of analys s with a MiSeq next generation sequencer

| | TCRα | | | | | |
|---|---|---|---|---|---|---|
| Well | Sanger method | MiSeq total | | Read | | Read |
| No. | results | reads | TOP | counts | 2nd | counts |
| 7 | TRAV21/149 | 48060 | TRAV21/149 | 28528 | TRAV24/149 | 16111 |
| 8 | TRAV21/149 | 51985 | TRAV21/149 | 40439 | TRAV24/149 | 11321 |
| [symbol] | TRAV21/149 | 52073 | TRAV21/149 | 40335 | TRAV24/149 | 11370 |
| [symbol] | TRAV24/149 | 45524 | TRAV24/149 | 45735 | TRAV21/149 | 10 |
| 13 | TRAV24/149 | 24340 | TRAV24/149 | 24617 | TRAV21/149 | 13 |
| 21 | TRAV21/149 | 31185 | TRAV [symbol] | 32129 | TRAV24/149 | 17872 |
| 22 | TRAV24/149 | 46530 | TRAV21/149 | 29533 | TRAV21/149 | 16840 |
| 30 | TRAV [symbol] | 34877 | TRAV [symbol] | 34751 | TRAV [symbol] | 5 |
| 46 | TRAV [symbol] | 30324 | TRAV21/149 | 19839 | TRAV24/149 | 10243 |
| 47 | TRAV21/149 | 35757 | TRAV [symbol] | 35427 | TRAV24/149 | 2 |
| 49 | TRAV21/149 | 30788 | TRAV [symbol] | [symbol] | [symbol] | 1 |
| 54 | TRAV21/149 | 25093 | TRAV21/149 | 28019 | TRAV24/149 | 3 |
| [symbol] | TRAV24/149 | 33272 | TRAV [symbol] | 16743 | TRAV [symbol] | 15342 |
| 78 | TRAV [symbol] | 24291 | TRAV [symbol] | [symbol] | TRAV [symbol] | 631 |
| 79 | TRAV [symbol] | 20899 | TRAV [symbol] | 20714 | TRAV [symbol] | 131 |
| 87 | TRAV [symbol] | 20245 | TRAV [symbol] | [symbol] | TRAV [symbol] | 4 |
| | TCRβ | | | | | |
| Well | Sanger method | MiSeq total | | Read | | Read |
| No. | results | reads | TOP | counts | 2nd | counts |
| 7 | TRAV6-5 [symbol] -2 | [symbol] | TRAV6-5 [symbol] -2 | 27640 | TRAV6-5 [symbol] -2 | [symbol] |
| 8 | TRAV6-5 [symbol] | [symbol] | TRAV6-5 [symbol] | 36143 | TRAV6-5 [symbol] | 38 |
| [symbol] | TRAV [symbol] -2 | 89609 | TRAV [symbol] -2 | 7924 | TRAV [symbol] -2 | 235 |
| [symbol] | TRAV6-5 [symbol] -2 | 94365 | TRAV6-5 [symbol] -2 | 13078 | TRAV6-5 [symbol] -2 | 31 |
| 13 | TRAV [symbol] -2 | 28721 | TRAV [symbol] -2 | 28100 | TRAV [symbol] -2 | 21 |
| 21 | TRAV6-5 [symbol] | [symbol] | TRAV6-5 [symbol] | 19264 | TRAV6-5 [symbol] | 10 |
| 22 | TRAV6-5 [symbol] -2 | 33737 | TRAV6-5 [symbol] -2 | [symbol] | TRAV6-5 [symbol] -2 | 18 |
| 30 | TRAV [symbol] -2 | 24367 | TRAV [symbol] -2 | [symbol] | TRAV [symbol] -2 | 1 |
| 46 | TRAV6-5 [symbol] | 89285 | TRAV6-5 [symbol] | 24852 | TRAV6-5 [symbol] | 156 |
| 47 | TRAV6-5 [symbol] | 81703 | TRAV6-5 [symbol] | 23360 | [symbol] and [symbol] | 3 |
| 49 | TRAV6-5 [symbol] -2 | 26300 | TRAV6-5 [symbol] -2 | 26921 | TRAV6-5 [symbol] -2 | 19 |
| 54 | TRAV6-5 [symbol] -2 | 36138 | TRAV6-5 [symbol] -2 | 31519 | TRAV6-5 [symbol] -2 | 126 |
| [symbol] | TRAV6-5 [symbol] -2 | 37973 | TRAV6-5 [symbol] -2 | 37400 | TRAV6-5 [symbol] -2 | 29 |
| 78 | TRAV6-5 [symbol] -1 | 364267 | TRAV6-5 [symbol] -1 | 34352 | TRAV6-5 [symbol] -1 | 5 |
| 79 | TRAV6-5 [symbol] -1 | [symbol] | TRAV6-5 [symbol] -1 | 60290 | [symbol] and [symbol] | 7 |
| 87 | TRAV [symbol] -3 | [symbol] | TRAV [symbol] -3 | 24945 | TRAV [symbol] -3 | 25 |

[symbol]

indicates data missing or illegible when filed

Next, read 1 (data for sequencing from the 3′ side C region side of TCR cDNA) and read 2 (data for sequencing from the 5′ side of TCR cDNA) of MiSeq sequencing data were joined with a fastq-join program, converted to a reverse complementary strand sequence, and converted to a sequence with the same orientation as normal cDNA. After picking up only clones with a primer sequence on both ends of the sequence and removing the primer sequences, the clones were randomly selected, and base sequences of amplified cDNA were obtained from base sequence assembly multiple alignment analysis. If a DNA fragment was long (longer than about 500 to 580 base pairs) leading to poor joining rate, a low quality region was removed while utilizing a reverse complementary sequence of read 1 for a sequence that failed to join for a read assigned to said clone and sequence of read 2 without modification, in addition to a sequence that was able to join, and then base sequence assembly multiple alignment analysis was performed to obtain the base sequence of amplified cDNA. The resulting cDNA base sequence was compared to genetic sequences on a public database such as Ensemble's Gene database to confirm whether a sequence was obtained from a 5′ amino acid sequence start codon. Further, after an amino acid sequence substitution, the amino acid sequences of reader regions and variable regions (four framework regions: FR1 to 4 and three variable regions: CDR1 to 3) were predicted. The resulting TCRα and TCRβ base sequences and amino acid translated sequences of Well No. 13 are shown as an example of analysis (underlines indicate translation start codon annotated on Ensemble's Gene database and CDR3 regions identified by computer analysis). Protein information was obtained in-frame from a translation start codon to the N-terminal amino acid sequence of a C region comprising a CDR3 region in each case. It was also confirmed from studying the base sequence that TCRβ of Well No. 13 has a C1 form C region.

Proteins can be expressed or synthesized in a microbial/cellular system or artificially synthesized system and applied to protein sample creation, epitope search, TCR introduced T cell therapy, or the like by optionally subjecting the cDNA base sequence or cDNA fragment of a variable region obtained by such analysis to reamplification, artificial gene synthesis, or the like, and cloning or joining them with a sequence of a C region with few functional differences to obtain a full length coding region.

>Well No. 13 TCRα base sequence
(SEQ ID NO: 14)
GGGCTGCAAAACGTTTTTCTGCTGTGGGTACGTGAGCAGGAAACATGGAG
AAGAATCCTTTGGCAGCCCCATTACTAATCCTCTGGTTTCATCTTGACTG
CGTGAGCAGCATACTGAACGTGGAACAAAGTCCTCAGTCACTGCATGTTC
AGGAGGGAGACAGCACCAATTTCACCTGCAGCTTCCCTTCCAGCAATTTT
TATGCCTTACACTGGTACAGATGGGAAACTGCAAAAAGCCCCGAGGCCTT
GTTTGTAATGACTTTAAATGGGGATGAAAAGAAGAAAGGACGAATAAGTG
CCACTCTTAATACCAAGGAGGGTTACAGCTATTTGTACATCAAAGGATCC
CAGCCTGAAGACTCAGCCACATACCTCTGTGCCCGGAACACCGGTAACCA
GTTCTATTTTGGGACAGGGACAAGTTTGACGGTCATTCCAAATA
>Well No. 13 TCRα amino acid translated sequence
(SEQ ID NO: 15)
AAKRFSAVGT*AGNMEKNPLAAPLLILWFHLDCVSSILNVEQSPQSLHVQ
EGDSTNFTCSFPSSNFYALHWYRWETAKSPEALFVMTLNGDEKKKGRISA
TLNTKEGYSYLYIKGSQPEDSATYLCARNTGNQFYFGTGTSLTVIPN
>Well No. 13 TCRβ base sequence
(SEQ ID NO: 16)
GGTCTCAGAATGACTTCCTTGAGAGTCCTGCTCCCCTTTCATCAATGCAC
AGATACAGAAGACCCCTCCGTCATGCAGCATCTGCCATGAGCATCGGCCT
CCTGTGCTGTGCAGCCTTGTCTCTCCTGTGGGCAGGTCCAGTGAATGCTG
GTGTCACTCAGACCCCAAAATTCCAGGTCCTGAAGACAGGACAGAGCATG
ACACTGCAGTGTGCCCAGGATATGAACCATGAATACATGTCCTGGTATCG
ACAAGACCCAGGCATGGGGCTGAGGCTGATTCATTACTCAGTTGGTGCTG
GTATCACTGACCAAGGAGAAGTCCCCAATGGCTACAATGTCTCCAGATCA
ACCACAGAGGATTTCCCGCTCAGGCTGCTGTCGGCTGCTCCCTCCCAGAC
ATCTGTGTACTTCTGTGCCAGCAGTCAACAGACAGGGACGATAGGTGGCT
ACACCTTCGGTTCGGGGACCAGGTTAACCGTTGTAGAGGACCTGAACAAG
GTGTTCCCACCC
>Well No. 13 TCRβ amino acid translated sequence
(SEQ ID NO: 17)
SQNDFLESPAPLSSMHRYRRPLRHAASAMSIGLLCCAALSLLWAGPVNAG
VTQTPKFQVLKTGQSMTLQCAQDMNHEYMSWYRQDPGMGLRLIHYSVGAG
ITDQGEVPNGYNVSRSTTEDFPLRLLSAAPSQTSVYFCASSQQTGTIGGY
TFGSGTRLTVVEDLNKVFPP

A3-6. Bulk Cell Repertoire Analysis

TCRα and TCRβ repertoire analysis was performed with (1) PBMCs (approximately 1 million cells) (2) peptide stimulated cells cultured for 1 week (1 million cells), (3) P2 region CD8+ MHC pp65 epitope tetramer positive cells (approximately 350 thousand cells), and (4) P3 region CD8+ MHC pp65 epitope tetramer negative cell calls, immediately after culture. The results thereof (list of top 50 species in clone ranking) are shown in Tables 6 to 13. Peptide stimulation and culture increased the ratio of specific sequences (TCRα: TRAV21/J49, TRAV24/J49, TRAV3/J26, TRAV5/J20, and the like, TCRβ: TCRBV6-5/J1-2, TRBV28/J1-1, TRBV6-6/J1-2, and the like) of cell clones believed to have grown due to the stimulation in the cell group of (2), which were fractionated and concentrated in (3) P2 region CD8+ MHC pp65 epitope tetramer positive cells by cell sorting, but were hardly observed in (4) P2 region CD8+ MHC pp65 epitope tetramer negative cells. It can be understood from comparing these data to single cell repertoire analysis results that TRAV21/J49 & TCRBV6-5/J1-2 pair sequences and TRAV24/J49 & TCRBV6-5/J1-2 pair sequences were actually obtained, so that success/failure of the experiment was able to be confirmed. In this manner, repertoire analysis data for the entire cell group can be used as reference data to confirm success/failure of experimental processes, whether positive clones of interest are obtained, or what extent of single cell screening needs to be performed. For example, if diversity of TCR sequences is low (high clonality), the number of resulting TCRα/β pair sequences would be limited even after analysis of a large number of cells, but if diversity is high, it is expected that a greater number of TCRα/β pair sequences would be obtained proportionally to the number of single cells that are analyzed.

TABLE 6
Results of TCRα repertoire analysis on (1) PBMCs

| | | | | | |
|---|---|---|---|---|---|
| Rank | TRAV | TRAJ | CDR3(SEQ ID NO) | Reads | [symbol] (Total Reads) |

[Table 6-1]

| | | | | | |
|---|---|---|---|---|---|
| 1 | TRAV36-2/DV8 | TRAJ40 | CAHITSGTYKYIF (18) | 2078 | 2.13 |
| 2 | TRAV12-2 | TRAJ21 | CADRNFNKFYF (19) | 1868 | 1.92 |
| 3 | TRAV21 | TRAJ49 | SSGNQFYF (20) | 1535 | 1.57 |
| 4 | TRAV2 | TRAJ34 | CAVLSYNTDKLIF (21) | 1345 | 1.38 |
| 5 | TRAV14/DV4 | TRAJ37 | CAMREVYGSSNTGKLIF (22) | 1080 | 1.11 |
| 6 | TRAV21 | TRAJ47 | CAAKEEYGNKLVF (23) | 984 | 1.01 |
| 7 | TRAV10 | TRAJ45 | CVVTTSMYSGGGADGLTF (24) | 691 | 0.71 |
| 8 | TRAV21 | TRAJ43 | CACDDNNDMRP (25) | 687 | 0.70 |
| 9 | TRAV8-4 | TRAJ17 | CAVRVVVKAAGNKLTF (26) | 569 | 0.58 |
| 10 | TRAV1-2 | TRAJ33 | CAVMDSNYQLIW (27) | 547 | 0.56 |
| 11 | TRAV21 | TRAJ10 | CAGYILTGGGNKLTF (28) | 539 | 0.55 |
| 12 | TRAV5 | TRAJ36 | CAESMQTGANNLFF (29) | 462 | 0.49 |
| 13 | TRAV1-2 | TRAJ33 | CAAMDSNYQLIW (30) | 337 | 0.35 |
| 14 | TRAV2 | TRAJ15 | CVVRTALIF (31) | 331 | 0.34 |
| 15 | TRAV35 | TRAJ45 | WG*RSGGGADGLTF (32) | 289 | 0.30 |
| 16 | TRAV24 | TRAJ49 | CARNTGNQFYF (33) | 268 | 0.27 |
| 17 | TRAV20 | TRAJ45 | CAVQAGGGADGLTF (34) | 266 | 0.27 |
| 18 | TRAV8-4 | TRAJ4 | CAVTPSGGYNKLIF (35) | 255 | 0.26 |
| 19 | TRAV2 | TRAJ22 | CAFPGGSARQLTF (36) | 250 | 0.26 |
| 20 | TRAV3 | TRAJ26 | CADYYGQNFVF (37) | 245 | 0.25 |
| 21 | TRAV13-1 | TRAJ36 | CAGNDQTGANNLFF (38) | 244 | 0.25 |
| 22 | TRAV16 | TRAJ26 | CALTPNYGQNFVF (39) | 238 | 0.24 |
| 23 | TRAV9-2 | TRAJ30 | CALTPNRDDKIIF (40) | 235 | 0.24 |

[Table 6-2]

| | | | | | |
|---|---|---|---|---|---|
| 24 | TRAV1-1 | TRAJ6 | CAARGSYIFTF (41) | 235 | 0.24 |
| 25 | TRAV17 | TRAJ56 | CATVPGANSKLTF (42) | 233 | 0.24 |
| 26 | TRAV14/DV4 | TRAJ26 | CAMRELINYGQNFVF (43) | 226 | 0.23 |
| 27 | TRAV13-1 | TRAJ6 | CAASKGGSYIFTF (44) | 214 | 0.22 |
| 28 | TRAV14/DV4 | TRAJ13 | CAMSLPRGYQKVTF (45) | 213 | 0.22 |
| 29 | TRAV8-3 | TRAJ52 | CAHGGGTSYGKLTF (46) | 212 | 0.22 |
| 30 | TRAV13-1 | TRAJ29 | CAAVNSGNTPLVF (47) | 208 | 0.21 |
| 31 | TRAV38-2/DV8 | TRAJ45 | CAYRRYSGGGADGLTF (48) | 207 | 0.21 |
| 32 | TRAV1-1 | TRAJ26 | CAVRRDGQNFVF (49) | 205 | 0.21 |
| 33 | TRAV13-1 | TRAJ5 | CAAPKGDGHGQESTY*F (50) | 202 | 0.21 |
| 34 | TRAV13-1 | TRAJ17 | CAASRKAAGNKLTF (51) | 202 | 0.21 |
| 35 | TRAV1-1 | TRAJ5 | CAVPMNTGRRALTF (52) | 199 | 0.20 |
| 36 | TRAV19 | TRAJ49 | CALSETNTGNQFYF (53) | 198 | 0.20 |
| 37 | TRAV2 | TRAJ16 | CAVSLSDGQKLLF (54) | 197 | 0.20 |
| 38 | TRAV29/DV5 | TRAJ54 | CAASALQGAQKLVF (55) | 195 | 0.20 |
| 39 | TRAV26-1 | TRAJ26 | CIVRPGGYGQNFVF (56) | 195 | 0.20 |
| 40 | TRAV29/DV5 | TRAJ45 | CAASGQEEVLTDS**F (57) | 194 | 0.20 |
| 41 | TRAV13-2 | TRAJ28 | CAEKGESGAGSYQLTF (58) | 194 | 0.20 |
| 42 | TRAV1-1 | TRAJ10 | CAVLTGGGNKLTF (59) | 193 | 0.20 |
| 43 | TRAV9-2 | TRAJ35 | CALRTSIGFGNVLHC (60) | 192 | 0.20 |
| 44 | TRAV13-1 | TRAJ52 | CAAGKGGTSYGKLTF (61) | 192 | 0.20 |
| 45 | TRAV1-1 | TRAJ30 | CAVRARDDKIIF (62) | 190 | 0.19 |
| 46 | TRAV13-1 | TRAJ16 | CAASRGGQKLLF (63) | 188 | 0.19 |
| 47 | TRAV23/DV6 | TRAJ16 | CAAGKSDGQKLLF (64) | 187 | 0.19 |
| 48 | TRAV41 | TRAJ58 | CAALRAETSGSRLTF (65) | 183 | 0.19 |
| 49 | TRAV13-1 | TRAJ6 | CAAGSYIPTF (66) | 183 | 0.19 |
| 50 | TRAV23/DV6 | TRAJ48 | CAASRSNFGNEKLTF (67) | 177 | 0.18 |

[symbol]

indicates data missing or illegible when filed

TABLE 7
Results of TCRβ repertoire analysis on (1) PRMCs

| | | | | | |
|---|---|---|---|---|---|
| Rank | TRBV | TRBJ | CDR3(SEQ ID NO) | Reads | [symbol] (Total Reads) |

[Table 7-1]

| | | | | | |
|---|---|---|---|---|---|
| 1 | TRBV30 | TRBJ2-1 | CALIRENPYNEQFF (68) | 7676 | 3.24 |
| 2 | TRBV10-3 | TRBJ2-1 | CAISEFGGAYDNEQFF (69) | 4878 | 2.06 |
| 3 | TRBV7-9 | TRBJ2-5 | CASSLAGTSGQTQYF (70) | 2842 | 1.20 |
| 4 | TRBV29-1 | TRBJ2-7 | CSVGRLPTSS**F (71) | 1151 | 0.49 |
| 5 | TRBV6-1 | TRBJ2-3 | CASSRRDTQYF (72) | 1062 | 0.45 |
| 6 | TRBV15 | TRBJ1-4 | CATSRQGATNEKLFF (73) | 1002 | 0.42 |
| 7 | TRBV20-1 | TRBJ2-7 | CSAKDLQENTREQYF (74) | 987 | 0.4 |
| 8 | TRBV7-9 | TRBJ1-5 | CASSQGQHDGEPQHF (75) | 909 | 0.38 |
| 9 | TRBV6-5 | TRBJ1-2 | CASSQQTGTIFFYTF (76) | 886 | 0.37 |
| 10 | TRBV4-1 | TRBJ1-1 | CASSHSGQGRSEAFF (77) | 865 | 0.36 |
| 11 | TRBV25-1 | TRBJ2-5 | CASESGQETQYF (78) | 858 | 0.36 |
| 12 | TRBV9 | TRBJ2-5 | CASSANQETQYF (79) | 791 | 0.33 |
| 13 | TRBV6-3 | TRBJ2-2 | CASSYSPDRVAGELFF (80) | 782 | 0.33 |
| 14 | TRBV6-1 | TRBJ1-1 | CASSENGTYTEAFF (81) | 778 | 0.33 |
| 15 | TRBV4-2 | TRBJ2-2 | CASRTRDKNTGSLFF (82) | 686 | 0.29 |
| 16 | TRBV4-2 | TRBJ2-7 | CASTLGGGALSYEQYF (83) | 627 | 0.26 |
| 17 | TRBV3-1 | TRBJ2-5 | CASSQGQLQETQYF (84) | 623 | 0.26 |
| 18 | TRBV5-5 | TRBJ1-1 | CASSFQGDTEAFF (85) | 621 | 0.26 |
| 19 | TRBV4-3 | TRBJ1-2 | CASSHRDRNPYGYTF (86) | 620 | 0.26 |
| 20 | TRBV2 | TRBJ2-3 | CASSDYGSTDYTQYF (87) | 561 | 0.24 |
| 21 | TRBV16 | TRBJ2-1 | CASSQYRGWNEQFF (88) | 531 | 0.22 |
| 22 | TRBV2 | TRBJ1-4 | CASSDEQGGEKLFF (89) | 535 | 0.22 |
| 23 | TRBV2 | TRBJ1-1 | CASSFPGRRAEAFF (90) | 525 | 0.22 |
| 24 | TRBV28 | TRBJ1-1 | CASKGQGDTGELFF (91) | 515 | 0.22 |
| 25 | TRBV28 | TRBJ2-2 | CASRQSGQEISYGYTF (92) | 480 | 0.20 |
| 26 | TRBV6-5 | TRBJ1-3 | CASRSGAGYQETQYF (93) | 476 | 0.20 |
| 27 | TRBV3-1 | TRBJ2-5 | CSAADGSSYNEQFF (94) | 471 | 0.20 |
| 28 | TRBV20-1 | TRBJ2-1 | CSAADGSSYNEQFF (95) | 471 | 0.20 |

[Table 7-2]

| | | | | | |
|---|---|---|---|---|---|
| 29 | TRBV6-6 | TRBJ2-7 | CASETGGDEQYF (96) | 455 | 0.19 |
| 30 | TRBV29-1 | TRBJ2-1 | CSVSLGREQFF (97) | 455 | 0.19 |
| 31 | TRBV23-1 | TRBJ2-7 | CASSLCLSHKDGRAV*F (98) | 435 | 0.18 |
| 32 | TRBV12-3 | TRBJ2-7 | CASRERTGMLHEQYF 999) | 431 | 0.18 |
| 33 | TRBV6-4 | TRBJ1-1 | CASSPVFLEGEVAEAFF (100) | 415 | 0.18 |
| 34 | TRBV2 | TRBJ1-2 | CASSVTGGAYGYTF (101) | 406 | 0.17 |
| 35 | TRBV5-6 | TRBJ2-6 | CASSSGTYYSGANVLTF (102) | 404 | 0.17 |
| 36 | TRBV6-1 | TRBJ12 | CASSEGDNYGYTF (103) | 396 | 0.17 |
| 37 | TRBV6-1 | TRBJ2-5 | CASSAPPLEETQYF (104) | 393 | 0.17 |
| 38 | TRBV3-1 | TRBJ1-6 | CASSLDFYSPLHF (105) | 390 | 0.16 |
| 39 | TRBV6-1 | TRBJ1-4 | CASSGWTETNEKLFF (106) | 384 | 0.16 |
| 40 | TRBV23-1 | TRBJ2-5 | CASSQLDRV**ETQYF (107) | 384 | 0.16 |
| 41 | TRBV3-1 | TRBJ2-7 | CASSPGPGQGTSRARYEQYF (108) | 381 | 0.16 |
| 42 | TRBV7-9 | TRBJ2-2 | CASSLGQGSTGELFF (109) | 374 | 0.16 |
| 43 | TRBV6-3 | TRBJ2-5 | CASQDRYQETQYF (110) | 374 | 0.16 |
| 44 | TRBV3-1 | TRBJ1-1 | CASSETGGRTEAFF (111) | 353 | 0.15 |
| 45 | TRBV6-1 | TRBJ2-1 | CASSVAGGSWGNEQFF (112) | 341 | 0.14 |
| 46 | TRBV15 | TRBJ2-3 | CATSRGGSTDTQYF (113) | 339 | 0.14 |
| 47 | TRBV5-6 | TRBJ1-6 | CASSVQGKSSPLHF (114) | 336 | 0.14 |
| 48 | TRBV28 | TRBJ1-6 | CASRGTADIPAPYNSPLHF (115) | 336 | 0.14 |
| 49 | TRBV-19 | TRBJ1-1 | CASRNRENTEAFF (116) | 334 | 0.14 |
| 50 | TRBV6-5 | TRBJ1-5 | CASNAPQGPQHF (117) | 332 | 0.14 |

[symbol]

indicates data missing or illegible when filed

TABLE 8
Results of TCRα repertoire analysis on (2) peptide stimulated cells cultured
for 1 week

| | | | | | |
|---|---|---|---|---|---|
| Rank | TRAV | TRAJ | CDR3(SEQ ID NO) | Reads | [symbol] (Total Reads) |

[Table 8-1]

| | | | | | |
|---|---|---|---|---|---|
| 1 | TRAV21 | TRAJ49 | SSGNQFYF (118) | 83409 | 42.76 |
| 2 | TRAV24 | TRAJ49 | CARNTGNQFYF (119) | 38830 | 19.93 |
| 3 | TRAV3 | TRAJ26 | CADYYGQNFYF (120) | 11888 | 6.09 |
| 4 | TRAV5 | TRAJ20 | CAETPTNDYKLSF (121) | 5804 | 2.98 |
| 5 | TRAV4 | TRAJ40 | CLVGDYSQEPTNT**F (122) | 2525 | 1.29 |
| 6 | TRAV27 | TRAJ43 | CAGAVGNDMRF (123) | 924 | 0.47 |
| 7 | TRAV21 | TRAJ47 | CAAKEEYGNKLVF (124) | 713 | 0.37 |
| 8 | TRAV10 | TRAJ45 | CVVTTSMYSGGGADGLTF (125) | 565 | 0.29 |
| 9 | TRAV21 | TRAJ43 | CACDDNNDMRP (126) | 487 | 0.25 |
| 10 | TRAV38-2/DV8 | TRAJ40 | CAHITSGTYKYIF (127) | 305 | 0.15 |
| 11 | TRAV17 | TRAJ26 | CATDGNYGQNFVF (128) | 281 | 0.14 |
| 12 | TRAV12-1 | TRAJ21 | CVVNWSFNKFYF (129) | 219 | 0.11 |
| 13 | TRAV21 | TRAJ49 | SAGNQFYF (130) | 217 | 0.11 |
| 14 | TRAV24 | TRAJ49 | SSGNQFYF (131) | 217 | 0.11 |
| 15 | TRAV13-1 | TRAJ27 | CAASRARTNAGKSTF (132) | 144 | 0.07 |
| 16 | TRAV1-1 | TRAJ27 | CAVRDQGPMQANQ**F (133) | 144 | 0.07 |
| 17 | TRAV6 | TRAJ3 | CALSYSSASKIIF (134) | 143 | 0.07 |
| 18 | TRAV2 | TRAJ34 | CAVLSYNTDKLIF (135) | 133 | 0.07 |
| 19 | TRAV21 | TRAJ49 | CARNTGNQFYF (136) | 122 | 0.06 |
| 20 | TRAV1-3 | TRAJ33 | CALMDNYQLIW (137) | 121 | 0.06 |
| 21 | TRAV20 | TRAJ29 | CAVRNSGNTPLVF (138) | 106 | 0.05 |
| 22 | TRAV13-1 | TRAJ5 | CAASNTGRRALTF (139) | 102 | 0.05 |
| 23 | TRAV3 | TRAJ23 | CAVRDSNQGGKLIF (140) | 101 | 0.05 |
| 24 | TRAV10 | TRAJ18 | CVVSDRGSTLGRLYF (141) | 97 | 0.05 |
| 25 | TRAV1-2 | TRAJ33 | CASMDSNYQLIW (142) | 91 | 0.05 |
| 26 | TRAV19 | TRAJ22 | CALSEGYRVLQGN**TF (143} | 88 | 0.05 |
| 27 | TRAV24 | TRAJ49 | CVRNTGNQFYF (144) | 87 | 0.04 |
| 28 | TRAV1-2 | TRAJ33 | CAVRDSNYQLIW (145) | 85 | 0.04 |
| 29 | TRAV1-2 | TRAJ33 | CAVMDSNYQLIW (146) | 85 | 0.04 |

[Table 8-2]

| | | | | | |
|---|---|---|---|---|---|
| 30 | TRAV16 | TRAJ20 | CALNDYKLSF (147) | 83 | 0.04 |
| 31 | TRAV1-2 | TRAJ33 | CAAMDSNYQLIW (148) | 83 | 0.04 |
| 32 | TRAV13-1 | TRAJ6 | CAAKSGGSYIPTF (149) | 82 | 0.04 |
| 33 | TRAV27 | TRAJ49 | CAGATGNQFYF (150) | 78 | 0.04 |
| 34 | TRAV13-1 | TRAJ54 | CAAPVGQGAQKLVF (151) | 77 | 0.04 |
| 35 | TRAV13-1 | TRAJ39 | CRQHAH*F (152) | 76 | 0.04 |
| 36 | TRAV9-2 | TRAJ58 | CALSGAAETSGSRLTF (153) | 74 | 0.04 |
| 37 | TRAV27 | TRAJ47 | CAGALGNKLVF (154) | 73 | 0.04 |
| 38 | TRAV12-1 | TRAJ26 | CVVRDNYGQNFVF (155) | 72 | 0.04 |
| 39 | TRAV19 | TRAJ24 | CALSGSTDSWGKFQF (156) | 71 | 0.04 |
| 40 | TRAV19 | TRAJ16 | CALSEAGWPEAA*F (157) | 71 | 0.04 |
| 41 | TRAV17 | TRAJ56 | CATVPGANSKLTF (158) | 71 | 0.04 |
| 42 | TRAV13-1 | TRAJ36 | CAAQWGANNLFF (159) | 71 | 0.04 |
| 43 | TRAV19 | TRAJ30 | CALTQAPDDKIIF (160) | 70 | 0.04 |
| 44 | TRAV39 | TRAJ39 | CAVPRMQATCS**F (161) | 69 | 0.04 |
| 45 | TRAV2 | TRAJ34 | CAVGLNTDKLIF (162) | 69 | 0.04 |
| 46 | TRAV13-1 | TRAJ42 | CAATNGGSQGNLIF (163) | 68 | 0.03 |
| 47 | TRAV13-1 | TRAJ26 | CAASRSDDYSLSF (164) | 68 | 0.03 |
| 48 | TRAV6 | TRAJ30 | CALEIDDKIIF (165) | 67 | 0.03 |
| 49 | TRAV8-4 | TRAJ3 | CAVSERTAVLPR**IF (166) | 65 | 0.03 |
| 50 | TRAV19 | TRAJ28 | CALSEAGYSGAGSYQLTF (167) | 65 | 0.03 |

[symbol]

indicates data missing or illegible when filed

TABLE 9
Results of TCRβ repertoire analysis on (2) peptide stimulated cells cultured
for 1 week

| | | | | | |
|---|---|---|---|---|---|
| Rank | TRBV | TRBJ | CDR3(SEQ ID NO) | Reads | [symbol] (Total Reads) |

[Table 9-1]

| | | | | | |
|---|---|---|---|---|---|
| 1 | TRBV6-5 | TRBJ1-2 | CASSQQTGTIFFYTF (168) | 66506 | 35.63 |
| 2 | TRBV28 | TRBJ1-1 | CASSFQGFTEAFF (169) | 34912 | 18.71 |
| 3 | TRBV6-6 | TRBJ1-2 | CASSSETELLYYGYTF (170) | 2852 | 1.53 |
| 4 | TRBV29-1 | TRBJ2-7 | CSVGRLPTSS**F (171) | 2528 | 1.35 |
| 5 | TRBV4-2 | TRBJ2-7 | CASSQGTSHSYEQYF (172) | 672 | 0.36 |
| 6 | TRBV7-9 | TRBJ1-3 | CASSLRPDGDPSGNTIYF (173) | 655 | 0.35 |
| 7 | TRBV28 | TRBJ1-2 | CASSPTGEDYGYTF (174) | 477 | 0.26 |
| 8 | TRBV30 | TRBJ1-1 | CAWSENTEAFF (175) | 476 | 0.26 |
| 9 | TRBV4-3 | TRBJ2-3 | CASSQDPGQGSDTQYF (176) | 469 | 0.25 |
| 10 | TRBV6-5 | TRBJ1-2 | CASSQITGTGYYGYTF (177) | 285 | 0.15 |
| 11 | TRBV30 | TRBJ2-1 | CALIRENPYNEQFF (178) | 259 | 0.14 |
| 12 | TRBV15 | TRBJ1-4 | CATSRQGATNEKLFF (179) | 210 | 0.11 |
| 13 | TRBV3-1 | TRBJ1-5 | CASSQGAGGYGSNQPQHF (180) | 203 | 0.11 |
| 14 | TRBV5-1 | TRBJ2-7 | CASSLEGRVTYEQYF (181) | 197 | 0.11 |
| 15 | TRBV30 | TRBJ2-5 | CAWSGDYNQETQYF (182) | 197 | 0.11 |
| 16 | TRBV5-4 | TRBJ2-1 | CASSELAEALNNEQFF (183) | 192 | 0.10 |
| 17 | TRBV9 | TRBJ2-1 | CASSVGASGSIDEQFF (184) | 163 | 0.09 |
| 18 | TRBV6-1 | TRBJ2-2 | CASSRTSGDTGELFF (185) | 163 | 0.09 |
| 19 | TRBV7-2 | TRBJ2-4 | CASSSSGTVAKNIQYF (186) | 162 | 0.09 |
| 20 | TRBV6-6 | TRBJ1-2 | CASSQQTGTIGGYTF (187) | 160 | 0.09 |
| 21 | TRBV7-9 | TRBJ2-5 | CASSLAGTSGQTQYF (188) | 145 | 0.08 |
| 22 | TRBV7-9 | TRBJ2-3 | CASRFGASGDRLHTQYF (189) | 143 | 0.08 |
| 23 | TRBV6-5 | TRBJ1-2 | CANSQQTGTIGGYTF (190) | 138 | 0.07 |
| 24 | TRBV7-2 | TRBJ3-2 | CASSLAVNTGELFF (191) | 136 | 0.07 |
| 25 | TRBV28 | TRBJ1-1 | CASRTGDGTEAFP (192) | 132 | 0.07 |
| 26 | TRBV6-1 | TRBJ2-1 | CASSELAAVYNEQFF (193) | 126 | 0.07 |
| 27 | TRBV6-1 | TRBJ1-5 | CASTGAGGNQPQHF (194) | 117 | 0.06 |
| 28 | TRBV6-5 | TRBJ1-5 | CASSTQGGHQPQHF (195) | 116 | 0.06 |
| 29 | TRBV6-5 | TRBJ1-2 | CVSSQQTGTIGGYTF (196) | 116 | 0.06 |

[Table 9-2]

| | | | | | |
|---|---|---|---|---|---|
| 30 | TRBV3-1 | TRBJ2-7 | CASSFGPGQGTSRARYEQYF (197) | 116 | 0.06 |
| 31 | TRBV20-1 | TRBJ2-1 | CSARGEEPRDSNYNEQFF (198) | 116 | 0.06 |
| 32 | TRBV6-5 | TRBJ2-7 | CASSNPY*SGGLSYEQYF (199) | 114 | 0.06 |
| 33 | TRBV5-1 | TRBJ2-2 | CASSTGTDNTGELFP (200) | 113 | 0.06 |
| 34 | TRBV5-4 | TRBJ2-6 | CASSFSSGANVLTF (201) | 111 | 0.06 |
| 35 | TRBV5-4 | TRBJ2-1 | CASSPGQGIREQFF (202) | 111 | 0.06 |
| 36 | TRBV20-1 | TRBJ2-1 | CSAPVPPYNEQFF (203) | 111 | 0.06 |
| 37 | TRBV2 | TRBJ2-3 | CASSDTGLAGGGYTDTQYF (204) | 111 | 0.06 |
| 35 | TRBV7-2 | TRBJ2-1 | CASSLWTQQFF (205) | 109 | 0.06 |
| 39 | TRBV25-1 | TRBJ1-1 | CASSDIAGKENTEAFF (206) | 109 | 0.06 |
| 40 | TRBV11-2 | TRBJ2-7 | CASSLYRGVEQYF (207) | 109 | 0.06 |
| 41 | TRBV7-9 | TRBJ2-5 | CASSPTGKQETQYF (208) | 108 | 0.06 |
| 42 | TRBV6-1 | TRBJ2-7 | CASSETSEQYF (209) | 107 | 0.06 |
| 43 | TRBV29-1 | TRBJ2-7 | CSVDLDTSSYEQYF (210) | 106 | 0.06 |
| 44 | TRBV28 | TRBJ2-3 | CASRPREGRATDTQYF (211) | 103 | 0.06 |
| 45 | TRBV6-1 | TRBJ2-3 | CASSDGGGSGADTQYF (212) | 99 | 0.05 |
| 46 | TRBV4-1 | TRBJ1-1 | CASSHSGQGRSEAFF (213) | 99 | 0.05 |
| 47 | TRBV6-1 | TRBJ2-1 | CASNRDRGYNEQFF (214) | 98 | 0.05 |
| 48 | TRBV7-9 | TRBJ2-7 | CASSPGGSYEQYF (215) | 96 | 0.05 |
| 49 | TRBV6-5 | TRBJ1-1 | CASSFLMNTEAFF (216) | 96 | 0.05 |
| 50 | TRBV11-2 | TRBJ1-1 | CASSLDRGANTEAFF (217) | 95 | 0.05 |

[symbol]

indicates data missing or illegible when filed

TABLE 10
Results of TCRα repertoire analysis on (3) P2 region CD8+, MHC pp65 epitope
tetramer positive cells

| | | | | | |
|---|---|---|---|---|---|
| Rank | TRBV | TRBJ | CDR3(SEQ ID NO) | Reads | [symbol] (Total Reads) |

[Table 10-1]

| | | | | | |
|---|---|---|---|---|---|
| 1 | TRAV21 | TRAJ49 | SSGNQFYF (218) | 98892 | 46.93 |
| 2 | TRAV24 | TRAJ49 | CARNTGNQFYF (219) | 53706 | 25.49 |
| 3 | TRAV3 | TRAJ26 | CADYYGQNFVF (220) | 17599 | 8.35 |
| 4 | TRAV5 | TRAJ20 | CAETPTNDYKLSF (221) | 8513 | 4.04 |
| 5 | TRAV4 | TRAJ40 | CLVGDYSQEPTNT**F (222) | 2706 | 1.28 |
| 6 | TRAV24 | TRAJ49 | SSGNQFYF (223) | 431 | 0.20 |
| 7 | TRAV21 | TRAJ49 | CARNTGNQFYF (224) | 271 | 0.13 |
| 8 | TRAV21 | TRAJ49 | SAGNQFYF (225) | 224 | 0.11 |
| 9 | TRAV24 | TRAJ49 | CAFITGNQFYF (226) | 111 | 0.05 |
| 10 | TRAV24 | TRAJ49 | CVRNTGNQFYF (227) | 101 | 0.05 |
| 11 | TRAV24 | TRAJ49 | CARNTGNQFY**W (228) | 100 | 0.05 |
| 12 | TRAV3 | TRAJ34 | CAVRDMGTPTSS*F (229) | 90 | 0.04 |
| 13 | TRAV24 | TRAJ49 | CARNTGNQFYL (230) | 73 | 0.03 |
| 14 | TRAV24 | TRAJ49 | CAQNTGNQFYF (231) | 69 | 0.03 |
| 15 | TRAV24 | TRAJ49 | CARNTGNKFYF(232) | 60 | 0.03 |
| 16 | TRAV8-6 | TRAJ13 | CAVRYQKVTF (233) | 59 | 0.03 |
| 17 | TRAV24 | TRAJ49 | CDRNTGNQFYF (234) | 59 | 0.03 |
| 18 | TRAV24 | TRAJ49 | CARNTDNQFYF (235) | 53 | 0.03 |
| 19 | TRAV24 | TRAJ49 | CARNTSNQFYF (236) | 45 | 0.02 |
| 20 | TRAV24 | TRAJ49 | CAWNTGNQFYF (237) | 44 | 0.02 |
| 21 | TRAV24 | TRAJ49 | CTRNTGNQFYF (238) | 41 | 0.02 |
| 22 | TRAV4 | TRAJ40 | CLVGDYSQEPTNTSW (239) | 40 | 0.02 |
| 23 | TRAV | TRAJ31 | CAVRDTSARLMF (240) | 39 | 0.02 |
| 24 | TRAV21 | TRAJ49 | CF | 34 | 0.02 |
| 25 | TRAV24 | TRAJ49 | CARKTGNQFYF (241) | 32 | 0.02 |
| 26 | TRAV21 | TRAJ49 | CPSSGNQFYF (242) | 32 | 0.02 |
| 27 | TRAV24 | TRAJ49 | CARNTGNQF.F (243) | 31 | 0.01 |
| 28 | TRAV24 | TRAJ49 | ARNTGNQFYF (244) | 28 | 0.01 |
| 29 | TRAV24 | TRAJ49 | CARNR*NQFYF (245) | 27 | 0.01 |

[Table 10-2]

| | | | | | |
|---|---|---|---|---|---|
| 30 | TRAV24 | TRAJ49 | YARNTGNQFYF (246) | 26 | 0.01 |
| 31 | TRAV24 | TRAJ49 | CARNTGNQFCF (247) | 26 | 0.01 |
| 32 | TRAV24 | TRAJ49 | RARNTGNQFYF (248) | 25 | 0.01 |
| 33 | TRAV21 | TRAJ49 | C**F (249) | 25 | 0.01 |
| 34 | TRAV24 | TRAJ49 | CARNTGNQLYF (250) | 24 | 0.01 |
| 35 | TRAV24 | TRAJ49 | CARNTGNQF*P (251) | 24 | 0.01 |
| 36 | TRAV24 | TRAJ49 | CARNIGNQFYF (252) | 24 | 0.01 |
| 37 | TRAV21 | TRAJ49 | CNQFYF (253) | 24 | 0.01 |
| 38 | TRAV24 | TRAJ49 | CARNNGNQFYF (254) | 23 | 0.01 |
| 39 | TRAV3 | TRAJ26 | CVDYYGQNFVF (255) | 22 | 0.01 |
| 40 | TRAV24 | TRAJ49 | CARNTCNQFYF (256) | 22 | 0.01 |
| 41 | TRAV24 | TRAJ49 | CARDTGNQFYP (257) | 22 | 0.01 |
| 42 | TRAV24 | TRAJ49 | SARNTGNQFYF (258) | 21 | 0.01 |
| 43 | TRAV24 | TRAJ49 | CARSTGNQFYF (259) | 21 | 0.01 |
| 44 | TRAV24 | TRAJ49 | CARNAGNQFYF (260) | 20 | 0.01 |
| 45 | TRAV24 | TRAJ49 | CARNTGNQYYF (261) | 19 | 0.01 |
| 46 | TRAV5 | TRAJ20 | CAEAPTNDYKLSF (262) | 18 | 0.01 |
| 47 | TRAV24 | TRAJ49 | WARNTGNQFYP (263) | 18 | 0.01 |
| 48 | TRAV24 | TRAJ49 | CSRNTGNQFYF (264) | 18 | 0.01 |
| 49 | TRAV24 | TRAJ49 | CARNTGSQFYF (265) | 18 | 0.01 |
| 50 | TRAV3 | TRAJ26 | CAGYYGQNFVF (266) | 17 | 0.01 |

[symbol]

indicates data missing or illegible when filed

TABLE 11
Results of TCRβ repertoire analysis on (3) P2 region CD8+, MHC pp65 epitope
positive cells

| | | | | | |
|---|---|---|---|---|---|
| Rank | TRBV | TRBJ | CDR3(SEQ ID NO) | Reads | [symbol] (Total Reads) |

[Table 11-1]

| | | | | | |
|---|---|---|---|---|---|
| 1 | TRBV6-5 | TRBJ1-2 | CASSQQTGTIGGYTF (267) | 81763 | 42.83 |
| 2 | TRBV28 | TRBJ1-1 | CASSFQGFTEAFF (268) | 59805 | 31.33 |
| 3 | TRBV6-6 | TRBJ1-2 | CASSSETELLYYGYTF (269) | 4071 | 2.13 |
| 4 | TRBV6-6 | TRBJ1-2 | CASSQQTGTIGGYTF (270) | 440 | 0.23 |
| 5 | TRBV28 | TRBJ1-2 | CASSQQTGTIGGYTF (271) | 263 | 0.14 |
| 6 | TRBV6-5 | TRBJ1-1 | CASSFQGFTEAFF (272) | 250 | 0.13 |
| 7 | TRBV6-5 | TRBJ1-2 | CANSQQTGTIGGYTF (273) | 182 | 0.10 |
| 8 | TRBV6-5 | TRBJ1-2 | SVSSQQTGTIGGYTF (274) | 143 | 0.07 |
| 9 | TRBV6-5 | TRBJ1-2 | CASSSETELLYYGYTF (275) | 143 | 0.07 |
| 10 | TRBV28 | TRBJ1-1 | CANSFQGFTEAFF (276) | 128 | 0.07 |
| 11 | TRBV28 | TRBJ1-1 | RASSFQGFTEAFF (277) | 101 | 0.05 |
| 12 | TRBV6-5 | TRBJ1-2 | CASSQ.TGTIGGYTF (278) | 98 | 0.05 |
| 13 | TRBV28 | TRBJ1-1 | CVSSFQFFTEAFF (279) | 95 | 0.05 |
| 14 | TRBV28 | TRBJ1-1 | CTSSFQGFTEAFF (280) | 71 | 0.04 |
| 15 | TRBV6-5 | TRBJ1-2 | CASSQQTGTIGGHTF (281) | 70 | 0.04 |
| 16 | TRBV6-5 | TRBJ1-2 | CTSSQQTGTIGGYTF (282) | 68 | 0.04 |
| 17 | TRBV6-5 | TRBJ1-2 | CASSQQTGAIGGYTF (283) | 68 | 0.04 |
| 18 | TRBV6-5 | TRBJ1-2 | CASRQQTGTIGGYTF (284) | 68 | 0.04 |
| 19 | TRBV6-5 | TRBJ1-2 | CASSQQTGMIGGYTF (285) | 60 | 0.03 |
| 20 | TRBV28 | TRBJ1-1 | CASSFQGFTEDFF (286) | 59 | 0.03 |
| 21 | TRBV6-5 | TRBJ1-2 | CASSQQTGTIGGYTL (287) | 54 | 0.03 |
| 22 | TRBV6-5 | TRBJ1-2 | CASSQQTGTIGGYAF (288) | 48 | 0.03 |
| 23 | TRBV28 | TRBJ1-1 | CASSFQGFTEAFL (289) | 48 | 0.03 |
| 24 | TRBV28 | TRBJ1-1 | CASSFQGFIEAFF (290) | 47 | 0.02 |
| 25 | TRBV28 | TRBJ1-1 | CASSLQGFTEAFF (291) | 45 | 0.02 |
| 26 | TRBV28 | TRBJ1-1 | CASSFRGFTEAFP (292) | 45 | 0.02 |
| 27 | TRBV6-5 | TRBJ1-2 | CASSQITGTGYYGYTF (293) | 44 | 0.02 |
| 28 | TRBV6-5 | TRBJ1-2 | CASSQQTGTMGGYTF (294) | 43 | 0.02 |
| 29 | TRBV28 | TRBJ1-1 | CASSFQGFNEAFF (295) | 43 | 0.02 |

[Table 11-2]

| | | | | | |
|---|---|---|---|---|---|
| 30 | TRBV6-5 | TRBJ1-2 | WASSQQTGTIGGYTF (296) | 40 | 0.02 |
| 31 | TRBV6-5 | TRBJ1-2 | RASSQQTGTIGGYTF (297) | 40 | 0.02 |
| 32 | TRBV6-5 | TRBJ1-2 | CASSQQTGTIGGYPF (298) | 40 | 0.02 |
| 33 | TRBV6-5 | TRBJ1-2 | CASSQQTGTIGGYTI (299) | 39 | 0.02 |
| 34 | TRBV6-5 | TRBJ1-2 | CASNQQTGTIGGYTF (300) | 37 | 0.02 |
| 35 | TRBV28 | TRBJ1-4 | CASSFQGFTEAFFGQGTRLTVV | 37 | 0.02 |
| | | | GLKFY*KEQERTWQVDLGRQE | | |
| | | | WKAAGRGFPSSL.CCATNEKLF | | |
| | | | F (301) | | |
| 36 | TRBV28 | TRBJ1-1 | CASRFQGFTEAFF (302) | 37 | 0.02 |
| 37 | TRBV6-5 | TRBJ1-2 | CASSQQAGTIGGYTF (303) | 36 | 0.02 |
| 38 | TRBV6-5 | TRBJ1-2 | .ASSQQTGTIGGYTF (304) | 36 | 0.02 |
| 39 | TRBV6-5 | TRBJ1-2 | CSSSQQTGTIGGYTF (305) | 35 | 0.02 |
| 40 | TRBV6-5 | TRBJ1-2 | CASSQQTVTIGGYTF (306) | 35 | 0.02 |
| 41 | TRBV6-5 | TRBJ1-2 | CASSQQTETIGGYTF (307) | 35 | 0.02 |
| 42 | TRBV6-3 | TRBJ1-2 | CASSWDGLYGYTF (308) | 35 | 0.02 |
| 43 | TRBV28 | TRBJ1-1 | CASSFQGLTEAFF (309) | 35 | 0.02 |
| 44 | TRBV28 | TRBJ1-1 | CASSFQEFTEAFF (310) | 35 | 0.02 |
| 45 | TRBV6-5 | TRBJ1-2 | CASSQQTGTIGGYSF (311) | 34 | 0.02 |
| 46 | TRBV6-5 | TRBJ1-2 | CASSQQTGTIGDYTF (312) | 34 | 0.02 |
| 47 | TRBV6-5 | TRBJ2-2 | CASTESTGVSTGELPF (313) | 33 | 0.02 |
| 48 | TRBV6-5 | TRBJ1-2 | CASSKQTGTIGGYTF (314) | 33 | 0.02 |
| 49 | TRBV6-5 | TRBJ1-2 | CASSQQTRTIGGYTF (315) | 32 | 0.02 |
| 50 | TRBV6-5 | TRBJ1-2 | CASSQQTGTIGGYIF (315) | 32 | 0.02 |

[symbol]

indicates data missing or illegible when filed

TABLE 12
Results of TCRa repertoire analysis on (4) P2 region CD8+, MHC eptiope tetramer
negative cells

| | | | | | |
|---|---|---|---|---|---|
| Rank | TRBV | TRBJ | CDR3(SEQ ID NO) | Reads | [symbol] (Total Reads) |

[Table 12-1]

| | | | | | |
|---|---|---|---|---|---|
| 1 | TRAV21 | TRAJ47 | CAAKEEYGNKLVF (317) | 17591 | 11.44 |
| 2 | TRAV27 | TRAJ43 | CAGAVGNDMRF (318) | 15023 | 9.77 |
| 3 | TRAV21 | TRAJ43 | CACDDNNDMRF(319) | 7842 | 5.10 |
| 4 | TRAV10 | TRAJ45 | CVVTTSMYSGGGADGLTF (320) | 4350 | 2.83 |
| 5 | TRAV1-2 | TRAJ33 | CAVRDSNYQLIW (321) | 2346 | 1.53 |
| 6 | TRAV19 | TRAJ42 | CALSNYGGSQGNLIF (322) | 2257 | 1.47 |
| 7 | TRAV27 | TRAJ49 | CAGATGNQFYF (323) | 2172 | 1.41 |
| 8 | TRAV1-2 | TRAJ33 | CAVMDSNYQLIW (324) | 2168 | 1.41 |
| 9 | TRAV27 | TRAJ49 | CAGGTGNQFYF (325) | 2056 | 1.34 |
| 10 | TRAV17 | TRAJ26 | CATDGNYGQNFVF (326) | 1832 | 1.19 |
| 11 | TRAV12-1 | TRAJ21 | CVVSWSFNKFYF (327) | 1810 | 1.18 |
| 12 | TRAV19 | TRAJ52 | CALSEAGTSYGKLTF (328) | 1760 | 1.14 |
| 13 | TRAV13-2 | TRAJ28 | CAEKGESGAGSVQLTF (329) | 1516 | 0.99 |
| 14 | TRAV19 | TRAJ52 | CALSELTGGTSYGKLTF (330) | 1500 | 0.98 |
| 15 | TRAV8-4 | TRAJ3 | CAVSERTAVLPR**IF (331) | 1391 | 0.90 |
| 16 | TRAV1-2 | TRAJ33 | CASMDSNYQLIW (332) | 1374 | 0.89 |
| 17 | TRAV13-1 | TRAJ27 | CAASRARTNAGKSTF (333) | 1361 | 0.89 |
| 18 | TRAV13-1 | TRAJ29 | CAASIRGNSGNTPLVF (334) | 1303 | 0.85 |
| 19 | TRAV29/DV5 | TRAJ29 | CAAGNSGNTPLVF (335) | 1241 | 0.81 |
| 20 | TRAV13-1 | TRAJ43 | CAASNNNDMRF (336) | 1167 | 0.76 |
| 21 | TRAV22 | TRAJ53 | CAAPKSISGGSNYKLTF (337) | 1116 | 0.73 |
| 22 | TRAV8-4 | TRAJ49 | CAVTLRNGHR*NQFYF (338) | 1092 | 0.71 |
| 23 | TRAV8-2 | TRAJ4 | CVTFSGGVNKLIF (339) | 1068 | 0.69 |
| 24 | TRAV1-1 | TRAJ33 | CAVRDEGSNYQLIW (340) | 1021 | 0.66 |
| 25 | TRAV1-2 | TRAJ33 | CAVTDSNYQLIW (341) | 939 | 0.61 |
| 26 | TRAV39 | TRAJ39 | CADPWGHNAGNMLTF (342) | 925 | 0.60 |
| 27 | TRAV13-1 | TRAJ7 | .SEGNNRLAF (343) | 913 | 0.59 |
| 28 | TRAV27 | TRAJ47 | CAGAVGNKLVF (344) | 900 | 0.59 |
| 29 | TRAV1-2 | TRAJ33 | CAVIDSNYQLIW (345) | 811 | 0.53 |

[Table 12-2]

| | | | | | |
|---|---|---|---|---|---|
| 30 | TRAV26-2 | TRAJ48 | CILRDVPLWK*EKLTF (346) | 779 | 0.51 |
| 31 | TRAV12-2 | TRAJ54 | CAVSTQGAQKLVP (347) | 778 | 0.51 |
| 32 | TRAV12-3 | TRAJ12 | CAWVGDSSYKLIF (348) | 775 | 0.50 |
| 33 | TRAV12-3 | TRAJ6 | CAMSASGGSYIFTF (349) | 768 | 0.50 |
| 34 | TRAV2 | TRAJ34 | CAVGLNTDKLIF (350) | 761 | 0.49 |
| 35 | TRAV21 | TRAJ49 | SSGNQFYF (351) | 742 | 0.48 |
| 36 | TRAV38-2/DV8 | TRAJ40 | CAHITSGTYKYIF (352) | 736 | 0.48 |
| 37 | TRAV5 | TRAJ42 | CARVWRKPRKSH*F (353) | 716 | 0.47 |
| 38 | TRAV21 | TRAJ21 | CAVNVFNKFYF (354) | 714 | 0.46 |
| 39 | TRAV21 | TRAJ20 | CAVRNDYKLSF (355) | 706 | 0.46 |
| 40 | TRAV9-2 | TRAJ10 | CALSDHGDSREEETNS**F (356) | 690 | 0.45 |
| 41 | TRAV3 | TRAJ32 | CAVRDILGGATNKLIF (357) | 676 | 0.44 |
| 42 | TRAV14/DV4 | TRAJ52 | CAMGLMLVVLAMES**TF (358) | 664 | 0.43 |
| 43 | TRAV20 | TRAJ12 | CAVRVDSSYKLIF (359) | 636 | 0.41 |
| 44 | TRAV19 | TRAJ45 | CALSEAADGLTF (360) | 608 | 0.40 |
| 45 | TRAV21 | TRAJ10 | CAGYILTGGGNKLTF (361) | 555 | 0.36 |
| 46 | TRAV21 | TRAJ18 | CAVNRGSTLGRLYF (362) | 529 | 0.34 |
| 47 | TRAV17 | TRAJ56 | CATVPGANSKLTF (363) | 525 | 0.34 |
| 48 | TRAV3 | TRAJ11 | CAVRDRAQDTAPS**F (364) | 518 | 0.34 |
| 49 | TRAV6 | TRAJ26 | CARPLGQNFVF (365) | 501 | 0.33 |
| 50 | TRAV1-1 | TRAJ33 | CAVRSDSNYQLIW (366) | 591 | 0.32 |

[symbol]

indicates data missing or illegible when filed

TABLE 13
Results of TCRβ repertoire analysis on (4) P2 region CD8+, MHC pp65 eptiope
tetramer negative cells

| | | | | | |
|---|---|---|---|---|---|
| Rank | TRBV | TRBJ | CDR3(SEQ ID NO) | Reads | [symbol] (Total Reads) |

[Table 13-1]

| | | | | | |
|---|---|---|---|---|---|
| 1 | TRBV4-3 | TRBJ2-3 | CASSQDPGQGSDTQYF (367) | 10440 | 4.82 |
| 2 | TRBV7-9 | TRBJ1-3 | CASSLRPDGDPSGNTIYF (368) | 8655 | 4.00 |
| 3 | TRBV28 | TRBJ1-2 | CASSPTGEDYGYTF (369) | 7631 | 3.53 |
| 4 | TRBV4-2 | TRBJ2-7 | CASSQGTSHSYEQYF (370) | 7422 | 3.43 |
| 5 | TRBV30 | TRBJ1-1 | CAWSENTEAFF (371) | 6314 | 2.92 |
| 6 | TRBV15 | TRBJ1-4 | CATSRQGATNEKLFF (372) | 4596 | 2.12 |
| 7 | TRBV30 | TRBJ2-1 | CALIRENPYNEQFF (373) | 3417 | 1.58 |
| 8 | TRBV4-1 | TRBJ2-5 | CASRGGRVRETQYF (374) | 2270 | 1.05 |
| 9 | TRBV7-9 | TRBJ1-6 | CASSLMGSSYNSPLHP (375) | 2000 | 0.92 |
| 10 | TRBV6-1 | TRBJ2-3 | CASSEGLAGADTQYF (376) | 1875 | 0.87 |
| 11 | TRBV10-3 | TRBJ2-6 | CAISEVEGSGANVLTF (377) | 1693 | 0.78 |
| 12 | TRBV79 | TRBJ2-1 | CASSPPSGGPNEQFF (378) | 1677 | 0.78 |
| 13 | TRBV4-1 | TRBJ1-1 | CASSHSGQGRSEAFF (379) | 1630 | 0.75 |
| 14 | TRBV6-1 | TRBJ2-1 | CASSEIGGLHNEEQFF (380) | 1338 | 0.62 |
| 15 | TRBV20-1 | TRBJ2-2 | CSAKTQGDTGELFF (381) | 1333 | 0.62 |
| 16 | TRBV27 | TRBJ2-2 | CASSLSTVGELFF (382) | 1331 | 0.62 |
| 17 | TRBV7-9 | TRBJ2-5 | CASSPTGKQETQYF (383) | 1326 | 0.61 |
| 18 | TRBV7-9 | TRBJ2-5 | CASSAPLSQETQYF (384) | 1300 | 0.60 |
| 19 | TRBV29-1 | TRBJ2-7 | CSVGRLPTSS**F (385) | 1255 | 0.58 |
| 20 | TRBV11-2 | TRBJ2-7 | CASSLFRGYEQYF (386) | 1065 | 0.49 |
| 21 | TRBV5-1 | TRBJ1-1 | CASSLDRGH*EAFF (387) | 1064 | 0.49 |
| 22 | TRBV27 | TRBJ2-1 | CASSLSGRSSYNEQFF (388) | 1043 | 0.48 |
| 23 | TRBV6-4 | TRBJ2-1 | CASSEKASGADEQFF (389) | 1030 | 0.48 |
| 24 | TRBV7-6 | TRBJ2-1 | CASSLTSGSGAEQFF (390) | 1018 | 0.47 |
| 25 | TRBV3-1 | TRBJ1-1 | CASSQEFSDRGPEAFF (391) | 935 | 0.43 |
| 26 | TRBV7-2 | TRBJ2-4 | CASSSGTVAKNIQYF (392) | 923 | 0.43 |
| 27 | TRBV2 | TRBJ1-2 | CASSVTGGAYGYTP (393) | 882 | 0.41 |
| 28 | TRBV29-1 | TRBJ2-7 | CSVETDGYEQYF (394) | 805 | 0.37 |
| 29 | TRBV6-5 | TRBJ2-3 | CASSPLAGGADTQYF (395) | 770 | 0.36 |

[Table 13-2]

| | | | | | |
|---|---|---|---|---|---|
| 30 | TRBV27 | TRBJ2-1 | CASFGLAGHSYNEQFF (396) | 760 | 0.35 |
| 31 | TRBV27 | TRBJ2-7 | CASSPFYQGDDEQYF (397) | 751 | 0.35 |
| 32 | TRBV7-9 | TRBJ1-2 | CASSPNRGGGYTF (398) | 675 | 0.31 |
| 33 | TRBV4-1 | TRBJ1-5 | CASSQESVSSFSNQPQHF (399) | 667 | 0.31 |
| 34 | TRBV27 | TRBJ2-2 | CASRTGDTGELFF (400) | 659 | 0.30 |
| 35 | TRBV30 | TRBJ2-5 | CAWSGDYNQETQYF (401) | 657 | 0.30 |
| 36 | TRBV30 | TRBJ1-1 | CAWSGNTEAFF (402) | 644 | 0.30 |
| 37 | TRBV7-9 | TRBJ1-1 | CASSAWDRGAEAPF (403) | 628 | .029 |
| 38 | TRBV4-1 | TRBJ2-7 | CASSPGPGTSYEQYP (404) | 626 | 0.29 |
| 39 | TRBV5-1 | TRBJ2-1 | CASSFRLAGSTYNEQFF (405) | 615 | 0.28 |
| 40 | TRBV4-3 | TRBJ1-1 | CASSQAYGTGASEAPF (406) | 609 | 0.28 |
| 41 | TRBV11-2 | TRBJ2-2 | CASSFGTGNTGELFF (407) | 601 | 0.28 |
| 42 | TRBV28 | TRBJ2-1 | CASSPRPGQGEDNEQFF (408) | 586 | 0.27 |
| 43 | TRBV19 | TRBJ1-1 | CASSTGNTEAFF (409) | 586 | 0.27 |
| 44 | TRBV4-1 | TRBJ11 | CASSQGTEAFF (410) | 572 | 0.26 |
| 45 | TRBV28 | TRBJ2-1 | CASSLRADGYNEQFF (411) | 572 | 0.26 |
| 46 | TRBV7-9 | TRBJ2-7 | CASSPNTGGEQYP 9412) | 566 | 0.26 |
| 47 | TRBV11-2 | TRBJ2-7 | CASSLRGRNYEQYF (413) | 559 | 0.26 |
| 48 | TRBV28 | TRBJ1-5 | CASSPTGGVQPQHF (414) | 548 | 0.25 |
| 49 | TRBV29-1 | TRBJ2-1 | CSVEVGRELFF (415) | 521 | 0.24 |
| 50 | TRBV7-6 | TRBJ2-1 | CASSTRGAGRTYNEQFF (416) | 513 | 0.24 |

[symbol]

indicates data missing or illegible when filed

(“*” in Tables 6 to 13 indicates a stop codon)

### Example 2: Amplification of TCRα and TCRβ cDNA by One-Step RT-TS-PCR

Experiment B

(Reagent)

The additional reagents used in Experiment B are the following.
FITC labeled antihuman CD8 antibody (Beckman Coulter)
Alexa 647 labeled antihuman CD3 antibody (Beckman Coulter)
eFluor 780 (65-0865-14, eBioscience)

B1. Cell Preparation and Culture

B1-1. Cell Isolation

Peripheral blood was collected from a human volunteer (HLA-A*02 carrier). Peripheral blood mononuclear cells (PBMC) were isolated by a Ficoll density gradient method, and used in the experiment.

B1-2. Cell Culture

PBMCs were treated with a CMV pp65 protein epitope peptide, cultured for 2 weeks, then treated again with the peptide, and used in a single cell sorting repertoire analysis experiment on day 26.

B2-1. Staining with Cell Marker Antibody and Epitope Peptide

PBMCs treated with an epitope peptide and cultured were centrifuged and resuspended in PBS for washing. The cells were incubated for 30 minutes on ice with a dead cell staining fluorescence reagent eFluor 780 in PBS, resuspended in PBS, and centrifuged to allow the cells to precipitate, and the supernatant was removed for washing. The cells were then incubated for 10 minutes on ice with a background suppressor reagent (human FcR blocking reagent) Clear Back, resuspended in PBS, and centrifuged to allow the cells to precipitate, and the supernatant was removed for washing. The cells were then incubated for 30 minutes on ice with a phycoerythrin (PE) labeled MHC pp65 epitope tetramer, resuspended in PBS, and centrifuged to allow the cells to precipitate, and the supernatant was removed for washing. The cells were then incubated for 30 minutes on ice with an alexa 647 labeled anti-CD3 antibody and FITC labeled anti-CD8 antibody and subjected to single cell sorting.

B2-2. Single Cell Sorting

The cells were stained, then washed, and resuspended in PBS. The eFluor 780 negative, alexa 647 positive (CD3 positive), FITC positive (CD8 positive), PE positive (MHC epitope tetramer positive) cell groups among the lymophocyte populations were dispensed in a 96-well plate at one cell or a plurality of cells (5, 25, and 100 cells) each in a cell sorter (SONY, SH800).

B3. Amplification of TCRα and TCRβ cDNA by One-Step RT-TS-PCR

B3-1. One-Step RT-TS-PCR Reaction

A plate to which cells were dispensed was stored in a −80° C. ultra low temperature freezer. After 5 days, the plate was packaged with dry ice, transported to a research facility that was about 500 km away over one day by utilizing a frozen parcel shipping service provided by a shipping carrier, and stored in a −80° C. ultra low temperature freezer. After storing the plate in the −80° C. ultra low temperature freezer for 8 days, the plate was taken out, and a reaction solution was added at 10 μl/well on ice to perform a one-step RT-TS-PCR reaction (Table 14).

B3-2. Semi-Nested PCR

1.5 μl of one-step RT-TS-PCR reaction solution was fractionated after adding double the amount of pure water (DW). A PCR reaction was performed in a semi-nested form using a primer added with an adaptor sequence (sequence for adding an index sequence for sequencing) to the sequence on the inside of a block primer on the 3′ side and a template switching sequence on the 5′ side to add a sequencing primer recognition sequence to a DNA fragment in the same tube or separate tubes for each of TCRα and TCRβ (Table 15). 3 μl of reaction solution was fractionated to study a band by agarose gel electrophoresis.

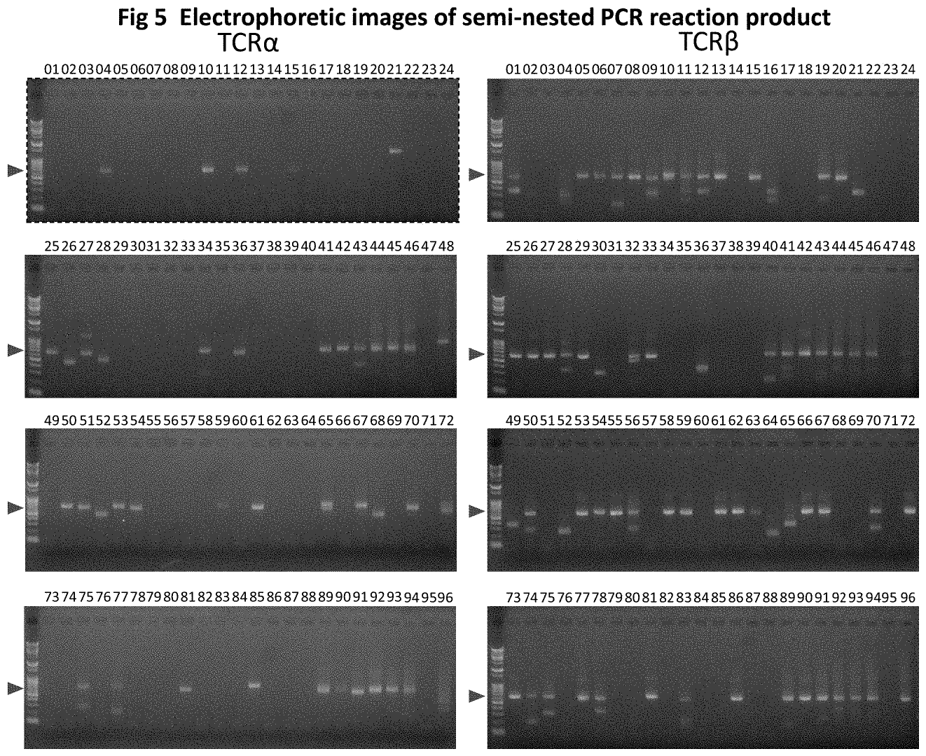

FIG. 5 shows the results of reacting cells that were stimulated with a CMV pp65 peptide and sorted, separately for TCRα and TCRβ. With wells confirmed to have a product with a length of about 400 base pairs or greater and 800 bases or less as positive, a DNA band was observed in 26 out of a total of 80 wells infused with one cell per well for TCRα, and in 45 wells for TCRβ. A cDNA band was observed for both TCRα and TCRβ in 20 of the wells.

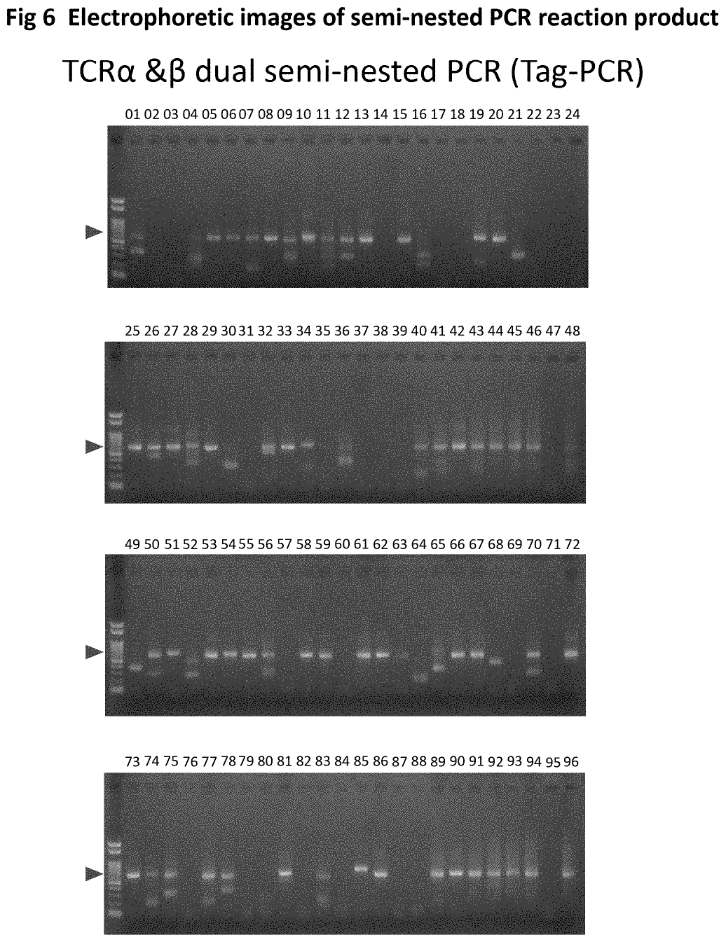

FIG. 6 shows results of reacting cells that were stimulated with a CMV pp65 peptide and sorted, in the same reaction solution for TCRα and TCRβ. With wells confirmed to have a product with a length of about 400 base pairs or greater and 800 bases or less as positive in the same manner, a DNA band was observed in 50 out of a total of 80 wells infused with one cell per well.

Excellent amplification was observed under almost all conditions in wells infused with 5 cells (wells 41, 42, 89, and 90), 25 cells (wells 43, 44, 91, and 92), and 100 cells (wells 45, 46, 93, and 94) or wells infused with established T cell derived RNA (well 48: Jurkat cell derived RNA and well 96: MOLT4 cell derived RNA were inputted) that were set as a positive control of a reaction under each condition. Cells were not infused into wells 47 and 95.

The above analysis demonstrated that TCRα/β pair sequences of T cells can be identified by one-step RT-TS-PCR for cells that were isolated then cryopreserved on a plate.

B3-3. Sequencing (Using MiSeq Next Generation Sequencer)

The samples that were amplified by PCR and confirmed to have a band (26 samples for TCRα and 45 samples for TCRβ in samples that were separately subjected to PCR in FIG. 5 and 50 samples among samples that were subjected to PCR in the same reaction solution in FIG. 6) were purified with an AMPure XP DNA kit. To sequence the samples with Illumina's MiSeq next generation sequencer, index addition PCR was performed with the PCR reaction solution and reaction cycle shown in Table 16. PCR products were purified with an AMPure XP DNA kit, and sequencing was run with a MiSeq next generation sequencer in accordance with the manufacturer's protocol. The resulting sequencing data was analyzed with Repertoire Genesis software.

Table 17 shows results of samples that were amplified and sequenced separately for TCRα and TCRβ, and Table 18 shows results of amplifying and sequencing in the same tube. In both cases, the same 18 samples of TCRα/β pair sequences (V region, J region, and CDR3 region) were able to be identified.

TABLE 17
Results of MiSeq sequencing from amplifying TCRα
and TCRβ separately

| Well No. | TRAVJ&TRBVJ pair | TRAV | TRAJ | CDR3 (SEQ ID NO) | TRBV | TRBJ | CDR3 (SEQ ID NO) |
|---|---|---|---|---|---|---|---|
| 01 | | | | | TRBV6-5 | TRBJ1-2 | CASSYQTGASYGYTF (443) |
| 04 | | TRAV24 | TRA.149 | CARNTGNQFYF (417) | | | |
| 05 | | | | | TRBV6-5 | TRBJ1-2 | CASSYQTGASYGYTF (444) |
| 06 | | | | | TRBV20-1 | TRBJ2-1 | CSARREYGTVAGEGYNEQFF (445) |
| 07 | | | | | TRBV20-1 | TRBJ2-1 | CSARREYGTVAGEGYNEQFF (446) |
| 08 | | | | | TRBV20-1 | TRBJ2-1 | CSARREYGTVAGEGYNEQFF (447) |
| 09 | | | | | TRBV20-1 | TRBJ2-1 | CSARREYGTVAGEGYNEQFF (448) |
| 10 | TRAV12-3/J34&TRBV20-1/J2-1 | TRAV12-3 | TRA.134 | CAISGNTDKLIF (418) | TRBV20-1 | TRBJ2-1 | CSARREYGTVAGEGYNEQFF (449) |
| 11 | | | | | TRBV20-1 | TRBJ2-1 | CSARREYGTVAGEGYNEQFF (450) |
| 12 | TRAV12-3/J34&TRBV20-1/J2-1 | TRAV12-3 | TRA.134 | CAISGNTDKLIF (419) | TRBV20-1 | TRBJ2-1 | CSARREYGTVAGEGYNEQFF (451) |
| 13 | | | | | TRBV6-5 | TRBJ1-2 | CASSYQTGASYGYTF (452) |
| 15 | TRAV12-3/J34&TRBV20-1/J2-1 | TRAV12-3 | TRA.134 | CAISGNTDKLIF (420) | TRBV20-1 | TRBJ2-1 | CSARREYGTVAGEGYNEQFF (453) |
| 19 | | | | | TRBV20-1 | TRBJ2-1 | CSARREYGTVAGEGYNEQFF (454) |
| 20 | | | | | TRBV20-1 | TRBJ2-1 | CSARREYGTVAGEGYNEQFF (455) |
| 25 | TRAV12-3/J34&TRBV20-1/J2-1 | TRAV12-3 | TRA.134 | CAISGNTDKLIF (421) | TRBV20-1 | TRBJ2-1 | CSARREYGTVAGEGYNEQFF (456) |
| 26 | TRAV12-3/J34&TRBV20-1/J2-1 | TRAV12-3 | TRA.134 | CAISGNTDKLIF (422) | TRBV20-1 | TRBJ2-1 | CSARREYGTVAGEGYNEQFF (457) |
| 27 | TRAV24/J49&TRBV6-5/11-2 | TRAV24 | TRA.149 | CARNTGNQFYF (423) | TRBV6-5 | TRBJ1-2 | CASSYQTGASYGYTF (458) |
| 28 | TRAV12-3/J34&TRBV20-1/J2-1 | TRAV12-3 | TRA.134 | CAISGNTDKLIF (424) | TRBV20-1 | TRBJ2-1 | CSARREYGTVAGEGYNEQFF (459) |
| 29 | | | | | TRBV6-5 | TRBJ1-2 | CASSYQTGASYGYTF (460) |
| 32 | | | | | TRBV6-5 | TRBJ1-2 | CASSYQTGASYGYTF (461) |
| 33 | | | | | TRBV6-5 | TRBJ1-2 | CASSYQTGASYGYTF (462) |
| 34 | | TRAV24 | TRA.149 | CARNTGNQFYF (425) | | | |
| 36 | | TRAV24 | TRA.149 | CARNTGNQFYF (426) | N.D. | N.D. | N.D. |
| 40 | | | | | TRBV20-1 | TRBJ2-1 | CSARREYGTVAGEGYNEQFF (463) |
| 50 | TRAV12-3/J34&TRBV20-1/J2-1 | TRAV12-3 | TRA.134 | CAISGNTDKLIF (427) | TRBV20-1 | TRBJ2-1 | CSARREYGTVAGEGYNEQFF (464) |
| 51 | | TRAV24 | TRA.149 | CARNTGNQFYF (428) | | | |
| 52 | | TRAV24 | TRA.149 | CARNTGNQFYF (429) | | | |
| 53 | TRAV12-3/J34&TRBV20-1/J2-1 | TRAV12-3 | TRA.134 | CAISGNTDKLIF (430) | TRBV20-1 | TRBJ2-1 | CSARREYGTVAGEGYNEQFF (465) |
| 54 | TRAV12-3/J34&TRBV20-1/J2-1 | TRAV12-3 | TRA.134 | CAISGNTDKLIF (431) | TRBV20-1 | TRBJ2-1 | CSARREYGTVAGEGYNEQFF (466) |
| 55 | | | | | TRBV6-5 | TRBJ1-2 | CASSYQTGASYGYTF (467) |
| 56 | | | | | TRBV20-1 | TRBJ2-1 | CSARREYGTVAGEGYNEQFF (468) |
| 58 | | | | | TRBV20-1 | TRBJ2-1 | CSARREYGTVAGEGYNEQFF (469) |
| 59 | TRAV12-3/J34&TRBV20-1/J2-1 | TRAV12-3 | TRA.134 | CAISGNTDKLIF (432) | TRBV20-1 | TRBJ2-1 | CSARREYGTVAGEGYNEQFF (470) |
| 61 | TRAV12-3/J34&TRBV20-1/J2-1 | TRAV12-3 | TRA.134 | CAISGNTDKLIF (433) | TRBV20-1 | TRBJ2-1 | CSARREYGTVAGEGYNEQFF (471) |
| 62 | | | | | TRBV20-1 | TRBJ2-1 | CSARREYGTVAGEGYNEQFF (472) |
| 63 | | | | | TRBV20-1 | TRBJ2-1 | CSARREYGTVAGEGYNEQFF (473) |
| 65 | | TRAV24 | TRA.149 | CARNTGNQFYF (434) | N.D. | N.D. | N.D. |
| 66 | | | | | TRBV20-1 | TRBJ2-1 | CSARREYGTVAGEGYNEQFF (474) |
| 67 | TRAV12-3/J34&TRBV20-1/J2-1 | TRAV12-3 | TRA.134 | CAISGNTDKLIF (435) | TRBV20-1 | TRBJ2-1 | CSARREYGTVAGEGYNEQFF (475) |
| 68 | TRAV24 | | TRA.149 | CARNTGNQFYF (436) | | | |
| 70 | TRAV12-3/J34&TRBV20-1/J2-1 | TRAV12-3 | TRA.134 | CAISGNTDKLIF (437) | TRBV20-1 | TRBJ2-1 | CSARREYGTVAGEGYNEQFF (476) |
| 72 | TRAV12-3/J34&TRBV20-1/J2-1 | TRAV12-3 | TRA.134 | CAISGNTDKLIF (438) | TRBV20-1 | TRBJ2-1 | CSARREYGTVAGEGYNEQFF (477) |
| 73 | | | | | TRBV6-5 | TRBJ1-2 | CASSYQTGASYGYTF (478) |
| 74 | | | | | TRBV6-5 | TRBJ1-2 | CASSYQTGASYGYTF (479) |
| 75 | TRAV12-3/J34&TRBV20-1/J2-1 | TRAV12-3 | TRA.134 | CAISGNTDKLIF (439) | TRBV20-1 | TRBJ2-1 | CSARREYGTVAGEGYNEQFF (480) |
| 77 | TRAV12-3/J34&TRBV20-1/J2-1 | TRAV12-3 | TRA.134 | CAISGNTDKLIF (440) | TRBV20-1 | TRBJ2-1 | CSARREYGTVAGEGYNEQFF (481) |
| 78 | | | | | TRBV6-5 | TRBJ1-2 | CASSYQTGASYGYTF (482) |
| 81 | TRAV24/149&TRBV6-5/11-2 | TRAV24 | TRA.149 | CARNTGNQFYF (441) | TRBV6-5 | TRBJ1-2 | CASSYQTGASYGYTF (483) |
| 83 | | | | | TRBV27 | TRBJ2-2 | CASSEVAGVPGELFF (484) |
| 85 | | TRAV12-3 | TRA.134 | CAISGNTDKLIF (442) | | | |
| 86 | | | | | TRBV20-1 | TRBJ2-1 | CSARREYGTVAGEGYNEQFF (485) |

TABLE 18
Results of MiSeq sequencing from amplifying and
analyzing TCRα and TCRβ in the same tube with the same index

| Well No. | TRAVJ&TRBVJ pair | TRAV | TRAJ | CDR3 (SEQ ID NO) | TRBV | TRBJ | CDR3 (SEQ ID NO) |
|---|---|---|---|---|---|---|---|
| 01 | | | | | TRBV6-5 | TRBJ1-2 | CASSYQTGASYGYTF (443) |
| 05 | | | | | TRBV6-5 | TRBJ1-2 | CASSYQTGASYGYTF (444) |
| 06 | | | | | TRBV20-1 | TRBJ2-1 | CSARREYGTVAGEGYNEQFF (445) |
| 07 | | | | | TRBV20-1 | TRBJ2-1 | CSARREYGTVAGEGYNEQFF (446) |
| 08 | | | | | TRBV20-1 | TRBJ2-1 | CSARREYGTVAGEGYNEQFF (447) |
| 09 | | | | | TRBV20-1 | TRBJ2-1 | CSARREYGTVAGEGYNEQFF (448) |
| 10 | TRAV12-3/J34&TRBV20-1/J2-1 | TRAV12-3 | TRAJ34 | CAISGNTDKLIF (418) | TRBV20-1 | TRBJ2-1 | CSARREYGTVAGEGYNEQFF (449) |
| 11 | | | | | TRBV20-1 | TRBJ2-1 | CSARREYGTVAGEGYNEQFF (450) |
| 12 | TRAV12-3/J34&TRBV20-1/J2-1 | TRAV12-3 | TRAJ34 | CAISGNTDKLIF (419) | TRBV20-1 | TRBJ2-1 | CSARREYGTVAGEGYNEQFF (451) |
| 13 | | | | | TRBV6-5 | TRBJ1-2 | CASSYQTGASYGYTF (452) |
| 15 | TRAV12-3/J34&TRBV20-1/J2-1 | TRAV12-3 | TRAJ34 | CAISGNTDKLIF (420) | TRBV20-1 | TRBJ2-1 | CSARREYGTVAGEGYNEQFF (453) |
| 19 | | | | | TRBV20-1 | TRBJ2-1 | CSARREYGTVAGEGYNEQFF (454) |
| 20 | | | | | TRBV20-1 | TRBJ2-1 | CSARREYGTVAGEGYNEQFF (455) |
| 25 | TRAV12-3/J34&TRBV20-1/J2-1 | TRAV12-3 | TRAJ34 | CAISGNTDKLIF (421) | TRBV20-1 | TRBJ2-1 | CSARREYGTVAGEGYNEQFF (456) |
| 26 | TRAV12-3/J34&TRBV20-1/J2-1 | TRAV12-3 | TRAJ34 | CAISGNTDKLIF (422) | TRBV20-1 | TRBJ2-1 | CSARREYGTVAGEGYNEQFF (457) |
| 27 | TRAV24/J49&TRBV6-5/J1-2 | TRAV24 | TRAJ49 | CARNTGNQFYF (423) | TRBV6-5 | TRBJ1-2 | CASSYQTGASYGYTF (458) |
| 28 | TRAV12-3/J34&TRBV20-1/J2-1 | TRAV12-3 | TRAJ34 | CAISGNTDKLIF (424) | TRBV20-1 | TRBJ2-1 | CSARREYGTVAGEGYNEQFF (459) |
| 29 | | | | | TRBV6-5 | TRBJ1-2 | CASSYQTGASYGYTF (460) |
| 32 | | | | | TRBV6-5 | TRBJ1-2 | CASSYQTGASYGYTF (461) |
| 33 | | | | | TRBV6-5 | TRBJ1-2 | CASSYQTGASYGYTF (462) |
| 34 | | TRAV24 | TRAJ49 | CARNTGNQFYF (425) | | | |
| 36 | | TRAV24 | TRAJ49 | CARNTGNQFYF (426) | | | |
| 40 | | | | | TRBV20-1 | TRBJ2-1 | CSARREYGTVAGEGYNEQFF (463) |
| 50 | TRAV12-3/J34&TRBV20-1/J2-1 | TRAV12-3 | TRAJ34 | CAISGNTDKLIF (427) | TRBV20-1 | TRBJ2-1 | CSARREYGTVAGEGYNEQFF (464) |
| 51 | | TRAV24 | TRAJ49 | CARNTGNQFYF (428) | | | |
| 52 | | TRAV24 | TRAJ49 | CARNTGNQFYF (429) | | | |
| 53 | TRAV12-3/J34&TRBV20-1/J2-1 | TRAV12-3 | TRAJ34 | CAISGNTDKLIF (430) | TRBV20-1 | TRBJ2-1 | CSARREYGTVAGEGYNEQFF (465) |
| 54 | TRAV12-3/J34&TRBV20-1/J2-1 | TRAV12-3 | TRAJ34 | CAISGNTDKLIF (431) | TRBV20-1 | TRBJ2-1 | CSARREYGTVAGEGYNEQFF (466) |
| 55 | | | | | TRBV6-5 | TRBJ1-2 | CASSYQTGASYGYTF (467) |
| 56 | | | | | TRBV20-1 | TRBJ2-1 | CSARREYGTVAGEGYNEQFF (468) |
| 58 | | | | | TRBV20-1 | TRBJ2-1 | CSARREYGTVAGEGYNEQFF (469) |
| 59 | TRAV12-3/J34&TRBV20-1/J2-1 | TRAV12-3 | TRAJ34 | CAISGNTDKLIF (432) | TRBV20-1 | TRBJ2-1 | CSARREYGTVAGEGYNEQFF (470) |
| 61 | TRAV12-3/J34&TRBV20-1/J2-1 | TRAV12-3 | TRAJ34 | CAISGNTDKLIF (433) | TRBV20-1 | TRBJ2-1 | CSARREYGTVAGEGYNEQFF (471) |
| 62 | | | | | TRBV20-1 | TRBJ2-1 | CSARREYGTVAGEGYNEQFF (472) |
| 63 | | | | | TRBV20-1 | TRBJ2-1 | CSARREYGTVAGEGYNEQFF (473) |
| 65 | | TRAV24 | TRAJ49 | CARNTGNQFYF (434) | | | |
| 66 | | | | | TRBV20-1 | TRBJ2-1 | CSARREYGTVAGEGYNEQFF (474) |
| 67 | TRAV12-3/J34&TRBV20-1/J2-1 | TRAV12-3 | TRAJ34 | CAISGNTDKLIF(435) | TRBV20-1 | TRBJ2-1 | CSARREYGTVAGEGYNEQFF (475) |
| 68 | | TRAV24 | TRAJ49 | CARNTGNQFYF (436) | | | |
| 70 | TRAV12-3/J34&TRBV20-1/J2-1 | TRAV12-3 | TRAJ34 | CAISGNTDKLIF (437) | TRBV20-1 | TRBJ2-1 | CSARREYGTVAGEGYNEQFF (476) |
| 72 | TRAV12-3/J34&TRBV20-1/J2-1 | TRAV12-3 | TRAJ34 | CAISGNTDKLIF (438) | TRBV20-1 | TRBJ2-1 | CSARREYGTVAGEGYNEQFF (477) |
| 73 | | | | | TRBV6-5 | TRBJ1-2 | CASSYQTGASYGYTF (478) |
| 74 | | | | | TRBV6-5 | TRBJ1-2 | CASSYQTGASYGYTF (479) |
| 75 | TRAV12-3/J34&TRBV20-1/J2-1 | TRAV12-3 | TRAJ34 | CAISGNTDKLIF (439) | TRBV20-1 | TRBJ2-1 | CSARREYGTVAGEGYNEQFF (480) |
| 77 | TRAV12-3/J34&TRBV20-1/J2-1 | TRAV12-3 | TRAJ34 | CAISGNTDKLIF (440) | TRBV20-1 | TRBJ2-1 | CSARREYGTVAGEGYNEQFF (481) |
| 78 | | | | | TRBV6-5 | TRBJ1-2 | CASSYQTGASYGYTF (482) |
| 81 | TRAV24/J49&TRBV6-5/J1-2 | TRAV24 | TRAJ49 | CARNTGNQFYF (441) | TRBV6-5 | TRBJ1-2 | CASSYQTGASYGYTF (483) |
| 83 | | | | | TRBV27 | TRBJ2-2 | CASSEVAGVPGELFF (484) |
| 85 | | TRAV 12-3 | TRAJ34 | CAISGNTDKLIF (442) | | | |
| 86 | | | | | TRBV20-1 | TRBJ2-1 | CSARREYGTVAGEGYNEQFF (485) |

### (Example 3: Amplification of TCRα and TCRβ, cDNA by One-Step RT-TS-PCR (2))

Experiment C

C1. Cell Preparation and Culture

C1-1. Cell Isolation

Peripheral blood was collected from human volunteers (two HLA-A*02 carriers). Peripheral blood mononuclear cells (PBMC) were isolated by a Ficoll density gradient method, and used in the experiment (same experiment as Experiment B).

C1-2. Cell Culture

PBMCs from one of the volunteers were treated with a CMV pp65 protein epitope peptide (subsequent CMV treatment and staining are the same as Experiment B) and PBMCs from the other volunteer were treated with an Influenza M1 protein epitope peptide. After two weeks of culture, the PBMCs were treated again with the peptides and used in a single cell sorting repertoire analysis experiment on day 26.

The full length amino acid sequence of Influenza M1 protein (P03485) is the following. The underlined 9 amino acid residues are the peptide epitope used in this Example.

(SEQ ID NO: 805)
MSLLTEVETYVLSIIPSGPLKAEIAQRLEDVFAGKNTDLEVLMEWLKTRP
ILSPLTKGILGFVFTLTVPSERGLQRRRFVQNALNGNGDPNNMDKAVKLY
RKLKREITFHGAKEISLSYSAGALASCMGLIYNRMGAVTTEVAFGLVCAT
CEQTADSQHRSHRQMVTTTNPLIRHENRMVLASTTAKAMEQMAGSSEQAA
EAMEVASQARQMVQAMRTIGTHPSSSAGLKNDLLENLQAYQKRMGVQMQR
FK

C2. Cell Marker Staining, Epitope Staining, and Single Cell Sorting

C2-1. Staining with Cell Marker Antibody and Epitope Peptide

PBMCs treated with an epitope peptide and cultured were centrifuged and resuspended in PBS for washing. The cells were incubated for 30 minutes on ice with a dead cell staining fluorescence reagent eFluor 780 in PBS, resuspended in PBS, and centrifuged to allow the cells to precipitate, and the supernatant was removed for washing. The cells were then incubated for 10 minutes on ice with a background suppressor reagent (human FcR blocking reagent) Clear Back, resuspended in PBS, and centrifuged to allow the cells to precipitate, and the supernatant was removed for washing. The cells were then incubated for 30 minutes on ice with a phycoerythrin (PE) labeled MHC pp65 epitope tetramer or PE labeled MHC M1 epitope tetramer, resuspended in PBS, and centrifuged to allow the cells to precipitate, and the supernatant was removed for washing. The cells were then incubated for 30 minute on ice with an alexa 647 labeled anti-CD3 antibody and FITC labeled anti-CD8 antibody and subjected to single cell sorting.

C2-2. Single Cell Sorting

The cells were stained, then washed, and resuspended in PBS. The eFluor 780 negative, alexa 647 positive (CD3 positive), FITC positive (CD8 positive), PE positive (MHC epitope tetramer positive) cell groups among the lymophocyte populations were dispensed in a 96-well plate at one cell or a plurality of cells (10 and 100) each in a cell sorter (SONY, SH800) (cells that were treated with CMV and stained were immediately used in a one-step RT-TS-PCR experiment without storage at −80° C. with the same specimen as Experiment B).

C3. Amplification of TCRα and TCRβ cDNA by One-Step RT-TS-PCR

C3-1. One-Step RT-TS-PCR Reaction

Cells were dispensed and immediately subjected to a one-step RT-TS-PCR reaction in the reaction reagents shown in Table 19.

1.5 μl of one-step RT-TS-PCR reaction solution was fractionated after adding double the amount of pure water (DW). A PCR reaction was performed in a semi-nested form using a primer added with an adaptor sequence (sequence for adding an index sequence for sequencing) to the sequence on the inside of a block primer on the 3′ side and a template switching sequence on the 5′ side to add a sequencing primer recognition sequence to a DNA fragment in the same tube or separate tubes for each of TCRα and TCRβ (Table 20). 3 μl of reaction solution was fractionated to study a band by agarose gel electrophoresis.

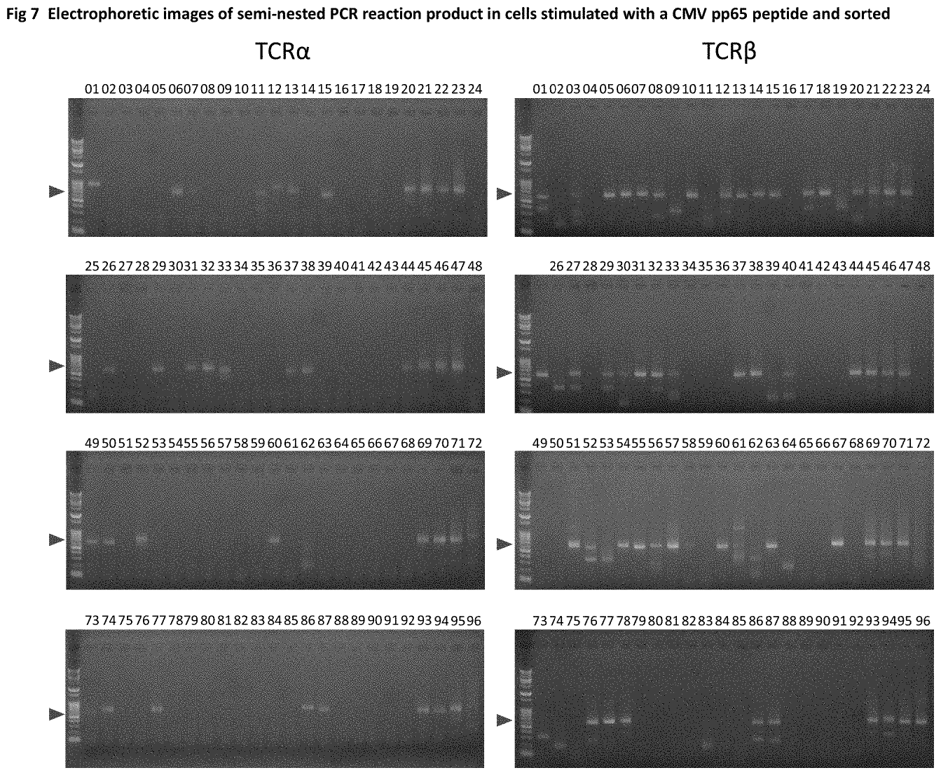

FIG. 7 shows the results for cells that were stimulated with a CMV pp65 peptide and sorted (separately for TCRα and TCRβ). With wells confirmed to have a product with a length of about 400 base pairs or greater and 800 bases or less as positive, a DNA band was observed in 25 out of a total of 80 wells infused with one cell per well for TCRα, and in 41 wells for TCRβ. A cDNA band was observed for both TCRα and TCRβ in 19 of the wells.

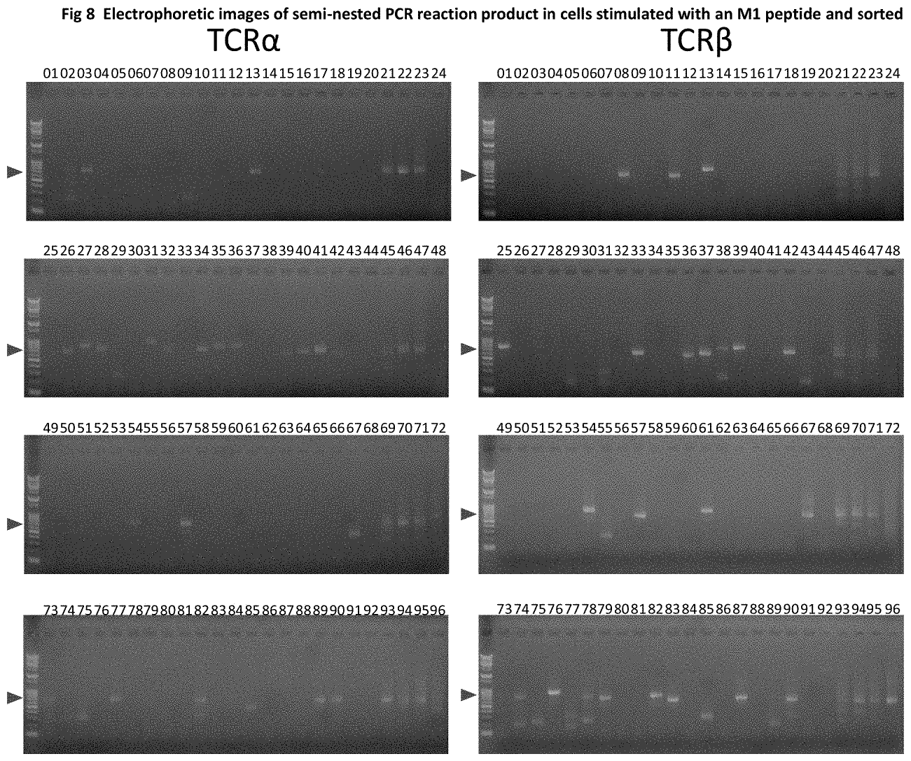

FIG. 8 shows results for cells that were stimulated with an M1 peptide and sorted (separately for TCRα and TCRβ). With wells confirmed to have a product with a length of about 400 base pairs or greater and 800 bases or less as positive, a DNA band was observed in 24 out of a total of 80 wells infused with one cell per well for TCRα, and in 21 wells for TCRβ. A cDNA band was observed for both TCRα and TCRβ in 9 of the wells.

Excellent amplification was observed under almost all conditions in wells infused with 10 cells (wells 21 and 22) and 100 cells (well 23) or wells with established T cell derived RNA (well 72: Jurkat cell derived RNA and well 96: MOLT4 cell derived RNA were inputted) that were set as a positive control of a reaction under each condition.

C3-3. Sequencing

The samples that were amplified by PCR and confirmed to have a band (25 samples for TCRα and 41 samples for TCR among samples that were stimulated with a CMV pp65 peptide and sorted in FIGS. 7 and 24 samples for TCRα and 21 samples for TCRβ in samples that were stimulated with an M1 peptide and sorted in FIG. 8) were purified with an AMPure XP DNA kit and sequenced by the Sanger method with a P5-seq primer. The resulting sequencing data was analyzed with Repertoire Genesis software.

Table 21 shows results of sequencing samples that were stimulated with a CMV pp65 peptide and sorted and analysis thereof with Repertoire Genesis software. In all 25 sequenced TCRα samples and 41 sequenced TCRβ samples, some type of TCR cDNA sequence was confirmed, and TCRα/β pair sequences were analyzed in 19 samples (identified at least V regions of both TCRα/β).

Table 22 shows results for samples that were stimulated with an M1 peptide and sorted. In 22 out of 24 sequenced TCRα samples and all 21 sequenced TCRβ samples, some type of TCR cDNA sequence was confirmed, and TCRα/β pair sequences were analyzed in 7 samples.

TABLE 21
Results of Sanger method sequencing and software analysis on cells that
were stimulated with a CMV pp65 peptide and sorted
(X and ? mean unidentified, * means stop codon)

| Well No | TRAVJ&&TRBV1 pair | TRAV | TRAJ | TRA CDR3 (SEQ ID NO) | TRBV | TRBJ | TRB CDR3 (SEQ ID NO) |
|---|---|---|---|---|---|---|---|
| 01 | TRAV24/149&TRBV6-5/J2-3 | TRAV24 | TRAJ49 | CARNTGNQFYF (486) | TRBV6-5 | TRBJ2-3 | ? |
| 03 | | | | | TRBV6-5 | TRBJ1-5 | CNSQKQWYQRRVHGASG*SNQPQH |
| | | | | | | | F (510) |
| 05 | | | | | TRBV6-5 | TRBJ1-2 | CASSYQTGASYGYTF (511) |
| 06 | TRAV12-3/J34&TRBV20-1/J2-1 | TRAV12-3 | TRAJ34 | CAISGNTDKLIF (487) | TRBV20-1 | TRBJ2-1 | CSARREYGTVAGEGYNEQFF (512) |
| 07 | TRAV12-3/J34&TRBV20-1/J2-1 | TRAV12-3 | TRAJ34 | CAISGNTDKLIF (488) | TRBV20-1 | TRBJ2-1 | CSARREYGTVAGEGYNEQFF (513) |
| 08 | | | | | TRBV6-5 | TRBJ1-2 | CVSSYQTGASYGYTL (514) |
| 10 | | | | | TRBV20-1 | TRBJ2-1 | CSARREYGTVAGEGYNEQFF (515) |
| 11 | | TRAV24 | TRAJ49 | CARNTGNQFYF (489) | | | |
| 12 | TRAV24/149&TRBV6-5/11-2 | TRAV24 | TRAJ49 | CARNTGNQFYF (490) | TRBV6-5 | TRBJ1-2 | CASSYQTGASRY**F (516) |
| 13 | TRAV12-3/J34&TRBV6-5/11-2 | TRAV12-3 | TRAJ34 | CAISGNTDKLIF (491) | TRBV6-5 | TRBJ1-2 | CASSYQTGASYGYTF (517) |
| 14 | | | | | TRBV20-1 | TRBJ2-1 | CSARREYGTVAGEGYNEQFF (518) |
| 15 | TRAV12-3/J34&TRBV20-1/J2-1 | TRAV12-3 | TRAJ34 | CAISGNTDKLIF (492) | TRBV20-1 | TRBJ2-1 | CGQLDVNTGPYRERDTMSS**F (519) |
| 17 | | | | | TRBV20-1 | TRBJ2-1 | CSARREYGTVAGEGYNEQFF (520) |
| 18 | TRAV12-3/J34&TRBV20-1/J2-1 | TRAV12-3 | TRAJ34 | CAISGNTDKLIF (493) | TRBV20-1 | TRBJ2-1 | CSARREWGTVGGQGTNEQYF (521) |
| 20 | | | TRAJ34 | ? | TRBV20-1 | ? | ? |
| 25 | | | | | TRBV20-1 | TRBJ2-3 | RAARREYGGLPGEGTNEQFF (522) |
| 26 | | TRAV24 | TRAJ49 | CARNTGNQFYF (494) | | | |
| 27 | | | | | TRBV6-5 | TRBJ2-3 | ? |
| 29 | TRAV24/149&TRBV6-5/? | TRAV24 | TRAJ49 | CARNTGNQFYF (495) | TRBV6-5 | ? | ? |
| 30 | | | | | TRBV20-1 | TRBJ2-1 | CSARREYGTVAGEGYNEQFF (523) |
| 31 | TRAV12-3/J34&TRBV20-1/J2-1 | TRAV12-3 | TRAJ34 | CAISGNTDKLIF (496) | TRBV20-1 | TRBJ2-1 | CSARREYGTVAGEGYNEQ**F (524) |
| 32 | TRAV12-3/J34&TRBV6-5/12-3 | TRAV12-3 | TRAJ34 | CAISGNTDKLIF (497) | TRBV6-5 | TRBJ2-3 | RAAPVFVLGLQAVSTDTQYF (525) |
| 33 | TRAV24/149&TRBV6-5/12-3 | TRAV24 | TRAJ49 | CARNTGNQFYF (498) | TRBV6-5 | TRBJ2-3 | RAAPVFVLGLQAVSTDTQYF (526) |
| 37 | TRAV12-3/J34&TRBV20-1/J2-1 | TRAV12-3 | TRAJ34 | CAISGNTDKLIF (499) | TRBV20-1 | TRBJ2-1 | CSARREYGTVAGEGYNEQFF (527) |
| 38 | TRAV12-3/J34&TRBV20-1/J2-1 | TRAV12-3 | TRAJ34 | CAISGNTDKLIF (500) | TRBV20-1 | TRBJ2-1 | CSARREYGTVAGEGYNEQFF (528) |
| 40 | | | | | TRBV6-5 | ? | ? |
| 44 | TRAV12-3/J34&TRBV20-1/J2-1 | TRAV12-3 | TRAJ34 | CAISGNTDKLIF (501) | TRBV20-1 | TRBJ2-1 | CSARREYGTVAGEGYNEQFF (529) |
| 49 | | | | | TRAV24 | TRAJ49 | CARNTGNQFYF (502) |
| 50 | | | | | TRAV24 | TRAJ49 | CARNTGNQFYF (503) |
| 51 | | | | | TRBV6-5 | TRBJ1-2 | CASSYQTGAR**KYTF (530) |
| 52 | TRAV12-3/J34&TRBV20-1/J2-3 | TRAV12-3 | TRAJ34 | CAISGNTDKLIF (504) | TRBV20-1 | TRBJ2-3 | x |
| 54 | | | | | TRBV6-5 | TRBJ1-2 | CASSYQTGASYGYTF (531) |
| 55 | | | | | TRBV6-5 | TRBJ1-2 | CASSYQTGASYGYTF (532) |
| 56 | | | | | TRBV20-1 | ? | ? |
| 57 | | | | | TRBV27 | ? | ? |
| 58 | | | | | TRBV20-1 | TRBJ2-1 | CSARREYGTVAGEGYNEQFF (533) |
| 60 | TRAV12-3/J34&TRBV20-1/J2-1 | TRAV12-3 | TRAJ34 | CAISGNTDKLIF (505) | TRBV20-1 | TRBJ2-1 | CSARREYGTVAGEGYNEQFF (534) |
| 61 | | | | | ? | TRBJ2-3 | ? |
| 63 | | | | | TRBV6-5 | TRBJ1-2 | CASSYQTGASYGYTF (535) |
| 67 | | | | | TRBV20-1 | TRBJ2-1 | CSARREYGTVAGEGYNEQFF (536) |
| 74 | | TRAV12-3 | TRAJ15 | CAILRPTRQELL**IF (506) | | | |
| 76 | | | | | TRBV6-5 | TRBJ1-2 | WASSYQTGASYGYTF (537) |
| 77 | TRAV12-3/J34&TRBV20-1/J2-1 | TRAV12-3 | TRAJ34 | CAISGNTDKLIF (507) | TRBV20-1 | TRBJ2-1 | CSARREYGTVAGEGYNEQFF (538) |
| 78 | | | | | TRBV6-5 | TRBJ1-2 | CASSYQTGASYGYTF (539) |
| 86 | TRAV12-3/J34&TRBV20-1/J2-3 | TRAV12-3 | TRAJ34 | CAISGNTDKLIF (508) | TRBV20-1 | TRBJ2-3 | RAARGEVGGLQGESTNKQYF (540) |
| 87 | TRAV12-3/J34&TRBV20-1/J2-1 | TRAV12-3 | TRAJ34 | CAISGNTDKLIF (509) | TRBV20-1 | TRBJ2-1 | CRPFPGGSYNEQFF (541) |

TABLE 22
Results of Sanger method sequencing and software analysis on cells that
were stimulated with an M1 peptide and sorted
(X mean unidentified, * means stop codon)

| Well No | TRAV1&&TRBV1 pair | TRAV | TRAJ | TRA CDR3 (SEQ ID NO) | TRBV | TRBJ | TRB CDR3 (SEQ ID NO) |
|---|---|---|---|---|---|---|---|
| 02 | | TRAV14/DV4 | TRAJ23 | CIRDRSSGINAEYMGV**IYNQ | | | |
| | | | | GGKLIF (542) | | | |
| 03 | | TRAV19 | TRAJ40 | CALGGTSGTYKYIF (543) | | | |
| 08 | | | | | TRBV20-1 | TRBJ2-4 | CSAPGIVLAKNIQYF (561) |
| 11 | | | | | TRBV20-1 | TRBJ2-4 | CSAPGIVLAKNIQYF (562) |
| 13 | TRAV35/J42&TRBV19/J1-1 | TRAV35 | TRAJ42 | CAGTDGGSQGNLIF (544) | TRBV19 | TRW 1-1 | CASSIFGGAEAFF (563) |
| 26 | | TRAV27 | TRAJ42 | CAGGGSQGNLIF (545) | | | |
| 27 | | TRAV8-6 | TRAJ42 | CAVDGSQGNLIF (546) | | | |
| 28 | | TRAV27 | TRAJ37 | CAGASGNTGKLIF (547) | | | |
| 32 | | TRAV27 | TRAJ42 | CAGAGGGSQGNLIF (548) | | | |
| 33 | | | | | TRBV19 | TRBJ2-3 | CASSIRSTDTQYF (564) |
| 34 | | TRAV5 | TRAJ42 | CAETRGGGSQGNLIF (549) | | | |
| 35 | | TRAV8-6 | TRAJ42 | CAVDGSQGNLIF (550) | | | |
| 36 | | ? | TRAJ35 | ? | TRBV19 | TRBJ2-7 | CASSLRSSYEQYF (565) |
| 37 | | | | | TRBV20-1 | TRBJ2-4 | CSAPGIVLAKNIQYF (566) |
| 38 | | | | | TRBV19 | TRW1-4 | CTKL*NIAGTPWGDEKLFF (567) |
| 39 | TRAV5/J42&TRBV19/J2-1 | TRAV5 | TRAJ42 | CAETRGGGSQGNLIF (551) | TRBV19 | TRBJ2-1 | CASSPRAGVEQFF (568) |
| 40 | | TRAV27 | TRAJ42 | CAGGGSQGNLIF (552) | | | |
| 41 | | TRAV5 | TRAJ42 | CAETRGGGSQGNLIF (553) | | | |
| 42 | TRAV25/J42&TRBV19/J2-3 | TRAV25 | TRAJ42 | CAGNYGGSQGNLIF (554) | TRBV19 | TRBJ2-3 | CASSIRSTDTQYF (569) |
| 54 | TRAV5/J42&TRBV19/J2-1 | TRAV5 | TRAJ42 | CAETRGGGSQGNLIF (555) | TRBV19 | TRBJ2-1 | CASSPRAGVEQFF (570) |
| 57 | TRAV26-1/J27&TRBV20-1/J2-4 | TRAV26-1 | TRAJ27 | CMVSIGNAGKSTF (556) | TRBV20-1 | TRBJ2-4 | CSAPGIVLAKNIQYF (571) |
| 61 | | | | | TRBV19 | TRW1-5 | CASSIFGGNQPQHF (572) |
| 67 | | ? | TRAJ5 | ? | TRBV19 | TRBJ2-3 | CASSIRSTDTQYF (573) |
| 74 | | | | | TRBV19 | TRW1-5 | ? |
| 75 | | TRAV25 | TRAJ42 | (CAGNYGGSQGNLIF 557) | | | |
| 76 | | | | | TRBV19 | TRBJ2-2 | CASSLRSTGELFF (574) |
| 77 | | TRAV19 | ? | ? | | | |
| 78 | | | | | TRBV19 | ? | ? |
| 79 | | | | | TRBV19 | TRBJ2-1 | CASSPRAGVEQFF (575) |
| 82 | TRAV5/J42&TRBV19/J2-1 | TRAV5 | TRAJ42 | CAETRGGGSQGNLIF (558) | TRBV19 | TRBJ2-1 | CASSPRAGVEQFF (576) |
| 83 | | | | | TRBV19 | TRBJ2-3 | CASSGRSTDTQYF (577) |
| 87 | | | | | TRBV19 | TRBJ2-3 | CASSPRSTDTQYF (578) |
| 89 | | TRAV27 | TRAJ42 | SAGAGGGSQGNLIF (559) | | | |
| 90 | TRAV27/J42&TRBV19/J2-1 | TRAV27 | TRAJ42 | CAGAGGGSQGNLIF (560) | TRBV19 | TRBJ2-1 | CASSIRSSDEQFF (579) |

C3-4. Sequencing-2

Index addition PCR was performed with the PCR reaction solution and reaction cycle shown in Table 23 to sequence purified PCR products (DNA samples amplified by PCR in “C3-3. Sequencing”) derived from 4 wells (Well No. 6, 12, 15, and 20) of cells that were stimulated with a CMV pp65 peptide and sorted and from 3 wells (Well No. 13, 36, and 39) of cells that were stimulated with an M1 peptide and sorted, including samples with sequence information that is partially unclear among samples whose TCRα/β pair sequences were able to be analyzed in “C3-3. Sequencing”, with Illumina's MiSeq next generation sequencer. PCR products were purified with an AMPure XP DNA kit, and sequencing was run with a MiSeq next generation sequencer in accordance with the manufacturer's protocol.

The resulting sequencing data was analyzed with Repertoire Genesis software. Table 24 shows a result of comparing the result thereof with Sanger method sequencing data. An accurate analysis result was obtained for sequences of a sample that could not be accurately analyzed with the Sanger method (CMV-No.20 TCRα/β) and samples that could not be determined to be CDR3 comprising a stop codon (CMV-No.12 TCRβ, CMV-No.15 TCRβ), and additionally, CDR3 amino acid sequences were correctly identified. While there are samples that cannot be analyzed well by the Sanger method due to sequencing errors, contamination of non-specifically amplified DNA fragment, or the like, it was confirmed that accurate sequence data is obtained by analysis of a plurality of reads at the molecular level with a so-called next generation sequencer such as Illumina's MiSeq.

TABLE 24
Results of MiSeq sequencing and software analysis on 4 wells of cells that were stimulated with a CMV
pp65 peptide and sorted and 3 wells of cells that were stimulated with an M1 peptide and sorted (X and ? mean
unidentified, * means stop codon)

| | | CDR3 | | CDR3 | TRA | CDR3 | TRB | CDR3 |
|---|---|---|---|---|---|---|---|---|
| Well No | TRA(MiSeq) | (SEQ ID NO) | TRB(MiSeq) | (SEQ ID NO) | (Sanger method) | (SEQ ID NO) | (Sanger method) | (SEQ ID NO) |
| CMV-No. 06 | TRAV12-3/J34 | CAISGNTDKLIF | TRBV20-1/J2-1 | CSARREYGTVAGEGYN | TRAV12-3/J34 | CAISGNTDKLIF | TRBV20-1/J2-1 | CSARREYGTVAGEGYN |
| | | (580) | | EQFF (586) | | (593) | | EQFF (598) |
| CMV-No. 12 | TRAV24/J49 | CARNTGNQFYF | TRBV6-5/J1-2 | CASSYQTGASYGYTF | TRAV24/J49 | CARNTGNQFYF | TRBV6-5/J1-2 | CASSYQTGASRY**F |
| | | (581) | | (587) | | (594) | | (599) |
| CMV-No. 15 | TRAV12-3/J34 | CAISGNTDKLIF | TRBV20-1/J2-1 | CSARREYGTVAGEGYN | TRAV12-3/J34 | CAISGNTDKLIF | TRBV20-1/J2-1 | CGQLDVNTGPYRERDT |
| | | (582) | | EQFF (588) | | (595) | | MSS**F (600) |
| CMV-No. 20 | TRAV12-3/J34 | CAISGNTDKLIF | TRBV20-1/J2-1 | CSARREYGTVAGEGYN | ?/J34 | x | TRBV20-1/? | x |
| | | (583) | | EQFF (589) | | | | |
| M1-No. 13 | TRAV35/J42 | CAGTDGGSQGNLIF | TRBV19/J1-1 | CASSIFGGAEAFF | TRAV35/J42 | CAGTDGGSQGNLIF | TRBV19/J1-1 | CASSIFGGAEAFF |
| | | (584) | | (590) | | (596) | | (601) |
| M1-No. 36 | ?/J35 | x | TRBV19/J2-7 | CASSLRSSYEQYF | ?/J35 | x | TRBV19/J2-7 | CASSLRSSYEQYF |
| | | | | (591) | | | | (602) |
| M1-No. 39 | TRAV5/J42 | CAETRGGGSQGNLIF | TRBV19/J2-1 | CASSPRAGVEQFF | TRAV5/J42 | CAETRGGGSQGNLIF | TRBV19/J2-1 | CASSPRAGVEQFF |
| | | (585) | | (592) | | (597) | | |

### Example 4: Amplification of BCR by One-Step RT-TS-PCR

Experiment D

D1. Cell Preparation and Culture

### Preparation Example

A preparation example of reagents used in the Examples is shown below.

*List of reagents (additional reagents for Experiment D)
-FITC labeled antihuman CD19 antibody (560994, Nippon Becton
Dickinson Company)
-PE-Cy5 labeled antihuman CD8 antibody (557746, Nippon Becton
Dickinson Company)
-Clear back Human Fc receptor blocking reagent (MTG-001,
Medical & Biological Laboratories)
-Resiquimod (SML0196, Sigma-Aldrich)
-7-AAD (51-88981E, Nippon Becton Dickinson Company)
*Oligonucleotide sequences used
-hBCRμ Block primer (CM2 (2); 19 bases):
(SEQ ID NO: 604)
TCCTGTGCGAGGCAGCCAA
-hBCRμ RT primer (CM1(2); 23 bases):
(SEQ ID NO: 605)
TGATGTCAGAGTTGTTCTTG
-hBCR Kappa Block primer (CK2; 18 bases):
(SEQ ID NO: 606)
CTGTACTTTGGCCTCTCT
-hBCR Kappa RT primer (CK1; 19 bases):
(SEQ ID NO: 607)
TTGTGTTTCTCGTAGTCTG
-hBCR Lambda Block primer (CL2; 16 bases):
(SEQ ID NO: 608)
CCGGGTAGAAGTCACT
-hBCR Lambda RT primer (CL1; 19 bases):
(SEQ ID NO: 609)
TGTCTTCTCCACGGTGCTC
-hBCRp Reverse Tag primer (CM-ST1-R-(3), 53 bases):
(SEQ ID NO: 610)
TCGTC{GGCAGCGTCAGATGTGTATAA}GAGACAGGTATCCGACGGGGAATTCTC
-hBCR Kappa Reverse Tag primer (CK-ST1-R, 51 bases):
(SEQ ID NO: 611)
TCGTC{GGCAGCGTCAGATGTGTATAA}GAGACAGTTATTCAGCAGGCACACA
-hBCR Lambda Reverse Tag primer (CL-ST1-R, 51 bases):
(SEQ ID NO: 612)
TCGTC{GGCAGCGTCAGATGTGTATAA}GAGACAGTGTGGCCTTGTTGGCTTG

D1-1. Cell Isolation

Peripheral blood was collected from human volunteers (two). Peripheral blood mononuclear cells (PBMC) were isolated by a Ficoll density gradient method and used in the experiment.

D1-2. Cell Culture

Both of the two specimens of PBMCs were cultured overnight after preparation, cultured for 2 days in a medium comprising 10 pg/ml of Resiquimod, and used in a single cell sorting repertoire analysis experiment. It was confirmed whether a B cell was activated in advance by treating the cell with Resiquimod for 2 days and confirming an elevation in the mRNA level of IL-6, BCRμ, BCRκ, and BCRλ (reference document: Eur J Immunol. 2018 February; 48(2): 283-292. BAFF augments IgA2 and IL-10 production by TLR7/8 stimulated total peripheral blood B cells). CD19 was used as a cell marker for B cells.

D2. Cell Marker Staining and Single Cell Sorting

D2-1. Staining with Cell Marker Antibody

PBMCs cultured in a medium comprising Resiquimod were centrifuged to allow the cells to precipitate, resuspended in MACS buffer, and centrifuged for washing. The cells were then treated for 5 minutes on ice with Clear Back Human Fc receptor blocking reagent in MACS buffer, resuspended in MACS buffer, and centrifuged for washing twice. The cells were then stained for 30 minutes on ice with an FITC labeled CD19 antibody and PE-Cy7 labeled anti-CD8 antibody, resuspended in MACS buffer, and centrifuged for washing. The cells were then stained for 10 minutes on ice with a dead cell staining reagent 7-AAD, resuspended in MACS buffer, and centrifuged for washing. The cells were then ultimately suspended in 1 ml of MACS buffer and subjected to single cell sorting.

D2-2. Single Cell Sorting

From the stained cells, 7-AAD negative, FITC positive (CD19 positive), PE-Cy7 negative (CD8 negative) cell groups in a lymphocyte population gate were inputted in a 96-well plate added with an RT-TS-PCR reaction solution (D3-1) at one cell (24 wells for each donor), or a plurality of cells (3 wells each of 5 cells or 25 cells for each donor) as a positive control of the experiment in a cell sorter (Nippon Becton Dickinson Company, FACSMelody). 4 wells without any cell input were used as a negative control.

D3. Amplification of BCRμ (Ig heavy chain μ), BCRκ (Ig light chain κ), and BCRλ (Ig light chain λ) by One-step RT-TS-PCR

D3-1. One-Step RT-TS-PCR Reaction

A one-step RT-TS-PCR reaction was immediately performed on a plate to which cells were dispensed in the reaction reagents of Table 25.

1.5 μl of one-step RT-TS-PCR reaction solution was fractionated after adding double the amount of pure water (DW). A PCR reaction was performed in a semi-nested form in the same tube or separate tubes for BCRμ, BCRκ, and BCRλ in the reaction solutions of Table 24. A primer added with an adaptor sequence (sequence for adding an index sequence for sequencing) to the sequence on the inside of a block primer on the 3′ side and a template switching sequence on the 5′ side was used to add a sequencing primer recognition sequence to a DNA fragment. 3 μl of reaction solution was fractionated to study a band by agarose gel electrophoresis.

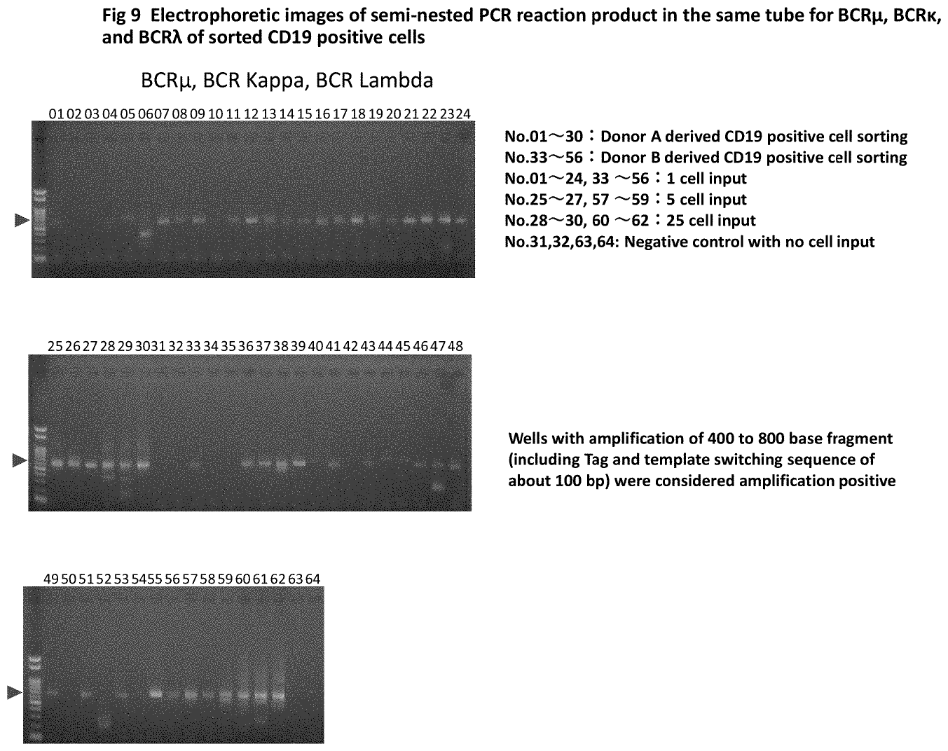

FIG. 9 shows the results of semi-nested PCR within the same tube. With wells confirmed to have a product with a length of about 400 base pairs or greater and 800 bases or less as positive, a DNA band with a length of interest was observed in 37 (donor A: 20 wells, donor B: 17 wells) out of 48 wells subjected to single cell analysis (infused with one cell per well). Excellent amplification was observed in all wells infused with 5 cells (wells 25 to 27 and 57 to 59) and 25 cells (wells 38 to 30 and 60 to 62) that were set as a positive control of a reaction. Cells were not infused into wells 31, 32, 63, and 64.

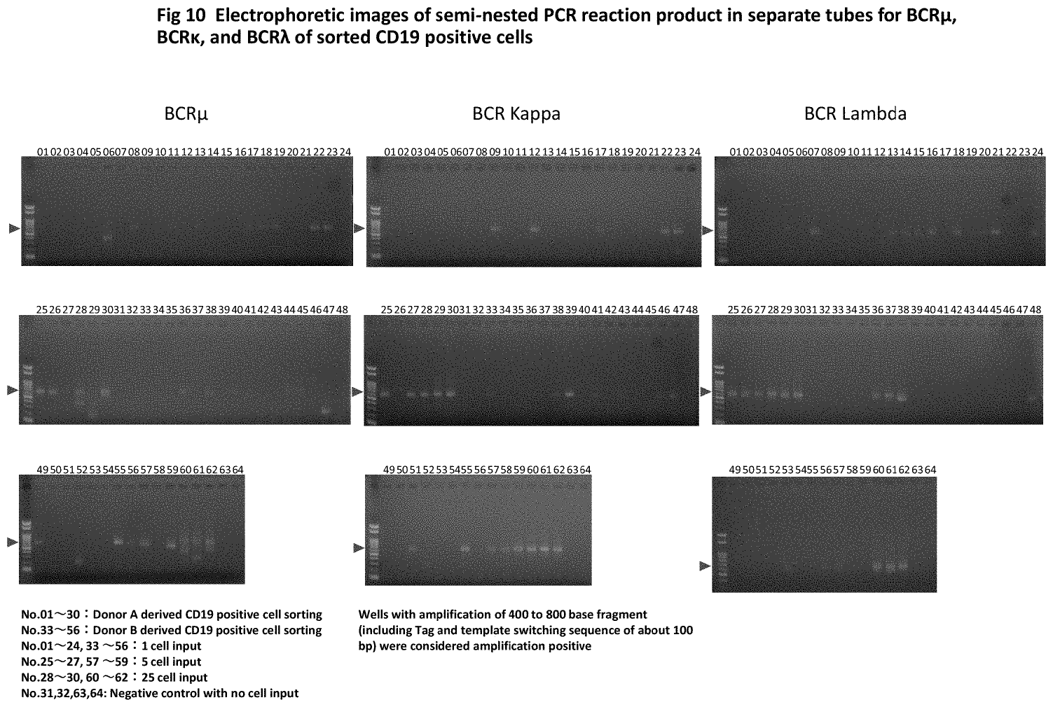

FIG. 10 shows the results of semi-nested PCR on BCRμ, BCR Kappa, and BCR Lambda within separate tubes. With wells confirmed to have a product with a length of about 400 base pairs or greater and 800 bases or less as positive, a DNA band with a length of interest was observed in 15 out of 48 wells subjected to single cell analysis for BCRμ, in 14 out of 48 wells for BCR Kappa, and in 15 out of 48 wells for BCR Lambda. Amplification of pair genes (heavy chain BCRμ and either one or both of light chain BCR Kappa and BCR Lambda) was observed in 9 wells (No. 13, 17, 18, 22, 23, 36, 38, 55, and 56).

D3-3. Sequencing

wells of semi-nested PCR samples in the same tube reaction system that were amplified by PCR and confirmed to have a band, and 19 semi-nested PCR samples from 9 wells that were observed to have pair amplification in separate reaction systems were purified with an AMPure XP DNA kit. To sequence the samples with Illumina's MiSeq next generation sequencer, index addition PCR was performed with the following PCR reaction solution and reaction cycle. PCR products were purified with an AMPure XP DNA kit, and sequencing was run with a MiSeq next generation sequencer in accordance with the manufacturer's protocol. The resulting sequencing data was analyzed with Repertoire Genesis software.

Upon sequencing clones for which 10% of the number of all reads was obtained in the result of sequencing in the same tube, a genetic sequence comprising in-frame CDR3 was able to be analyzed in 16 wells for BCRμ, 13 wells for BCR Kappa, and 17 wells (in three of the wells, two sequences understood to be derived from two chromosomes were identified) for BCR Lambda among the 37 wells of sequenced samples, and pair genes of BCR heavy chain/light chain in 10 wells (No.) were able to be analyzed (multiple V, D, or J regions displayed in the table indicate that homology scores are identical leading to two candidates. It is understood this is because in most cases, there is hardly any difference in these two sequences so that the sequences are indistinguishable on cDNA although the positions on the genome differ).

Upon sequencing clones for which 20% of the number of all reads was obtained in the result of separately analyzed sequencing results, all 19 analyzed BCR cDNA fragments were able to be sequenced, and protein translatable in-frame CDR3 sequence was obtained in 18 sequences. As a result, a stop codon was inserted in all of the 9 wells of samples, and pair genes of BCR heavy chain/light chain were able to be analyzed. While it was understood that No. 38 BCR Kappa had a stop codon in CDR3 and was not an in-frame sequence clone, it was understood that BCR Lambda was an in-frame sequence, which is joined to a heavy chain as a light chain. Separately analyzed 19 sequences were completely identical to the results analyzed in the same tube. A reaction in the same tube is more simple and requires less effort for electrophoretic checking, but the total number of specimens that are sequenced increases. Reactions in separate tubes would require more effort during an experiment, but would require less number of sequencing, so that each has advantages. In the same manner as TCR analysis, it is understood that it is effective to use different approaches, e.g., when the identity ratio of pair genes is expected to be high, samples in the same tube are sequenced, and samples that are difficult to analyze are individually amplified when the identity ratio appears low.

TABLE 28
Results of amplifying and sequencing BCRμ, BCRκ, and BCRλ in the same tube
[Table 28-1]
(? means unidentified, * is a stop codon, yellow is BCR whose gene was identified, gray is BCR
whose gene could not be identified, and blue has an identified stop codon insertion sequence)

| | | BCRH | Top | | | | | | Top | | | BCRKappa- |
|---|---|---|---|---|---|---|---|---|---|---|---|---|
| Well | Total | A- | BCRμ | | | BCRμ- | | BCRKappa | BCRKappa | BCRKappa- | BCRKappa- | CDR3 |
| No. | Reads | ssigned | Reads | BCRμ-V | BCRμ-D | J | BCRμ CDR3 (SEQ ID NO) | Assigned | Reads | V | J | (SEQ Id NO) |
| 1 | 57815 | 12 | 5 | IGHV4-34 | IGHD3-22 | IGHJ4 | CASGRTGYYDSSGYHDYW (613) | | — | — | — | — |
| 5 | 48047 | 15 | 7 | IGHV3-49 | IGHD4-23 | IGHJ4 | CTADHDYGALGLLGWYW (614) | 19 | 8 | IGKV4-1 | IGKJ4 | CQQYYSTPLTF (650) |
| | | | | | or | | | | | | | |
| | | | | | IGHD4-17 | | | | | | | |
| 6 | 150220 | 123962 | 120966 | IGHV3-23D | ? | IGHJ4 | CANQAWLHYW (615) | 22 | 7 | IGKV3-15 | IGKJ5 | CQQYNNWPPLITF (651) |
| 7 | 66006 | 3 | 1 | IGHV5-51 | ? | IGHJ4 | CAGLDVTGDCNDDWSYFHYW | 14 | 7 | IGKV3-15 | IGKJ4 | CQQYNNWPPLTF (652) |
| | | | | | | | (616) | | | | | |
| 8 | 57199 | 56655 | 53945 | IGHV1-69D | IGHD5-24 | IGHJ4 | CARGPVEMAKLYW (617) | 23 | 9 | IGKV3D-11 | IGKJ4 | CQQRSNIWGR*F (653) |
| 9 | 37698 | 18 | 5 | IGHV3-23D | IGHD3-22 | IGHJ4 | CAKVESYSDRRRGYYPNRFFDYW | 36969 | 36106 | IGKV2D-29 | IGKJ3 | CMCISIQLPPTF (654) |
| | | | | | | | (618) | | | | | |
| 11 | 60316 | 34399 | 32690 | IGHV3-23D | ? | IGHJ4 | CAIEFPIHYNSW (619) | 8 | 5 | IGKV3D-15 | IGKJ1 | CQQYNNWPRTF (655) |
| 12 | 63292 | 5 | 1 | IGHV3-23D | ? | IGHJ4 | CANQAWLHYW (620) | 61800 | 60077 | IGKV3-11 | IGKJ5 | CQQRSNWPNTF (656) |
| 13 | 69528 | 21156 | 19895 | IGHV3-11 | IGHD2-15 | IGHJ4 | CAREARYCSGGSCYPYPDYW (621) | 5 | 4 | IGKV3D-11 | IGKJ4 | CQQRSNIWGR*F (657) |
| 14 | 91752 | 11 | 5 | IGHV3-49 | IGHD4-23 | IGHJ4 | CTADHDYGALGLLGWYW (622) | 24 | 10 | IGKV1D-33 | IGKJ4 | CQQYDNLPLTF (658) |
| | | | | | or | | | | | | | |
| | | | | | IGHD4-17 | | | | | | | |
| 15 | 79688 | 36 | 29 | IGHV3-23D | ? | IGHJ4 | CANQAWLHYW (623) | 28 | 8 | IGKV3-15 | IGKJ5 | CQQYNNWPPLITF (659) |
| 16 | 87161 | 2 | 1 | IGHV5-51 | ? | IGHJ4 | WARMDVTGDWKDDWYYFEDW | 19 | 11 | IGKV2-29 | IGKJ1 | CMQGIHLPWTF (660) |
| | | | | | | | (624) | | | | | |
| 17 | 73059 | 24573 | 23727 | IGHV3-49 | IGHD5-24 | IGHJ4 | CTRVGVDGYNYFGNYW (625) | 47698 | 46298 | IGKV1D-33 | IGKJ4 | CQQYDNLPLTF (661) |
| 18 | 76800 | 20515 | 18531 | IGHV3-30-5 | IGHD2-15 | IGHJ4 | CAKSRPLKVVAATFLDYW (626) | 26 | 7 | IGKV2D-29 | IGKJ3 | CMQSIQLPPTF (662) |
| 19 | 76807 | 41524 | 38248 | IGHV3-21 | ? | IGHJ6 | CARDEGQQLPLRPYGMDVW (627) | 28 | 7 | IGKV3D-15 | IGKJ1 | CQQYNNWPRTF (663) |
| 20 | 79400 | 6 | 2 | IGHV2-70 | ? | IGHJ6 | CARIGLGGYSYYYYYGMDVW (628) | 22 | 11 | IGKV3-11 | IGKJ5 | CQQRSNWPNTF (664) |
| 21 | 79576 | 15 | 4 | IGHV4-34 | IGHD3-22 | IGHJ4 | CASGRTGYYDSSGYHDYW (629) | 1 | 1 | IGKV3D-11 | IGKJ4 | CQQRSNIWGR*F (665) |
| 22 | 69702 | 31244 | 29421 | IGHV5-51 | IGHD1-20 | IGHJ4 | CARLDVTGDWNDDWYYFDYW | 37475 | 36158 | IGKV1-5 | IGKJ1 | CQQYNISYGTF (666) |
| | | | | | or | | (630) | | | | | |
| | | | | | IGHD1-1 | | | | | | | |
| 23 | 68715 | 16585 | 15724 | IGHV1-2 | IGHD1-26 | IGHJ4 | CASGGDRWELLPYFDYW (631) | 40320 | 38584 | IGKV3-15 | IGKJ4 | CQQYNNWPPLTF (667) |
| 24 | 79077 | 2 | 1 | IGHV5-51 | IGHD1-20 | IGHJ4 | GARREGGGDWNDQLYYFDYW | 24 | 10 | IGKV2-29 | IGKJ1 | CMQGIHLPWTF (668) |
| | | | | | or | | (632) | | | | | |
| | | | | | IGHD1-1 | | | | | | | |
| 33 | 86545 | 23 | 4 | IGHV4-34 | IGHD3-22 | IGHJ4 | CASGRTGYYDSSGYHDYW (633) | 85751 | 83211 | IGKV1D-12 | IGKJ1 | CQQANSFPGTF (669) |
| 36 | 83766 | 36870 | 34365 | IGHV4-59 | IGHD6-13 | IGHJ6 | CARLSSSWYYYYYGMDVW (634) | 16 | 7 | IGKV3D-15 | IGKJ1 | CQQYNNWPRTF (670) |
| 37 | 78389 | 3 | 2 | IGHV2-70 | ? | IGHJ6 | CARIGLGGYSYYYYYGMDVW (635) | 15 | 8 | IGKV3-11 | IGKJ5 | CQQRSNWPNTF (671) |
| 38 | 81597 | 11028 | 10524 | IGHV1-3 | ? | IGHJ5 | CARQTMIRHRWFDPW (636) | 17093 | 16623 | IGKV3D-11 | IGKJ4 | CQQRSNIWGR*F (672) |
| 39 | 71880 | 21 | 7 | IGHV3-49 | IGHD4-23 | IGHJ4 | CTADHDYGALGLLGWYW (637) | 70997 | 68525 | IGKV4-1 | IGKJ4 | CQQYYSTPLT (673) |
| | | | | | or | | | | | | | |
| | | | | | IGHD4-17 | | | | | | | |
| 41 | 86119 | 32 | 21 | IGHV3-23D | ? | IGHJ4 | CANQAWLHYW (638) | 50764 | 49413 | IGKV1D-39 | IGKJ2 | CQQSYSTPYTF (674) |
| 43 | 60712 | 6 | 3 | IGHV5-51 | IGHD1-20 | IGHJ4 | CARLDVTGDWNDDWYYFDYW | 59833 | 57909 | IGKV2-29 | IGKJ1 | CMQGIHLPWTF (675) |
| | | | | | or | | (639) | | | | | |
| | | | | | IGHD1-1 | | | | | | | |
| 44 | 47280 | 46476 | 39055 | IGHV5-51 | ? | IGHJ6 | CARHLVAPOSPRRNYYYYGMDVW | 48 | 15 | IGKV3D-11 | IGKJ4 | CQQRSNIWGR*F (676) |
| | | | | | | | (640) | | | | | |
| 45 | 58937 | 58348 | 48572 | IGHV3-23D | ? | IGHJ4 | CAKVESYSDRRRGYYPNRFFDYW | 54 | 11 | IGKV3-15 | IGKJ2 | CQQYNNWPPEYTF (677) |
| | | | | | | | (641) | | | | | |
| 46 | 68226 | 19 | 4 | IGHV4-59 | IGHD6-13 | IGHJ6 | CARLSSSWYYYYYGMDVW (642) | 67596 | 65535 | IGKV3D-15 | IGKJ1 | CQQYNNWPRTF (678) |
| 47 | 134229 | 72 | 3 | IGHV2-70 | ? | IGHJ6 | CARIGLGGYSYYYYYGMDVW (643) | 41673 | 39784 | IGKV3-15 | IGKJ1 | CQQYNNWPPWTF (679) |
| 48 | 95986 | 26 | 9 | IGHV4-34 | IGHD3-22 | IGHJ4 | CASGRTGYYDSSGYHDYW (644) | 4 | 4 | IGKV3D-11 | IGKJ4 | CQQRSNIWGR*F (680) |
| 49 | 81390 | 76469 | 76469 | IGHV3-49 | IGHD4-23 | IGHJ4 | CTADHDYGALGLLGWYW (645) | 38 | 13 | IGKV4-1 | IGKJ4 | CQQYYSTPLTF (681) |
| | | | | | or | | | | | | | |
| | | | | | IGHD4-17 | | | | | | | |
| 51 | 84237 | 38 | 28 | IGHV3-23D | ? | IGHJ4 | CANQAWLHYW (646) | 83152 | 78924 | IGKV3-15 | IGKJ5 | CQQYNNWPPLITF (682) |
| 53 | 106334 | 5 | 3 | IGHV5-51 | IGHD1-20 | IGHJ4 | CARLDVTGDWNDDWYYFDYW | 42 | 18 | IGKV3-15 | IGKJ4 | CQQYNNWPPLTF (683) |
| | | | | | or | | (647) | | | | | |
| | | | | | IGHD1-1 | | | | | | | |
| 55 | 100980 | 41443 | 38100 | IGHV2-70 | ? | IGHJ6 | CARIGLGGYSYYYYYGMDVW (648) | 58699 | 56124 | IGKV3-15 | IGKJ2 | CQQYNNWPPEYTF (684) |
| 56 | 109148 | 47720 | 45862 | IGHV4-34 | IGHD3-22 | IGHJ4 | CASGRTGYYDSSGYHDYW (649) | | | | | |

| | | Top | | | | | | | | | |
|---|---|---|---|---|---|---|---|---|---|---|---|
| | BCRLambda | BCRA | | | | | 2nd BCR | | | | |
| Well | A- | Lambda | BCRLambda- | BCRLambda- | BCRLambda CDR3 | BCRLambda- | Lambda | BCRLambda- | BCRLambda- | | BCRLambda- |
| No. | ssigned | Reads | V | J | (SEQ ID NO) | C | Reads | V | J | BCRLambda CDR3 | C |
| 1 | | 54882 | IGLV1-44 | IGL13 or | CAAWDDSLNGQVF (686) | IGLC7 or | | | | | |
| | | | | IGLI2 | | IGLC3 | | | | | |
| 5 | 47448 | 45510 | IGLV1-44 | IGL13 or | CAAWDDSLNGVVF (687) | IGLC3 or | | | | | |
| | | | | IGLI2 | | IGLC2 | | | | | |
| 6 | 16 | 6 | I6LV3-21 | IGL11 | CQVWDSSSDHNYVF | IGLC1 | | | | | |
| | | | | | | (688) | | | | | |
| 7 | 65457 | 42602 | IGLV2-14 | IGL13 or | CSSTTGSSTVVF (689) | IGLC7 or | 19570 | IGLV3-27 | IGL13 | CYSAADNNRVF (723) | IGLC3 or |
| | | | | IGLI2 | | IGLC3 | | IGLC2 | | | |
| 8 | 4 | 2 | IGLV1-44 | IGL13 | CAAWDDSLMGVF (690) | IGLC3 or | | | | | |
| | | | | | | IGLC2 | | | | | |
| 9 | 27 | 9 | IGLV2-14 | IGL13 or | CSSYTSSSTLGVF (691) | IGLC3 or | | | | | |
| | | | | IGLI2 | | IGLC2 | | | | | |
| 11 | 25144 | 24168 | IGLV2-23 | IGL13 | CCSYPRGTSFG**F (692) | IGLC3 or | | | | | |
| | | | | | | IGLC2 | | | | | |
| 12 | 45 | 13 | IGLV2-8 | IGL13 or | CSSYAGSNNLVF (693) | IGLC3 or | | | | | |
| | | | | IGLI2 | | IGLC2 | | | | | |
| 13 | 47525 | 45448 | IGLV1-40 | IGL13 or | CQSYDSSLSVVF (694) | IGLC3 or | | | | | |
| | | | | IGLI2 | | IGLC2 | | | | | |
| 14 | 90360 | 86661 | IGLV1-40 | IGL13 | CQSYDSSLSGCVF (695) | IGLC7 or | | | | | |
| | | | | | | IGLC3 | | | | | |
| 15 | 78543 | 75085 | 1GLV3-21 | IGL11 | CQVWDSSSDHNYVF | IGLC1 | | | | | |
| | | | | | (696) | | | | | | |
| 16 | 292 | 60 | IGLV6-57 | IGL13 | CQTSDSRITVF (697) | IGLC3 or | | | | | |
| | | | | | | IGLC2 | | | | | |
| 17 | 10 | 1 | IGLV8-61 | IGL13 | CVSYMGSGTWVF (698) | IGLC3 or | | | | | |
| | | | | | | IGLC2 | | | | | |
| 18 | 55573 | 53294 | IGLV2-14 | IGL13 or | CSSYTSSSTLGVF (699) | IGLC3 or | | | | | |
| | | | | IGLI2 | | IGLC2 | | | | | |
| 19 | 30978 | 29613 | IGLV3-21 | IGL13 | CQVWDRSSDHWVF (700) | IGLC3 or | | | | | |
| | | | | | | IGLC2 | | | | | |
| 20 | 78790 | 51024 | IGLV2-8 | IGL13 or | CSSYAGSNNLVF (701) | IGLC3 or | 24101 | IGLV8-61 | IGL13 | CVLYMGSGIWVF (724) | IGLC3 or |
| | | | | IGLI2 | | IGLC2 | | | | | IGLC2 |
| 21 | 78183 | 75248 | IGLV2-14 | IGL11 | CSSYTGSTTLDVF (702) | IGLC1 | | | | | |
| 22 | 50 | 10 | IGLV1-44 | IGL13 or | CAAWDDSLNGVVF (703) | IGLC3 or | | | | | |
| | | | | IGLI2 | | IGLC2 | | | | | |
| 23 | 29 | 8 | IGLV3-21 | IGL11 | CQVWDSSSDHNYVF | IGLC1 | | | | | |
| | | | | | | (704) | | | | | |
| 24 | 78342 | 75711 | IGLV2-14 | IGL13 or | CSSYTSSSTLVF (705) | IGLC3 or | | | | | |
| | | | | IGLI2 | | IGLC2 | | | | | |
| 33 | 34 | 10 | IGLV2-14 | IGL11 | CSSYTSSSTYVF (706) | IGLC1 | | | | | |
| 36 | 46184 | 43984 | IGLV1-40 | IGL13 or | CQSYDSSLSGPVVF (707) | IGLC3 or | | | | | |
| | | | | IGLI2 | | IGLC2 | | | | | |
| 37 | 2 | 74708 | IGLV2-14 | IGL11 | CSSYTSSSTYVF (708) | IGLC1 | | | | | |
| 38 | 52548 | 50579 | IGLV2-11 | IGL13 | CCSYAGSYTWVF (709) | IGLC3 or | | | | | |
| | | | | | | IGLC2 | | | | | |
| 39 | 25 | 8 | IGLV2-11 | IGL13 | CCSYAGSYTWVF (710) | IGLC3 or | | | | | |
| | | | | | | IGLC2 | | | | | |
| 41 | 13 | 8 | IGLV3-21 | IGL11 | CQVWDSSSDHNYVF | IGLC1 | | | | | |
| | | | | | (711) | | | | | | |
| 43 | 18 | 6 | IGLV3-21 | IGL13 | CQVWDSSSDHPNWVF | IGLC3 or | | | | | |
| | | | | | (712) | IGLC2 | | | | | |
| 44 | 3 | 2 | IGLV2-14 | IGL11 | CSSYTSSSTYVF (713) | IGLC1 | | | | | |
| 45 | 18 | 6 | IGLV2-14 | IGL11 | CSSYTSSSTYVF (714) | IGLC1 | | | | | |
| 46 | 17 | 6 | IGLV1-40 | IGL13 or | CQSYDSSLSVVF (715) | IGLC3 or | | | | | |
| | | | | IGLI2 | | IGLC2 | | | | | |
| 47 | 23 | 6 | IGLV2-8 | IGL13 or | CSSYAGSNNLVF (716) | IGLC3 or | | | | | |
| | | | | IGLI2 | | IGLC2 | | | | | |
| 48 | 94817 | 58614 | IGLV4-69 | IGL13 | CQTWGTGIQVF (717) | IGLC3 or | 31347 | IGLV1-47 | IGL13 | CAAWDDSLSGQVF | IGLC3 or |
| | | | | | | IGLC2 | | | | (725) | IGLC2 |
| 49 | 47 | 10 | IGLV1-40 | IGL13 | CQSYDSSLSGCVF (718) | IGLC7 or | | | | | |
| | | | | | | IGLC3 | | | | | |
| 51 | 36 | 13 | IGLV2-14 | IGL11 | CSSYTSSSTYVF (719) | IGLC1 | | | | | |
| 53 | 100586 | 95854 | 1GLV3-21 | 1GL13 | CQVWDSSSDHPNWVF | IGLC3 or | | | | | |
| | | | | | (720) | IGLC2 | | | | | |
| 55 | 10 | 2 | IGLV2-14 | IGL13 or | CSSYTSSSTPVVF (721) | IGLC7 or | | | | | |
| | | | | IGLI2 | | IGLC3 | | | | | |
| 56 | 59793 | 57838 | IGLV2-14 | IGL11 | CSSYTSSSTYVF (722) | IGLC1 | | | | | |

TABLE 29
Results of separately amplifying and sequencing BCRμ, BCRκ, and BCRλ
(? means unidentified, * is a stop codon)

| Well | Amplified | Total | BCRH/L | Top | | | | |
|---|---|---|---|---|---|---|---|---|
| No. | BCR type | Reads | Assigned | Reads | BCR-V | BCR-D | BCR-J | CDR3 (SEQ ID NO) |
| 13 | BCRμ | 34705 | 34199 | 32076 | IGHV3-11 | IGHD2-15 | IGHJ4 | CAREARYCSGGSCYPYPDYW (726) |
| 17 | BCRμ | 38257 | 37729 | 36316 | IGHV3-49 | IGHD5-24 | IGHJ4 | CTRVGVDGYNYFGNYW (727) |
| 18 | BCRμ | 35992 | 35583 | 32306 | IGHV3-30-5 | IGHD2-15 | IGHJ4 | CAKSRPLKVVAATFLDYW (728) |
| 22 | BCRμ | 41689 | 41248 | 38885 | IGHV5 | IGHD1-20 or | IGHJ4 | CARLDVTGDWNDDWYYFDYW (729) |
| | | | | | | IGHD1-1 | | |
| 23 | BCRμ | 21998 | 21731 | 20598 | IGHV1-2 | IGHD1-26 | IGHJ4 | CASGGDRWELLPYFDYW (730) |
| 36 | BCRμ | 30070 | 29857 | 27834 | IGHV4-59 | IGHD6-13 | IGHJ6 | CARLSSSWYYYYYGMDVW (731) |
| 38 | BCRμ | 33098 | 32753 | 31381 | IGHV1-3 | ? | IGHJ5 | CARQTMIRHRWFDPW (732) |
| 55 | BCRμ | 29910 | 29644 | 27181 | IGHV2-70 | ? | IGHJ6 | CARIGLGGYSYYYYYGMDVW (733) |
| 56 | BCRμ | 21154 | 20931 | 20112 | IGHV4-34 | IGHD3-22 | IGHJ4 | CASGRTGYYDSSGYHDYW (734) |
| 17 | BCRKappa | 27937 | 27594 | 26780 | IGKV1D-33 | — | IGHJ4 | CQQYDNLPLTF (735) |
| 22 | BCRKappa | 26478 | 26247 | 25464 | IGKV1-5 | — | IGHJ1 | CQQYNSYGTF (736) |
| 23 | BCRKappa | 30249 | 23215 | 22303 | IGKV3-15 | — | IGHJ4 | CQQYNNWPPLTF (737) |
| 38 | BCRKappa | 25304 | 24898 | 24313 | IGKV3D-11 | — | IGHJ4 | CQQRSNWGR*F (738) |
| 55 | BCRKappa | 28095 | 27830 | 26736 | IGKV3-15 | — | IGHJ2 | CQQYNNWPPEYTF (739) |
| 13 | BCRLambda | 30897 | 30642 | 29579 | IGLV1-40 | — | IGLI3 or | CQSYDSSLSVVF (740) |
| | | | | | | | IGLJ2 | |
| 18 | BCRLambda | 25848 | 25615 | 24654 | IGLV2-14 | —— | IGLI3 or | CSSYTSSSTLGVF (741) |
| | | | | | | | IGLJ2 | |
| 36 | BCRLambda | 41623 | 41117 | 39218 | IGLV1-40 | | IGLI3 or | CQSYDSSLSGPVVF (742) |
| | | | | | | | IGLJ2 | |
| 38 | BCRLambda | 47708 | 47083 | 45322 | IGLV2-11 | — | IGLJ3 | CCSYAGSYTWVF (743) |
| 56 | BCRLambda | 37083 | 36383 | 35159 | IGLV2-14 | — | IGLJ1 | CSSYTSSSTYVF (744) |

Next, read 1 (data for sequencing from the 3′ side C region side of BCR cDNA) and read 2 (data for sequencing from the 5′ side of BCR cDNA) of MiSeq sequencing data were joined with a fastq-join program, converted to a reverse complementary strand sequence, and converted to a sequence with the same orientation as normal cDNA. After picking up only clones with a primer sequence on both ends and removing the primer sequences, the clones were randomly selected, and base sequences of amplified cDNA were obtained from base sequence assembly multiple alignment analysis. If a DNA fragment was long (longer than about 500 to 580 base pairs) leading to poor joining rate, a low quality region was removed while utilizing a reverse complementary sequence of read 1 for a sequence that failed to join for a read assigned to said clone and sequence of read 2 without modification, in addition to a sequence that was able to join, and then base sequence assembly multiple alignment analysis was performed to obtain the base sequence of amplified cDNA. The resulting cDNA base sequence was compared to genetic sequences on a public database such as Ensemble's Gene database to confirm whether a sequence was obtained from a 5′ amino acid sequence start codon. Further, after an amino acid sequence substitution, the amino acid sequences of reader regions and variable regions (four framework regions: FR1 to 4 and three variable regions: CDR1 to 3) were predicted. The resulting base sequences and amino acid translated sequences of BCRμ and BCR Lambda of well No. 18 and BCRμ and BCR Lambda of Well No. 55 are shown as examples of analysis (underlines indicate translation start codon annotated on Ensemble's Gene database and CDR3 regions identified by computer analysis). Protein information was obtained in-frame from a translation start codon to the N-terminal amino acid sequence of a C region comprising a CDR3 region in each case.

>Well No. 18 BCRμ base sequence
(SEQ ID NO: 796)
GGCACTAGAAGTCGGCGGTGTTTCCATTCGGTGATCAGCACTGAACACAGAGGACTCACCA
TGGAGTTTGGGCTGAGCTGGGTTTTCCTCGTTGCTCTTTTAAGGGGTGTCCAGTGTCAGGT
GCAGCTGGTGGAGTCTGGGGGAGGCGTGGTCCAGCCTGGGAGGTCCCTGAGACTCTCCTGT
GCAGCCTCTGGATTCACCTTCAGTAGCTATGGCATGCACTGGGTCCGCCAGGCTCCAGGCA
AGGGGCTGGAGTGGGIGGCAGTTATATCATATGATGGAAGTAATAAATACTATGCAGACTC
CGTGAAGGGCCGATTCACCATCTCCAGAGACAATTCCAAGAACACGCTGTATCTGCAAATG
AACAGCCTGAGAGCTGAGGACACGGCTGTGTATTACTGTGCCAAATCCCGACCCCTGAAGG
TGGTAGCTGCTACTTTTCTTGACTACTGGGGCCAGGGAACCCTGGTCACCGTCTCCTCAGG
GAGTGCATCCGCCCCAACCCTTTTCCCCCTCGTCTCCTGT
>Well No. 18 BCRμ amino acid translated sequence
(SEQ ID NO: 797)
GTRSRRCFHSVISTEHRGLTMEFGLSWVFLVALLRGVQCQVQLVESGGGVVQPGRSLRLSC
AASGFTFSSYGMHWVRQAPGKGLEWVAVISYDGSNKYYADSVKGRFTISRDNSKNTLYLQM
NSLRAEDTAVYYCAKSRPLKVVAATFLDYWGQGTLVTVSSGSASAPTLFPLVSC
>Well No. 18 BCR Lambda base sequence
(SEQ ID NO: 798)
GGGGGTCTCAGGAGGCAGCGCTCTCGGGACGTCTCCACCATGGCCTGGGCTCTGCTATTCC
TCACCCTCCTCACTCAGGGCACAGGGTCCTGGGCCCAGTCTGCCCTGACTCAGCCTGCCTC
CGTGTCTGGGTCTCCTGGACAGTCGATCACCATCTCCTGCACTGGAACCAGCAGTGACGTT
GGTGGTTATAACTATGTCTCCTGGTACCAACAGCACCCAGGCAAAGCCCCCAAACTCATGA
TTTATGATGTCAGTAATCGGCCCTCAGGGGTTTCTAATCGCTTCTCTGGCTCCAAGTCTGG
CAACACGGCCTCCCTGACCATCTCTGGGCTCCAGGCTGAGGACGAGGCTGATTATTACTGC
AGCTCATATACAAGCAGCAGCACTCTCGGGGTATTCGGCGGAGGGACCAAGCTGACCGTCC
TAGGTCAGCCCAAGGCTGCCCCCTCGGTCACTCTGTTCCCGCCCTCCTCTGAGGAGCTT
>Well No. 18 BCR Lambda amino acid translated sequence
(SEQ ID NO: 799)
GGLRRQRSRDVSTMAWALLFLTLLTQGTGSWAQSALTQPASVSGSPGQSITISCTGTSSDV
GGYNYVSWYQQHPGKAPKLMIYDVSNRPSGVSNRFSGSKSGNTASLTISGLQAEDEADYYC
SSYTSSSTLGVFGGGTKLTVLGQPKAAPSVTLFPPSSEEL
>Well No. 55 BCRμ base sequence
(SEQ ID NO: 800)
GCCTCGCACAGTGAATCCTGCTCCCCACCATGGACATACTTTGTTCCACGCTCCTGCTACT
GACTGTCCCGTCCTGGGTCTTATCCCAGGTCACCTTGAGGGAGTCTGGTCCTGCGCTGGTG
AAACCCACACAGACCCTCACACTGACCTGCACCTTCTCTGGGTTCTCACTCAGCACTAGTG
GAATGTGTGTGAGCTGGATCCGTCAGCCCCCAGGGAAGGCCCTGGAGTGGCTTGCACGCAT
TGATTGGGATGATGATAAATACTACAGCACATCTCTGAAGACCAGGCTCACCATCTCCAAG
GACACCTCCAAAAACCAGGTGGTCCTTACAATGACCAACATGGACCCTGTGGACACAGCCA
CGTATTACTGTGCACGGATAGGTCTAGGTGGCTATTCTTACTACTACTACTACGGTATGGA
CGTCTGGGGCCAAGGGACCACGGTCACCGTCTCCTCAGGGAGTGCATCCGCCCCAACCCTT
TTCCCCCTCGTCTCCTGT
>Well No. 55 BCRμ amino acid translated sequence
(SEQ ID NO: 801)
LAQ*ILLPTMDILCSTLLLLTVPSWVLSQVTLRESGPALVKPTQTLTLICTFSGFSLSTSG
MCVSWIRQPPGKALEWLARIDWDDDKYYSTSLKTRLTISKDTSKNQVVLTMTNMDPVDTAT
YYCARIGLGGYSYYYYYGMDVWGQGTTVTVSSGSASAPTLFPLVSC
>Well No. 55 BCR Kappa base sequence
(SEQ ID NO: 802)
GGAGGAACTGCTCAGTTAGGACCCAGACGGAACCATGGAAGCCCCAGCGCAGCTTCTCTTC
CTCCTGCTACTCTGGCTCCCAGATACCACTGGAGAAATAGTGATGACGCAGTCTCCAGCCA
CCCTGTCTGTGTCTCCAGGGGAAAGAGCCACCCTCTCCTGCAGGGCCAGTCAGAGTGTTAG
CAGCAACTTAGCCTGGTACCAGCAGAAACCTGGCCAGGCTCCCAGGCTCCTCATCTATGGT
GCATCCACCAGGGCCACTGGTATCCCAGCCAGGTTCAGTGGCAGTGGGTCTGGGACAGAGT
TCACTCTCACCATCAGCAGCCTGCAGTCTGAAGATTTTGCAGTTTATTACTGTCAGCAGTA
TAATAACTGGCCTCCGGAGTACACTTTTGGCCAGGGGACCAAGCTGGAGATCAAACGAACT
GTGGCTGCACCATCTGTCTTCATCTTCCCGCCATCTGATGAGCAGTTGAAATCTGGAACTG
CCTCTGT
>Well No. 55 BCR Kappa amino acid translated sequence
(SEQ ID NO: 803)
EELLS*DPDGTMEAPAQLLFLLLLWLPDTTGEIVMTQSPATLSVSPGERATLSCRASQSVS
SNLAWYQQKPGQAPRLLIYGASTRATGIPARFSGSGSGTEFTLTISSLQSEDFAVYYCQQY
NNWPPEYTFGQGTKLEIKRTVAAPSVFIFPPSDEQLKSGTAS

Proteins can be expressed or synthesized in a microbial/cellular system or artificially synthesized system and applied to antibody creation, antibody drug, CART therapy, or the like by optionally subjecting the cDNA base sequence or cDNA fragment of a variable region obtained by such analysis to reamplification, artificial gene synthesis, or the like, and cloning or joining them with a sequence of a C region with few functional differences to obtain a full length coding region, or joining them with intracellular regions of various proteins such as CD3 intracellular region, CD28 intracellular region, 4-1BB intracellular region, ICOS intracellular region, and OX40 intracellular region, signaling domain, functional domain, protein fragment, artificially designed protein fragment, and chimeric proteins thereof to obtain a chimeric protein coding region.

### (Example 5: TCR (or BCR) Pair Gene Cloning)

Experiment E

(Reagent)

The additional reagents used in Experiment E are the following.
Competent E. coli cell (Nippon Gene)
pcDNA3.1(+)/V5-His (B) vector (Thermo Fisher Scientific)
pMXs vector (CELL BIOLABS)
Restriction enzyme EcoRI (Nippon Gene)
Restriction enzyme EcoRV (Nippon Gene)
NEBuilder HiFi DNA Assembly Master Mix (NEW England BioLabs)
NucleoSpin Plasmid (Takara Bio)

*Oligonucleotide sequences used
TCR cloning pcDNA3.1(+)/V5-His (B) forward primer-TS-AdpD3EV:
(SEQ ID NO: 745)
TGGTGGAATTCTGCAGATAAGCAGTGGTATCAACGCA
-hTCRa-VX-F-AdpD3EV:
(SEQ ID NO: 746)
TGGTGGAATTCTGCAGAT
NNNN------NNNN (″NNNN------NNNN″ is a sequence of about 16 to
35 bases of a TCRα V region 5′ untranslated region)
-hTCRa-V24-F-AdpD3EV:
(SEQ ID NO: 747)
TGGTGGAATTCTGCAGATTTTCTGCTGTGGGTACGTGAG
-hTCRb-VX-F-AdpD3EV:
(SEQ ID NO: 748)
TGGTGGAATTCTGCAGAT
NNNN------NNNN (″NNNN------NNNN″ is a sequence of about 16 to
35 bases of a TCRβ V region 5′ untranslated region)
-hTCRb-V6.5-F-AdpD3EV:
(SEQ ID NO: 749)
TGGTGGAATTCTGCAGATGAGAGTCCTGCTCCCCTTTC
TCR cloning pMXs forward primer-TS-AdpMXEI:
(SEQ ID NO: 750)
CCAGTGTGGTGGTACGGGAAGCAGTGGTATCAACGCA
-hTCRa-VX-F-AdpMXEI:
(SEQ ID NO: 751)
CCAGTGTGGTGGTACGGG
NNNN------NNNN (″NNNN------NNNN″ is a sequence of about 16 to
35 bases of a TCRα V region 5′ untranslated region)
-hTCRb-VX-F-AdpMXEI:
(SEQ ID NO: 752)
CCAGTGTGGTGGTACGGG
NNNN------NNNN (″NNNN------NNNN″ is a sequence of about 16 to
35 bases of a TCRβ region 5′ untranslated region)
-hTCRa-V24-F-AdpMXEI:
(SEQ ID NO: 753)
CCAGTGTGGTGGTACGGGTTTCTGCTGTGGGTACGTGAG
-hTCRb-V6.5-F-AdpMXEI:
(SEQ ID NO: 754)
CCAGTGTGGTGGTACGGGGAGAGTCCTGCTCCCCTTTC
(underlined portions are adaptor sequences for assembly and
ligation that are homologous to an arm region of a vector)
TCR cloning common reverse primer
-hTCRa-C-R:
(SEQ ID NO: 755)
AGGGTCAGGGTTCTGGATAT
hTCRb-C1-R:
(SEQ ID NO: 756)
GAACACCTTGTTCAGGTCCT
hTCRb-C2-R:
(SEQ ID NO: 757)
GAACACGTTTTTCAGGTCCT
hTCRα sequencing primer
hTCRaC-F0:
(SEQ ID NO: 758)
TCTGCCTATTCACCGATTTTG
hTCRaC-F1:
(SEQ ID NO: 759)
GGACTTCAAGAGCAACAGTGC
hTCRaC-F2:
(SEQ ID NO: 760)
TGTCAAGCTGGTCGAGAAAAG
hTCRaC-F3:
(SEQ ID NO: 761)
ACGCCTTCAACAACAGCATTA
hTCRaC-R1v1:
(SEQ ID NO: 762)
CTTTTCTCGACCAGCTTGACA
hTCRaC-R2:
(SEQ ID NO: 763)
CCACTTTCAGGAGGAGGATTC
hTCRaC-R4:
(SEQ ID NO: 764)
CAAAATCGGTGAATAGGCAGA
hTCRaC-R5:
(SEQ ID NO: 765)
ATAGACCTCATGTCTAGCACAG
hTCRβ, sequencing primer
hTCR-CB-F2:
(SEQ ID NO: 766)
GCTGTGTTTGAGCCATCAGAA
hTCRbC-F5:
(SEQ ID NO: 767)
TGAATGGGAAGGAGGTGCACAGT
hTCR-CB-R2:
(SEQ ID NO: 768)
TTCTGATGGCTCAAACACAGC
hTCR-CB2:
(SEQ ID NO: 769)
AGGCAGTATCTGGAGTCATTGAG
hTCR-CB3:
(SEQ ID NO: 770)
ACTGTGCACCTCCTTCCCATTCA
hTCRbC2-CloR1:
(SEQ ID NO: 771)
GTTTAGCCTATTTCGTACTTGG
hTCRbC1-R1:
(SEQ ID NO: 772)
AGTCACTTAGGCATGCTAAGGTC

*Synthetic gene fragment used (custom synthetic double stranded DNA; can be synthesized at Thermo Fisher Scientific, Gene Design, INTEGRATED DNA TECHNOLOGIES, or the like. As an alternative method, RNA can be prepared from bulk PBMC cells, and cDNA can be amplified by PCR. Genes are known to have polymorphisms at racial or individual levels. The base sequence of the genotype of interest is changed as needed.)

For pcDNA3.1(+)/V5-His (B)
-hTCRaCFull-AdpD3EV:
(SEQ ID NO: 773)
ATATCCAGAACCCTGACCCTGCCGTGTACCAGCTGAGAGACTCTAAATCCAGTGACAAGTC
TGTCTGCCTATTCACCGATTTTGATTCTCAAACAAATGTGTCACAAAGTAAGGATTCTGAT
GTGTATATCACAGACAAAACTGTGCTAGACATGAGGTCTATGGACTTCAAGAGCAACAGTG
CTGTGGCCTGGAGCAACAAATCTGACTTTGCATGTGCAAACGCCTTCAACAACAGCATTAT
TCCAGAAGACACCTTCTTCCCCAGCCCAGAAAGTTCCTGTGATGTCAAGCTGGTCGAGAAA
AGCTTTGAAACAGATACGAACCTAAACTTTCAAAACCTGTCAGTGATTGGGTTCCGAATCC
TCCTCCTGAAAGTGGCCGGGTTTAATCTGCTCATGACGCTGCGGCTGTGGTCCAGCTGAAT
CCAGCACAGTGGCGGC
-hTCRaCFull_noSTOP-AdpD3EV:
(SEQ ID NO: 774)
ATATCCAGAACCCTGACCCTGCCGTGTACCAGCTGAGAGACTCTAAATCCAGTGACAAGTC
TGTCTGCCTATTCACCGATTTTGATTCTCAAACAAATGTGTCACAAAGTAAGGATTCTGAT
GTGTATATCACAGACAAAACTGTGCTAGACATGAGGTCTATGGACTTCAAGAGCAACAGTG
CTGTGGCCTGGAGCAACAAATCTGACTTTGCATGTGCAAACGCCTTCAACAACAGCATTAT
TCCAGAAGACACCTTCTTCCCCAGCCCAGAAAGTTCCTGTGATGTCAAGCTGGTCGAGAAA
AGCTTTGAAACAGATACGAACCTAAACTTTCAAAACCTGTCAGTGATTGGGTTCCGAATCC
TCCTCCTGAAAGTGGCCGGGTTTAATCTGCTCATGACGCTGCGGCTGTGGTCCAGCTTAAT
CCAGCACAGTGGCGGC
-hTCRbC1Full-AdpD3EV:
(SEQ ID NO: 775)
AGGACCTGAACAAGGTGTTCCCACCCGAGGTCGCTGTGTTTGAGCCATCAGAAGCAGAGAT
CTCCCACACCCAAAAGGCCACACTGGTGTGCCTGGCCACAGGCTTCTTCCCTGACCACGTG
GAGCTGAGCTGGTGGGTGAATGGGAAGGAGGTGCACAGTGGGGTCAGCACGGACCCGCAGC
CCCTCAAGGAGCAGCCCGCCCTCAATGACTCCAGATACTGCCTGAGCAGCCGCCTGAGGGT
CTCGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCCGTCACCCAGATCGTCAGCG
CCGAGGCCTGGGGTAGAGCAGACTGTGGCTTTACCTCGGTGTCCTACCAGCAAGGGGTCCT
GTCTGCCACCATCCTCTATGAGATCCTGCTAGGGAAGGCCACCCTGTATGCTGTGCTGGTC
AGCGCCCTTGTGTTGATGGCCATGGTCAAGAGAAAGGATTTCTGAATCCAGCACAGTGGCG
GC
-hTCRbC1Full_noSTOP-AdpD3EV:
(SEQ ID NO: 776)
AGGACCTGAACAAGGTGTTCCCACCCGAGGTCGCTGTGTTTGAGCCATCAGAAGCAGAGAT
CTCCCACACCCAAAAGGCCACACTGGTGTGCCTGGCCACAGGCTTCTTCCCTGACCACGTG
GAGCTGAGCTGGTGGGTGAATGGGAAGGAGGTGCACAGTGGGGTCAGCACGGACCCGCAGC
CCCTCAAGGAGCAGCCCGCCCTCAATGACTCCAGATACTGCCTGAGCAGCCGCCTGAGGGT
CTCGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCCGTCACCCAGATCGTCAGCG
CCGAGGCCTGGGGTAGAGCAGACTGTGGCTTTACCTCGGTGTCCTACCAGCAAGGGGTCCT
GTCTGCCACCATCCTCTATGAGATCCTGCTAGGGAAGGCCACCCTGTATGCTGTGCTGGTC
AGCGCCCTTGTGTTGATGGCCATGGTCAAGAGAAAGGATTTCTTAATCCAGCACAGTGGCG
GC
-hTCRbC2Full-AdpD3EV:
(SEQ ID NO: 777)
AGGACCTGAAAAACGTGTTCCCACCCGAGGTCGCTGTGTTTGAGCCATCAGAAGCAGAGAT
CTCCCACACCCAAAAGGCCACACTGGTGTGCCTGGCCACAGGCTTCTACCCCGACCACGTG
GAGCTGAGCTGGTGGGTGAATGGGAAGGAGGTGCACAGTGGGGTCAGCACAGACCCGCAGC
CCCTCAAGGAGCAGCCCGCCCTCAATGACTCCAGATACTGCCTGAGCAGCCGCCTGAGGGT
CTCGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCTGTCACCCAGATCGTCAGCG
CCGAGGCCTGGGGTAGAGCAGACTGTGGCTTCACCTCCGAGTCTTACCAGCAAGGGGTCCT
GTCTGCCACCATCCTCTATGAGATCTTGCTAGGGAAGGCCACCTTGTATGCCGTGCTGGTC
AGTGCCCTCGTGCTGATGGCCATGGTCAAGAGAAAGGATTCCAGAGGCTAGATCCAGCACA
GTGGCGGC
-hTCRbC2Full_noSTOP-AdpD3EV:
(SEQ ID NO: 778)
AGGACCTGAAAAACGTGTTCCCACCCGAGGTCGCTGTGTTTGAGCCATCAGAAGCAGAGAT
CTCCCACACCCAAAAGGCCACACTGGTGTGCCTGGCCACAGGCTTCTACCCCGACCACGTG
GAGCTGAGCTGGTGGGTGAATGGGAAGGAGGTGCACAGTGGGGTCAGCACAGACCCGCAGC
CCCTCAAGGAGCAGCCCGCCCTCAATGACTCCAGATACTGCCTGAGCAGCCGCCTGAGGGT
CTCGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCTGTCACCCAGATCGTCAGCG
CCGAGGCCTGGGGTAGAGCAGACTGTGGCTTCACCTCCGAGTCTTACCAGCAAGGGGTCCT
GTCTGCCACCATCCTCTATGAGATCTTGCTAGGGAAGGCCACCTTGTATGCCGTGCTGGTC
AGTGCCCTCGTGCTGATGGCCATGGTCAAGAGAAAGGATTCCAGAGGCTTAATCCAGCACA
GTGGCGGC
For pMXs
-hTCRaCFull-AdpMXEI:
(SEQ ID NO: 779)
ATATCCAGAACCCTGACCCTGCCGTGTACCAGCTGAGAGACTCTAAATCCAGTGACAAGTC
TGTCTGCCTATTCACCGATTTTGATTCTCAAACAAATGTGTCACAAAGTAAGGATTCTGAT
GTGTATATCACAGACAAAACTGTGCTAGACATGAGGTCTATGGACTTCAAGAGCAACAGTG
CTGTGGCCTGGAGCAACAAATCTGACTTTGCATGTGCAAACGCCTTCAACAACAGCATTAT
TCCAGAAGACACCTTCTTCCCCAGCCCAGAAAGTTCCTGTGATGTCAAGCTGGTCGAGAAA
AGCTTTGAAACAGATACGAACCTAAACTTTCAAAACCTGTCAGTGATTGGGTTCCGAATCC
TCCTCCTGAAAGTGGCCGGGTTTAATCTGCTCATGACGCTGCGGCTGTGGTCCAGCTGACC
AGCTGAGCGCCGGTCG
-hTCRbC1Full-AdpMXEI:
(SEQ ID NO: 780)
AGGACCTGAACAAGGTGTTCCCACCCGAGGTCGCTGTGTTTGAGCCATCAGAAGCAGAGAT
CTCCCACACCCAAAAGGCCACACTGGTGTGCCTGGCCACAGGCTTCTTCCCTGACCACGTG
GAGCTGAGCTGGTGGGTGAATGGGAAGGAGGTGCACAGTGGGGTCAGCACGGACCCGCAGC
CCCTCAAGGAGCAGCCCGCCCTCAATGACTCCAGATACTGCCTGAGCAGCCGCCTGAGGGT
CTCGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCCGTCACCCAGATCGTCAGCG
CCGAGGCCTGGGGTAGAGCAGACTGTGGCTTTACCTCGGTGTCCTACCAGCAAGGGGTCCT
GTCTGCCACCATCCTCTATGAGATCCTGCTAGGGAAGGCCACCCTGTATGCTGTGCTGGTC
AGCGCCCTTGTGTTGATGGCCATGGTCAAGAGAAAGGATTTCTGACCAGCTGAGCGCCGGT
CG
-hTCRbC2Full-AdpMXEI:
(SEQ ID NO: 781)
AGGACCTGAAAAACGTGTTCCCACCCGAGGTCGCTGTGTTTGAGCCATCAGAAGCAGAGAT
CTCCCACACCCAAAAGGCCACACTGGTGTGCCTGGCCACAGGCTTCTACCCCGACCACGTG
GAGCTGAGCTGGTGGGTGAATGGGAAGGAGGTGCACAGTGGGGTCAGCACAGACCCGCAGC
CCCTCAAGGAGCAGCCCGCCCTCAATGACTCCAGATACTGCCTGAGCAGCCGCCTGAGGGT
CTCGGCCACCTTCTGGCAGAACCCCCGCAACCACTTCCGCTGTCAAGTCCAGTTCTACGGG
CTCTCGGAGAATGACGAGTGGACCCAGGATAGGGCCAAACCTGTCACCCAGATCGTCAGCG
CCGAGGCCTGGGGTAGAGCAGACTGTGGCTTCACCTCCGAGTCTTACCAGCAAGGGGTCCT
GTCTGCCACCATCCTCTATGAGATCTTGCTAGGGAAGGCCACCTTGTATGCCGTGCTGGTC
AGTGCCCTCGTGCTGATGGCCATGGTCAAGAGAAAGGATTCCAGAGGCTAGCCAGCTGAGC
GCCGGTCG

E1. Preparation of DNA Fragment for Cloning

E1-1. Preparation of Variable Region DNA Fragment

A variable region is amplified for functional pair genes of a single cell of TCR (or BCR) by a one-step RT-TS-PCR reaction or the following PCR reaction using a semi-nested PCR amplicon purified DNA as a template. TCRβ has two types of C regions. A C1 clone and C2 clone are distinguished from sequencing data or J region data (J1-1 to 6 are joined to C1, and J2-1 to 7 are joined to C2) to select a reverse side primer. (However, it is believed that there is no significant functional difference between the C1 sequence and C2 sequence of TCRβ, so that either can be used in most cases.) DNA amplification is confirmed through agarose gel electrophoresis or the like. Any V region can be amplified at once by using a sequence added to the 5′ side as the forward side primer and a sequence of a C region of TCR as the reverse side primer in one-step RT-TS-PCR. If it appears that there is a short fragment that does not comprise a start codon or a non-specific sequence or if a transcription product from two chromosomes is found from the result of electrophoresis or sequencing, amplification using a relevant V region specific sequence results in a DNA fragment with a high purity. An amplicon is purified with AMPure XP and DNA purification column. If a short DNA fragment of 400 bases or less, a long fragment of 7000 bases or greater, or the like is observed, a DNA fragment with a length of interest is cut out through agarose gel electrophoresis and purified as needed. While this protocol only amplifies a variable region, a reverse transcription primer and a block primer of one-step-RT-TS PCR or a primer of semi-nested PCR can be designed to be more downstream towards 3′ than a stop codon (this can be in a form comprising a coding region, as long as a PCR product contains a stop codon site) and subjected to PCR, so that this can include from a translation start codon to a stop codon (when incorporating into pcDNA3.1(+)/V5-His (B) to construct a construct for a tagged protein, a stop codon is converted into some type of amino acid coding codon). Similarly, a DNA fragment comprising a full length coding region can be synthesized without PCR.

For example, it is understood that the TCRα (V24.J49.C clone)/TCRβ, (V6-5.D1.J1-2.C1 clone) pair of well No. 13 obtained in Example 1 (Experiment A) can be amplified in the following reaction.

TABLE 31
Amplification of TCRα (V24 · J49 · C clone)

| | | Amount of |
|---|---|---|
| | Amount of | solution upon |
| | solution upon use | use of TCRα |
| | of TS adapter | variable region |
| Reagents, etc. | primer (μl) | primer (μl) |
| One-step RT-TS-PCR reaction | 9.2 | 9.2 |
| solution, or semi-nested PCR | | |
| product, or index PCR product of | | |
| well No. 13 + DW | | |
| 2 × KAPA HiFi Hot Start Ready Mix | 10.0 | 10.0 |
| Forward primer (10 μm) | 0.4 | — |
| TS-AdpD3EV or TS-AdpMXEI | | |
| Forward primer (10 μm) | — | 0.4 |
| hTCRa-V24-F-AdpD3EV | | |
| or TCRa-V24-F-AdpMXEI | | |
| htCRa-C-R (10 uM) | 0.4 | 0.4 |
| Total | 20.0 | 20.0 |

E1-2. Preparation of Cloning Vector

Expression vector pcDNA3.1(+)/V5-His (B) is digested with EcoRV, and retroviral vector pMXs is digested with EcoRI in accordance with the manufacturer's protocol. Optionally, agarose gel electrophoresis is performed, and a linear DNA band digested with an enzyme is cut out, and DNA is purified with a DNA purification column.

E2. DNA Fragment Ligation and Introduction into E. coli to Obtain a Clone, and Sequence Verification

E2-1. DNA Fragment Ligation and Introduction into E. coli

Three types of DNA fragments, i.e., prepared variable region DNA fragment, genetically synthesized C region fragment (also can be amplified by PCR), and vector DNA digested by a restriction enzyme, are assembled and ligated using NEBuilder HiFi DNA Assembly Master Mix or the like.

Assembly and ligation uses corresponding DNA fragments in a reaction because DNA is joined by an overlap of the same 15 to 25 bases of the 5′ terminus and the 3′ terminus of DNA. When a vector to which a protein tag sequence can be added to the C-terminus such as pcDNA3.1(+)/V5-His (B) is utilized, if a stop codon of a C region is directly used, a normal TCR changes the stop codon into a translatable amino acid codon. This results in a construct capable of expressing a protein with a tag sequence added to TCR.

The composition of reaction solution and reaction conditions of ligation are in accordance with the manufacturer's protocol. A ligation product is partially fractionated, and a competent cell is transformed and plated on an agar medium to obtain genetically recombinant E. coli.

The following tables show the TCRα/β variable region amplification primer set, type of C region DNA fragment, and type of protein expressed from an expression vector or prepared virus upon using expression vector pcDNA3.1(+)/V5-His (B) and retroviral vector pMXs.

TABLE 33
oUsing pcDNA3.1 (+)/V5-His (B)

| Variable region amplification | | C region DNA | Expressed TCR |
|---|---|---|---|
| primer (F & R primes) | | fragment | product |
| TS-AdpD3EV | hTCRa- | hTCRaCFull-AdpD3EV | Standard TCRα |
| | C-R | | protein |
| hTCRa-VX-F- | hTCRa- | hTCRaCFull-AdpD3EV | Standard TCRα |
| AdpD3EV | C-R | | protein |
| TS-AdpD3EV | hTCRa- | hTCRaCFull_noSTOP- | Tag protein with |
| | C-R | AdpD3EV | TCRα V5 His |
| hTCRa-VX-F- | hTCRa- | hTCRaCFull_noSTOP- | Tag protein with |
| AdpD3EV | C-R | AdpD3EV | TCRα V5 His |
| TS-AdpD3EV | hTCRb- | hTCRbC1Full-AdpD3EV | Standard TCRβ |
| | C1-R | | (C1) protein |
| hTCRb-VX-F- | hTCRb- | hTCRbC1Full-AdpD3EV | Standard TCRβ |
| AdpD3EV | C1-R | | (C1) protein |
| TS-AdpD3EV | hTCRb- | hTCRbC1Full_noSTOP- | Tag protein with |
| | C1-R | AdpD3EV | TCRβ (type C1) |
| | | | V5 His |
| hTCRb-VX-F- | hTCRb- | hTCRbC1Full_noSTOP- | Tag protein with |
| AdpD3EV | C1-R | AdpD3EV | TCRβ (type C1) |
| | | | V5 His |
| TS-AdpD3EV | hTCRb- | hTCRbC2Full-AdpD3EV | Standard TCRβ |
| | C2-R | | (C2) protein |
| hTCRb-VX-F- | hTCRb- | hTCRbC2Full-AdpD3EV | Standard TCRβ |
| AdpD3EV | C2-R | | (C2) protein |
| TS-AdpD3EV | hTCRb- | hTCRbC2Full_noSTOP- | Tag protein with |
| | C2-R | AdpD3EV | TCRβ (C2) V5 |
| | | | His |
| hTCRb-VX-F- | hTCRb- | hTCRbC2Full_noSTOP- | Tag protein with |
| AdpD3EV | C2-R | AdpD3EV | TCRβ (C2) V5 |
| | | | His |
| hTCRa-V24-F- | hTCRa- | hTCRaCFull-AdpD3EV | Standard TCRα |
| AdpD3EV | C-R | | (V24) protein |
| hTCRa-V24-F- | hTCRa- | hTCRaCFull_noSTOP- | Tag protein with |
| AdpD3EV | C-R | AdpD3EV | TCRα (V24) |
| | | | V5 His |
| hTCRb-V6.5-F- | hTCRb- | hTCRbC1Full-AdpD3EV | Standard TCRβ |
| AdpD3EV | C1-R | | (V6-5 & C1) |
| | | | protein |
| hTCRb-V6.5-F- | hTCRb- | hTCRbC1Full_noSTOP- | Tag protein with |
| AdpD3EV | C1-R | AdpD3EV | TCRβ (V6-5 & |
| | | | C1) V5 His |
| hTCRb-V6.5-F- | hTCRb- | hTCRbC2Full-AdpD3EV | Standard TCRβ |
| AdpD3EV | C2-R | | (V6-5 & C2) |
| | | | protein |
| hTCRb-V6.5-F- | hTCRb- | hTCRbC2Full_noSTOP- | Tag protein with |
| AdpD3EV | C2-R | AdpD3EV | TCRβ (V6-5 & |
| | | | C2) V5 His |

TABLE 34
oUsing pMXs

| V(D)J region PCR product | | C region | Expressed |
|---|---|---|---|
| (F & R primes) | | DNA fragment | TCR product |
| TS-AdpMXEI | hTCRa-C-R | hTCRaCFull- | Standard TCRα protein |
| | | AdpMXEI | |
| hTCRa-VX-F- | hTCRa-C-R | hTCRaCFull- | Standard TCRα protein |
| AdpMXEI | | AdpMXEI | |
| TS-AdpMXEI | hTCRb-C1-R | hTCRbC1Full- | Standard TCRβ (C1) |
| | | AdpMXEI | protein |
| hTCRb-VX-F- | hTCRb-C1-R | hTCRbC1Full- | Standard TCRβ (C1) |
| AdpMXEI | | AdpMXEI | protein |
| TS-AdpMXEI | hTCRb-C2-R | hTCRbC2Full- | Standard TCRβ (C2) |
| | | AdpMXEI | protein |
| hTCRb-VX-F- | hTCRb-C2-R | hTCRbC2Full- | Standard TCRβ (C2) |
| AdpMXEI | | AdpMXEI | protein |
| hTCRa-V24-F- | hTCRa-C-R | hTCRaCFull- | Standard TCRα (V24) |
| AdpMXEI | | AdpMXEI | protein |
| hTCRb-V6.5-F- | hTCRb-C1-R | hTCRbC1Full- | Standard TCRβ |
| AdpMXEI | | AdpMXEI | (V6-5 & C1) protein |
| hTCRb-V6.5-F- | hTCRb-C2-R | hTCRbC2Full- | Standard TCRβ |
| AdpMXEI | | AdpMXEI | (V6-5 & C2) protein |

E2-2. Plasmid DNA Preparation

A single colony of plasmid gene recombinant organisms is cultured in a liquid medium and harvested, and the plasmid DNA is prepared with a common alkaline extraction method or a commercially available kit such as NucleoSpin Plasmid.

E2-3. Verification of Sequence of Plasmid DNA Insert

For the resulting plasmids, the insert sequence is sequenced by a common Sanger sequencing method using each of a TCRα sequencing primer, TCRβ sequencing primer, and vector primer. A clone without a substitution (at least without a missense mutation or nonsense mutation), insertion, or deletion in the base sequence is used as a TCR expression construct.

E3. Use of TCR (or BCR) Expression Construct

A TCR expression construct is introduced, as a set of two pair genes or only one of the two depending on the objective, into lymphocytes, various primary culture cells or established cells, microbes, fungi, or the like, which are optionally pretreated to enhance the introducability, by calcium phosphate transfection, lipofection, electroporation, heat shock method, or the like to allow gene expression and enable use in epitope search, functional analysis, protein synthesis, or the like through various cell-based bioassays.

A viral vector such as pMXs incorporating TCR can prepare a virus for gene transfer by cotransfection and culture thereof with another virus constituting protein expression construct that is required to a packaging cell such as 293T cells. A cell can be infected with a created virus, as a set of two pair genes or only one of the two depending on the objective, to allow expression of a protein. A construct with an in vitro transcription initiating promoter upstream of the translation start site such as a pcDNA3.1(+)/V5-His (B) vector with a T7 promoter incorporated therein can synthesize an mRNA of TCRα/β or protein in accordance with a common molecular biology or biochemistry textbook or experimental protocol book or by utilizing a commercially available kit or the like, without gene transfer into a cell or microbe.

A synthesized protein can be broadly utilized, directly or after required treatment such as refolding in pairs, as a sample for searching for an epitope, ligand, or binding protein, or conformation analysis and determination, a positive control sample for Western blotting, or the like.

### Example 6: BCR (or TCR) Pair Gene Cloning

Experiment F
List of reagents (additional reagents for Experiment F)
Competent E. coli cell (Nippon Gene)
pcDNA3.1(+)/V5-His (B) vector (Thermo Fisher Scientific)
pMXs vector (CELL BIOLABS)
Restriction enzyme EcoRI (Nippon Gene)
Restriction EcoRV (Nippon Gene)
NEBuilder HiFi DNA Assembly Master Mix (NEW England BioLabs)
NucleoSpin Plasmid (Takara Bio)

*Oligonucleotide sequences used
BCR cloning pcDNA3.1(+)/V5-His (B) forward primer
-TS-AdpD3EV:
(SEQ ID NO: 782)
TGGTGGAATTCTGCAGATAAGCAGTGGTATCAACGCA
-hBCRH-VX-F-AdpD3EV:
(SEQ ID NO: 783)
TGGTGGAATTCTGCAGAT
NNNN------NNNN (″NNNN------NNNN″ is a sequence of about 16 to
35 bases of a BCR heavy chain V region 5′ untranslated
region)
-hBCRH-V3.30-F-AdpD3EV:
(SEQ ID NO: 784)
TGGTGGAATTCTGCAGATACTAGAAGTCGGCGGTGTTTC
-hBCRH-V2.70-F-AdpD3EV:
(SEQ ID NO: 785)
TGGTGGAATTCTGCAGATACAGTGAATCCTGCTCCCCAC
-hBCRLam-VX-F-AdpD3EV:
(SEQ ID NO: 786)
TGGTGGAATTCTGCAGAT
NNNN------NNNN (″NNNN------NNNN″ is a sequence of about 16 to
35 bases of a BCR light chain A, V region 5′ untranslated
region)
-hBCRLam-V2.14-F-AdpD3EV:
(SEQ ID NO: 787)
TGGTGGAATTCTGCAGATTCTCAGGAGGCAGCGCTCTC
-hBCRKap-VX-F-AdpD3EV:
(SEQ ID NO: 788)
TGGTGGAATTCTGCAGAT
NNNN------NNNN (″NNNN------NNNN″ is a sequence of about 16 to
35 bases of a BCR light chain K, V region 5′ untranslated
region)
-hBCRK-V3.15-F-AdpD3EV:
(SEQ ID NO: 789)
TGGTGGAATTCTGCAGATAGGAACTGCTCAGTTAGGAC
(underlined portions are adaptor sequences for assembly and
ligation that are homologous to an arm region of a vector)
BCR cloning common reverse primer
-hBCRM-C-R:
(SEQ ID NO: 790)
GTTGGGGCGGATGCACTCCC
-hBCRLam-C2-R:
(SEQ ID NO: 791)
GGCAGCCTTGGGCTGACC
-hBCRKap-C1-R:
(SEQ ID NO: 792)
CAGATGGTGCAGCCACAGTTC

*Synthetic gene fragment used (custom synthetic double stranded DNA; can be synthesized at Thermo Fisher Scientific, Gene Design, INTEGRATED DNA TECHNOLOGIES, or the like. As an alternative method, RNA can be prepared from bulk PBMC cells, and cDNA can be amplified by PCR. Genes are known to have polymorphisms at racial or individual levels. The base sequence of the genotype of interest is changed as needed.)

For pcDNA3.1(+)/V5-His (B)
-hBCRKapCFull-AdpD3EV:
(SEQ ID NO: 793)
GAACTGTGGCTGCACCATCTGTCTTCATCTTCCCGCCATCTGATGAGCA
GTTGAAATCTGGAACTGCCTCTGTTGTGTGCCTGCTGAATAACTTCTAT
CCCAGAGAGGCCAAAGTACAGTGGAAGGTGGATAACGCCCTCCAATCGG
GTAACTCCCAGGAGAGTGTCACAGAGCAGGACAGCAAGGACAGCACCTA
CAGCCTCAGCAGCACCCTGACGCTGAGCAAAGCAGACTACGAGAAACAC
AAAGTCTACGCCTGCGAAGTCACCCATCAGGGCCTGAGCTCGCCCGTCA
CAAAGAGCTTCAACAGGGGAGAGTGTTAGATCCAGCACAGTGGCGGC
-hBCRLamC2Full-AdpD3EV:
(SEQ ID NO: 794)
GGTCAGCCCAAGGCTGCCCCCTCGGTCACTCTGTTCCCGCCCTCCTCTG
AGGAGCTTCAAGCCAACAAGGCCACACTGGTGTGTCTCATAAGTGACTT
CTACCCGGGAGCCGTGACAGTGGCTTGGAAAGCAGATAGCAGCCCCGTC
AAGGCGGGAGTGGAGACCACCACACCCTCCAAACAAAGCAACAACAAGT
ACGCGGCCAGCAGCTATCTGAGCCTGACGCCTGAGCAGTGGAAGTCCCA
CAGAAGCTACAGCTGCCAGGTCACGCATGAAGGGAGCACCGTGGAGAAG
ACAGTGGCCCCTACAGAATGTTCATAGATCCAGCACAGTGGCGGC
-hBCRpCFull-AdpD3EV:
(SEQ ID NO: 795)
GGGAGTGCATCCGCCCCAACCCTTTTCCCCCTCGTCTCCTGTGAGAATT
CCCCGTCGGATACGAGCAGCGTGGCCGTTGGCTGCCTCGCACAGGACTT
CCTTCCCGACTCCATCACTTTCTCCTGGAAATACAAGAACAACTCTGAC
ATCAGCAGCACCCGGGGCTTCCCATCAGTCCTGAGAGGGGGCAAGTACG
CAGCCACCTCACAGGTGCTGCTGCCTTCCAAGGACGTCATGCAGGGCAC
AGACGAACACGTGGTGTGCAAAGTCCAGCACCCCAACGGCAACAAAGAA
AAGAACGTGCCTCTTCCAGTGATTGCTGAGCTGCCTCCCAAAGTGAGCG
TCTTCGTCCCACCCCGCGACGGCTTCTTCGGCAACCCCCGCAAGTCCAA
GCTCATCTGCCAGGCCACGGGTTTCAGTCCCCGGCAGATTCAGGTGTCC
TGGCTGCGCGAGGGGAAGCAGGTGGGGTCTGGCGTCACCACGGACCAGG
TGCAGGCTGAGGCCAAAGAGTCTGGGCCCACGACCTACAAGGTGACCAG
CACACTGACCATCAAAGAGAGCGACTGGCTCGGCCAGAGCATGTTCACC
TGCCGCGTGGATCACAGGGGCCTGACCTTCCAGCAGAATGCGTCCTCCA
TGTGTGTCCCCGATCAAGACACAGCCATCCGGGTCTTCGCCATCCCCCC
ATCCTTTGCCAGCATCTTCCTCACCAAGTCCACCAAGTTGACCTGCCTG
GTCACAGACCTGACCACCTATGACAGCGTGACCATCTCCTGGACCCGCC
AGAATGGCGAAGCTGTGAAAACCCACACCAACATCTCCGAGAGCCACCC
CAATGCCACTTTCAGCGCCGTGGGTGAGGCCAGCATCTGCGAGGATGAC
TGGAATTCCGGGGAGAGGTTCACGTGCACCGTGACCCACACAGACCTGC
CCTCGCCACTGAAGCAGACCATCTCCCGGCCCAAGGGGGTGGCCCTGCA
CAGGCCCGATGTCTACTTGCTGCCACCAGCCCGGGAGCAGCTGAACCTG
CGGGAGTCGGCCACCATCACGTGCCTGGTGACGGGCTTCTCTCCCGCGG
ACGTCTTCGTGCAGTGGATGCAGAGGGGGCAGCCCTTGTCCCCGGAGAA
GTATGTGACCAGCGCCCCAATGCCTGAGCCCCAGGCCCCAGGCCGGTAC
TTCGCCCACAGCATCCTGACCGTGTCCGAAGAGGAATGGAACACGGGGG
AGACCTACACCTGCGTGGTGGCCCATGAGGCCCTGCCCAACAGGGTCAC
CGAGAGGACCGTGGACAAGTCCACCGGTAAACCCACCCTGTACAACGTG
TCCCTGGTCATGTCCGACACAGCTGGCACCTGCTACTGAATCCAGCACA
GTGGCGGC
(underlined portions are adaptor sequences for assembly and ligation that are homologous to an arm region of a vector)

F1. Preparation of DNA Fragment for Cloning

A variable region is amplified for functional pair genes of a single cell of BCR (or TCR) by a one-step RT-TS-PCR reaction or the following PCR reaction using a semi-nested PCR amplicon purified DNA as a template. Any V region can be amplified at once by using a sequence added to the 5′ side as the forward side primer and a sequence of a C region of TCR/BCR as the reverse side primer in one-step RT-TS-PCR. If it appears that there is a short fragment that does not comprise a start codon or a non-specific sequence or if a transcription product from two chromosomes is found from the result of electrophoresis or sequencing, amplification using a relevant V region specific sequence results in a DNA fragment with a higher purity. An amplicon is purified with AMPure XP and DNA purification column. If a short DNA fragment of 400 bases or less, a long fragment of 7000 bases or greater, or the like is observed, a DNA fragment with a length of interest is cut out through agarose gel electrophoresis and purified as needed. While this protocol only amplifies a variable region, a reverse transcription primer and a block primer of one-step-RT-TS PCR or a primer of semi-nested PCR can be designed to be more downstream towards 3′ than a stop codon (this can be in a form comprising a coding region, as long as a PCR product contains a stop codon site) and subjected to PCR, so that this can include from a translation start codon to a stop codon (when incorporating into pcDNA3.1(+)/V5-His (B) to construct a construct for a tagged protein, a stop codon is converted into some type of amino acid coding codon). Similarly, a DNA fragment comprising a full length coding region can be synthesized without PCR.

For example, it is understood that the BCR heavy chain (V3-30.D2-15.J4 clone)/BCR light chain λ (V2-14.J2.C2 clone) pair of well No. 18 and the BCR heavy chain (V2-70.D?.J6 clone)/BCR light chain κ (V3-15.J2 clone) pair of well No. 55 obtained in Example 4 (Experiment D) can be amplified in the following reaction.

TABLE 35
Amplification of BCR heavy chain (V3-30 · D2-15 ·
J4 clone) of well No. 18

| | | Amount of |
|---|---|---|
| | Amount of | solution upon |
| | solution | use of BCR |
| | upon use of | heavy chain |
| | TS adapter | variable region |
| Reagents, etc. | primer (μl) | primer (μl) |
| One-step RT-TS-PCR reaction | 9.2 | 9.2 |
| solution, or semi-nested PCR | | |
| product, or Index PCR product of | | |
| well No. 18 + DW | | |
| 2 × KAPA HiFi Hot Start | 10.0 | 10.0 |
| Ready Mix | | |
| Forward primer (10 μm) | 0.4 | — |
| TS-AdpD3EV | | |
| Forward primer (10 μm) | — | 0.4 |
| hBCRH-V3.30-F-AdpD3EV | | |
| hBCRM-C-R (10 uM) | 0.4 | 0.4 |
| Total | 20.0 | 20.0 |

TABLE 36
Amplification of BCR light chain λ (V2-14 · J2 ·
C2 clone) of well No. 18

| | | Amount of |
|---|---|---|
| | Amount of | solution upon |
| | solution | use of BCR |
| | upon use of | light chain |
| | TS adapter | variable region |
| Reagents, etc. | primer (μl) | primer (μl) |
| One-step RT-TS-PCR reaction solution, | 9.2 | 9.2 |
| or semi-nested PCR product, or Index | | |
| PCR product of well No. 18 + DW | | |
| 2 × KAPA HiFi Hot Start Ready Mix | 10.0 | 10.0 |
| Forward primer (10 μm) | 0.4 | — |
| TS-AdpD3EV | | |
| Forward primer (10 μm) | — | 0.4 |
| hBCRLam-V2.14-F-AdpD3EV | | |
| hBCRLam-C2-R (10 uM) | 0.4 | 0.4 |
| Total | 20.0 | 20.0 |

TABLE 37
Amplification of BCR heavy chain (V2-70 · D? ·
J6 clone) of well No. 55

| | | Amount of |
|---|---|---|
| | Amount of | solution upon |
| | solution | use of BCR |
| | upon use of | heavy chain |
| | TS adapter | variable region |
| Reagents, etc. | primer (μl) | primer (μl) |
| One-step RT-TS-PCR reaction solution, or | 9.2 | 9.2 |
| semi-nested PCR product, or Index PCR | | |
| product of well No. 55 + DW | | |
| 2 × KAPA HiFi Hot Start Ready Mix | 10.0 | 10.0 |
| Forward primer (10 μm) | 0.4 | — |
| TS-AdpD3EV | | |
| Forward primer (10 μm) | — | 0.4 |
| hBCRH-V2.70-F-AdpD3EV | | |
| hBCRM-C-R (10 uM) | 0.4 | 0.4 |
| Total | 20.0 | 20.0 |

F1-2. Preparation of Cloning Vector

Expression vector pcDNA3.1(+)/V5-His (B) is digested with EcoRV in accordance with the manufacturer's protocol. Optionally, agarose gel electrophoresis is performed, and a linear DNA band digested with an enzyme is cut out, and DNA is purified with a DNA purification column or the like.

F2. DNA Fragment Ligation and Introduction into E. coli to Obtain a Clone, and Sequence Verification

F2-1. DNA Fragment Ligation and Introduction into E. coli

Three types of DNA fragments, i.e., prepared variable region DNA fragment, genetically synthesized C region fragment (also can be amplified by PCR), and vector DNA digested by a restriction enzyme, are assembled and ligated using NEBuilder HiFi DNA Assembly Master Mix or the like.

Assembly and ligation uses corresponding DNA fragments in a reaction because DNA is joined by an overlap of the same 15 to 25 bases of the 5′ terminus and the 3′ terminus of DNA.

This Example shows a protocol for cloning the sequenced full length sequence of the original BCR class (BCRμ) for the heavy chain, but a full length sequence of BCR of a different class can also be prepared by using synthetic DNA of a C region as the sequence of BCRγ, α, δ, and ε and genetically synthesizing a variable region or amplifying a variable region using a chimeric primer of a J region 3′ terminus and C region 5′ terminus of another class by PCR.

When a vector to which a protein tag sequence can be added to the C-terminus such as pcDNA3.1(+)/V5-His (B) is utilized, if a stop codon of a C region is directly used, a normal BCR changes the stop codon into a translatable amino acid codon. This results in a construct capable of expressing a protein with a tag sequence added to BCR.

The composition of reaction solution and reaction conditions of ligation are in accordance with the manufacturer's protocol. A ligation product is partially fractionated, and a competent cell is transformed and plated on an agar medium to obtain genetic recombinant E. coli.

F2-2. Plasmid DNA Preparation

A single colony of plasmid gene recombinant organisms is cultured in a liquid medium is cultured and harvested, and the plasmid DNA is prepared with a common alkaline extraction method or a commercially available kit such as NucleoSpin Plasmid.

F2-3. Verification of Sequence of Plasmid DNA Insert

For the resulting plasmids, the insert sequence is sequenced by a common Sanger sequencing method using a vector primer and a sequencing primer designed to be shared by clones in each of BCR heavy chain C region, light chain κ C region, and light chain λ C region. (BCR is known to be introduced with a base substitution by a somatic cell mutation, so that a primer of a site without a mutation or a clone specific mutation tolerating primer can be designed.) A clone without a substitution (at least without a missense mutation or nonsense mutation), insertion, or deletion in the base sequence is used as a BCR expression construct.

F3. Use of BCR (or TCR) Expression Construct

A BCR expression construct is introduced, as a set of two pair genes or only one of the two depending on the objective, into lymphocytes, various primary culture cells or established cells, microbes, fungi, or the like, which are optionally pretreated to enhance the introducability, by calcium phosphate transfection, lipofection, electroporation, heat shock method, or the like to allow gene expression and enable use in epitope search, functional analysis, protein synthesis, or the like through various cell-based bioassays.

A viral vector such as pMXs incorporating BCR can prepare a virus for gene transfer by cotransfection and culture thereof with another virus constituting protein expression construct that is required to a packaging cell such as 293T cells. A cell can be infected with a created virus, as a set of two pair genes or only one of the two depending on the objective, to allow expression of a protein. A construct with an in vitro transcription initiating promoter upstream of the translation start site such as a pcDNA3.1(+)/V5-His (B) vector with a T7 promoter incorporated therein can synthesize a BCR heavy chain/light chain mRNA or protein in accordance with a common molecular biology or biochemistry textbook or experimental protocol book or by utilizing a commercially available kit or the like, without gene transfer into a cell or microbe. An artificial phage that can be used in phage display method or the like for searching for an antigen peptide can be manufactured by creating a vector for creating a phage that expresses a chimeric protein of a BCR heavy chain/light chain and a phage protein.

A synthesized protein can be broadly utilized, directly or after required treatment such as refolding in pairs, as a sample for searching an epitope, ligand, or binding protein, or conformation analysis and determination, a positive control sample for Western blotting, or the like. An immunoglobulin with a known epitope or antigen can be applied to CART therapy or the like by joining a detection antibody (Western blotting or immunostaining) or a heavy chain variable region and light chain variable region of such a protein with intracellular regions of various proteins such as CD3 intracellular region, CD28 intracellular region, 4-1BB intracellular region, ICOS intracellular region, and OX40 intracellular region, signaling domain, functional domain, protein fragment, artificially designed protein fragment, and chimeric proteins thereof to prepare a chimeric protein expressing construct.

### Example 7: Amplification of TCRα and TCRβ cDNA by Semi-One-Step RT-TS-PCR

Experiment G

G1. Cell Preparation and Culture

### Preparation Example

A preparation example of reagents used in this Example is shown below.
List of reagents (additional reagents for Experiment G)
D-PBS (14249-24, Nacalai Tesque)
Bambanker (CS-02-001, Nippon Genetics)
Dynabeads Human T-Activator CD3/CD28 (DB11161, Thermo Fisher Scientific)
PE-Cy7 labeled antihuman CD8 antibody (557746, Nippon Becton Dickinson Company)
FITC labeled antihuman C4 antibody (560994, Nippon Becton Dickinson Company)
40 U/μl RNasin RNase Inhibitor (N211B, Promega)
40 U/μl RNase Inhibitor (315-08121, Nippon Gene)
ECOS X Competent E. coli DH5 α (310-07733, Nippon Gene)
NucleoSpin Plasmid Transfection-grade (U0490B, Takara Bio)

*Oligonucleotide sequences used
RT-TS-PCR primer
-hTCRα RT primer (CA1; 23 bases):
(SEQ ID NO: 2)
TGTTGAAGGCGTTTGCACATGCA
-hTCRα RT primer (hTCRaC-CloR1; 21 bases):
(SEQ ID NO: 806)
ATAGCAGGGCCTCGATAATGA
-hTCRα RT primer (hTCRaC-CloR2; 22 bases):
(SEQ ID NO: 807)
AGGACCTAGAGCCCAAGAGAAC
-hTCRα PCR primer (hTCRaC-R4; 21 bases):
(SEQ ID NO: 764)
CAAAATCGGTGAATAGGCAGA
-hTCRα PCR primer (hTCRaC-R2v0; 21 bases):
(SEQ ID NO: 808)
GGAGCACAGGCTGTCTTACAA
-hTCRβ RT primer (CB1(3); 23 bases):
(SEQ ID NO: 4)
GAACTGGACTTGACAGCGGAAGT
-hTCRβ RT primer (hTCRbC1-R1; 23 bases):
(SEQ ID NO: 772)
AGTCACTTAGGCATGCTAAGGTC
-hTCRβ RT primer (hTCRbC1-R3; 21 bases):
(SEQ ID NO: 809)
CTGGGTTAGGGAGATTTCAGC
-hTCRβ RT primer (hTCRbC2-CloR1; 23 bases):
(SEQ ID NO: 771)
GGTTTAGCCTATTTCGTACTTGG
-hTCRβ RT primer (hTCRbC2-CloR2; 21 bases):
(SEQ ID NO: 810)
GTTGGGAGCTCAATCTTCAGG
-hTCRβ PCR primer (CB2; 23 bases):
(SEQ ID NO: 3)
AGGCAGTATCTGGAGTCATTGAG
-hTCRβ PCR primer (hTCRbC1-R2v2; 20 bases):
(SEQ ID NO: 811)
CTAAGGTCCCCCTGGGTTAG
-hTCRβ PCR primer (hTCRbC2-CloR3; 21 bases):
(SEQ ID NO: 812)
CTAGCCTCTGGAATCCTTTCT
-Forward TS-Tag primer v1 (64 bases):
(SEQ ID NO: 7)
GTCTC{GTGGGCTCGGAGATGTGTAT}AAGAGACAGAAGCAGTGGTATCAACGCAGAGTAC
ATGGG (underlined portion is a MiSeq
sequencing tag sequence site, sequence surrounded by { } is
the Sanger sequencing primer sequence)
-hTCRa Reverse Tag primer/hTCA-R-TP5-1 (53 bases):
(SEQ ID NO: 9)
TCGTC{GGCAGCGTCAGATGTGTATAA}GAGACAGACTTGTCACTGGATTTAGAG
-hTCR13 Reverse Tag primer (52 bases):
(SEQ ID NO: 10)
TCGTC{GGCAGCGTCAGATGTGTATAA}GAGACAGGCTCAAACACAGCGACCTC
(underlined portion is a MiSeq sequencing tag sequence site,
sequence surrounded by { } is the Sanger sequencing primer
sequence)
TCR cloning V sequence specific adaptor added forward primer
-hTCRaV38.2-F1-AdpD3EV:
(SEQ ID NO: 813)
TGGTGGAATTCTGCAGATAGCAGGGACCTGTGAGCAT
-hTCRbV14-F1-AdpD3EV:
(SEQ ID NO: 814)
TGGTGGAATTCTGCAGATGGGTCCTGCCATGGTTTCCA
-hTCRaV13.2-F2-AdpD3EV:
(SEQ ID NO: 815)
TGGTGGAATTCTGCAGATGGCTGGAGATTGCAGGTTTAT
-hTCRbV4.1-F1-AdpD3EV:
(SEQ ID NO: 816)
TGGTGGAATTCTGCAGATAGGCTAGCATGGGCTGCAGGCT
-hTCRaV27-F3-AdpD3EV:
(SEQ ID NO: 817)
TGGTGGAATTCTGCAGATGGCTCTTTCAGGAGCAGCTAA
-hTCRbV7.6-F1-AdpD3EV:
(SEQ ID NO: 818)
TGGTGGAATTCTGCAGATGGTAAAGCCCTCATCCTGTC
(underlined portions are adaptor sequences for assembly and
ligation that are homologous to an arm region of a vector)
TCR cloning adaptor added common reverse primer
-hTCRaC-AdpD3EV:
(SEQ ID NO: 819)
GCCGCCACTGTGCTGGATTCAGCTGGACCACAGCCGCAG
-hTCRaC-NT-AdpD3EV:
(SEQ ID NO: 820)
CCGCCACTGTGCTGGATTAAGCTGGACCACAGCCGCAG
-hTCRbC1-AdpD3EV:
(SEQ ID NO: 821)
GCCGCCACTGTGCTGGATTCAGAAATCCTTTCTCTTGAC
-hTCRbC1-NT-AdpD3EV:
(SEQ ID NO: 822)
GCCGCCACTGTGCTGGATTAAGAAATCCTTTCTCTTGAC
-hTCRbC2-AdpD3EV:
(SEQ ID NO: 823)
GCCGCCACTGTGCTGGATCTAGCCTCTGGAATCCTTTCT
-hTCRbC2-NT-AdpD3EV:
(SEQ ID NO: 824)
GCCGCCACTGTGCTGGATTAAGCCTCTGGAATCCTTTCT
(underlined portions are adaptor sequences for assembly and
ligation that are homologous to an arm region of a vector)
TCR C region cloning and sequencing common reverse primer
-hTCRa-C-F:
(SEQ ID NO: 825)
ATATCCAGAACCCTGACCCT
-hTCRb-C1-F:
(SEQ ID NO: 826)
AGGACCTGAACAAGGTGTTC
-hTCRb-C2-F:
(SEQ ID NO: 827)
AGGACCTGAAAAACGTGTTC
TCR-VJ region cloning common reverse primer
-hTCRa-C-R:
(SEQ ID NO: 755)
AGGGTCAGGGTTCTGGATAT
-hTCRb-C1-R:
(SEQ ID NO: 756)
GAACACCTTGTTCAGGTCCT
-hTCRb-C2-R:
(SEQ ID NO: 757)
GAACACGTTTTTCAGGTCCT
TCR C region cloning common forward primer
-hTCRaC1E4-R2:
CTTCCAAATCATTTTAATGAAGGCATC
-hTCRbC1-R1v2:
AGTCACTTAGGCATGCTAAGGTC
-hTCRbC2E4-R1:
GCAACCAGGCCCAACACACAA
TCR sequencing primer
hTCRaC-R4:
(SEQ ID NO: 764)
CAAAATCGGTGAATAGGCAGA
hTCRaC-R5:
(SEQ ID NO: 765)
ATAGACCTCATGTCTAGCACAG
hTCR-CB2-R2:
(SEQ ID NO: 828)
TTCTGATGGCTCAAACACAGC
hTCR-CB2:
(SEQ ID NO: 769)
AGGCAGTATCTGGAGTCATTGAG
hTCR-CB3:
(SEQ ID NO: 770)
ACTGTGCACCTCCTTCCCATTCA
Sequencing vector primer
T7:
(SEQ ID NO: 853)
TAATACGACTCACTATAGGG
CMV-Fwd2:
(SEQ ID NO: 854)
CGCAAATGGGCGGTAGGCGTG
BGH-Reverse:
(SEQ ID NO: 855)
TAGAAGGCACAGTCGAGG

G1-1. Cell Isolation

Peripheral blood was collected from human volunteers. Peripheral blood mononuclear cells (PBMC) were isolated by a Ficoll density gradient method, suspended in a cell cryopreservation reagent (Bambanker), cryopreserved at −80° C., and used in the experiment.

G1-2. Cell Culture

Cryopreserved PBMCs were thawed and then cultured for 3 days in a medium comprising Dynabeads Human T-Activator CD3/CD28. Dynabeads Human T-Activator CD3/CD28 was removed with a magnetic stand immediately prior to the experiment, and the cells were used in a single cell sorting repertoire analysis experiment.

G2. Cell Marker Staining and Single Cell Sorting

G2-1. Staining with Cell Marker Antibody

PBMCs cultured in a medium comprising Dynabeads Human T-Activator CD3/CD28 were centrifuged to allow the cells to precipitate, resuspended in D-PBS buffer and centrifuged for washing. The cells were then treated for 30 minutes on ice with a PE-Cy7 labeled antihuman CD8 antibody and FITC labeled antihuman CD4 antibody in D-PBS buffer. After the cells were resuspended in D-PBS buffer and centrifuged for washing twice, the cells were then ultimately suspended in 0.7 ml of D-PBS buffer and subjected to single cell sorting.

G2-2. Single Cell Sorting

For the stained cells, a PE-Cy7 positive (CD8 positive), FITC negative (CD8 negative) cell groups in a lymophocyte population gate were inputted in a 96-well plate added with an RT-TS-PCR reaction solution (G3-2) using a reverse transcription primer with different recognition sites for each of TCRα/β at one cell (15 wells or 19 wells for each RT primer set group), or a plurality of cells (10 cells, 1 well each) as a positive control of the experiment in a cell sorter (Nippon Becton Dickinson Company, FACSMelody).

G3. Amplification of TCRα and TCRβ cDNA by Semi-One-Step RT-TS-PCR

G3-1. Semi-One-Step RT-TS-PCR Reaction

An RT-TS reaction was immediately performed by heating for 30 minutes at 45° C. in the reaction reagents of Table 39 on a plate to which cells were dispensed. Condition 1 in Table 39 used an RT primer and a PCR primer that anneals to a TCR C region coding region in the same manner as the experiments up to this point. Meanwhile under conditions 2 and 3, an RT primer and a PCR primer were used that were designed for a 3′ downstream site of a stop codon or a sequence site comprising a stop codon, so that a full length protein coding region (full length open reading frame; ORF region) can be cloned in subsequent experiments.

Subsequently, the reaction reagents comprising the PCR primer of Table 40 were added and mixed in each well to perform a PCR reaction. Since the method performs a reverse transcription reaction, momentarily paused the reaction, and simply adds the same buffer comprising the PCR primer without changing the buffer or the like, the series of the methodologies of analysis was named a semi-one step RT-TS PCR reaction, and the resulting reaction solution was referred to as a semi-one step RT-TS PCR reaction solution.

G3-2. Semi-Nested PCR

2.0 μl of semi-one-step RT-TS-PCR reaction solution was fractionated after adding three times the amount of pure water (DW). A PCR reaction was performed in a semi-nested form in the reaction solution of Table 41 in separate tubes for TCRα and TCRβ. To add a sequencing primer recognition sequence to a DNA fragment, a primer added with an adaptor sequence (sequence for adding an index sequence for MiSeq sequencing) to the sequence on the inside of a block primer on the 3′ side and a template switching sequence on the 5′ side was used. 3 μl of reaction solution was fractionated to study a band by agarose gel electrophoresis.

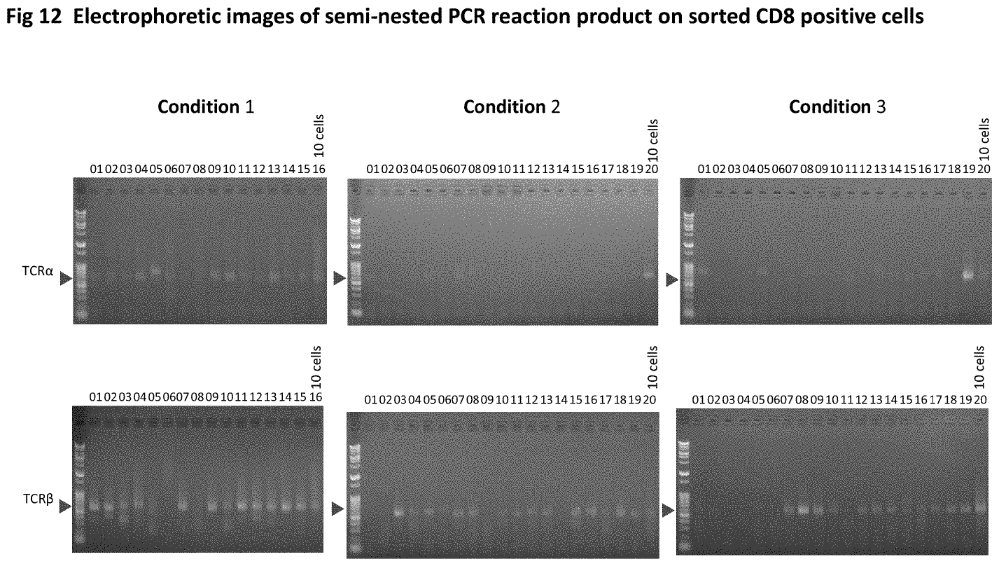

FIG. 12 shows results of semi-nested PCR. For wells subjected to single cell analysis (infused with one cell per well), a DNA band was observed for both TCRα and TCRβ in 14 out of 15 wells under condition 1, 12 out of 19 wells under condition 2, and 10 out of 19 wells under condition 3. For wells infused with 10 cells that were set as a positive control of a reaction, excellent amplification was observed for both TCRα and TCRβ under all three conditions.

G3-3 Sequencing Repertoire Sequencing

The resulting semi-nested PCR reaction solution was then purified and sequenced by the Sanger method. Since the objective of this experiment included cloning a full length protein coding region (full length ORF region) from the RT-PCR reaction solution described below, condition 2 which is relatively better between conditions 2 and 3 that met the requirements was subjected to analysis. Since the band signal intensity was weak overall in this experiment, all 19 samples for single cell analysis under condition 2 and samples yielding a certain amount of DNA or more for both TCRα and TCRβ from purifying DNA in TCRα and TCRβ semi-nested PCR reaction solutions of No 1 to 4 under condition 1 as comparative subjects (16 samples from No 1 to 12 and 16 to 19 under condition 2 and 4 samples from No 1 to 4 under condition 1) were subjected to sequencing analysis. For the resulting sequences, blast homology search was performed with respect to a TCRα/β sequence database to identify the presence/absence of TCR amplification and VJ repertoire sequences (also C region for TCRβ) of the amplified base sequences. Table 42 shows the determined TCRα/β sequence pair information (? Indicates unidentified).

TABLE 42

| RT& PCR | | TCRα | | TCRβ | | |
|---|---|---|---|---|---|---|
| primer | | TRAV | TRAJ | TRBV | TRBJ | TRBC |
| Condition 3 | No1 | TRAV38-2DV8 | TRAJ45 | TRBV14 | ? | C2 |
| | No2 | TRAV22 | ? | ? | TRBJ2-1 | C2 |
| | No3 | TRAV13-2 | TRAJ16 | TRBV4-1 | TRBJ2-7 | C2 |
| | No4 | TRAV27 | ? | TRBV7-6 | TRBJ2-3 | C2 |
| | No5 | TRAV17 | TRAJ49 | TRBV13 | TRBJ2-7 | C2 |
| | No6 | TRAV19 | TRAJ44 | TRBV19 | TRBJ2-1 | C2 |
| | No7 | TRAV29DV5 | TRAJ52 | TRBV4-1 | TRBJ2-6 | C1 |
| | No8 | TRAV8-2 | TRAJ12 | TRBV7-2 | TRBJ2-5 | C2 |
| | No9 | TRAV13-2 | TRAJ16 | ? | TRBJ2-3 | C2 |
| | No10 | TRAV12-2 | TRAJ12 | TRBV11-2 | TRBJ2-7 | C2 |
| | No11 | TRAV21 | TRAJ54 | TRBV28 | TRBJ2-3 | C2 |
| | No12 | TRAV8-3 | TRAJ9 | TRBV6-1 | TRBJ2-1 | C1 |
| | No16 | TRAV3 | ? | TRBV7-9 | TRBJ2-7 | C2 |
| | No17 | TRAV14DV4 | TRAJ45 | TRBV25-1 | TRBJ2-3 | C2 |
| | No18 | TRAV14DV4 | TRAJ9 | TRBV6-2 | TRBJ2-7 | C2 |
| | No19 | TRAV27 | TRAJ34 | TRBV28 | TRBJ2-7 | C2 |
| Condition 1 | No1 | TRAV11 | TRAJ53 | TRBV27 | TRBJ2-1 | C2 |
| | No2 | TRAV14DV4 | TRAJ10 | TRBV7-3 | TRBJ2-1 | C1 |
| | No3 | TRAV14DV4 | TRAJ21 | TRBV20-1 | TRBJ2-3 | C2 |
| | No4 | TRAV29DV5 | TRAJ52 | TRBV18 | TRBJ2-5 | C2 |

As a result, a sequence derived from TCR was able to be found in all purified DNA of semi-nested PCR reactants that were sequenced, including samples with an unclear DNA band. The DNA band detection limit in electrophoresis was about several nanograms/band. It was understood from this result that the success/failure of TCR amplification should not be determined from only the result of electrophoresis, and it is effective in some cases to proceed with sequencing for all analyzed wells from the viewpoint of cost effectiveness.

G4 Cloning of Full Length Protein Coding Region cDNA of TCR Pair Genes and Construction of Expression Vector

G4-1 PCR Reaction

To clone TCRα and TCRβ, pair genes of No 1, 3, and 4 under condition 3, primers were designed for upstream of a start codon of a variable region (V region) or upstream of a start codon including the start codon and ORF region. Translation start codon was assumed to be the site annotated on Ensemble's Gene database. As shown in Example 5, the same sequence as a vector sequence was added to the designed V region primer for assembly and ligation with a vector as shown in Example 5 (designed for pcDNA3.1(+)V5-His (B) in this Example in the same manner as Example 5). For the stop codon side primer, a primer with an addition of the same sequence as a vector sequence to a sequence of about 20 bases on the upstream side from the stop codon was created. A primer with a stop codon changed to an amino acid coding codon (TCRα C and TCRβ C1 were substituted from TGA and TCR C2 was substituted from TAG to TTA/leucine coding) to create a DNA sequence for expressing a fusion protein with a V5-His tag sequence of the vector was also created and used in the experiment.

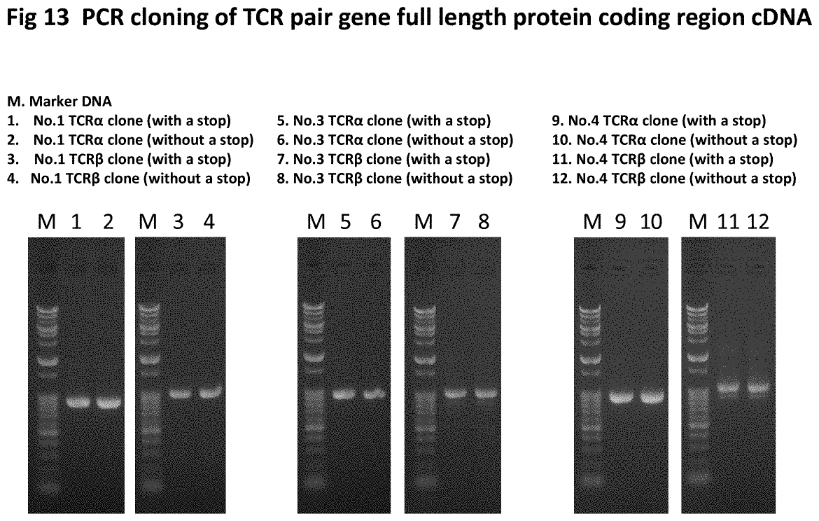

2.0 μl of semi-one-step RT-TS-PCR reaction solution diluted 4-fold with pure water was fractionated. A PCR reaction was performed in the reaction solution of Table 43 in separate tubes for TCRα and TCRβ to attempt cloning of a full length protein coding region cDNA. While a V region F primer needs to be changed for each clone, primers were used as described in the list of Table 44. 3 μl of reaction solution was fractionated to study a band by agarose gel electrophoresis (FIG. 13). For this reaction, a reaction for both TCRα and TCRβ and primers with and without a stop codon can be performed in the same tube. In such a case, assembly and ligation to a vector is expected to be able to construct four types of TCR expression constructs (for pcDNA3.1 V5-His (B), hTCRα expression plasmid, hTCRα V5-His fusion protein expression plasmid, hTCRβ expression plasmid, and hTCRβ V5-His fusion protein expression plasmid of a pcDNA3.1 vector) more readily in a single reaction in a single tube.

TABLE 43

| | Amount of | Amount of | Amount of | Amount of |
|---|---|---|---|---|
| | solution | solution for | solution | solution for |
| | for | TCRα | for | TCRβ |
| | TCRα | (no stop) | TCRβ | (no stop) |
| | amplifi- | amplifi- | amplifi- | amplifi- |
| | cation | cation | cation | cation |
| Reagents, etc. | (μl) | (μl) | (μl) | (μl) |
| 4 × diluted Semi- | 2.0 | 2.0 | 2.0 | 2.0 |
| one-step RT-TS-PCR | | | | |
| reaction solution | | | | |
| 2 × KAPA HiFi Hot | 12.5 | 12.5 | 12.5 | 12.5 |
| Start Ready Mix | | | | |
| hTCRaVXX-F#- | 0.5 | 0.5 | — | — |
| AdpD3EV (10 uM)* | | | | |
| hTCRaC-AdpD3EV | 0.5 | — | — | — |
| (10 uM) | | | | |
| hTCRaC-NT- | — | 0.5 | — | — |
| AdpD3EV (10 uM) | | | | |
| hTCRbVXX-F#- | — | | 0.5 | 0.5 |
| AdpD3EV (10 uM)* | | | | |
| hTCRbC2-AdpD3EV | — | | 0.5 | — |
| (10 uM) | | | | |
| hTCRbC2-NT- | — | | — | 0.5 |
| AdpD3EV (10 uM) | | | | |
| DW | 9.5 | 9.5 | 9.5 | 9.5 |
| Total | 25.0 | 25.0 | 25.0 | 25.0 |

*collectively referring to V region specific adaptor added primers. XX is the V number, and # is a number.

G4-2 Sequencing Reaction

The resulting DNA fragment was purified with AMPure XP DNA, and the base sequence was sequenced by a common Sanger sequencing method using TCRα sequencing primers and (hTCRα-C-F, hTCRaC-R4, and hTCRaC-R5) and TCRβ sequencing primers (hTCRb-C2-F, hTCR-CR-R2, hTCR-CB2, and hTCR-CB3). The base sequence of the full length protein coding region cDNA was determined based on the resulting sequencing data as a reference, using the sequencing data for semi-nested PCR reaction purified DNA in G3-3 as reference data. The determined base sequences and expected amino acid sequences are shown below (an underlined portion indicates a start codon ATG or stop codon, * indicates a site where protein synthesis stops due to a stop codon, and // indicates a frameshift site). The table shows updated version of TCR VJ repertoire information obtained from the analysis results.

DNA of a TCR pair comprising full length protein coding region was able to be cloned in No. 1 and No. 3. TCRβ of No. 4 included a full length protein coding region, but TCRα had a frameshift in a VJ conjugation region. Although it was understood that a normal full length protein could not be expressed, this is a result that could be biologically significant and interesting.

>Condition 3 No. 1 TCRα PCR product (with a stop) base
sequence
(SEQ ID NO: 829)
AGCAGGGACCTGTGAGCATGGCATGCCCTGGCTTCCTGTGGGCACTTGTGATCTCCACCTG
TCTTGAATTTAGCATGGCTCAGACAGTCACTCAGTCTCAACCAGAGATGTCTGTGCAGGAG
GCAGAGACCGTGACCCTGAGCTGCACATATGACACCAGTGAGAGTGATTATTATTTATTCT
GGTACAAGCAGCCTCCCAGCAGGCAGATGATTCTCGTTATTCGCCAAGAAGCTTATAAGCA
ACAGAATGCAACAGAGAATCGTTTCTCTGTGAACTTCCAGAAAGCAGCCAAATCCTTCAGT
CTCAAGATCTCAGACTCACAGCTGGGGGATGCCGCGATGTATTTCTGTGCCCGGTATTCAG
GAGGAGGTGCTGACGGACTCACCTTTGGCAAAGGGACTCATCTAATCATCCAGCCCTATAT
CCAGAACCCTGACCCTGCCGTGTACCAGCTGAGAGACTCTAAATCCAGTGACAAGTCTGTC
TGCCTATTCACCGATTTTGATTCTCAAACAAATGTGTCACAAAGTAAGGATTCTGATGTGT
ATATCACAGACAAAACTGTGCTAGACATGAGGTCTATGGACTTCAAGAGCAACAGTGCTGT
GGCCTGGAGCAACAAATCTGACTTTGCATGTGCAAACGCCTTCAACAACAGCATTATTCCA
GAAGACACCTTCTTCCCCAGCCCAGAAAGTTCCTGTGATGTCAAGCTGGTCGAGAAAAGCT
TTGAAACAGATACGAACCTAAACTTTCAAAACCTGTCAGTGATTGGGTTCCGAATCCTCCT
CCTGAAAGTGGCCGGGTTTAATCTGCTCATGACGCTGCGGCTGTGGTCCAGCTGA
>Condition 3 No. 1 TCRα PCR product (with a stop) amino acid
sequence
(SEQ ID NO: 830)
MACPGFLWALVISTCLEFSMAQTVTQSQPEMSVQEAETVTLSCTYDTSESDYYLFWYKQPP
SRQMILVIRQEAYKQQNATENRFSVNFQKAAKSFSLKISDSQLGDAAMYFCARYSGGGADG
LTFGKGTHLIIQPYIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETDTN
LNFQNLSVIGFRILLLKVAGFNLLMTLRLWSS*
>Condition 3 No. 1 TCRα PCR product (without a stop) base
sequence
(SEQ ID NO: 831)
AGCAGGGACCTGTGAGCATGGCATGCCCTGGCTTCCTGTGGGCACTTGTGATCTCCACCTG
TCTTGAATTTAGCATGGCTCAGACAGTCACTCAGTCTCAACCAGAGATGTCTGTGCAGGAG
GCAGAGACCGTGACCCTGAGCTGCACATATGACACCAGTGAGAGTGATTATTATTTATTCT
GGTACAAGCAGCCTCCCAGCAGGCAGATGATTCTCGTTATTCGCCAAGAAGCTTATAAGCA
ACAGAATGCAACAGAGAATCGTTTCTCTGTGAACTTCCAGAAAGCAGCCAAATCCTTCAGT
CTCAAGATCTCAGACTCACAGCTGGGGGATGCCGCGATGTATTTCTGTGCCCGGTATTCAG
GAGGAGGTGCTGACGGACTCACCTTTGGCAAAGGGACTCATCTAATCATCCAGCCCTATAT
CCAGAACCCTGACCCTGCCGTGTACCAGCTGAGAGACTCTAAATCCAGTGACAAGTCTGTC
TGCCTATTCACCGATTTTGATTCTCAAACAAATGTGTCACAAAGTAAGGATTCTGATGTGT
ATATCACAGACAAAACTGTGCTAGACATGAGGTCTATGGACTTCAAGAGCAACAGTGCTGT
GGCCTGGAGCAACAAATCTGACTTTGCATGTGCAAACGCCTTCAACAACAGCATTATTCCA
GAAGACACCTTCTTCCCCAGCCCAGAAAGTTCCTGTGATGTCAAGCTGGTCGAGAAAAGCT
TTGAAACAGATACGAACCTAAACTTTCAAAACCTGTCAGTGATTGGGTTCCGAATCCTCCT
CCTGAAAGTGGCCGGGTTTAATCTGCTCATGACGCTGCGGCTGTGGTCCAGCTTA
>Condition 3 No. 1 TCRα PCR product (without a stop) amino
acid sequence
(SEQ ID NO: 832)
MACPGFLWALVISTCLEFSMAQTVTQSQPEMSVQEAETVTLSCTYDTSESDYYLFWYKQPP
SRQMILVIRQEAYKQQNATENRFSVNFQKAAKSFSLKISDSQLGDAAMYFCARYSGGGADG
LTFGKGTHLIIQPYIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETDTN
LNFQNLSVIGFRILLLKVAGFNLLMTLRLWSSL
>Condition 3 No. 1 TCRβ PCR product (with a stop) base
sequence
(SEQ ID NO: 833)
GGGTCCTGCCATGGTTTCCAGGCTTCTCAGTTTAGTGTCCCTTTGTCTCCTGGGAGCAAAG
CACATAGAAGCTGGAGTTACTCAGTTCCCCAGCCACAGCGTAATAGAGAAGGGCCAGACTG
TGACTCTGAGATGTGACCCAATTTCTGGACATGATAATCTTTATTGGTATCGACGTGTTAT
GGGAAAAGAAATAAAATTTCTGTTACATTTTGTGAAAGAGTCTAAACAGGATGAGTCCGGT
ATGCCCAACAATCGATTCTTAGCTGAAAGGACTGGAGGGACGTATTCTACTCTGAAGGTGC
AGCCTGCAGAACTGGAGGATTCTGGAGTTTATTTCTGTGCCAGCAGCCAAGATCGCATCGA
GCAGTACTTCGGGCCGGGCACCAGGCTCACGGTCACAGAGGACCTGAAAAACGTGTTCCCA
CCCGAGGTCGCTGTGTTTGAGCCATCAGAAGCAGAGATCTCCCACACCCAAAAGGCCACAC
TGGTGTGCCTGGCCACAGGCTTCTACCCCGACCACGTGGAGCTGAGCTGGTGGGTGAATGG
GAAGGAGGTGCACAGTGGGGTCAGCACAGACCCGCAGCCCCTCAAGGAGCAGCCCGCCCTC
AATGACTCCAGATACTGCCTGAGCAGCCGCCTGAGGGTCTCGGCCACCTTCTGGCAGAACC
CCCGCAACCACTTCCGCTGTCAAGTCCAGTTCTACGGGCTCTCGGAGAATGACGAGTGGAC
CCAGGATAGGGCCAAACCTGTCACCCAGATCGTCAGCGCCGAGGCCTGGGGTAGAGCAGAC
TGTGGCTTCACCTCCGAGTCTTACCAGCAAGGGGTCCTGTCTGCCACCATCCTCTATGAGA
TCTTGCTAGGGAAGGCCACCTTGTATGCCGTGCTGGTCAGTGCCCTCGTGCTGATGGCCAT
GGTCAAGAGAAAGGATTCCAGAGGCTAG
>Condition 3 No. 1 TCRβ PCR product (with a stop) amino acid
sequence
(SEQ ID NO: 834)
MVSRLLSLVSLCLLGAKHIEAGVTQFPSHSVIEKGQTVTLRCDPISGHDNLYWYRRVMGKE
IKFLLHFVKESKQDESGMPNNRFLAERTGGTYSTLKVQPAELEDSGVYFCASSQDRIEQYF
GPGTRLTVTEDLKNVFPPEVAVFEPSEAEISHTQKATLVCLATGFYPDHVELSWWVNGKEV
HSGVSTDPQPLKEQPALNDSRYCLSSRLRVSATFWQNPRNHFRCQVQFYGLSENDEWTQDR
AKPVTQIVSAEAWGRADCGFTSESYQQGVLSATILYEILLGKATLYAVLVSALVLMAMVKR
KDSRG*
>Condition 3 No. 1 TCRβ PCR product (without a stop) base
sequence
(SEQ ID NO: 835)
GGGTCCTGCCATGGTTTCCAGGCTTCTCAGTTTAGTGTCCCTTTGTCTCCTGGGAGCAAAG
CACATAGAAGCTGGAGTTACTCAGTTCCCCAGCCACAGCGTAATAGAGAAGGGCCAGACTG
TGACTCTGAGATGTGACCCAATTTCTGGACATGATAATCTTTATTGGTATCGACGTGTTAT
GGGAAAAGAAATAAAATTTCTGTTACATTTTGTGAAAGAGTCTAAACAGGATGAGTCCGGT
ATGCCCAACAATCGATTCTTAGCTGAAAGGACTGGAGGGACGTATTCTACTCTGAAGGTGC
AGCCTGCAGAACTGGAGGATTCTGGAGTTTATTTCTGTGCCAGCAGCCAAGATCGCATCGA
GCAGTACTTCGGGCCGGGCACCAGGCTCACGGTCACAGAGGACCTGAAAAACGTGTTCCCA
CCCGAGGTCGCTGTGTTTGAGCCATCAGAAGCAGAGATCTCCCACACCCAAAAGGCCACAC
TGGTGTGCCTGGCCACAGGCTTCTACCCCGACCACGTGGAGCTGAGCTGGTGGGTGAATGG
GAAGGAGGTGCACAGTGGGGTCAGCACAGACCCGCAGCCCCTCAAGGAGCAGCCCGCCCTC
AATGACTCCAGATACTGCCTGAGCAGCCGCCTGAGGGTCTCGGCCACCTTCTGGCAGAACC
CCCGCAACCACTTCCGCTGTCAAGTCCAGTTCTACGGGCTCTCGGAGAATGACGAGTGGAC
CCAGGATAGGGCCAAACCTGTCACCCAGATCGTCAGCGCCGAGGCCTGGGGTAGAGCAGAC
TGTGGCTTCACCTCCGAGTCTTACCAGCAAGGGGTCCTGTCTGCCACCATCCTCTATGAGA
TCTTGCTAGGGAAGGCCACCTTGTATGCCGTGCTGGTCAGTGCCCTCGTGCTGATGGCCAT
GGTCAAGAGAAAGGATTCCAGAGGCTTA
>Condition 3 No. 1 TCRβ PCR product (without a stop) amino
acid sequence
(SEQ ID NO: 836)
MVSRLLSLVSLCLLGAKHIEAGVTQFPSHSVIEKGQTVTLRCDPISGHDNLYWYRRVMGKE
IKFLLHFVKESKQDESGMPNNRFLAERTGGTYSTLKVQPAELEDSGVYFCASSQDRIEQYF
GPGTRLTVTEDLKNVFPPEVAVFEPSEAEISHTQKATLVCLATGFYPDHVELSWWVNGKEV
HSGVSTDPQPLKEQPALNDSRYCLSSRLRVSATFWQNPRNHFRCQVQFYGLSENDEWTQDR
AKPVTQIVSAEAWGRADCGFTSESYQQGVLSATILYEILLGKATLYAVLVSALVLMAMVKR
KDSRGL
>Condition 3 No. 3 TCRα PCR product (with a stop) base
sequence
(SEQ ID NO: 837)
GGCTGGAGATTGCAGGTTTATGACTGATCCTATTTGGGAAGAACAATGATGGCAGGCATTC
GAGCTTTATTTATGTACTTGTGGCTGCAGCTGGACTGGGTGAGCAGAGGAGAGAGTGTGGG
GCTGCATCTTCCTACCCTGAGTGTCCAGGAGGGTGACAACTCTATTATCAACTGTGCTTAT
TCAAACAGCGCCTCAGACTACTTCATTTGGTACAAGCAAGAATCTGGAAAAGGTCCTCAAT
TCATTATAGACATTCGTTCAAATATGGACAAAAGGCAAGGCCAAAGAGTCACCGTTTTATT
GAATAAGACAGTGAAACATCTCTCTCTGCAAATTGCAGCTACTCAACCTGGAGACTCAGCT
GTCTACTTTTGTGCAGAGACCTCCCCCTTTTCAGATGGCCAGAAGCTGCTCTTTGCAAGGG
GGACCATGTTAAAGGTGGATCTTAATATCCAGAACCCTGACCCTGCCGTGTACCAGCTGAG
AGACTCTAAATCCAGTGACAAGTCTGTCTGCCTATTCACCGATTTTGATTCTCAAACAAAT
GTGTCACAAAGTAAGGATTCTGATGTGTATATCACAGACAAAACTGTGCTAGACATGAGGT
CTATGGACTTCAAGAGCAACAGTGCTGTGGCCTGGAGCAACAAATCTGACTTTGCATGTGC
AAACGCCTTCAACAACAGCATTATTCCAGAAGACACCTTCTTCCCCAGCCCAGAAAGTTCC
TGTGATGTCAAGCTGGTCGAGAAAAGCTTTGAAACAGATACGAACCTAAACTTTCAAAACC
TGTCAGTGATTGGGTTCCGAATCCTCCTCCTGAAAGTGGCCGGGTTTAATCTGCTCATGAC
GCTGCGGCTGTGGTCCAGCTGA
>Condition 3 No. 3 TCRα PCR product (with a stop) amino acid
sequence
(SEQ ID NO: 838)
MAGIRALFMYLWLQLDWVSRGESVGLHLPTLSVQEGDNSIINCAYSNSASDYFIWYKQESG
KGPQFIIDIRSNMDKRQGQRVTVLLNKTVKHLSLQIAATQPGDSAVYFCAETSPFSDGQKL
LFARGTMLKVDLNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKLVEKSFETDTNL
NFQNLSVIGFRILLLKVAGFNLLMTLRLWSS*
>Condition 3 No. 3 TCRα PCR product (without a stop) base
sequence
(SEQ ID NO: 839)
GGCTGGAGATTGCAGGTTTATGACTGATCCTATTTGGGAAGAACAATGATGGCAGGCATTC
GAGCTTTATTTATGTACTTGTGGCTGCAGCTGGACTGGGTGAGCAGAGGAGAGAGTGTGGG
GCTGCATCTTCCTACCCTGAGTGTCCAGGAGGGTGACAACTCTATTATCAACTGTGCTTAT
TCAAACAGCGCCTCAGACTACTTCATTTGGTACAAGCAAGAATCTGGAAAAGGTCCTCAAT
TCATTATAGACATTCGTTCAAATATGGACAAAAGGCAAGGCCAAAGAGTCACCGTTTTATT
GAATAAGACAGTGAAACATCTCTCTCTGCAAATTGCAGCTACTCAACCTGGAGACTCAGCT
GTCTACTTTTGTGCAGAGACCTCCCCCTTTTCAGATGGCCAGAAGCTGCTCTTTGCAAGGG
GGACCATGTTAAAGGTGGATCTTAATATCCAGAACCCTGACCCTGCCGTGTACCAGCTGAG
AGACTCTAAATCCAGTGACAAGTCTGTCTGCCTATTCACCGATTTTGATTCTCAAACAAAT
GTGTCACAAAGTAAGGATTCTGATGTGTATATCACAGACAAAACTGTGCTAGACATGAGGT
CTATGGACTTCAAGAGCAACAGTGCTGTGGCCTGGAGCAACAAATCTGACTTTGCATGTGC
AAACGCCTTCAACAACAGCATTATTCCAGAAGACACCTTCTTCCCCAGCCCAGAAAGTTCC
TGTGATGTCAAGCTGGTCGAGAAAAGCTTTGAAACAGATACGAACCTAAACTTTCAAAACC
TGTCAGTGATTGGGTTCCGAATCCTCCTCCTGAAAGTGGCCGGGTTTAATCTGCTCATGAC
GCTGCGGCTGTGGTCCAGCTTA
>Condition 3 No. 3 TCRα PCR product (without a stop) amino
acid sequence
(SEQ ID NO: 840)
MAGIRALFMYLWLQLDWVSRGESVGLHLPTLSVQEGDNSIINCAYSNSASDYFIWYKQESG
KGPQFIIDIRSNMDKRQGQRVTVLLNKTVKHLSLQIAATQPGDSAVYFCAETSPFSDGQKL
LFARGTMLKVDLNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKLVEKSFETDTNL
NFQNLSVIGFRILLLKVAGFNLLMTLRLWSSL
>Condition 3 No. 3 TCRβ PCR product (with a stop) base
sequence
(SEQ ID NO: 841)
AGGCTAGCATGGGCTGCAGGCTGCTCTGCTGTGCGGTTCTCTGTCTCCTGGGAGCAGTTCC
CATAGACACTGAAGTTACCCAGACACCAAAACACCTGGTCATGGGAATGACAAATAAGAAG
TCTTTGAAATGTGAACAACATATGGGGCACAGGGCTATGTATTGGTACAAGCAGAAAGCTA
AGAAGCCACCGGAGCTCATGTTTGTCTACAGCTATGAGAAACTCTCTATAAATGAAAGTGT
GCCAAGTCGCTTCTCACCTGAATGCCCCAACAGCTCTCTCTTAAACCTTCACCTACACGCC
CTGCAGCCAGAAGACTCAGCCCTGTATCTCTGCGCCAGCAGCCAGGGGCGGAGAAACTACG
AGCAGTACTTCGGGCCGGGCACCAGGCTCACGGTCACAGAGGACCTGAAAAACGTGTTCCC
ACCCGAGGTCGCTGTGTTTGAGCCATCAGAAGCAGAGATCTCCCACACCCAAAAGGCCACA
CTGGTGTGCCTGGCCACAGGCTTCTACCCCGACCACGTGGAGCTGAGCTGGTGGGTGAATG
GGAAGGAGGTGCACAGTGGGGTCAGCACAGACCCGCAGCCCCTCAAGGAGCAGCCCGCCCT
CAATGACTCCAGATACTGCCTGAGCAGCCGCCTGAGGGTCTCGGCCACCTTCTGGCAGAAC
CCCCGCAACCACTTCCGCTGTCAAGTCCAGTTCTACGGGCTCTCGGAGAATGACGAGTGGA
CCCAGGATAGGGCCAAACCTGTCACCCAGATCGTCAGCGCCGAGGCCTGGGGTAGAGCAGA
CTGTGGCTTCACCTCCGAGTCTTACCAGCAAGGGGTCCTGTCTGCCACCATCCTCTATGAG
ATCTTGCTAGGGAAGGCCACCTTGTATGCCGTGCTGGTCAGTGCCCTCGTGCTGATGGCCA
TGGTCAAGAGAAAGGATTCCAGAGGCTAG
>Condition 3 No. 3 TCRβ PCR product (with a stop) amino acid
sequence
(SEQ ID NO: 842)
MGCRLLCCAVLCLLGAVPIDTEVTQTPKHLVMGMTNKKSLKCEQHMGHRAMYWYKQKAKKP
PELMFVYSYEKLSINESVPSRFSPECPNSSLLNLHLHALQPEDSALYLCASSQGRRNYEQY
FGPGTRLTVTEDLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMAMVK
RKDSRG*
>Condition 3 No. 3 TCRβ PCR product (without a stop) base
sequence
(SEQ ID NO: 843)
AGGCTAGCATGGGCTGCAGGCTGCTCTGCTGTGCGGTTCTCTGTCTCCTGGGAGCAGTTCC
CATAGACACTGAAGTTACCCAGACACCAAAACACCTGGTCATGGGAATGACAAATAAGAAG
TCTTTGAAATGTGAACAACATATGGGGCACAGGGCTATGTATTGGTACAAGCAGAAAGCTA
AGAAGCCACCGGAGCTCATGTTTGTCTACAGCTATGAGAAACTCTCTATAAATGAAAGTGT
GCCAAGTCGCTTCTCACCTGAATGCCCCAACAGCTCTCTCTTAAACCTTCACCTACACGCC
CTGCAGCCAGAAGACTCAGCCCTGTATCTCTGCGCCAGCAGCCAGGGGCGGAGAAACTACG
AGCAGTACTTCGGGCCGGGCACCAGGCTCACGGTCACAGAGGACCTGAAAAACGTGTTCCC
ACCCGAGGTCGCTGTGTTTGAGCCATCAGAAGCAGAGATCTCCCACACCCAAAAGGCCACA
CTGGTGTGCCTGGCCACAGGCTTCTACCCCGACCACGTGGAGCTGAGCTGGTGGGTGAATG
GGAAGGAGGTGCACAGTGGGGTCAGCACAGACCCGCAGCCCCTCAAGGAGCAGCCCGCCCT
CAATGACTCCAGATACTGCCTGAGCAGCCGCCTGAGGGTCTCGGCCACCTTCTGGCAGAAC
CCCCGCAACCACTTCCGCTGTCAAGTCCAGTTCTACGGGCTCTCGGAGAATGACGAGTGGA
CCCAGGATAGGGCCAAACCTGTCACCCAGATCGTCAGCGCCGAGGCCTGGGGTAGAGCAGA
CTGTGGCTTCACCTCCGAGTCTTACCAGCAAGGGGTCCTGTCTGCCACCATCCTCTATGAG
ATCTTGCTAGGGAAGGCCACCTTGTATGCCGTGCTGGTCAGTGCCCTCGTGCTGATGGCCA
TGGTCAAGAGAAAGGATTCCAGAGGCTTA
>Condition 3 No. 3 TCRβ PCR product (without a stop) amino
acid sequence
(SEQ ID NO: 844)
MGCRLLCCAVLCLLGAVPIDTEVTQTPKHLVMGMTNKKSLKCEQHMGHRAMYWYKQKAKKP
PELMFVYSYEKLSINESVPSRFSPECPNSSLLNLHLHALQPEDSALYLCASSQGRRNYEQY
FGPGTRLTVTEDLKNVFPPEVAVFEPSEAEISHTQKATLVCLATGFYPDHVELSWWVNGKE
VHSGVSTDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMAMVK
RKDSRGL
>Condition 3 No. 4 TCRα PCR product (with a stop) base
sequence
(SEQ ID NO: 845)
GGCTCTTTCAGGAGCAGCTAAAGTCAGGGGCCATGTCCACCATGTGATAGAAAGACAAGAT
GGTCCTGAAATTCTCCGTGTCCATTCTTTGGATTCAGTTGGCATGGGTGAGCACCCAGCTG
CTGGAGCAGAGCCCTCAGTTTCTAAGCATCCAAGAGGGAGAAAATCTCACTGTGTACTGCA
ACTCCTCAAGTGTTTTTTCCAGCTTACAATGGTACAGACAGGAGCCTGGGGAAGGTCCTGT
CCTCCTGGTGACAGTAGTTACGGGTGGAGAAGTGAAGAAGCTGAAGAGACTAACCTTTCAG
TTTGGTGATGCAAGAAAGGACAGTTCTCTCCACATCACTGCGGCCCAGCCTGGTGATACAG
GCCTCTACCTCTGTGCCTATCCGAGGAGGAGGTGCTGACGGACTCACCTTTGGCAAAGGGA
CTCATCTAATCATCCAGCCCTATATCCAGAACCCTGACCCTGCCGTGTACCAGCTGAGAGA
CTCTAAATCCAGTGACAAGTCTGTCTGCCTATTCACCGATTTTGATTCTCAAACAAATGTG
TCACAAAGTAAGGATTCTGATGTGTATATCACAGACAAAACTGTGCTAGACATGAGGTCTA
TGGACTTCAAGAGCAACAGTGCTGTGGCCTGGAGCAACAAATCTGACTTTGCATGTGCAAA
CGCCTTCAACAACAGCATTATTCCAGAAGACACCTTCTTCCCCAGCCCAGAAAGTTCCTGT
GATGTCAAGCTGGTCGAGAAAAGCTTTGAAACAGATACGAACCTAAACTTTCAAAACCTGT
CAGTGATTGGGTTCCGAATCCTCCTCCTGAAAGTGGCCGGGTTTAATCTGCTCATGACGCT
GCGGCTGTGGTCCAGCTGA
>Condition 3 No. 4 TCRα PCR product (with a stop) amino acid
sequence
(SEQ ID NO: 846)
MVLKFSVSILWIQLAWVSTQLLEQSPQFLSIQEGENLTVYCNSSSVFSSLQWYRQEPGEGP
VLLVTVVTGGEVKKLKRLTFQFGDARKDSSLHITAAQPGDTGLYLCA//IRQERTVLSTSL
RPSLVIQASTSVPIRGGGADGLTFGKGTHLIIQPYIQNPDPAVYQLRDSKSSDKSVCLFTD
FDSQTNVSQSKDSDVYITDKTVLDMRSMDFKSNSAVAWSNKSDFACANAFNNSIIPEDTFF
PSPESSCDVKLVEKSFETDINLNFQNLSVIGFRILLLKVAGFNLLMTLRLWSS*
>Condition 3 No. 4 TCRα PCR product (without a stop) base
sequence
(SEQ ID NO: 847)
GGCTCTTTCAGGAGCAGCTAAAGTCAGGGGCCATGTCCACCATGTGATAGAAAGACAAGAT
GGTCCTGAAATTCTCCGTGTCCATTCTTTGGATTCAGTTGGCATGGGTGAGCACCCAGCTG
CTGGAGCAGAGCCCTCAGTTTCTAAGCATCCAAGAGGGAGAAAATCTCACTGTGTACTGCA
ACTCCTCAAGTGTTTTTTCCAGCTTACAATGGTACAGACAGGAGCCTGGGGAAGGTCCTGT
CCTCCTGGTGACAGTAGTTACGGGTGGAGAAGTGAAGAAGCTGAAGAGACTAACCTTTCAG
TTTGGTGATGCAAGAAAGGACAGTTCTCTCCACATCACTGCGGCCCAGCCTGGTGATACAG
GCCTCTACCTCTGTGCCTATCCGAGGAGGAGGTGCTGACGGACTCACCTTTGGCAAAGGGA
CTCATCTAATCATCCAGCCCTATATCCAGAACCCTGACCCTGCCGTGTACCAGCTGAGAGA
CTCTAAATCCAGTGACAAGTCTGTCTGCCTATTCACCGATTTTGATTCTCAAACAAATGTG
TCACAAAGTAAGGATTCTGATGTGTATATCACAGACAAAACTGTGCTAGACATGAGGTCTA
TGGACTTCAAGAGCAACAGTGCTGTGGCCTGGAGCAACAAATCTGACTTTGCATGTGCAAA
CGCCTTCAACAACAGCATTATTCCAGAAGACACCTTCTTCCCCAGCCCAGAAAGTTCCTGT
GATGTCAAGCTGGTCGAGAAAAGCTTTGAAACAGATACGAACCTAAACTTTCAAAACCTGT
CAGTGATTGGGTTCCGAATCCTCCTCCTGAAAGTGGCCGGGTTTAATCTGCTCATGACGCT
GCGGCTGTGGTCCAGCTTA
>Condition 3 No. 4 TCRα PCR product (without a stop) amino
acid sequence
(SEQ ID NO: 848)
MVLKFSVSILWIQLAWVSTQLLEQSPQFLSIQEGENLTVYCNSSSVFSSLQWYRQEPGEGP
VLLVTVVTGGEVKKLKRLTFQFGDARKDSSLHITAAQPGDTGLYLCA//IRQERTVLSTSL
RPSLVIQASTSVPIRGGGADGLTFGKGTHLIIQPYIQNPDPAVYQLRDSKSSDKSVCLFTD
FDSQTNVSQSKDSDVYITDKTVLDMRSMDFKSNSAVAWSNKSDFACANAFNNSIIPEDTFF
PSPESSCDVKLVEKSFETDTNLNFQNLSVIGFRILLLKVAGFNLLMTLRLWSSL
>Condition 3 No. 4 TCRβ PCR product (with a stop) base
sequence
(SEQ ID NO: 849)
GGTAAAGCCCTCATCCTGTCCTGACCCTGCCATGGGCACCAGTCTCCTATGCTGGGTGGTC
CTGGGTTTCCTAGGGACAGATCACACAGGTGCTGGAGTCTCCCAGTCTCCCAGGTACAAAG
TCACAAAGAGGGGACAGGATGTAGCTCTCAGGTGTGATCCAATTTCGGGTCATGTATCCCT
TTATTGGTACCGACAGGCCCTGGGGCAGGGCCCAGAGTTTTTGACTTACTTCAATTATGAA
GCCCAACAAGACAAATCAGGGCTGCCCAATGATCGGTTTTTTGCAGAGAGGCCTGAGGGAT
CCATCTCCACTCTGACGATCCAGCGCACAGAGCAGCGGGACTCGGCCATGTATCGCTGTGC
CAGCAGCTCCTCTAGCGGGAGCTCCTACAATGAGCAGTTCTTCGGGCCAGGGACACGGCTC
ACCGTGCTAGAGGACCTGAAAAACGTGTTCCCACCCGAGGTCGCTGTGTTTGAGCCATCAG
AAGCAGAGATCTCCCACACCCAAAAGGCCACACTGGTGTGCCTGGCCACAGGCTTCTACCC
CGACCACGTGGAGCTGAGCTGGTGGGTGAATGGGAAGGAGGTGCACAGTGGGGTCAGCACA
GACCCGCAGCCCCTCAAGGAGCAGCCCGCCCTCAATGACTCCAGATACTGCCTGAGCAGCC
GCCTGAGGGTCTCGGCCACCTTCTGGCAGAACCCCCGCAACCACTTCCGCTGTCAAGTCCA
GTTCTACGGGCTCTCGGAGAATGACGAGTGGACCCAGGATAGGGCCAAACCTGTCACCCAG
ATCGTCAGCGCCGAGGCCTGGGGTAGAGCAGACTGTGGCTTCACCTCCGAGTCTTACCAGC
AAGGGGTCCTGTCTGCCACCATCCTCTATGAGATCTTGCTAGGGAAGGCCACCTTGTATGC
CGTGCTGGTCAGTGCCCTCGTGCTGATGGCCATGGTCAAGAGAAAGGATTCCAGAGGCTAG
>Condition 3 No. 4 TCRβ PCR product (with a stop) amino acid
sequence
(SEQ ID NO: 850)
MGTSLLCWVVLGFLGTDHTGAGVSQSPRYKVTKRGQDVALRCDPISGHVSLYWYRQALGQG
PEFLTYFNYEAQQDKSGLPNDRFFAERPEGSISTLTIQRTEQRDSAMYRCASSSSSGSSYN
EQFFGPGTRLTVLEDLKNVFPPEVAVFEPSEAEISHTQKATLVCLATGFYPDHVELSWWVN
GKEVHSGVSTDPQPLKEQPALNDSRYCLSSRLRVSATFWQNPRNHFRCQVQFYGLSENDEW
TQDRAKPVTQIVSAEAWGRADCGFTSESYQQGVLSATILYEILLGKATLYAVLVSALVLMA
MVKRKDSRG*
>Condition 3 No. 4 TCRβ PCR product (without a stop) base
sequence
(SEQ ID NO: 851)
GGTAAAGCCCTCATCCTGTCCTGACCCTGCCATGGGCACCAGTCTCCTATGCTGGGTGGTC
CTGGGTTTCCTAGGGACAGATCACACAGGTGCTGGAGTCTCCCAGTCTCCCAGGTACAAAG
TCACAAAGAGGGGACAGGATGTAGCTCTCAGGTGTGATCCAATTTCGGGTCATGTATCCCT
TTATTGGTACCGACAGGCCCTGGGGCAGGGCCCAGAGTTTTTGACTTACTTCAATTATGAA
GCCCAACAAGACAAATCAGGGCTGCCCAATGATCGGTTTTTTGCAGAGAGGCCTGAGGGAT
CCATCTCCACTCTGACGATCCAGCGCACAGAGCAGCGGGACTCGGCCATGTATCGCTGTGC
CAGCAGCTCCTCTAGCGGGAGCTCCTACAATGAGCAGTTCTTCGGGCCAGGGACACGGCTC
ACCGTGCTAGAGGACCTGAAAAACGTGTTCCCACCCGAGGTCGCTGTGTTTGAGCCATCAG
AAGCAGAGATCTCCCACACCCAAAAGGCCACACTGGTGTGCCTGGCCACAGGCTTCTACCC
CGACCACGTGGAGCTGAGCTGGTGGGTGAATGGGAAGGAGGTGCACAGTGGGGTCAGCACA
GACCCGCAGCCCCTCAAGGAGCAGCCCGCCCTCAATGACTCCAGATACTGCCTGAGCAGCC
GCCTGAGGGTCTCGGCCACCTTCTGGCAGAACCCCCGCAACCACTTCCGCTGTCAAGTCCA
GTTCTACGGGCTCTCGGAGAATGACGAGTGGACCCAGGATAGGGCCAAACCTGTCACCCAG
ATCGTCAGCGCCGAGGCCTGGGGTAGAGCAGACTGTGGCTTCACCTCCGAGTCTTACCAGC
AAGGGGTCCTGTCTGCCACCATCCTCTATGAGATCTTGCTAGGGAAGGCCACCTTGTATGC
CGTGCTGGTCAGTGCCCTCGTGCTGATGGCCATGGTCAAGAGAAAGGATTCCAGAGGCTTA
>Condition 3 No. 4 TCRβ PCR product (without a stop) amino
acid sequence
(SEQ ID NO: 852)
MGTSLLCWVVLGFLGTDHTGAGVSQSPRYKVTKRGQDVALRCDPISGHVSLYWYRQALGQG
PEFLTYFNYEAQQDKSGLPNDRFFAERPEGSISTLTIQRTEQRDSAMYRCASSSSSGSSYN
EQFFGPGTRLTVLEDLKNVFPPEVAVFEPSEAEISHTQKATLVCLATGFYPDHVELSWWVN
GKEVHSGVSTDPQPLKEQPALNDSRYCLSSRLRVSATFWQNPRNHFRCQVQFYGLSENDEW
TQDRAKPVTQIVSAEAWGRADCGFTSESYQQGVLSATILYEILLGKATLYAVLVSALVLMA
MVKRKDSRGL

TABLE 45

| RT& PCR | | TCRα | | TCRβ | | |
|---|---|---|---|---|---|---|
| primer | | TRAV | TRAJ | TRBV | TRBJ | TRBC |
| Condition 3 | No1 | TRAV38-2DV8 | TRAJ45 | TRBV14 | TRBJ2-7 | C2 |
| | No2 | TRAV13-2 | TRAJ16 | TRBV4-1 | TRBJ2-7 | C2 |
| | No4 | TRAV27 | TRAJ45 | TRBV7-6 | TRBJ2-1 | C2 |

G4-3. DNA Fragment Ligation and Introduction into E. coli

10 ng of DNA fragment (condition 3 No 1 TCRα-with a stop codon, TCRβ-with a stop codon) of full length protein coding region cDNA that was cloned was added to 20 ng of restriction enzyme EcoRV-digested and purified pcDNA3.1 V5-His (B) vector, and the volume thereof was increased to 4 μl with pure water. A TCR DNA fragment free condition with only the vector was used as a negative control. 4 μl of NEBuilder HiFi DNA Assembly Master Mix was added and mixed with the DNA solution and reacted for 30 minutes at 50° C. 1.65 μl of the reaction solution was fractionated. E. coli DH5a strain competent cells were transformed in accordance with the manufacturer's protocol. The transformed E. coli was cultured for 30 minutes at 37° C. after adding 19-fold volume of SOC medium. A portion thereof was fractionated and plated on an ampicillin-containing LB agar medium. When cultured overnight at 37° C., a greater number of colonies were observed in the sample to which a TCRα/β DNA fragment was added.

Since an EcoRV digested vector would be a blunt end DNA fragment and the PCR fragment would also be a blunt end, this experiment can be performed by ligation with a T4 ligase (however, in this case, it is necessary to digest the EcoRV digested pcDNA3.1 V5-His (B) vector with alkaline phosphatase in advance).

While some differences may result in the sequences of plasmid constructs depending on the method, a native TCR or V5-His tagged TCR expression vector can be constructed by any method.

The composition of reaction solution and reaction conditions of ligation are in accordance with the manufacturer's protocol. A ligation product is partially fractionated, and a competent cell is transformed and plated on an agar medium to obtain a genetically recombinant E. coli colony as shown in this Example.

G4-4. Plasmid DNA Preparation

Several single colonies of the resulting E. coli transformant (G4-3) were picked up from each reaction group and seeded in an ampicillin containing SOC medium and cultured for 8 hours at 37° C. Plasmids were prepared from samples suspended in the culture.

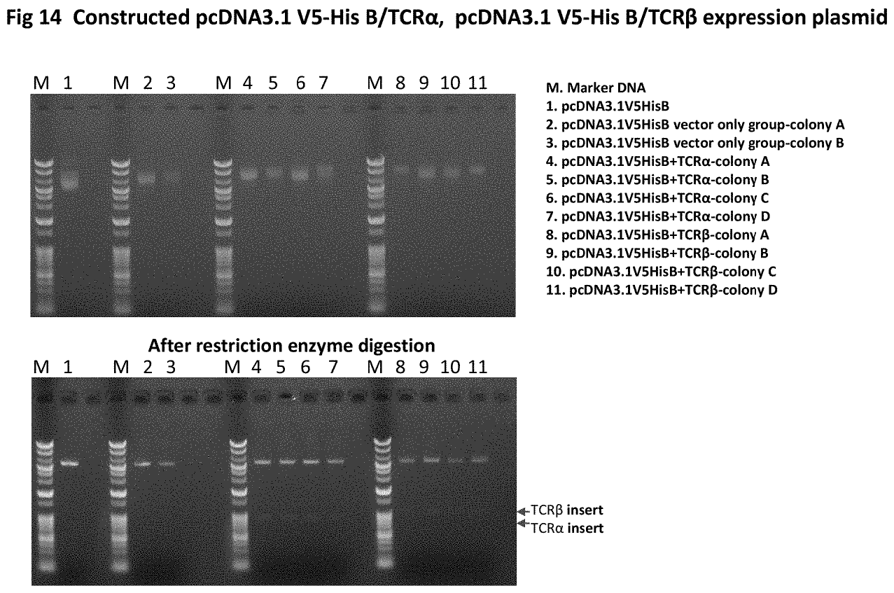

Electrophoresis was performed on the resulting plasmids without any treatment or after digestion with a restriction enzyme (EcoRI or XhoI) in a vector cloning site to study an insert. FIG. 14 shows electrophoretic images. For plasmids without digestion with a restriction enzyme, a band is shifted upwards in plasmids added with a TCRα DNA fragment (lanes 4 to 7) or TCRβ DNA fragment (lanes 8 to 11) compared to the original plasmid (lane 1) and plasmids assembled and ligated with only a vector (lanes 2 and 3). In restriction enzyme digested samples, an insert DNA fragment of slightly less than 1 kbp was found only with assembly and ligation of TCRα DNA fragment or TCRβ DNA fragment. When these plasmids were sequenced by the Sanger method using a vector primer or a primer in a TCR sequence, a sequence was found upon PCR amplified DNA fragment sequencing, except for one clone (pcDNA3.1 V5-His B+TCRα-colony B). The plasmid derived from pcDNA3.1 V5-His B+TCRα-colony B was a clone with a mutation on one base resulting in a missense mutation (substitution of aspartic acid with asparagine) of an amino acid in a protein ORF.

Since an EcoRV digested vector would be a blunt end DNA fragment and the PCR fragment would also be a blunt end, this experiment can be performed by ligation with a normal T4 ligase or the like (however, in this case, it is necessary to digest the EcoRV digested pcDNA3.1 V5-His (B) vector with alkaline phosphatase in advance). An expression plasmid is also obtained by incorporating a restriction enzyme site into an adaptor sequence and digesting with a restriction enzyme, and then ligating with a vector digested with the same enzyme.

A native TCR or V5-His tagged TCR expression vector can be constructed in these experimental systems by any method. The composition of reaction solution and reaction conditions of ligation are in accordance with the manufacturer's protocol. As shown in this Examples, a ligation product is partially fractionated, and a competent cell is transformed and plated on an agar medium to obtain genetically recombinant E. coli.

G4-5. Verification of Sequence of Plasmid DNA Inset

For the resulting plasmids, an insert sequence was sequenced by the common Sanger sequencing method using each of a TCRα sequencing primer, TCRβ sequencing primer, and vector primer. As a result, clones without a base sequence mutation were obtained at a high efficiency, i.e., 7 out of 8 clones. In one out of 8 clones (pcDNA3.1 V5-His B+TCRα-colony B), a mutation of one base resulting in a missense mutation (substitution of aspartic acid with asparagine) of an amino acid in a protein ORF was found. The sequences in a form comprising a V5-His tag sequence in a vector are shown below (an underline part indicates a start codon ATG or stop codon, and * indicates a site where protein synthesis stops due to a stop codon). Both the pcDNA3.1 V5-His TCRα and pcDNA3.1 V5-His TCRβ plasmid clones can express a TCR protein with a V5-His tag fused to the protein C-terminus from substituting the first stop codon with L (leucine) by inserting a stop codon free DNA fragment prepared in advance (G4-2.) by the same methodology into a pcDNA3.1 V5-His B vector.

These expression plasmids of TCRα/β pair genes can be introduced simultaneously into cells by electroporation, lipofection, calcium phosphate transfection, or the like to express pair genes. A protein can also be expressed by utilizing a T7 promoter in a vector and synthesizing TCRα/β pair gene mRNA in an in vitro transcription reaction and introducing the mRNA into a cell, or a protein can be synthesized by further using the TCRα/β pair gene mRNA in a TCRα/β pair gene synthesis in an in vitro translation system.

>pcDNA3.1 V5-His B/TCRα (with a stop) plasmid-(same sequence
in pcDNA3.1 V5-His B + TCRα--colony A, C, or D derived
plasmid)
(SEQ ID NO: 856)
AGCAGGGACCTGTGAGCATGGCATGCCCTGGCTTCCTGTGGGCACTTGTGATCTCCACCTG
TCTTGAATTTAGCATGGCTCAGACAGTCACTCAGTCTCAACCAGAGATGTCTGTGCAGGAG
GCAGAGACCGTGACCCTGAGCTGCACATATGACACCAGTGAGAGTGATTATTATTTATTCT
GGTACAAGCAGCCTCCCAGCAGGCAGATGATTCTCGTTATTCGCCAAGAAGCTTATAAGCA
ACAGAATGCAACAGAGAATCGTTTCTCTGTGAACTTCCAGAAAGCAGCCAAATCCTTCAGT
CTCAAGATCTCAGACTCACAGCTGGGGGATGCCGCGATGTATTTCTGTGCCCGGTATTCAG
GAGGAGGTGCTGACGGACTCACCTTTGGCAAAGGGACTCATCTAATCATCCAGCCCTATAT
CCAGAACCCTGACCCTGCCGTGTACCAGCTGAGAGACTCTAAATCCAGTGACAAGTCTGTC
TGCCTATTCACCGATTTTGATTCTCAAACAAATGTGTCACAAAGTAAGGATTCTGATGTGT
ATATCACAGACAAAACTGTGCTAGACATGAGGTCTATGGACTTCAAGAGCAACAGTGCTGT
GGCCTGGAGCAACAAATCTGACTTTGCATGTGCAAACGCCTTCAACAACAGCATTATTCCA
GAAGACACCTTCTTCCCCAGCCCAGAAAGTTCCTGTGATGTCAAGCTGGTCGAGAAAAGCT
TTGAAACAGATACGAACCTAAACTTTCAAAACCTGTCAGTGATTGGGTTCCGAATCCTCCT
CCTGAAAGTGGCCGGGTTTAATCTGCTCATGACGCTGCGGCTGTGGTCCAGCTGAATCCAG
CACAGTGGCGGCCGCTCGAGTCTAGAGGGCCCGCGGTTCGAAGGTAAGCCTATCCCTAACC
CTCTCCTCGGTCTCGATTCTACGCGTACCGGTCATCATCACCATCACCATTGA
>pcDNA3.1 V5-His B/TCRα (with a stop) plasmid expressing
protein sequence-(same sequence in pcDNA3.1 V5-His B + TCRα--
colony A, C, and D derived plasmid)
(SEQ ID NO: 857)
MACPGFLWALVISTCLEFSMAQTVTQSQPEMSVQEAETVTLSCTYDTSESDYYLFWYKQFP
SRQMILVIRQEAYKQQNATENRFSVNFQKAAKSFSLKISDSQLGDAAMYFCARYSGGGADG
LTFGKGTHLIIQPYIQNPDPAVYQLRDSKSSDKSVCLFTDFDSQTNVSQSKDSDVYITDKT
VLDMRSMDFKSNSAVAWSNKSDFACANAFNNSIIPEDTFFPSPESSCDVKLVEKSFETDTN
LNFQNLSVIGFRILLLKVAGFNLLMTLRLWSS*IQHSGGRSSLEGPRFEGKPIPNPLLGLD
STRTGHHHHHH*
>pcDNA3.1 V5-His B/TCRβ (with a stop) plasmid-(same sequence
in pcDNA3.1 V5-His B + TCRβ-colony A, B, C, and D derived
plasmid)
(SEQ ID NO: 858)
GGGTCCTGCCATGGTTTCCAGGCTTCTCAGTTTAGTGTCCCTTTGTCTCCTGGGAGCAAAG
CACATAGAAGCTGGAGTTACTCAGTTCCCCAGCCACAGCGTAATAGAGAAGGGCCAGACTG
TGACTCTGAGATGTGACCCAATTTCTGGACATGATAATCTTTATTGGTATCGACGTGTTAT
GGGAAAAGAAATAAAATTTCTGTTACATTTTGTGAAAGAGTCTAAACAGGATGAGTCCGGT
ATGCCCAACAATCGATTCTTAGCTGAAAGGACTGGAGGGACGTATTCTACTCTGAAGGTGC
AGCCTGCAGAACTGGAGGATTCTGGAGTTTATTTCTGTGCCAGCAGCCAAGATCGCATCGA
GCAGTACTTCGGGCCGGGCACCAGGCTCACGGTCACAGAGGACCTGAAAAACGTGTTCCCA
CCCGAGGTCGCTGTGTTTGAGCCATCAGAAGCAGAGATCTCCCACACCCAAAAGGCCACAC
TGGTGTGCCTGGCCACAGGCTTCTACCCCGACCACGTGGAGCTGAGCTGGTGGGTGAATGG
GAAGGAGGTGCACAGTGGGGTCAGCACAGACCCGCAGCCCCTCAAGGAGCAGCCCGCCCTC
AATGACTCCAGATACTGCCTGAGCAGCCGCCTGAGGGTCTCGGCCACCTTCTGGCAGAACC
CCCGCAACCACTTCCGCTGTCAAGTCCAGTTCTACGGGCTCTCGGAGAATGACGAGTGGAC
CCAGGATAGGGCCAAACCTGTCACCCAGATCGTCAGCGCCGAGGCCTGGGGTAGAGCAGAC
TGTGGCTTCACCTCCGAGTCTTACCAGCAAGGGGTCCTGTCTGCCACCATCCTCTATGAGA
TCTTGCTAGGGAAGGCCACCTTGTATGCCGTGCTGGTCAGTGCCCTCGTGCTGATGGCCAT
GGTCAAGAGAAAGGATTCCAGAGGCTAGATCCAGCACAGTGGCGGCCGCTCGAGTCTAGAG
GGCCCGCGGTTCGAAGGTAAGCCTATCCCTAACCCTCTCCTCGGTCTCGATTCTACGCGTA
CCGGTCATCATCACCATCACCATTGA
>pcDNA3.1 V5-His B/TCRβ (with a stop) plasmid expressing
protein sequence-(same sequence in pcDNA3.1 V5-His B + TCRβ-
colony A, B, C, and D derived plasmid)
(SEQ ID NO: 859)
MVSRLLSLVSLCLLGAKHIEAGVTQFPSHSVIEKGQTVTLRCDPISGHDNLYWYRRVMGKE
IKFLLHFVKESKQDESGMPNNRFLAERTGGTYSTLKVQPAELEDSGVYFCASSQDRIEQYF
GPGTRLTVTEDLKNVFPPEVAVFEPSEAEISHTQKATLVCLATGFYPDHVELSWWVNGKEV
HSGVSTDPQPLKEQPALNDSRYCLSSRLRVSATFWQNPRNHFRCQVQFYGLSENDEWTQDR
AKPVTQIVSAEAWGRADCGFTSESYQQGVLSATILYEILLGKATLYAVLVSALVLMAMVKR
KDSRG*IQHSGGRSSLEGPRFEGKPIPNPLLGLDSTRTGHHHHHH*

G5. cDNA Cloning of V(D)J Region Fragment or C Region Fragment of TCR Pair Genes and Full Length Protein Coding Region Expression Vector

G5-1 PCR Reaction-1

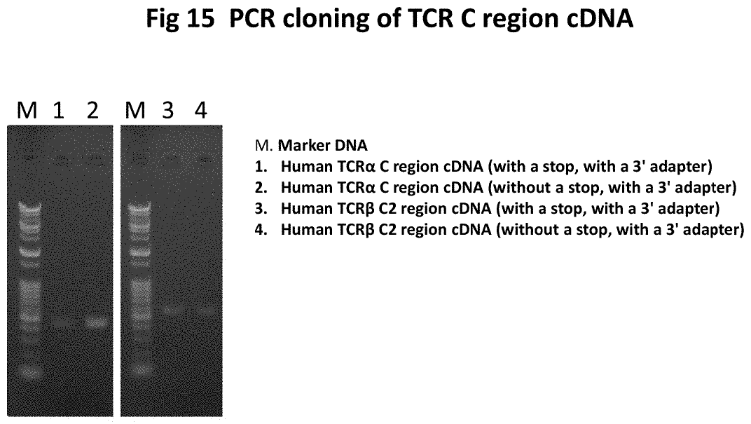

For cloning TCRα and TCRβ pair genes of No 1, 3, and 4 under condition 3, as shown in Example 5, a V(D)J region fragment and C region fragment were separately prepared. To construct a full length protein coding region expression vector, a PCR reaction was first performed in the reaction solution of Table 46 using human CD8 positive T cell derived cDNA as a template to perform cDNA cloning of a TCRα C region and TCRβ C2 region. A band was studied by agarose gel electrophoresis using 3 μl of the reaction solution (FIG. 15).

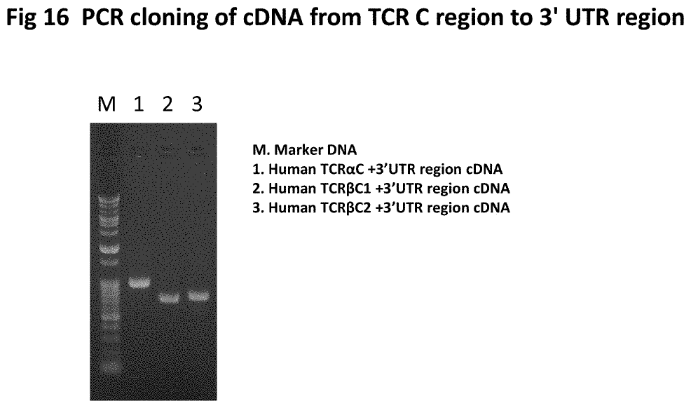

Although not used in this experiment, an attempt was made to amplify a C region fragment without an adapter to a relatively terminal region of 3′ UTR. While referring to annotation information on a public genome database or the like, a reverse side primer was designed at the relatively terminal region of 3′ UTR. A PCR reaction was performed in the reaction solution of Table 47 using human CD8 positive T cell derived cDNA as a template, and cDNA was cloned from the C region to the 3′ UTR region of TCR. A band was studied by agarose gel electrophoresis using 3 μl of reaction solution (FIG. 16). While an adapter is not added to this DNA, as a TCR C region sample, a cDNA fragment added with an adapter for ligation to various vectors or the like can be used as a template for PCR preparation.

G5-2 PCR Reaction-2

To clone TCRα and TCRβ pair genes of No 1, 3, and 4 under condition 3, primers were designed for upstream of a start codon in each variable region (V region) or upstream of a start codon including the start codon and ORF region (F4-1).

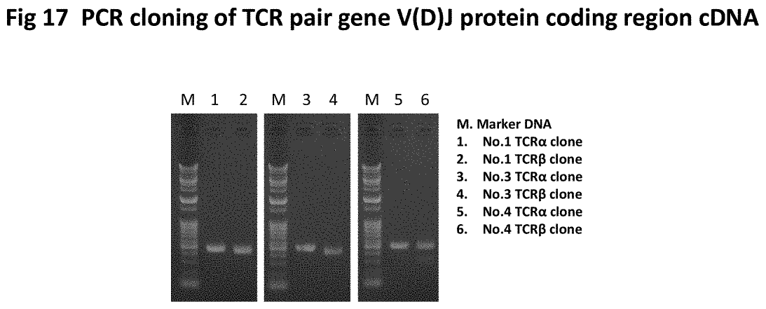

2.0 μl of semi-nested PCR reaction solution purified DNA solution (DNA sample prepared in G3-2 and purified for sequencing in G3-3) was fractionated. A PCR reaction was performed in the reaction solution of Table 48, and TCR V(D)J protein coding region cDNA was cloned. While a V region F primer needs to be changed for each clone, primers were used as specified in the list of Table 49. 3 μl of reaction solution was fractionated to study a band by agarose gel electrophoresis (FIG. 17).

TABLE 48

| | Amount of solution | Amount of solution |
|---|---|---|
| | for TCRα | for TCRβ |
| Reagents, etc. | amplification (μl) | amplification (μl) |
| Semi-nested PCR reaction | 2.0 | 2.0 |
| solution purified DNA | | |
| solution | | |
| 2 × KAPA HiFi Hot | 12.5 | 12.5 |
| Start Ready Mix | | |
| hTCRaVXX-F#-AdpD3EV | 0.5 | — |
| (10 uM)* | | |
| hTCRa-C-R (10 uM) | 0.5 | — |
| hTCRbVXX-F#-AdpD3EV | — | 0.5 |
| (10 uM)* | | |
| hTCRb-C2-R (10 uM) | — | 0.5 |
| DW | 9.5 | 9.5 |
| Total | 25.0 | 25.0 |

*collectively referring to V region specific adaptor added primers. XX is the V number, and # is a number.

G5-3. DNA Fragment Ligation and Introduction into E. coli

A 3′ adaptor added TCR C region DNA fragment (can be either with or without a stop codon) cloned in G5-1 and a 5′ adaptor added TCR V(D)J protein coding region cDNA cloned in F5-2 were assembled and ligated to a restriction enzyme EcoRV digested and purified pcDNA3.1 V5-His (B) vector using NEBuilder HiFi DNA Assembly Master Mix or the like.

A native TCR or V5-His tagged TCR expression vector can be constructed with this series of experimental systems.

The composition of reaction solution and reaction conditions of ligation are in accordance with the manufacturer's protocol. A ligation product is partially fractionated, and a competent cell is transformed and plated on an agar medium to obtain genetically recombinant E. coli.

G5-4. Plasmid DNA Preparation

A single colony of plasmid gene recombinant organisms is cultured in a liquid medium and harvested, and the plasmid DNA is prepared with a common alkaline extraction method or a commercially available kit such as NucleoSpin Plasmid.

G5-5. Verification of Sequence of Plasmid DNA Insert

For the resulting plasmids, the insert sequence is sequenced by a common Sanger sequencing method using each of a TCRα sequencing primer, TCRβ sequencing primer, and vector primer. A clone without a substitution (at least without a missense mutation or nonsense mutation), insertion, or deletion in the base sequence, in comparison to PCR amplified fragment sequencing data, is used as a TCR expression construct.

(Note)

While the present disclosure has been described with the emphasis on the preferred embodiments, it is evident to those skilled in the art that the preferred embodiments can be modified. The present disclosure is intended so that the present disclosure can be practiced by methods other than those described herein in detail. Therefore, the present disclosure encompasses all changed encompassed by the spirit and scope of the appended “Claims”.

The content of any document described herein including any patent and any patent application is incorporated herein by reference to the same extent as the contents are specifically described herein. The present application claims priority to Japanese Patent Application No. 2018-157760 filed on Aug. 24, 2018. The entire content thereof is incorporated herein by reference.

### INDUSTRIAL APPLICABILITY

The present disclosure provides a method of analyzing TCR or BCR functional pair genes very readily and at high accuracy, and is particularly useful in the manufacture of disease specific and functional TCR or BCR, or in clinical settings requiring a large scale quantitative analysis.

[Sequence Listing Free Text]
SEQ ID NO: 1: hTCRα Block primer (CA2; 23 bases)
SEQ ID NO: 2: hTCRα RT primer (CA1; 23 bases)
SEQ ID NO: 3: hTCRβ Block primer (CB2; 23 bases)
SEQ ID NO: 4: hTCRβ RT primer (CB1(3); 23 bases)
SEQ ID NO: 5: TS-Oligo (30 bases)
SEQ ID NO: 6: TS-Primer (30 bases)
SEQ ID NO: 7: Forward TS-Tag primer v1 (64 bases)
SEQ ID NO: 8: hTCRα Reverse Tag primer (51 bases)
SEQ ID NO: 9: hTCRα Reverse Tag primer/hTCA-R-TP5-1 (53
bases)
SEQ ID NO: 10: hTCRβ Reverse Tag primer (52 bases)
SEQ ID NO: 11: P5-seq (21 bases)
SEQ ID NO: 12: CA3 (23 bases)
SEQ ID NO: 13: CB3 (23 bases)
SEQ ID NO: 14: well No. 13 TCRα base sequence in Experiment A
SEQ ID NO: 15: well No. 13 TCRα amino acid translated
sequence in Experiment A
SEQ ID NO: 16: well No. 13 TCRβ base sequence in Experiment A
SEQ ID NO: 17: well No. 13 TCRβ amino acid translated
sequence in Experiment A
SEQ ID NO: 18 to 416: CDR3 sequences in Tables 6 to 13
SEQ ID NO: 417 to 485: CDR3 sequences in Tables 17 to 18
SEQ ID NO: 486 to 579: CDR3 sequences in Tables 21 to 22
SEQ ID NO: 580 to 603: CDR3 sequences in Table 24
SEQ ID NO: 604: hBCRμ Block primer (CM2(2); 19 bases)
SEQ ID NO: 605: hBCRμ RT primer (CM1(2); 23 bases)
SEQ ID NO: 606: hBCR Kappa Block primer (CK2; 18 bases)
SEQ ID NO: 607: hBCR Kappa RT primer (CK1; 19 bases)
SEQ ID NO: 608: hBCR Lambda Block primer (CL2; 16 bases)
SEQ ID NO: 609: hBCR Lambda RT primer (CL1; 19 bases)
SEQ ID NO: 610: hBCRμ Reverse Tag primer (CM-ST1-R-(3), 53
bases)
SEQ ID NO: 611: hBCR Kappa Reverse Tag primer (CK-ST1-R, 51
bases)
SEQ ID NO: 612: hBCR Lambda Reverse Tag primer (CL-ST1-R, 51
bases)
SEQ ID NO: 613 to 744: CDR3 sequences in Tables 28 to 29
SEQ ID NO: 745: TS-AdpD3EV
SEQ ID NO: 746: hTCRa-VX-F-AdpD3EV
SEQ ID NO: 747: hTCRa-V24-F-AdpD3EV
SEQ ID NO: 748: hTCRb-VX-F-AdpD3EV
SEQ ID NO: 749: hTCRb-V6.5-F-AdpD3EV
SEQ ID NO: 750: TS-AdpMXEI
SEQ ID NO: 751: hTCRa-VX-F-AdpMXEI
SEQ ID NO: 752: hTCRb-VX-F-AdpMXEI
SEQ ID NO: 753: hTCRa-V24-F-AdpMXEI
SEQ ID NO: 754: hTCRb-V6.5-F-AdpMXEI
SEQ ID NO: 755: hTCRa-C-R
SEQ ID NO: 756: hTCRb-Cl-R
SEQ ID NO: 757: hTCRb-C2-R
SEQ ID NO: 758: hTCRaC-F0
SEQ ID NO: 759: hTCRaC-F1
SEQ ID NO: 760: hTCRaC-F2
SEQ ID NO: 761: hTCRaC-F3
SEQ ID NO: 762: hTCRaC-R1v1
SEQ ID NO: 763: hTCRaC-R2
SEQ ID NO: 764: hTCRaC-R4
SEQ ID NO: 765: hTCRaC-R5
SEQ ID NO: 766: hTCR-CB-F2
SEQ ID NO: 767: hTCRbC-F5
SEQ ID NO: 768: hTCR-CB-R2
SEQ ID NO: 769: hTCR-CB2
SEQ ID NO: 770: hTCR-CB3
SEQ ID NO: 771: hTCRbC2-CloR1
SEQ ID NO: 772: hTCRbC1-R1
SEQ ID NO: 773: hTCRaCFull-AdpD3EV
SEQ ID NO: 774: hTCRaCFull_noSTOP-AdpD3EV
SEQ ID NO: 775: hTCRbC1Full-AdpD3EV
SEQ ID NO: 776: hTCRbC1Full_noSTOP-AdpD3EV
SEQ ID NO: 777: hTCRbC2Full-AdpD3EV
SEQ ID NO: 778: hTCRbC2Full_noSTOP-AdpD3EV
SEQ ID NO: 779: hTCRaCFull-AdpMXEI
SEQ ID NO: 780: hTCRbC1Full-AdpMXEI
SEQ ID NO: 781: hTCRbC2Full-AdpMXEI
SEQ ID NO: 782: TS-AdpD3EV
SEQ ID NO: 783: hBCRH-VX-F-AdpD3EV
SEQ ID NO: 784: hBCRH-V3.30-F-AdpD3EV
SEQ ID NO: 785: hBCRH-V2.70-F-AdpD3EV
SEQ ID NO: 786: hBCRLam-VX-F-AdpD3EV
SEQ ID NO: 787: hBCRLam-V2.14-F-AdpD3EV
SEQ ID NO: 788: hBCRKap-VX-F-AdpD3EV
SEQ ID NO: 789: hBCRK-V3.15-F-AdpD3EV
SEQ ID NO: 790: hBCRM-C-R
SEQ ID NO: 791: hBCRLam-C2-R
SEQ ID NO: 792: hBCRKap-C1-R
SEQ ID NO: 793: hBCRKapCFull-AdpD3EV
SEQ ID NO: 794: hBCRLamC2Full-AdpD3EV
SEQ ID NO: 795: hBCRμCFull-AdpD3EV
SEQ ID NO: 796: well No. 18 BCRμ base sequence in Experiment D
SEQ ID NO: 797: well No. 18 BCRμ amino acid translated
sequence in Experiment D
SEQ ID NO: 798: well No. 18 BCR Lambda base sequence in
Experiment D
SEQ ID NO: 799: well No. 18 BCR Lambda amino acid translated
sequence in Experiment D
SEQ ID NO: 800: well No. 55 BCRμ base sequence in Experiment D
SEQ ID NO: 801: well No. 55 BCRμ amino acid translated
sequence in Experiment D
SEQ ID NO: 802: well No. 55 BCR Kappa base sequence in
Experiment D
SEQ ID NO: 803: well No. 55 BCR Kappa amino acid translated
sequence in Experiment D
SEQ ID NO: 804: amino acid sequence of pp65 (AKI22842.1) of
CMV (Human betaherpesvirus 5)
SEQ ID NO: 805: amino acid sequence of Influenza M1 protein
(P03485)
SEQ ID NO: 806: hTCRα RT primer (hTCRaC-CloR1; 21 bases)
SEQ ID NO: 807: hTCRα RT primer (hTCRaC-CloR2; 22 bases)
SEQ ID NO: 808: hTCRα PCR primer (hTCRaC-R2v0; 21 bases)
SEQ ID NO: 809: hTCRβ RT primer (hTCRbC1-R3; 21 bases)
SEQ ID NO: 810: hTCRβ RT primer (hTCRbC2-CloR2; 21 bases)
SEQ ID NO: 811: hTCRβ PCR primer (hTCRbC1-R2v2; 20 bases)
SEQ ID NO: 812: hTCRβ PCR primer (hTCRbC2-CloR3; 21 bases)
SEQ ID NO: 813: hTCRaV38.2-F1-AdpD3EV
SEQ ID NO: 814: hTCRbV14-F1-AdpD3EV
SEQ ID NO: 815: hTCRaV13.2-F2-AdpD3EV
SEQ ID NO: 816: hTCRbV4.1-F1-AdpD3EV
SEQ ID NO: 817: hTCRaV27-F3-AdpD3EV
SEQ ID NO: 818: hTCRbV7.6-F1-AdpD3EV
SEQ ID NO: 819: hTCRaC-AdpD3EV
SEQ ID NO: 820: hTCRaC-NT-AdpD3EV
SEQ ID NO: 821: hTCRbC1-AdpD3EV
SEQ ID NO: 822: hTCRbC1-NT-AdpD3EV
SEQ ID NO: 823: hTCRbC2-AdpD3EV
SEQ ID NO: 824: hTCRbC2-NT-AdpD3EV
SEQ ID NO: 825: hTCRa-C-F
SEQ ID NO: 826: hTCRb-Cl-F
SEQ ID NO: 827: hTCRb-C2-F
SEQ ID NO: 828: hTCR-CB2-R2
SEQ ID NO: 829: No. 1 TCRα PCR product (with a stop) base
sequence
SEQ ID NO: 830: No. 1 TCRα PCR product (with a stop) amino
acid sequence
SEQ ID NO: 831: No. 1 TCRα PCR product (without a stop) base
sequence
SEQ ID NO: 832: No. 1 TCRα PCR product (without a stop) amino
acid sequence
SEQ ID NO: 833: No. 1 TCRβ PCR product (with a stop) base
sequence
SEQ ID NO: 834: No. 1 TCRβ PCR product (with a stop) amino
acid sequence
SEQ ID NO: 835: No. 1 TCRβ PCR product (without a stop) base
sequence
SEQ ID NO: 836: No. 1 TCRβ PCR product (without a stop) amino
acid sequence
SEQ ID NO: 837: No. 3 TCRα PCR product (with a stop) base
sequence
SEQ ID NO: 838: No. 3 TCRα PCR product (with a stop) amino
acid sequence
SEQ ID NO: 839: No. 3 TCRα PCR product (without a stop) base
sequence
SEQ ID NO: 840: No. 3 TCRα PCR product (without a stop) amino
acid sequence
SEQ ID NO: 841: No. 3 TCRβ PCR product (with a stop) base
sequence
SEQ ID NO: 842: No. 3 TCRβ PCR product (with a stop) amino
acid sequence
SEQ ID NO: 843: No. 3 TCRβ PCR product (without a stop) base
sequence
SEQ ID NO: 844: No. 3 TCRβ PCR product (without a stop) amino
acid sequence
SEQ ID NO: 845: No. 4 TCRα PCR product (with a stop) base
sequence
SEQ ID NO: 846: No. 4 TCRα PCR product (with a stop) amino
acid sequence
SEQ ID NO: 847: No. 4 TCRα PCR product (without a stop) base
sequence
SEQ ID NO: 848: No. 4 TCRα PCR product (without a stop) amino
acid sequence
SEQ ID NO: 849: No. 4 TCRβ PCR product (with a stop) base
sequence
SEQ ID NO: 850: No. 4 TCRβ PCR product (with a stop) amino
acid sequence
SEQ ID NO: 851: No. 4 TCRβ PCR product (without a stop) base
sequence
SEQ ID NO: 852: No. 4 TCRβ PCR product (without a stop) amino
acid sequence
SEQ ID NO: 853: T7
SEQ ID NO: 854: CMV-Fwd2
SEQ ID NO: 855: BGH-Reverse
SEQ ID NO: 856: pcDNA3.1 V5-His B/TCRα (with a stop) plasmid
SEQ ID NO: 857: pcDNA3.1 V5-His B/TCRα (with a stop) plasmid
expressing amino acid sequence
SEQ ID NO: 858: pcDNA3.1 V5-His B/TCRβ (with a stop) plasmid
SEQ ID NO: 859: pcDNA3.1 V5-His B/TCRβ (with a stop) plasmid
expressing amino acid sequence

1. A method of analyzing a sequence of functional subunit pair genes of a T cell receptor (TCR) or a B cell receptor (BCR), comprising:
(1) providing a nucleic acid sample comprising a nucleic acid of a TCR or a BCR from an activated cell expressing a TCR or a BCR;
(2) determining a nucleic acid sequence of the TCR or the BCR; and
(3) calculating a frequency of appearance of each gene or a combination thereof based on the determined nucleic acid sequence to identify TCR or BCR functional subunit pair genes.

2. The method of claim 1, further comprising activating at least a part of the cells expressing a TCR or a BCR prior to step (1).

3. The method of claim 1, wherein the activation comprises stimulating the cells with any antigen or an antigen MHC complex thereof, or an antigen presenting cell having an MHC bound to any antigen.

4. The method of claim 3, wherein the antigen is selected from the group consisting of a virus constituent substance, a microbe constituent substance, a non-autologous cell constituent substance, and a cancer cell specific constituent substance.

5. The method of claim 3, wherein the antigen is selected from the group consisting of a viral peptide, a viral peptide-glycolipid complex, a microbial peptide, a microbial peptide-glycolipid complex, a non-autologous cell peptide, a non-autologous cell peptide-glycolipid complex, a cancer cell specific peptide, and a cancer cell specific peptide-glycolipid complex.

6. The method of claim 1, wherein the activation comprises stimulating the cells with an agent selected from the group consisting of an anti-CD3 and/or anti-CD28 antibody, a phospholipase C (PLC) activating agent, a calcium ionophore, a protein kinase C (PKC) activating agent, a phytohemagglutinin (PHA), a concanavalin A (ConA), and a toll-like receptor activating agent.

7. The method of claim 1, wherein the cells are peripheral blood mononuclear cells.

8. The method of claim 1, wherein step (1) comprises separating the activated cell from an unactivated cell.

9. The method of claim 8, wherein the separation of the activated cell is performed with an antigen-MHC molecule complex or a polymer of 2 to 10,000 antigen-MHC molecule complexes.

10. The method of claim 9, wherein the polymer of antigen-MHC molecule complexes is selected from the group consisting of an epitope dimer, an epitope trimer, an epitope tetramer, and an epitope pentamer.

11. The method of claim 1, wherein the nucleic acid sample is obtained from a single activated cell.

12. The method of claim 1, wherein step (1) comprises:
a) mixing an RNA of the cells, a reagent required for reverse transcription, a reagent required for template switching, and a reagent required for a polymerase chain reaction, and subjecting the mixture to a reserve transcription inducing condition to provide a cDNA comprising nucleic acid sequences of a plurality of types of TCRs or BCRs; and
b) subjecting the cDNA obtained from step a) to a polymerase chain reaction inducing condition to provide a nucleic acid sample comprising the nucleic acid sequences of the plurality of types of TCRs or BCRs; wherein
the reagent required for template switching comprises a template switching oligonucleotide, and
the reagent required for a polymerase chain reaction comprises a primer specific to a C region, a primer specific to a 3′ untranslated region, or a primer spanning a C region and a 3′ untranslated region of the TCR or the BCR, wherein the primer specific to a C region, the primer specific to a 3′ untranslated region, or the primer spanning a C region and a 3′ untranslated region is a modified oligonucleotide primer designed to partially or completely block a primer function in step a) and to clear blocking of a primer function in step b).

13. The method of claim 12, wherein the reagent required for a polymerase chain reaction optionally further comprises a 5′ anchor oligonucleotide primer comprising at least a part of an anchor sequence contained in the template switching oligonucleotide.

14. The method of claim 13, wherein the reagent required for a polymerase chain reaction is free of the 5′ anchor oligonucleotide primer, and the template switching oligonucleotide functions as a 5′ anchor oligonucleotide primer.

15. The method of claim 12, wherein the reagent required for reverse transcription comprises:
(i) an oligonucleotide primer that initiates reverse transcription which is complementary to a C region, a 3′ untranslated region, or a region spanning a C region and a 3′ untranslated region of TCRα and an oligonucleotide primer that initiates reverse transcription which is complementary to a C region, a 3′ untranslated region, or a region spanning a C region and a 3′ untranslated region of TCRβ;
(ii) an oligonucleotide primer that initiates reverse transcription which is complementary to a C region, a 3′ untranslated region, or a region spanning a C region and a 3′ untranslated region of TCRδ and an oligonucleotide primer that initiates reverse transcription which is complementary to a C region, a 3′ untranslated region, or a region spanning a C region and a 3′ untranslated region of TCRγ; or
(iii) an oligonucleotide primer that initiates reverse transcription which is complementary to a C region, a 3′ untranslated region, or a region spanning a C region and a 3′ untranslated region of a BCR heavy chain and an oligonucleotide primer that initiates reverse transcription which is complementary to a C region, a 3′ untranslated region, or a region spanning a C region and a 3′ untranslated region of a BCR light chain.

16. The method of claim 12, wherein the reagent required for a polymerase chain reaction comprises:
a primer specific to a C region, a primer specific to a 3′ untranslated region, or a primer spanning a C region and a 3′ untranslated region of TCRα and a primer specific to a C region, a primer specific to a 3′ untranslated region, or a primer spanning a C region and a 3′ untranslated region of TCRβ;
(ii) a primer specific to a C region, a primer specific to a 3′ untranslated region, or a primer spanning a C region and a 3′ untranslated region of TCRδ and a primer specific to a C region, a primer specific to a 3′ untranslated region, or a primer spanning a C region and a 3′ untranslated region of TCRγ; or
(iii) a primer specific to a C region, a primer specific to a 3′ untranslated region, or a primer spanning a C region and a 3′ untranslated region of a BCR heavy chain and a primer specific to a C region, a primer specific to a 3′ untranslated region, or a primer spanning a C region and a 3′ untranslated region of a BCR light chain.

17. The method of claim 15, wherein both TCRα and TCRβ, both TCRδ and TCRγ, or both a BCR heavy chain and a BCR light chain are co-amplified.

18. The method of claim 12, wherein the modified oligonucleotide primer has one or more complementary regions on a sequence of the same modified oligonucleotide primer and has a folded structure due to the complementary regions prior to initial thermal denaturation processing of PCR, or comprises a thermolabile modifying group.

19. The method of claim 12, wherein a part of a modified oligonucleotide whose primer function has not been blocked functions as an oligonucleotide primer that initiates reverse transcription by hybridizing to a template RNA.

20. The method of claim 12, wherein step (3) comprises:
(3-1) providing a reference database for each of gene regions comprising at least one of a V region, a D region, a J region, and optionally a C region;
(3-2) providing an input sequence set prepared from optionally trimming and optionally extracting a sequence with a suitable length;
(3-3) searching for homology of the input sequence set with the reference database for each of the gene regions and recording an alignment with an approximate reference allele and/or a sequence of the reference allele;
(3-4) assigning the V region and the J region for the input sequence set and extracting a nucleic acid sequence of the D region based on a result of assigning;
(3-5) translating the nucleic acid sequence of the D region into an amino acid sequence and classifying the D region by utilizing the amino acid sequence; and
(3-6) calculating a frequency of appearance or a combination for each of the V region, the D region, the J region, and optionally the C region based on the classifying in step (3-5) to identify functional subunit pair genes of a TCR or a BCR.

21. A system for analyzing a sequence of functional subunit pair genes of a T cell receptor (TCR) or a B cell receptor (BCR) in cells expressing a TCR or a BCR, comprising:
(A) an activator for activating at least a part of the cells;
(B) a nucleic acid treating kit for providing a nucleic acid sample comprising a nucleic acid sequence of a TCR or a BCR obtained from the activated cell;
(C) a sequencer for determining a nucleic acid sequence contained in the activated cell; and
(D) an analyzer for calculating a frequency of appearance of each gene or a combination thereof based on the determined nucleic acid sequence to identify functional subunit pair genes of a TCR or a BCR.

22. A method of manufacturing a cell expressing a T cell receptor (TCR) or a B cell receptor (BCR) having a sequence of functional subunit pair genes analyzed in accordance with the method of claim 1, comprising:
(4) providing an expression construct comprising the nucleic acid sequence of the TCR or the BCR; and
(5) introducing the expression construct into the cell.

23. (canceled)

24. A method of manufacturing a virus or a phage comprising a T cell receptor (TCR) or B cell receptor (BCR) nucleic acid having a sequence of functional subunit pair genes analyzed in accordance with the method of claim 1, comprising:
(4) providing a virus producing vector or a phage producing vector comprising the nucleic acid sequence of the TCR or the BCR;
(5) introducing the vector into a cell; and
(6) culturing the cell to obtain a virus or a phage comprising a TCR or BCR nucleic acid from culture supernatant.

25. (canceled)

26. A method of manufacturing a T cell receptor (TCR) or B cell receptor (BCR) protein having a sequence of functional subunit pair genes analyzed in accordance with the method of claim 1, comprising:
(4) providing an expression construct comprising the nucleic acid sequence of the TCR or the BCR;
(5) introducing the expression construct into a cell; and
(6) subjecting the cell to a condition under which the TCR or the BCR is expressed.

27.-28. (canceled)
